(19) United States
(12) Patent Application Publication
(10) Pub. No.: US 2025/0084302 A1
(43) Pub. Date: Mar. 13, 2025

(54) ORGANIC COMPOUND AND LIGHT-EMITTING DEVICE

(71) Applicant: SEMICONDUCTOR ENERGY LABORATORY CO., LTD., Atsugi-shi (JP)

(72) Inventors: Miyako MORIKUBO, Ebina (JP); Yuki HAYASHI, Atsugi (JP); Tsunenori SUZUKI, Yokohama (JP); Takashi SHINGU, Atsugi (JP); Sachiko KAWAKAMI, Atsugi (JP); Harue OSAKA, Atsugi (JP); Satoshi SEO, Sagamihara (JP)

(21) Appl. No.: 18/814,801

(22) Filed: Aug. 26, 2024

(30) Foreign Application Priority Data
Aug. 31, 2023 (JP) 2023-141744

(51) Int. Cl. C09K 11/06; C07D 209/86; H10K 50/858

(52) U.S. Cl. CPC C09K 11/06; C07D 209/86; H10K 50/858; C09K 2211/1007; C09K 2211/1011; C09K 2211/1018

(57) ABSTRACT

An organic compound is represented by General Formula (G1). In the formula, each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1). Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3). At least one of R⁵ to R⁸ represents a substituent represented by General Formula (G1-3).

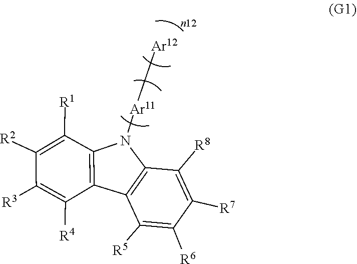

### BACKGROUND OF THE INVENTION

### 1. Field of the Invention

One embodiment of the present invention relates to an organic compound, a light-emitting element, a light-emitting device, a display module, a lighting module, a display apparatus, a light-emitting apparatus, an electronic apparatus, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display apparatus, a liquid crystal display apparatus, a light-emitting apparatus, a lighting device, a power storage device, a memory device, an image capturing device, a driving method thereof, and a manufacturing method thereof.

### 2. Description of the Related Art

Light-emitting devices (organic EL devices) that include organic compounds and utilize electroluminescence (EL) have been put to practical use. In the basic structure of such light-emitting devices, an organic compound layer (EL layer) including a light-emitting material is sandwiched between a pair of electrodes. Carriers are injected by application of a voltage to the device, and recombination energy of the carriers is used to obtain light emission from the light-emitting material.

Such light-emitting devices are of self-luminous type and thus have advantages over liquid crystal displays, such as high visibility and no need for backlight when used in pixels of a display, and are particularly suitable for flat panel displays. Displays that include such light-emitting devices are also highly advantageous in that they can be thin and lightweight. Another feature of such light-emitting devices is that they have an extremely fast response speed.

Since light-emitting layers of such light-emitting devices can be formed two-dimensionally as continuous planar layers, planar light emission can be achieved. This feature is difficult to realize with point light sources typified by incandescent lamps or LEDs or linear light sources typified by fluorescent lamps; thus, such light-emitting devices also have great potential as planar light sources, which can be applied to lighting devices and the like.

Displays or lighting devices including light-emitting devices can be used suitably for a variety of electronic apparatuses as described above, and research and development of light-emitting devices has progressed for more favorable characteristics.

Low outcoupling efficiency is often a problem in an organic EL device. In order to improve the outcoupling efficiency, a structure including a layer formed using a low refractive index material in an EL layer has been suggested (see Patent Document 1, for example).

### REFERENCE

[Patent Document 1] United States Patent Application Publication No. 2020/0176692

### SUMMARY OF THE INVENTION

An object of one embodiment of the present invention is to provide a light-emitting device having high emission efficiency. Another object of one embodiment of the present invention is to provide any of a light-emitting device, a light-emitting apparatus, an electronic apparatus, a display apparatus, and an electronic device each having low power consumption.

It is only necessary that at least one of the above-described objects be achieved in the present invention.

One embodiment of the present invention is an organic compound represented by General Formula (G1).

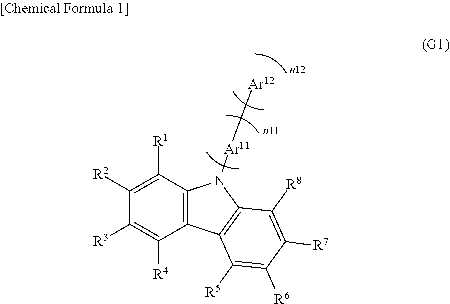

In the above formula, each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1). Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3). At least one of R⁵ to R⁸ represents the substituent represented by General Formula (G1-3). Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms. Ar¹² represents a substituted or unsubstituted monovalent aromatic hydrocarbon group having 6 to 30 carbon atoms or a substituted or unsubstituted monovalent aromatic heterocyclic group having 3 to 30 carbon atoms. In addition, n11 represents an integer greater than or equal to zero and less than or equal to three, and n12 represents an integer greater than or equal to one and less than or equal to three.

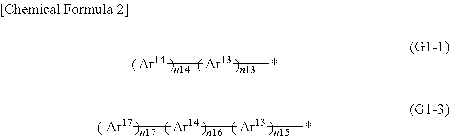

In the above formula, each of Ar¹³, Ar¹⁵, and Ar¹⁶ independently represents a substituted or unsubstituted divalent aromatic hydrocarbon group having 6 to 30 carbon atoms or a substituted or unsubstituted divalent aromatic heterocyclic group having 3 to 30 carbon atoms. Each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms. At least two of Ar⁵, Ar¹⁶ and Ar¹⁷ represent a monovalent or divalent bicyclic or tricyclic aromatic hydrocarbon group or a monovalent or divalent aromatic heterocyclic group. In addition, n13 represents an integer greater than or equal to zero and less than or equal to three, and each of n14, n15, n16, and n17 independently represents an integer greater than or equal to one and less than or equal to three. Furthermore, n15+n16+n17>n13+n14 is satisfied.

In General Formulae (G1), (G1-1), and (G1-3) above, at least two of Ar¹⁵, Ar¹⁶, and Ar¹⁷ represent naphthalene and a 2-position of each of the naphthalenes is bonded to another substituent.

In General Formulae (G1), (G1-1), and (G1-3) above, Ar¹⁶ represents naphthalene and Ar¹⁷ represents naphthalene, and 2,2′-binaphthalene in which 2-positions of the naphthalenes are bonded to each other is included in a partial structure.

One embodiment of the present invention is an organic compound represented by General Formula (G1).

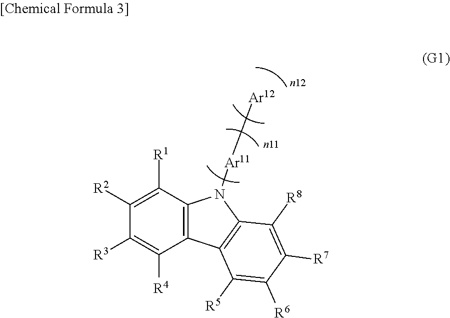

In the above formula, each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1). Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-2). At least one of R⁵ to R⁸ represents the substituent represented by General Formula (G1-2). Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms. Ar¹² represents a substituted or unsubstituted monovalent aromatic hydrocarbon group having 14 to 30 carbon atoms or a substituted or unsubstituted monovalent aromatic heterocyclic group having 3 to 30 carbon atoms. In addition, n11 represents an integer greater than or equal to zero and less than or equal to three, and n12 represents an integer greater than or equal to one and less than or equal to three.

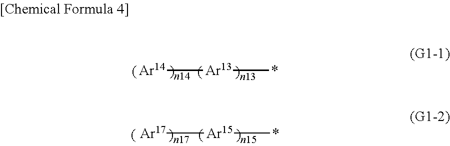

In the above formula, each of Ar¹³ and Ar¹⁵ independently represents a substituted or unsubstituted divalent aromatic hydrocarbon group having 6 to 30 carbon atoms or a substituted or unsubstituted divalent aromatic heterocyclic group having 3 to 30 carbon atoms. Each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms. At least one of Ar¹⁵ and Ar¹⁷ represents phenanthrene. When Ar¹⁷ represents phenanthrene, a 2-position or a 3-position of the phenanthrene is bonded to another group. In addition, n13 represents an integer greater than or equal to zero and less than or equal to three, and each of n14, n15, and n17 independently represents an integer greater than or equal to one and less than or equal to three. Furthermore, n15+n17>n13+n14 is satisfied.

In General Formulae (G1), (G1-1), and (G1-2) above, each of Ar¹¹ to Ar¹⁷ independently represents an aromatic hydrocarbon group.

In General Formulae (G1), (G1-1), and (G1-2) above, an evaporation temperature is lower than or equal to 330° C.

One embodiment of the present invention is an organic compound represented by Structural Formula (100), Structural Formula (101), Structural Formula (102), Structural Formula (123), or Structural Formula (150).

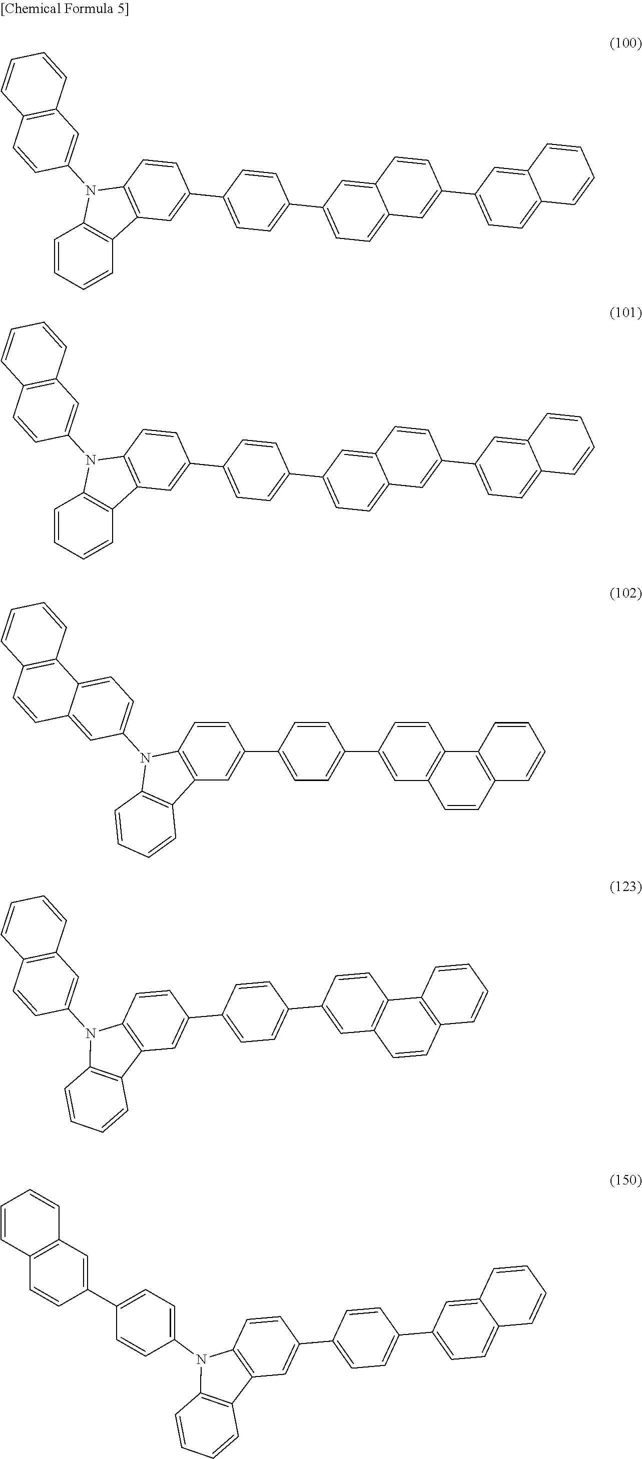 [SMILES: c1ccc2cc(-c3ccc4cc(-c5ccc(-c6ccc7c(c6)c6ccccc6n7-c6ccc7ccccc7c6)cc5)ccc4c3)ccc2c1 ; c1ccc2cc(-c3ccc4cc(-c5ccc(-c6ccc7c(c6)c6ccccc6n7-c6ccc7ccccc7c6)cc5)ccc4c3)ccc2c1 ; c1ccc2c(c1)ccc1cc(-c3ccc(-c4ccc5c(c4)c4ccccc4n5-c4ccc5c(ccc6ccccc65)c4)cc3)ccc12 ; c1ccc2cc(-n3c4ccccc4c4cc(-c5ccc(-c6ccc7c(ccc8ccccc87)c6)cc5)ccc43)ccc2c1 ; c1ccc2cc(-c3ccc(-c4ccc5c(c4)c4ccccc4n5-c4ccc(-c5ccc6ccccc6c5)cc4)cc3)ccc2c1]

One embodiment of the present invention is an electronic device including the organic compound represented by General Formula (G1).

One embodiment of the present invention is a light-emitting device including a cap layer including an organic compound having a carbazole ring and a phenanthrene ring.

One embodiment of the present invention is a light-emitting device including a cap layer including an organic compound having a carbazole ring and two to four naphthalene rings.

In the above light-emitting device, the organic compound used for the cap layer has an ordinary refractive index nₒ at 450 nm of higher than or equal to 1.9 and an ordinary extinction coefficient kₒ at 450 nm of lower than or equal to 0.2.

In the above light-emitting device, the organic compound used for the cap layer has an ordinary refractive index nₒ at 520 nm of higher than or equal to 1.8 and an ordinary extinction coefficient kₒ at 520 nm of lower than or equal to 0.2.

In the above light-emitting device, the organic compound used for the cap layer has an ordinary refractive index nₒ at 630 nm of higher than or equal to 1.75 and an ordinary extinction coefficient kₒ at 630 nm of lower than or equal to 0.2.

One embodiment of the present invention is a light-emitting device including an organic compound represented by General Formula (G1). A difference between an ordinary refractive index nₒ and an extraordinary refractive index nₑ at a wavelength higher than or equal to 360 nm and lower than or equal to 830 nm of the organic compound is greater than or equal to 0.1 and less than or equal to 0.4,

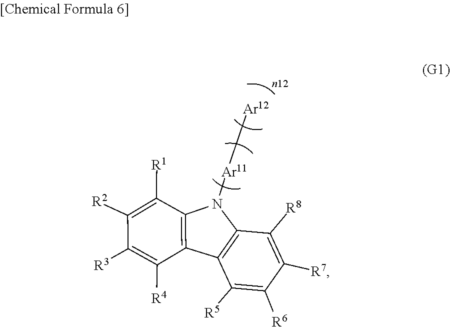

In the above formula, each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1). Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3). At least one of R⁵ to R⁸ represents the substituent represented by General Formula (G1-3). Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms. Ar¹² represents a substituted or unsubstituted monovalent aromatic hydrocarbon group having 6 to 30 carbon atoms or a substituted or unsubstituted monovalent aromatic heterocyclic group having 3 to 30 carbon atoms. In addition, n11 represents an integer greater than or equal to zero and less than or equal to three, and n12 represents an integer greater than or equal to one and less than or equal to three.

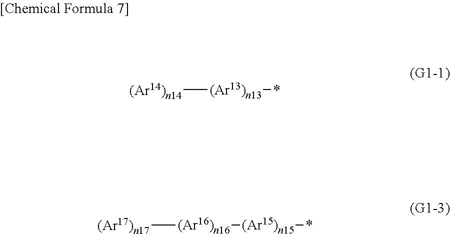

In the above formula, each of Ar¹³, Ar¹⁵, and Ar¹⁶ independently represents a substituted or unsubstituted divalent aromatic hydrocarbon group having 6 to 30 carbon atoms or a substituted or unsubstituted divalent aromatic heterocyclic group having 3 to 30 carbon atoms. Each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms. At least two of Ar¹⁵, Ar¹⁶ and Ar¹⁷ represent a monovalent or divalent bicyclic or tricyclic aromatic hydrocarbon group or a monovalent or divalent aromatic heterocyclic group. Each of n13, n15, and n16 independently represents an integer greater than or equal to zero and less than or equal to three, and each of n14 and n17 independently represents an integer greater than or equal to one and less than or equal to three. Furthermore, n15+n16+n17≥n13+n14 is satisfied.

One embodiment of the present invention is a light-emitting device in which the organic compound represented by General Formula (G1) above has an evaporation temperature of lower than or equal to 330° C.

One embodiment of the present invention is a light-emitting device including an organic compound represented by Structural Formula (100), Structural Formula (101), Structural Formula (102), Structural Formula (123), or Structural Formula (150).

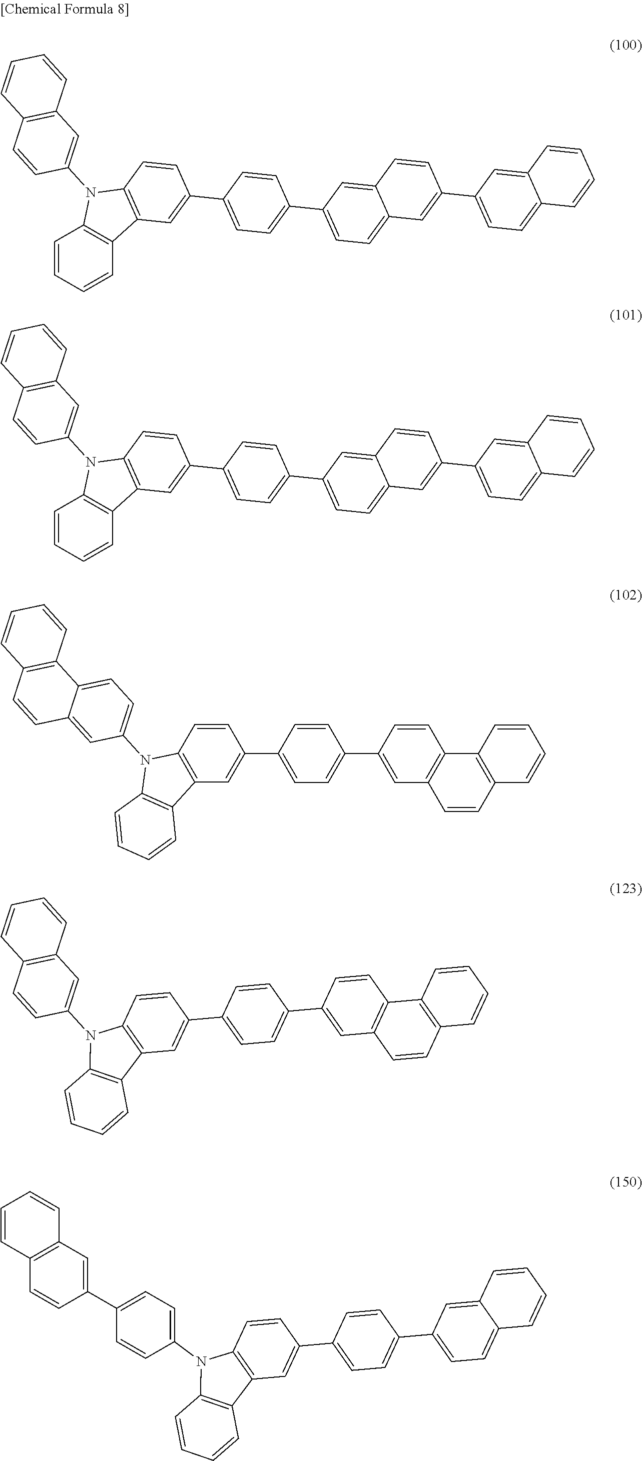 [SMILES: c1ccc2cc(-c3ccc4cc(-c5ccc(-c6ccc7c(c6)c6ccccc6n7-c6ccc7ccccc7c6)cc5)ccc4c3)ccc2c1 ; c1ccc2cc(-c3ccc4cc(-c5ccc(-c6ccc7c(c6)c6ccccc6n7-c6ccc7ccccc7c6)cc5)ccc4c3)ccc2c1 ; c1ccc2c(c1)ccc1cc(-c3ccc(-c4ccc5c(c4)c4ccccc4n5-c4ccc5c(ccc6ccccc65)c4)cc3)ccc12 ; c1ccc2cc(-n3c4ccccc4c4cc(-c5ccc(-c6ccc7c(ccc8ccccc87)c6)cc5)ccc43)ccc2c1 ; c1ccc2cc(-c3ccc(-c4ccc5c(c4)c4ccccc4n5-c4ccc(-c5ccc6ccccc6c5)cc4)cc3)ccc2c1]

Another embodiment of the present invention is an electronic apparatus including any of the above-described light-emitting devices, a sensor, an operation button, and a speaker or a microphone.

Another embodiment of the present invention is a light-emitting apparatus including any of the above-described light-emitting devices, and a transistor or a substrate.

Another embodiment of the present invention is a display apparatus including any of the above-described light-emitting devices, and a transistor or a substrate.

Another embodiment of the present invention is a lighting device including any of the above-described light-emitting devices and a housing.

Note that the light-emitting apparatus in this specification includes, in its category, an image display device that uses a light-emitting device. The light-emitting apparatus may also include a module in which a light-emitting device is provided with a connector such as an anisotropic conductive film or a tape carrier package (TCP), a module in which a printed wiring board is provided at the end of a TCP, and a module in which an integrated circuit (IC) is directly mounted on a light-emitting device by a chip on glass (COG) method. Furthermore, a lighting device or the like may include the light-emitting apparatus.

An electronic device in this specification includes, in its category, a light-emitting device, a light-receiving device (e.g., a sensor or a solar cell), a device in which a light-emitting device and a light-receiving device are combined, and the like.

The “light-emitting device”, “electronic device”, or “organic device” in this specification also includes a layer or structure provided outside a pair of electrodes. For example, a partition, a sealing film, a color filter, or the like provided over an electrode is sometimes included.

With one embodiment of the present invention, a light-emitting device having high emission efficiency can be provided. With one embodiment of the present invention, any of a light-emitting device, a light-emitting apparatus, an electronic apparatus, a display apparatus, and an electronic device each having low power consumption can be provided.

Note that the description of these effects does not preclude the existence of other effects. One embodiment of the present invention does not necessarily have all of these effects. Other effects will be apparent from and can be derived from the description of the specification, the drawings, the claims, and the like.

### BRIEF DESCRIPTION OF THE DRAWINGS

In the accompanying drawings:

FIGS. 1A to 1E illustrate structures of light-emitting devices according to an embodiment;

FIGS. 2A and 2B are a top view and a cross-sectional view of a light-emitting apparatus;

FIGS. 3A to 3D are cross-sectional views each illustrating a light-emitting device;

FIGS. 4A to 4E are cross-sectional views illustrating an example of a method for manufacturing a light-emitting apparatus;

FIGS. 5A to 5E are cross-sectional views illustrating the example of the method for manufacturing the light-emitting apparatus;

FIGS. 6A to 6C are cross-sectional views illustrating the example of the method for manufacturing the light-emitting apparatus;

FIGS. 7A to 7C are cross-sectional views illustrating the example of the method for manufacturing the light-emitting apparatus;

FIGS. 8A to 8C are cross-sectional views illustrating the example of the method for manufacturing the light-emitting apparatus;

FIGS. 9A to 9C are cross-sectional views illustrating the example of the method for manufacturing the light-emitting apparatus;

FIGS. 10A to 10C are cross-sectional views illustrating the example of the method for manufacturing the light-emitting apparatus;

FIGS. 11A to 11G are top views each illustrating a structure example of a pixel;

FIGS. 12A to 12I are top views each illustrating a structure example of a pixel;

FIGS. 13A and 13B are perspective views illustrating a structure example of a display module;

FIGS. 14A and 14B are cross-sectional views each illustrating a structure example of a light-emitting apparatus;

FIG. 15 is a perspective view illustrating a structure example of a light-emitting apparatus;

FIG. 16A is a cross-sectional view illustrating a structure example of a light-emitting apparatus, and FIGS. 16B and 16C are cross-sectional views each illustrating a structure example of a transistor;

FIG. 17 is a cross-sectional view illustrating a structure example of a light-emitting apparatus;

FIGS. 18A to 18D are cross-sectional views illustrating structure examples of a light-emitting apparatus;

FIGS. 19A to 19D illustrate examples of electronic apparatuses;

FIGS. 20A to 20F illustrate examples of electronic apparatuses;

FIGS. 21A to 21G illustrate examples of electronic apparatuses;

FIG. 22 shows a ¹H-NMR spectrum of an organic compound;

FIG. 23 shows the absorption and emission spectra of the organic compound in a toluene solution;

FIG. 24 shows the absorption and emission spectra of the organic compound in a solid thin film state;

FIG. 25 shows a ¹H-NMR spectrum of an organic compound;

FIG. 26 shows the absorption and emission spectra of the organic compound in a toluene solution;

FIG. 27 shows the absorption and emission spectra of the organic compound in a solid thin film state;

FIG. 28 shows a ¹H-NMR spectrum of an organic compound;

FIG. 29 shows the absorption and emission spectra of the organic compound in a toluene solution;

FIG. 30 shows the absorption and emission spectra of the organic compound in a solid thin film state;

FIG. 31 shows measurement results of the refractive indices and the extinction coefficients of an organic compound;

FIG. 32 shows measurement results of the refractive indices and the extinction coefficients of an organic compound;

FIG. 33 shows measurement results of the refractive indices and the extinction coefficients of an organic compound;

FIG. 34 shows measurement results of the refractive indices and the extinction coefficients of an organic compound;

FIG. 35 shows measurement results of the refractive indices and the extinction coefficients of an organic compound;

FIG. 36 illustrates a structure of a sample;

FIG. 37 is a graph showing luminance-voltage characteristics of samples;

FIG. 38 is a graph showing current density-voltage characteristics of the samples;

FIG. 39 is a graph showing current efficiency-luminance characteristics of the samples;

FIG. 40 is a graph showing power efficiency-luminance characteristics of the samples;

FIG. 41 is a graph showing BI-luminance characteristics of the samples;

FIG. 42 is a graph showing electroluminescence spectra of the samples;

FIG. 43 is a graph showing time dependence of normalized luminance of the samples;

FIG. 44 is a graph showing luminance-voltage characteristics of a sample;

FIG. 45 is a graph showing current density-voltage characteristics of the sample;

FIG. 46 is a graph showing current efficiency-luminance characteristics of the sample;

FIG. 47 is a graph showing power efficiency-luminance characteristics of the sample;

FIG. 48 is a graph showing BI-luminance characteristics of the sample;

FIG. 49 is a graph showing an electroluminescence spectrum of the sample;

FIG. 50 is a graph showing time dependence of normalized luminance of the sample;

FIG. 51 is a graph showing luminance-voltage characteristics of samples;

FIG. 52 is a graph showing current density-voltage characteristics of the samples;

FIG. 53 is a graph showing current efficiency-luminance characteristics of the samples;

FIG. 54 is a graph showing power efficiency-luminance characteristics of the samples;

FIG. 55 is a graph showing BI-luminance characteristics of the samples;

FIG. 56 is a graph showing electroluminescence spectra of the samples;

FIG. 57 shows a ¹H-NMR spectrum of an organic compound;

FIG. 58 shows the absorption and emission spectra of the organic compound in a toluene solution;

FIG. 59 shows the absorption and emission spectra of the organic compound in a solid thin film state;

FIG. 60 shows a ¹H-NMR spectrum of an organic compound;

FIG. 61 shows the absorption and emission spectra of the organic compound in a toluene solution; and

FIG. 62 shows the absorption and emission spectra of the organic compound in a solid thin film state.

### DETAILED DESCRIPTION OF THE INVENTION

Embodiments of the present invention will be described in detail below with reference to the drawings. Note that the embodiments of the present invention are not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Note that in the case where light is incident on a material having optical anisotropy, light with a plane of vibration parallel to the optical axis is referred to as extraordinary light (rays) and light with a plane of vibration perpendicular to the optical axis is referred to as ordinary light (rays); the refractive index of the material with respect to ordinary light might differ from that with respect to extraordinary light. In such a case, the ordinary refractive index and the extraordinary refractive index can be separately calculated by anisotropy analysis. Note that in the case where the measured material has both the ordinary refractive index and the extraordinary refractive index, the ordinary refractive index is used as an index in this specification. Furthermore, when simply mentioning a refractive index, the refractive index refers to the average value of the ordinary refractive index and the extraordinary refractive index.

As is the case with the refractive index, the extinction coefficient with respect to ordinary light might differ from that with respect to extraordinary light, and the ordinary extinction coefficient and the extraordinary extinction coefficient can be separately calculated by anisotropy analysis. In the case where the measured material has both the ordinary extinction coefficient and the extraordinary extinction coefficient, the ordinary extinction coefficient is used as an index in this specification. Furthermore, when simply mentioning an extinction coefficient, the extinction coefficient refers to the average value of the ordinary extinction coefficient and the extraordinary extinction coefficient.

Furthermore, an evaporated film in this specification refers to a film deposited by an evaporation method in the state where a substrate is at room temperature or is not heated.

### Embodiment 1

In this embodiment, an organic compound of one embodiment of the present invention will be described.

The organic compound described in this embodiment is specifically a compound having a carbazole ring (also referred to as a carbazole compound), particularly an organic compound having a carbazole ring and a phenanthrene ring, and further particularly an organic compound having a carbazole ring and two to four naphthalene rings.

The organic compound of one embodiment of the present invention has a high glass transition temperature, a high refractive index, and a low sublimation temperature (or a low evaporation temperature) and is unlikely to be thermally decomposed at the time of sublimation by having one to three phenanthrene rings or two to four naphthalene rings as a substituent on the carbazole ring. In terms of the sublimation temperature, the number of naphthalene rings is further preferably two or three.

An organic compound with a low evaporation temperature is helpful when being used in an industrial product because the organic compound can be deposited at a low temperature and thus is less thermally affected during the deposition and decomposition due to heat can be reduced.

In particular, in the mass production process, the same material is heated continuously for a long time; an organic compound having an excessively high evaporation temperature is easily decomposed by the heating. When the material is decomposed, the degree of vacuum in an evaporation atmosphere is degraded, the evaporation temperature is further increased, the deposited film has absorption in the visible region, and the emission efficiency or reliability of the formed element is decreased, for example, whereby a stable mass production system cannot be established. Thus, the organic compound material that can be deposited at a low temperature can be deposited without decomposition of the material, resulting in stable mass production.

Specifically, the evaporation temperature of the organic compound of one embodiment of the present invention in an evaporation apparatus is preferably lower than or equal to 350° C., further preferably lower than or equal to 330° C., still further preferably lower than or equal to 300° C., yet still further preferably lower than or equal to 270° C. Here, the evaporation temperature is defined as the temperature of an evaporation source at which the evaporation rate is 1×10⁻¹ nm/s under a pressure of the evaporation apparatus being greater than or equal to 1.0×10⁻⁶ Pa and less than 1.0×10⁻⁴ Pa. Note that the evaporation rate being 1×10⁻¹ nm/s indicates 1×10⁻¹-nm-thick deposition per second, and a film can be deposited faster at a higher evaporation rate. A reduction in takt time is required for a reduction in production cost in the mass production process; thus, it is preferred that high-speed deposition be possible.

The sublimation temperature of the organic compound of one embodiment of the present invention is preferably lower than or equal to 350° C., further preferably lower than or equal to 300° C., still further preferably lower than or equal to 290° C., yet still further preferably lower than or equal to 260° C. Note that the sublimation temperature can be evaluated by measurement with a thermogravimetry/differential thermal analysis (TG-DTA) apparatus. For example, in the case where the pressure of the TG-DTA apparatus is controlled to be higher than or equal to 1.0×10⁻¹ Pa and lower than or equal to 10 Pa, the measured weight of a compound used for the measurement is greater than or equal to 1 mg and less than or equal to 20 mg, and the temperature at which the weight obtained by thermogravimetry of the compound is reduced by 5% from the weight at the start of the measurement (the temperature is referred to as 5% weight loss temperature) is defined as the sublimation temperature.

It is also preferable that the temperature at which the weight becomes 50% of the weight at the start of the measurement (referred to as 50% weight loss temperature) be low in the measurement with the TG-DTA apparatus. The 50% weight loss temperature being low means that even when the sublimation weight increases, sublimation can be performed stably at a low temperature. The 50% weight loss temperature of the organic compound of one embodiment of the present invention is preferably lower than or equal to 390° C., further preferably lower than or equal to 350° C., still further preferably lower than or equal to 320° C., yet still further preferably lower than or equal to 300° C.

In order to lower the evaporation temperature, an organic compound with a small molecular weight is generally selected; however, in that case, the glass transition temperature (Tg) tends to be low and the heat resistance of the bulk or the film tends to be low. Heating at a temperature higher than or equal to Tg might change the film quality and the refractive index (the ordinary refractive index and the extraordinary refractive index); thus, a high Tg is desired. The organic compound of one embodiment of the present invention has a high glass transition temperature and a low evaporation temperature.

A deposited film of the organic compound of one embodiment of the present invention can have a high refractive index. First, the organic compound having a carbazole skeleton has a higher refractive index and larger refractive index anisotropy than the organic compound not having the skeleton.

When a condensed ring is included in the molecule, the refractive index can be further increased. For example, a phenanthrene ring or a naphthalene ring is preferably included. These rings are preferably bonded to the carbazole ring as a substituent, in which case the molecular weight can be low, sublimation at a low temperature is possible, and high heat resistance (high glass transition temperature) and an effect of increasing the refractive index can be obtained. When one phenanthrene ring or naphthalene ring is included in the molecule, the compound can have a high refractive index, and when two or more phenanthrene or naphthalene rings are included, the refractive index can be further increased. For example, a binaphthalene structure in which two naphthalene rings are directly connected to each other is suitable. In the case where the substituent has a binaphthalene structure, the glass transition temperature T_{g} of the compound can be increased. Furthermore, when a structure in which the 2-position of naphthalene is bonded to another group (i.e., a 2-naphthyl group) is included, the refractive index can be further increased. This is because the density of the organic compound in the deposited film is increased. In particular, when a 2,2′-binaphthalene structure in which the 2-positions of naphthalenes are bonded to each other is included in a partial structure, the refractive index can be further increased. However, without limitation to this, T_{g} can be improved when a structure in which the 1-position of naphthalene is bonded to another group (i.e., a 1-naphthyl group) is included, for example.

An increase in the number of condensed rings may increase the evaporation temperature; thus, the number of naphthalene rings is preferably less than or equal to four, further preferably less than or equal to three.

When the compound has benzene as a substituent, the compound preferably has paraphenylene (also referred to as p-phenylene). A compound having paraphenylene can have higher molecular orientation when deposited and thus have higher ordinary refractive index than a compound having metaphenylene (or orthophenylene). However, metaphenylene or orthophenylene can also be used, in which case effects of decreasing the sublimation temperature and inhibiting crystallization of a film can be expected.

As for the value of the refractive index, the ordinary refractive index nₒ at a wavelength of 450 nm, which is in a blue wavelength range, is preferably higher than or equal to 1.9, for example. Moreover, the ordinary extinction coefficient kₒ at a wavelength of 450 nm is preferably lower than or equal to 0.2, further preferably lower than or equal to 1×10⁻², still further preferably lower than or equal to 1×10⁻⁴. The organic compound of one embodiment of the present invention is an organic compound in which the difference between the ordinary refractive index (nₒ) and the extraordinary refractive index (nₑ) (Δn=|nₒ−nₑ|) at any of the wavelengths higher than or equal to 360 nm and lower than or equal to 830 nm (e.g., three wavelengths of 450 nm (a blue wavelength range), 520 nm (a green wavelength range), and 630 nm (a red wavelength range)), preferably at all of the three wavelengths is greater than or equal to 0.1 and less than or equal to 0.4. The details of the refractive index value are described in Embodiment 2 and Examples.

Here, the difference between the ordinary refractive index (nₒ) and the extraordinary refractive index (nₑ) (Δn=|nₒ−nₑ|) can be used as an indicator of anisotropy in the refractive index. In the visible light range (higher than or equal to 360 nm and lower than or equal to 830 nm), the refractive index anisotropy tends to be larger with a larger Δn.

For example, it is useful to use an organic compound with large refractive index anisotropy for any component in a light-emitting device in order to extract light from a light-emitting layer with high efficiency and increase light extraction efficiency.

The carbazole compound of one embodiment of the present invention may have a heterocycle as a substituent. Examples of heteroatoms included in the heterocycle include nitrogen, oxygen, and sulfur. When the heteroatoms are included, the ordinary refractive index nₒ can be increased or the glass transition temperature T_{g} can be increased in some cases. In order to improve the refractive index, the heterocycle preferably has a five-membered heteroaromatic ring skeleton including nitrogen, oxygen, and sulfur and preferably has a pyrrole skeleton, a furan skeleton, a thiophene skeleton, an azole (e.g., imidazole, oxazole, thiazole, oxadiazole, or triazole) skeleton, or the like, for example. In particular, a compound having a molecular structure including an atom with a large atomic radius, like a sulfur atom, is expected to have a high refractive index (e.g., Structural Formulae (210), (226), and (227) shown later). The condensed ring preferably has a structure including heteroatoms in order that the condensed ring can have an effect of increasing T_{g} and the heteroatoms can have an effect of increasing the refractive index at the same time (e.g., Structural Formulae (209), (210), and (222) to (229) shown later). That is, a five-membered condensed heteroaromatic ring including nitrogen, oxygen, and sulfur is preferable; examples include a carbazole ring, a dibenzofuran ring, a benzonaphthofuran ring, a dibenzothiophene ring, a benzonaphthothiophene ring, a benzoxazole ring, and a benzothiazole ring. However, when heteroatoms are included, the solubility in an organic solvent might be lowered depending on the kind of heterocycle, which might result in a decrease in yield of a reaction in synthesis or a decrease in purity in purification. In addition, having a heterocycle in a molecular structure probably increases intermolecular interaction, which leads to an increase in sublimation temperature. Thus, the total number of heteroatoms included in the compound other than the carbazole ring is preferably less than or equal to three, further preferably less than or equal to two, still further preferably less than or equal to one. Alternatively, the total number of heterocycles included in the compound other than the carbazole ring is preferably less than or equal to three, further preferably less than or equal to two, still further preferably less than or equal to one.

### Organic Compound Example 1

One embodiment of the present invention is an organic compound represented by General Formula (G1).

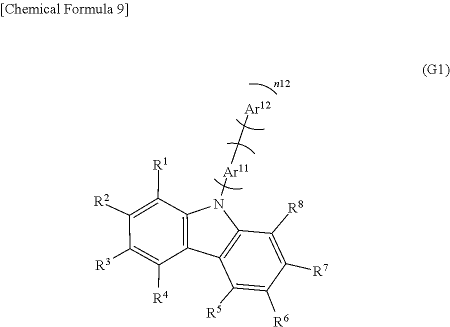

In General Formula (G1), each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1) below. Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3) below. At least one of R⁵ to R⁸ represents the substituent represented by General Formula (G1-3) below. Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms. Ar¹² represents a substituted or unsubstituted monovalent aromatic hydrocarbon group having 6 to 30 carbon atoms or a substituted or unsubstituted monovalent aromatic heterocyclic group having 3 to 30 carbon atoms. In addition, n11 represents an integer greater than or equal to zero and less than or equal to three and n12 represents an integer greater than or equal to one and less than or equal to three.

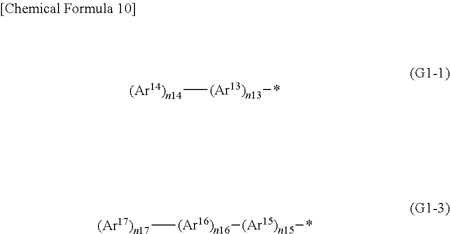

In General Formulae (G1-1) and (G1-3), each of Ar¹³, Ar¹⁵, and Ar¹⁶ independently represents a substituted or unsubstituted divalent aromatic hydrocarbon group having 6 to 30 carbon atoms (also referred to as an arylene group) or a substituted or unsubstituted divalent aromatic heterocyclic group having 3 to 30 carbon atoms (also referred to as a heteroarylene group). Each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms (also referred to as an aryl group). At least two of Ar¹⁵, Ar¹⁶, and Ar¹⁷ represent a monovalent or divalent bicyclic or tricyclic aromatic hydrocarbon group or a monovalent or divalent aromatic heterocyclic group (also referred to as a heteroaryl group). In addition, n13 represents an integer greater than or equal to zero and less than or equal to three and each of n14, n15, n16, and n17 independently represents an integer greater than or equal to one and less than or equal to three. Furthermore, n15+n16+n17>n13+n14 is satisfied.

Note that when n11 is greater than or equal to two, a plurality of Ar¹¹s may be the same or different substituents. The same applies to Ar¹² to Ar¹⁷. Each of Ar¹¹ to Ar¹⁷ preferably represents a substituent including carbon and hydrogen (including deuterium). Note that each of Ar¹², Ar¹⁴, and Ar¹⁷ may represent hydrogen (deuterium).

In General Formula (G1) above, the group(s) other than the substituent(s) represented by General Formula (G1-1) among R¹ to R⁴ or the group(s) other than the substituent(s) represented by General Formula (G1-3) among R⁵ to R⁸ preferably represent hydrogen, in which case the intermolecular interaction is increased and a higher refractive index is achieved owing to the more rigid molecular structure compared with the case where the group(s) other than the substituent(s) represented by General Formula (G1-1) among R¹ to R⁴ or the group(s) other than the substituent(s) represented by General Formula (G1-3) among R⁵ to R⁸ represent the substituents other than hydrogen. Furthermore, the synthesis cost can also be reduced, which is preferable.

In General Formula (G1), a ring including R¹ and R², a ring including R² and R³, a ring including R³ and R⁴, a ring including R⁴ and R⁵, a ring including R⁵ and R⁶, a ring including R⁶ and R⁷, and a ring including R⁷ and R⁸ may be formed. For example, in the case where a benzene ring including R² and R³ is formed, the benzene ring and the carbazole ring are condensed to form a structure including a benzocarbazole ring. Such a condensed structure can improve heat resistance and the glass transition temperature (T_{g}). By contrast, in the case where such a condensed structure is not included, the molecular weight is lowered and the sublimation temperature can be decreased.

Here, when General Formula (G1) above includes a naphthalene ring or a naphthalene skeleton, the ordinary refractive index can be increased. Note that in this specification, unless otherwise specified, a compound having a naphthalene ring refers to a compound having a naphthalene ring itself like Structure (100) described later, and does not refer to a compound having naphthalene as part of a ring like Structure (102) described later. A compound having a naphthalene skeleton refers to a compound having naphthalene in part of its skeleton like Structure (102) as well. A similar interpretation applies to rings or skeletons other than naphthalene.

When the 2-positions of the naphthalenes are bonded to each other, the ordinary refractive index can be further increased. No bonding at the 1-position of the naphthalene skeleton can increase the refractive index. When the condensed ring having three or less rings is used, the sublimation temperature can be low. Note that a bicyclic condensed ring refers to the one having two rings, such as naphthalene, quinoxaline, or benzoxazole, and a tricyclic condensed ring refers to the one having three rings, such as phenanthrene, anthracene, and carbazole. The same applies to condensed rings having four or more rings.

For example, at least two of Ar¹⁵, Ar¹⁶, and Ar¹⁷ preferably represent a substituent having a substituted or unsubstituted naphthalene ring. In particular, Ar¹⁶ and Ar¹⁷ preferably represent a substituent having a naphthalene ring. Furthermore, in the case where Ar¹⁶ and Ar¹⁷ represent naphthalene, the 2-positions of the naphthalenes are preferably bonded to each other. In other words, in the case where Ar¹⁶ and Ar¹⁷ represent naphthalene, 2,2′-binaphthalene in which the 2-positions of the naphthalenes are bonded to each other is preferably included in a partial structure (e.g., Structural Formula (100) below).

Furthermore, at least the 2-position of the naphthalene is preferably bonded to another group. Specifically, in the case where Ar¹⁵ represents naphthalene, at least the 2-position and the 6-position of the naphthalene are preferably bonded to the carbazolyl group and Ar¹⁶. Furthermore, in the case where Ar¹⁶ represents naphthalene, at least the 2-position and the 6-position of the naphthalene are preferably bonded to Ar¹⁵ and Ar¹⁷. Furthermore, in the case where Ar¹⁷ represents naphthalene, the 2-position of the naphthalene is preferably bonded to Ar¹⁶.

In particular, in the case where Ar¹⁶ and Ar¹⁷ are bonded to the 6-positions of Ar⁵ and Ar¹⁶, a material with a high ordinary refractive index can be provided. When this material is used for a device, the device can have high efficiency and low power consumption. A material with high heat resistance can be provided. When this material is used for a device, the device can have high efficiency and high heat resistance.

In the above-described organic compound example 1, n15+n16+n17>n13+n14 is satisfied. In this manner, it is preferable that the number of substituents bonded to one of the two benzene rings of the carbazole ring be larger than the number of substituents bonded to the other, in which case the refractive index anisotropy tends to be large. In the above-described organic compound example 1, it is preferable that the number of coupled arylene groups or heteroarylene groups that the substituent bonded to one of the two benzene rings of the carbazole ring has (the number of coupled Ar¹⁵ to Ar¹⁷ in the above-described organic compound example 1) be larger than the number of coupled arylene groups or heteroarylene groups that the substituent bonded to the other has (the number of coupled Ar¹³ and Ar¹⁴ in the above-described organic compound example 1), in which case the refractive index anisotropy tends to be large and the intermolecular interaction can be reduced and thus the sublimation temperature can be decreased. Furthermore, in the above-described example 1, n15+n16+n17>n11+n12 is preferable, in which case the refractive index anisotropy tends to be large. In addition, such a relation of n can increase T_{g}.

### Organic Compound Example 2

One embodiment of the present invention is an organic compound represented by General Formula (G1).

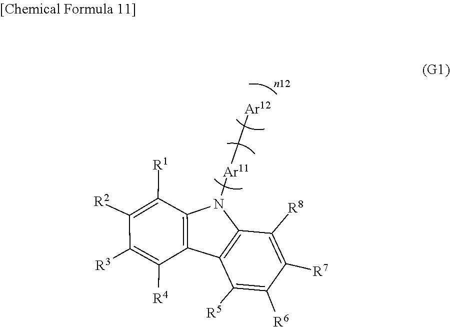

In General Formula (G1), each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1). Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-2). At least one of R⁵ to R⁸ represents the substituent represented by General Formula (G1-2). Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms. Ar¹² represents a substituted or unsubstituted monovalent aromatic hydrocarbon group having 14 to 30 carbon atoms or a substituted or unsubstituted monovalent aromatic heterocyclic group having 3 to 30 carbon atoms. In addition, n11 represents an integer greater than or equal to zero and less than or equal to three and n12 represents an integer greater than or equal to one and less than or equal to three.

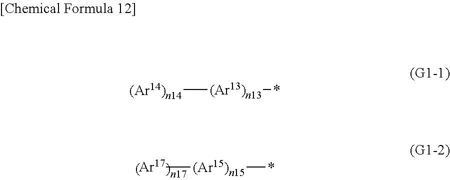

In General Formulae (G1-1) and (G1-2), each of Ar¹³ and Ar¹⁵ independently represents a substituted or unsubstituted divalent aromatic hydrocarbon group having 6 to 30 carbon atoms or a substituted or unsubstituted divalent aromatic heterocyclic group having 3 to 30 carbon atoms. Each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms. At least one of Ar¹⁵ and Ar¹⁷ represents phenanthrene. When Ar¹⁷ represents phenanthrene, a 2-position or a 3-position of the phenanthrene is bonded to another group. In addition, n13 represents an integer greater than or equal to zero and less than or equal to three and each of n14, n15, and n17 independently represents an integer greater than or equal to one and less than or equal to three. Furthermore, n15+n17>n13+n14 is satisfied.

Each of Ar¹² and Ar¹⁷ may be a condensed ring having two or more rings, preferably three or more rings.

Furthermore, at least one of Ar¹⁵ and Ar¹⁷ is preferably a condensed ring having two or more rings, preferably three or more rings, further preferably four or more rings. Examples of a condensed ring having four or more rings include a triphenylene ring. The use of a triphenylene ring is preferable because the refractive index is increased and T_{g} can be increased. Note that in order to lower the sublimation temperature, the number of rings is preferably small, and n15 may be zero.

In General Formulae (G1-1) and (G1-2), at least one of Ar¹¹ and Ar¹² represents a condensed ring, and at least one of Ar¹⁵ and Ar¹⁷ represents a condensed ring. Each of n11, n12, n15, and n17 independently represents an integer greater than or equal to one and less than or equal to three.

Note that when n15 is greater than or equal to two, a plurality of Ar¹⁵s may be the same or different substituents. The same applies to Ar¹⁷. Each of Ar¹⁵ and Ar¹⁷ preferably represents a substituent including carbon and hydrogen (including deuterium). Note that each of Ar¹², Ar¹⁴, and Ar¹⁷ may represent hydrogen (deuterium).

In General Formula (G1-2), Ar¹⁵ or Ar¹⁷ represents phenanthrene. When the 2-position or the 3-position of the phenanthrene is bonded to another group, the ordinary refractive index can be increased. Particularly when Ar¹⁷ represents phenanthrene, bonding of the 2-position or the 3-position of the phenanthrene to another group can further increase the ordinary refractive index (e.g., Structural Formula (103) below).

In the above-described organic compound example 2, n15+n17>n13+n14 is satisfied. In this manner, it is preferable that the number of substituents bonded to one of the two benzene rings of the carbazole ring be larger than the number of substituents bonded to the other, in which case the refractive index anisotropy tends to be large. In the above-described organic compound example 2, it is preferable that the number of coupled arylene groups or heteroarylene groups that the substituent bonded to one of the two benzene rings of the carbazole ring has (the number of coupled Ar¹⁵ and Ar¹⁷ in the above-described organic compound example 2) be larger than the number of coupled arylene groups or heteroarylene groups that the substituent bonded to the other has (the number of coupled Ar¹³ and Ar¹⁴ in the above-described organic compound example 2), in which case the refractive index anisotropy tends to be large and the intermolecular interaction can be reduced and thus the sublimation temperature can be decreased. Furthermore, in the above-described example 2, n15+n17>n11+n12 is preferable, in which case the refractive index anisotropy tends to be large. In addition, such a relation of n can increase T_{g}.

### Substituents for Each Organic Compound Example

For R¹ to R⁸, Ar¹¹ to Ar¹⁵, and Ar¹⁷ in General Formulae (G1), (G1-1), (G1-2), and (G1-3) described in <Organic compound example 1> and <Organic compound example 2>, the descriptions of the substituents represented by R^{m} (m is an arbitrary number) or Ar^{m} (m is an arbitrary number) described in <Organic compound example 1> and <Organic compound example 2> can be referred to, and vice versa.

As the alkyl group having 1 to 6 carbon atoms represented by R¹ to R⁸ in General Formula (G1), a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a 3-methylpentyl group, a 2-methylpentyl group, a 2-ethylbutyl group, a 1,2-dimethylbutyl group, a 2,3-dimethylbutyl group, or the like can be used, for example. In the case where the alkyl group having 1 to 6 carbon atoms has a substituent, the substituent can be a cycloalkyl group having 1 to 5 carbon atoms or an aryl group having 6 to 13 carbon atoms.

As the cycloalkyl group having 3 to 6 carbon atoms represented by R¹ to R⁸ in General Formula (G1), a cyclopropyl group, a cyclobutyl group, a cyclopentyl group, a cyclohexyl group, a 1-methylcyclohexyl group, a cycloheptyl group, an adamantyl group, a bicyclo[2,2,2]octyl group, a norbornanyl group, or the like can be used, for example. In the case where the cycloalkyl group having 3 to 6 carbon atoms has a substituent, the substituent can be a cycloalkyl group having 1 to 4 carbon atoms or an aryl group having 6 to 13 carbon atoms.

As the aromatic hydrocarbon group having 6 to 30 carbon atoms or the aromatic heterocyclic group having 3 to 30 carbon atoms represented by R¹ to R⁸ and Ar¹¹ to Ar¹⁷ in any of General Formulae (G1) and (G1-1) to (G1-3), a group having a structure that is obtained by removing a hydrogen atom at the bonding position from the aromatic hydrocarbon group or aromatic heterocycle represented by any of Structural Formulae (Ar-1) to (Ar-33) below can be used.

For example, a naphthyl group refers to a monovalent substituent that is obtained by removing one hydrogen atom from naphthalene (represented by Structural Formula (Ar-17) below). A naphthalenyl group refers to a divalent substituent that is obtained by removing two hydrogen atoms from naphthalene. The same applies to the others of Structural Formulae (Ar-1) to (Ar-33) and the like. Therefore, for example, in the case where Ar¹⁵ represents pyridine (represented by Structural Formula (Ar-5) below), Ar¹⁶ represents naphthalene, and Ar¹⁷ represents naphthalene in General Formula (G1-3) above, it is expressed that “Ar¹⁵ represents a pyridinyl group, Ar¹⁶ represents a naphthalenyl group, and Ar¹⁷ represents a naphthyl group” in a strict sense; however, it can also be expressed that “Ar¹⁵ represents pyridine and Ar¹⁶ and Ar¹⁷ represent naphthalene”. Furthermore, Structural Formula (100) and Structural Formula (101) can also be expressed as “a compound including a plurality of naphthalene rings”, “a compound including a plurality of naphthalenes”, or the like. The description of valence such as monovalent or divalent is omitted in some cases.

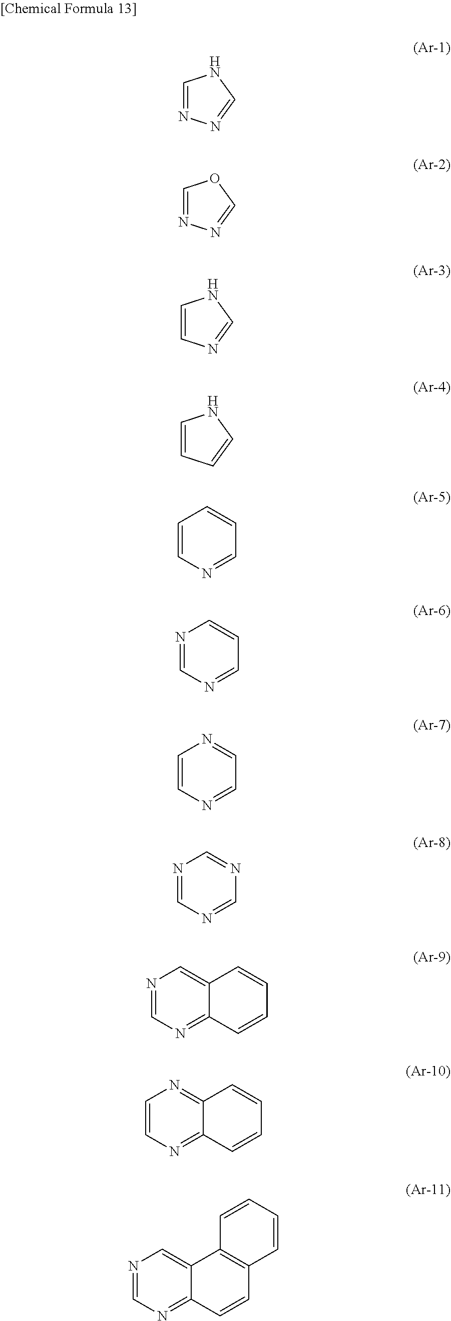

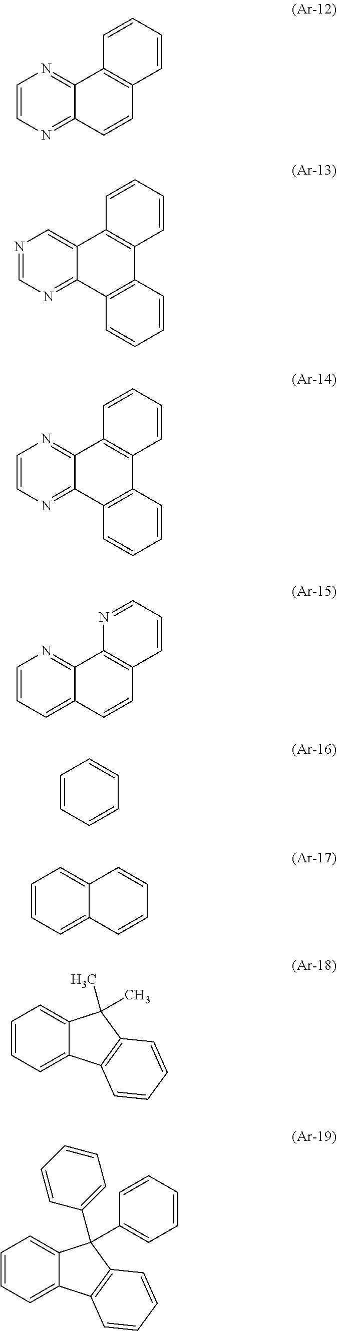

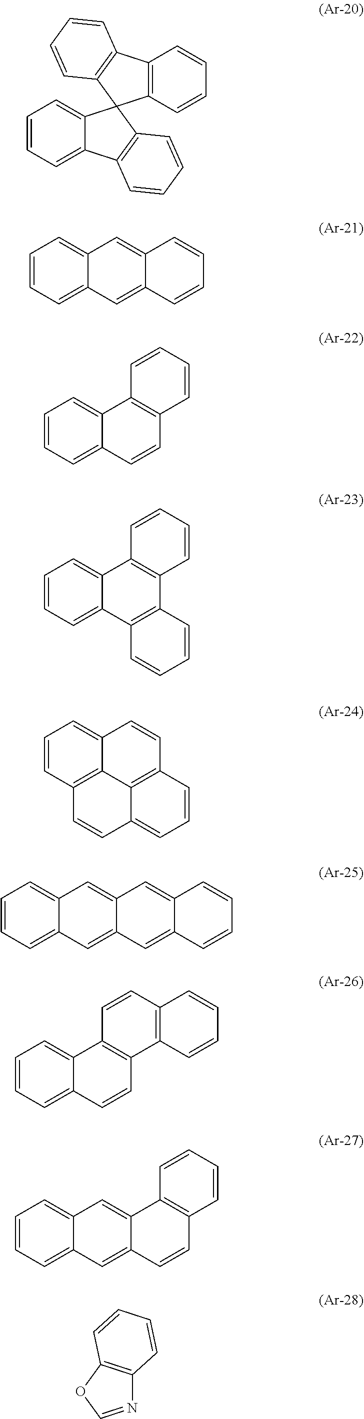

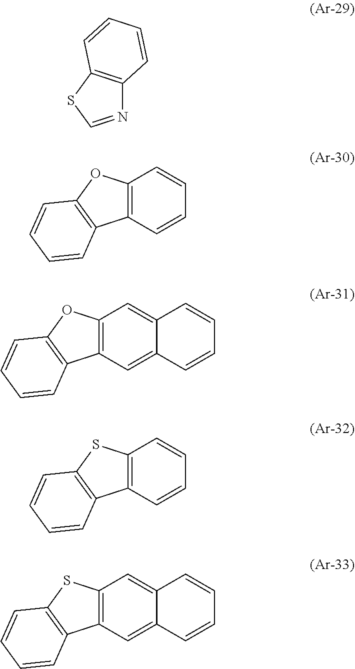

In the case where the aromatic hydrocarbon group or the aromatic heterocyclic group has a substituent, examples of the substituent include a cyano group, a halogen group, an alkyl group having 1 to 4 carbon atoms, a cycloalkyl group having 3 to 6 carbon atoms, an aryl group having 6 to 13 carbon atoms, and a heteroaryl group having 3 to 10 carbon atoms. An effect of reducing the driving voltage of the device can be expected by having a cyano group or a halogen group. Furthermore, with an alkyl group or a cycloalkyl group, an effect of decreasing the sublimation temperature can be expected; thus, such a compound can be used in a variety of layer such as a cap layer. Furthermore, with an alkyl group or a cycloalkyl group, the ordinary refractive index is sometimes lowered; thus, such a compound is particularly suitably used for a layer that is required to have a low ordinary refractive index, such as a transport layer (a hole-transport layer or an electron-transport layer). Note that similar effects can be expected when a cyano group, a halogen group, an alkyl group, or a cycloalkyl group is used as R¹ to R⁸.

### SPECIFIC EXAMPLES

The following are specific examples of the organic compound of one embodiment of the present invention having the structure represented by General Formula (G1) above.

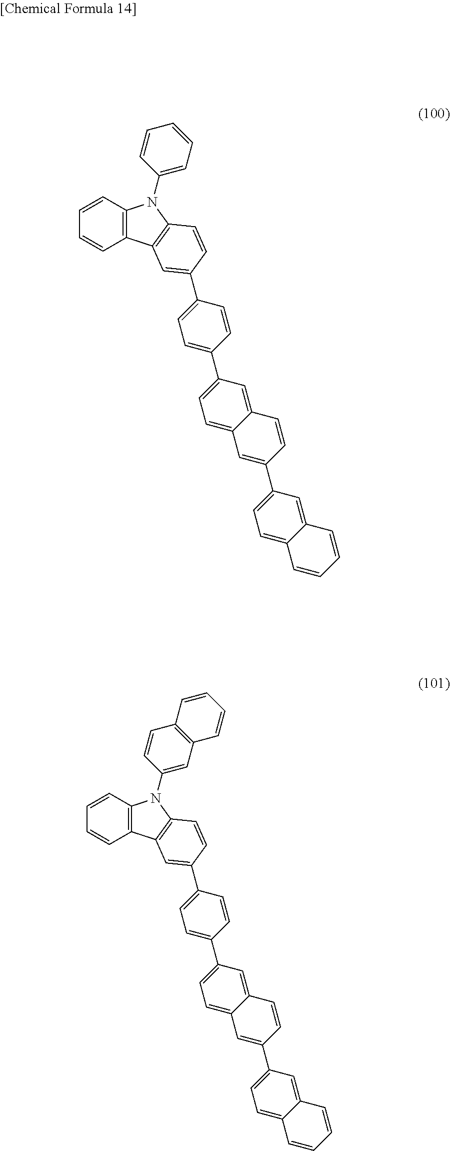

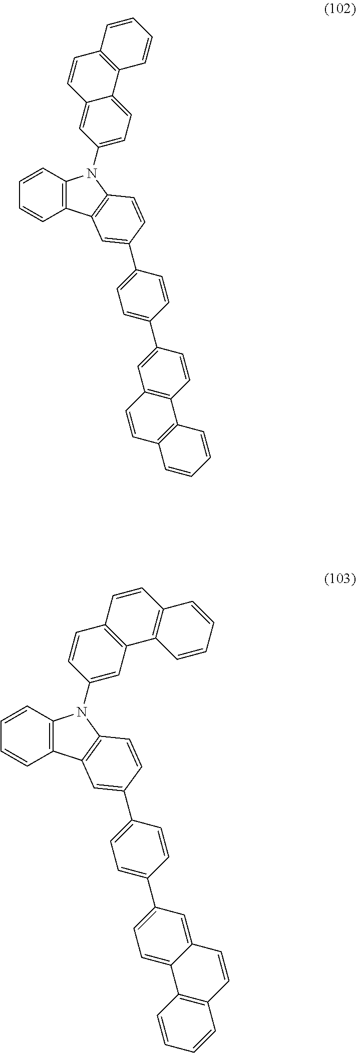

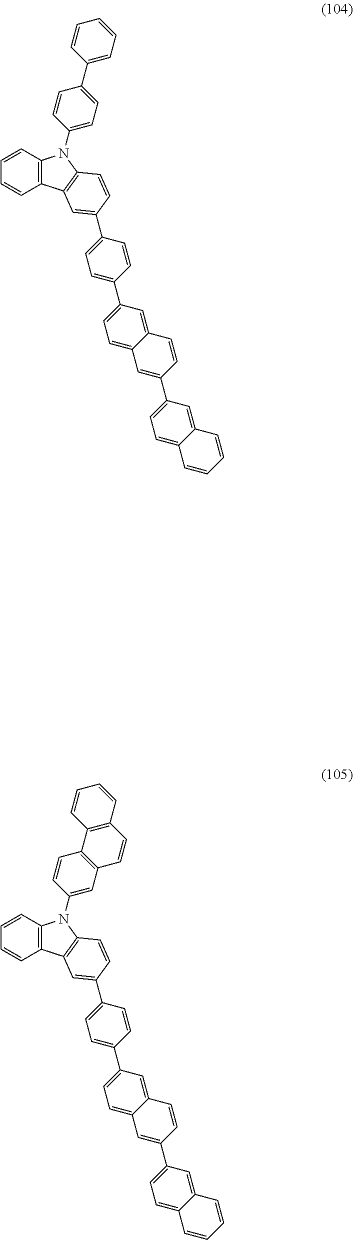

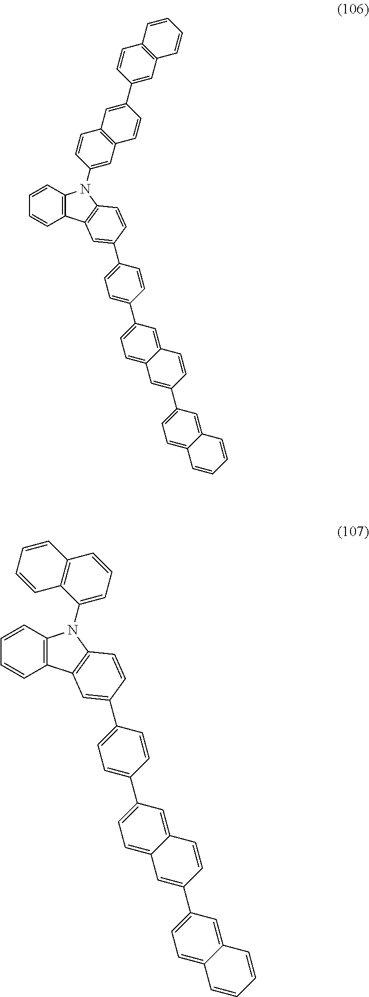

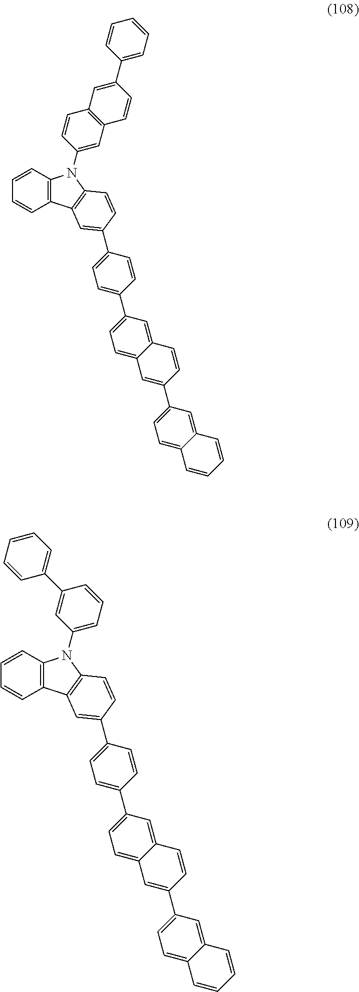

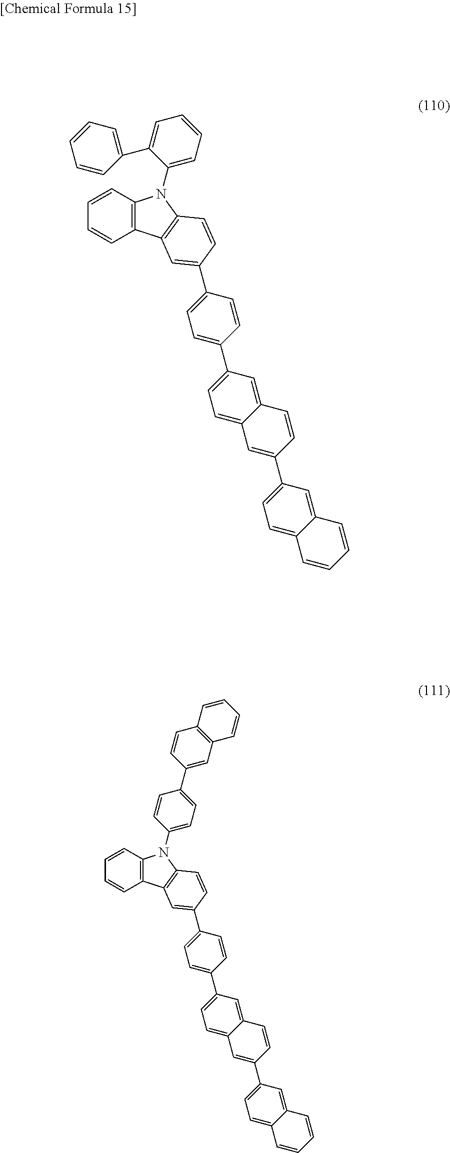

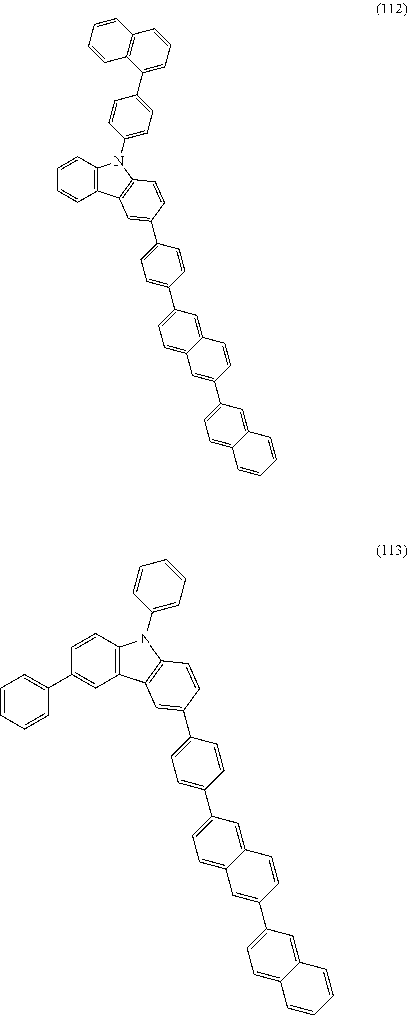

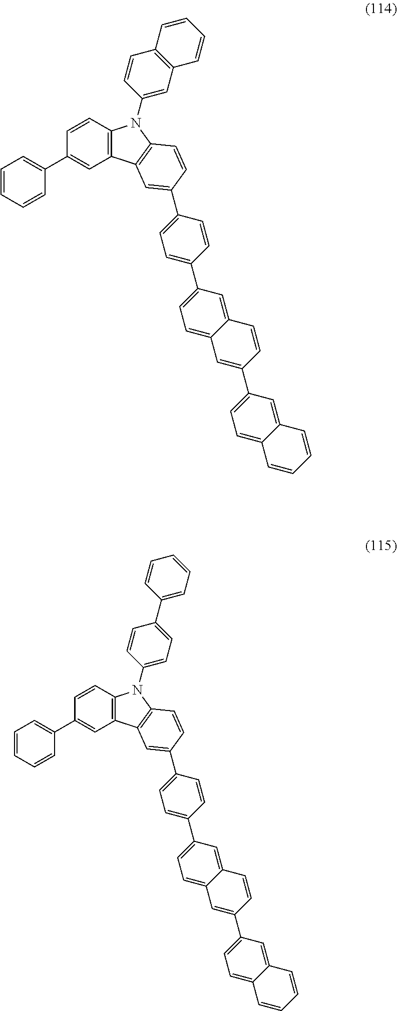

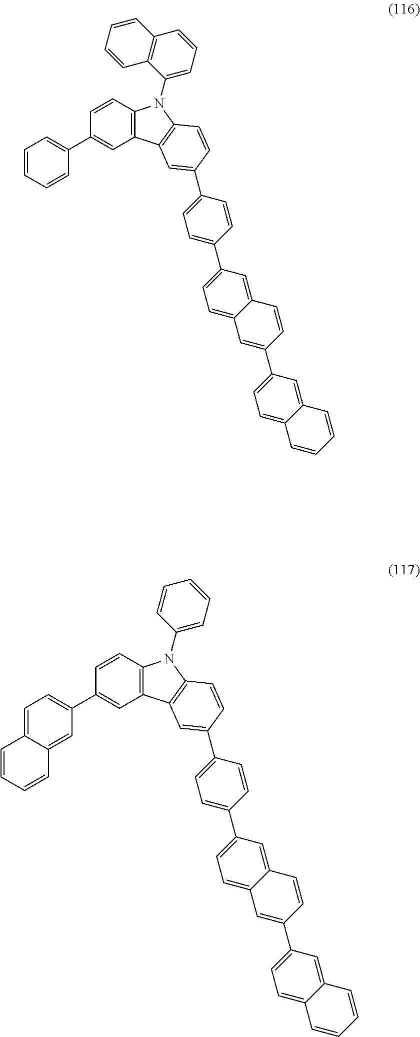

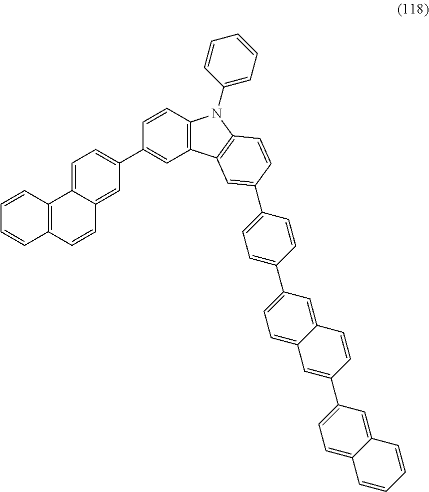

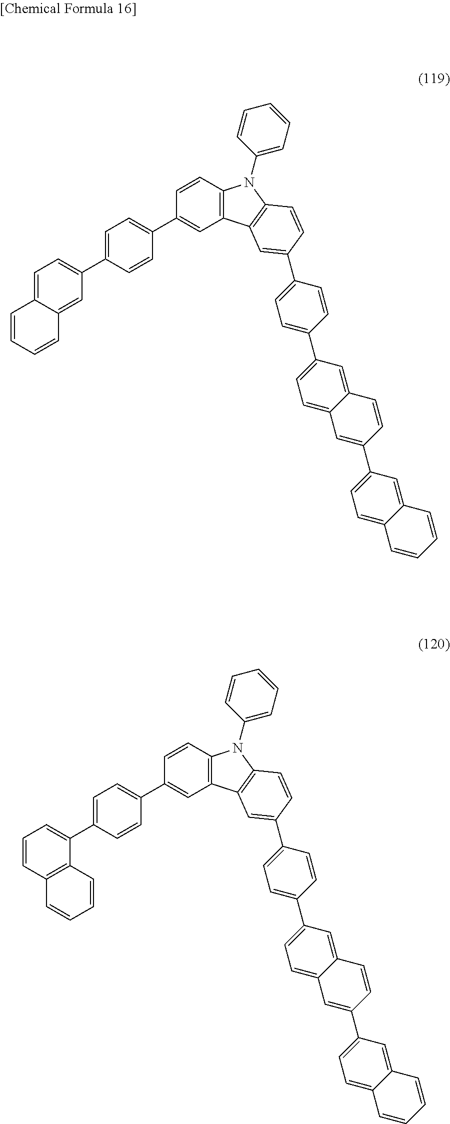

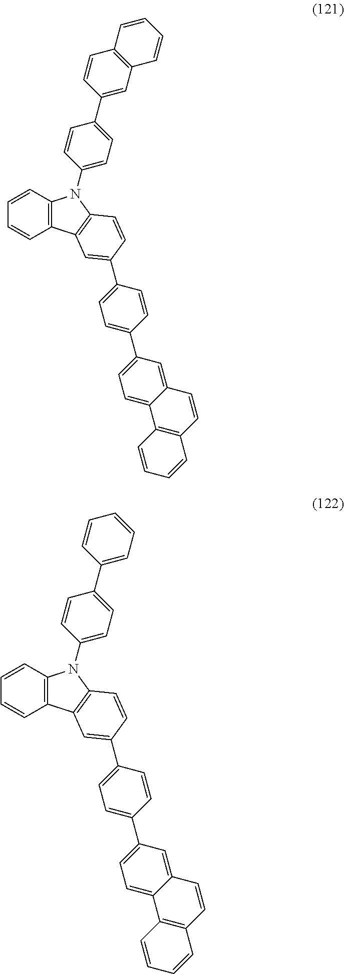

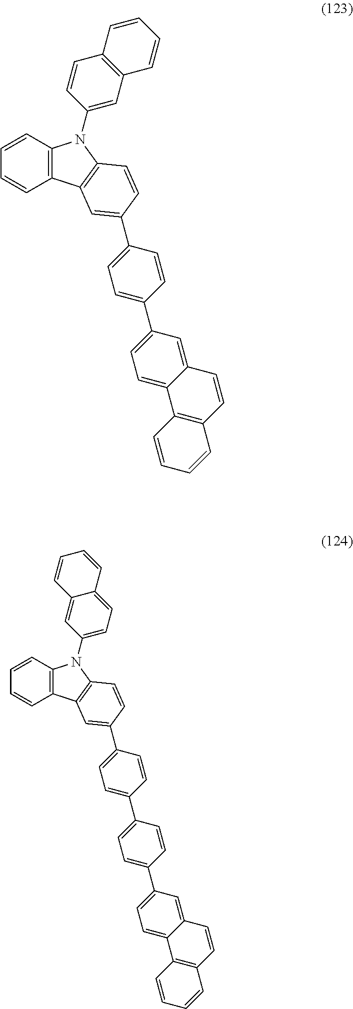

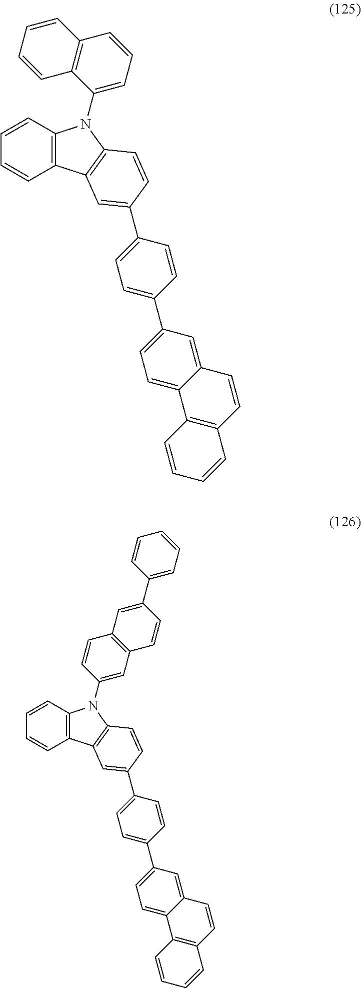

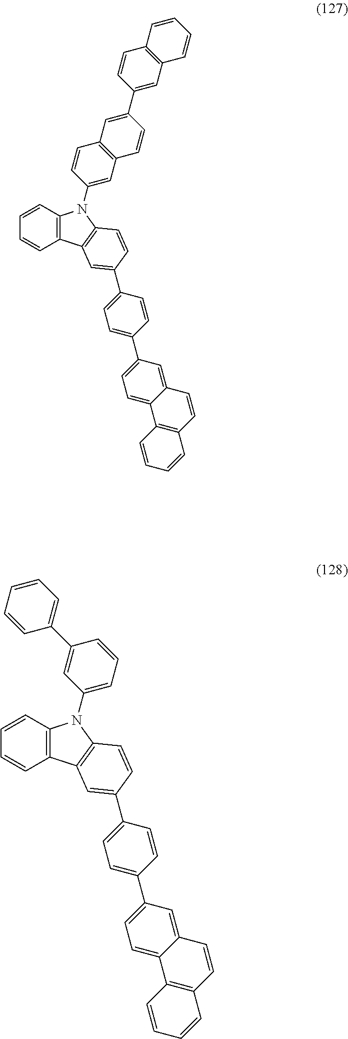

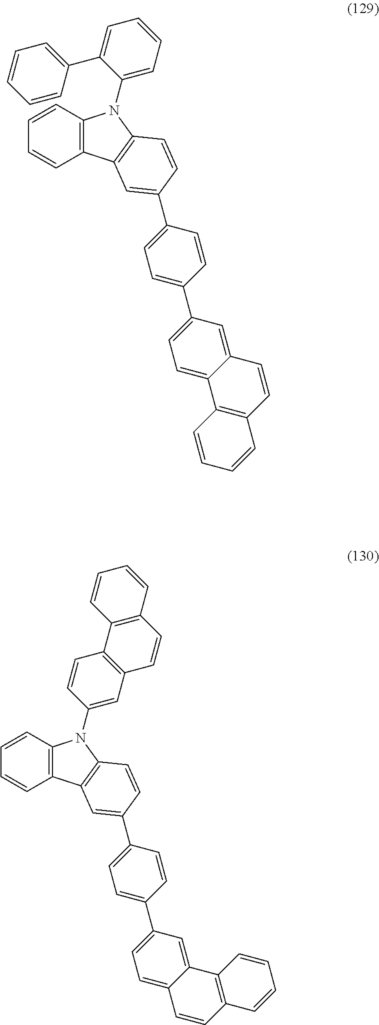

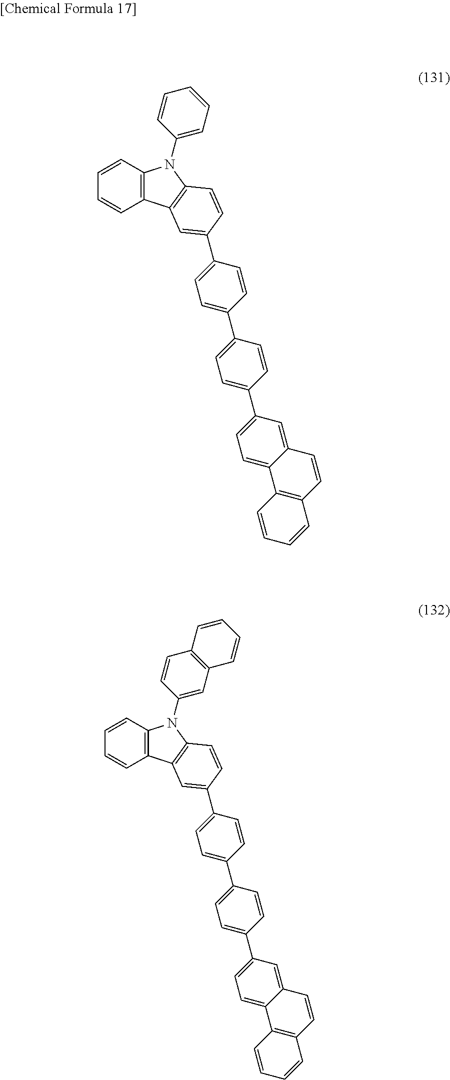

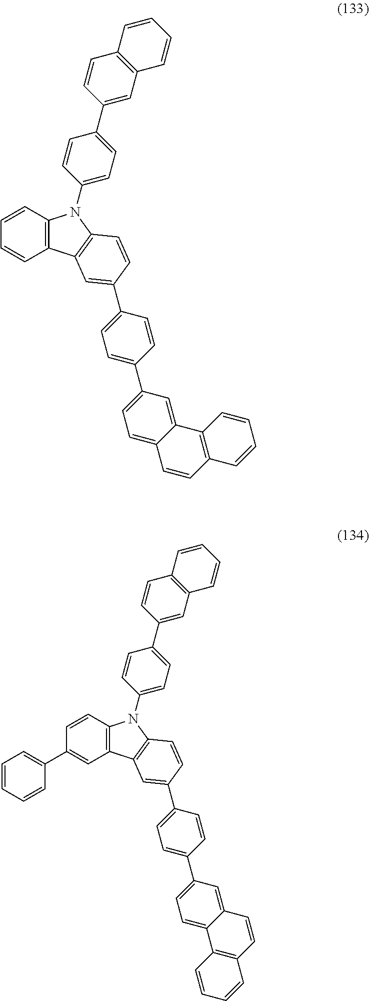

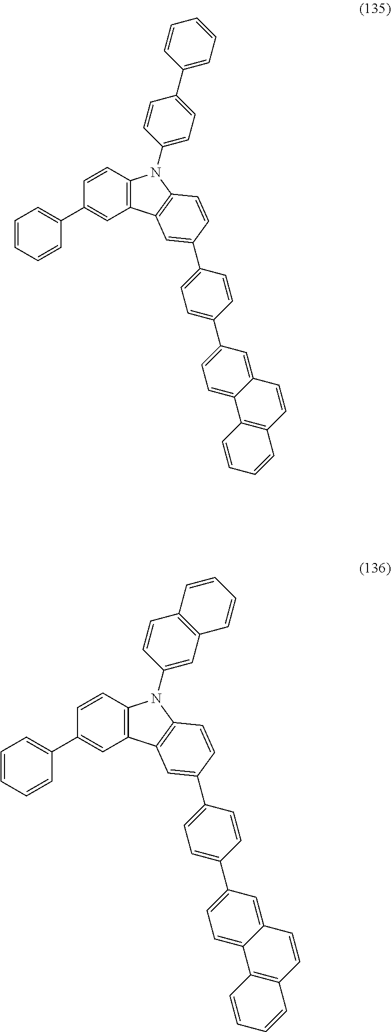

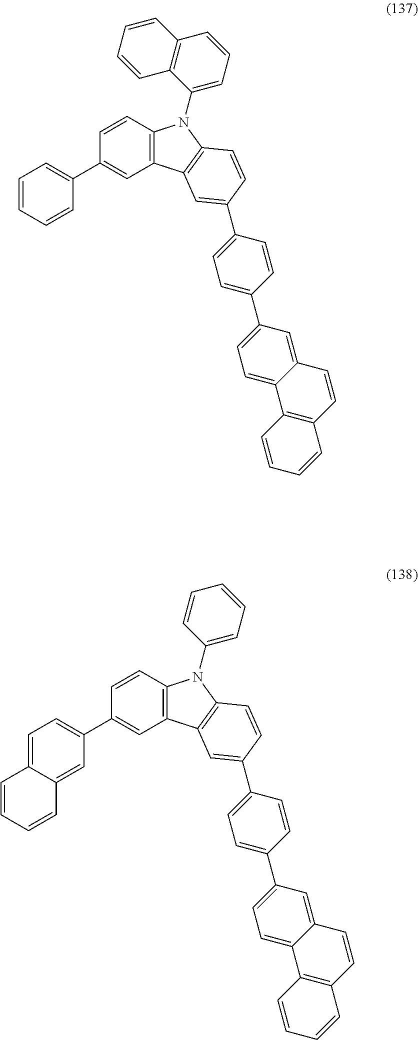

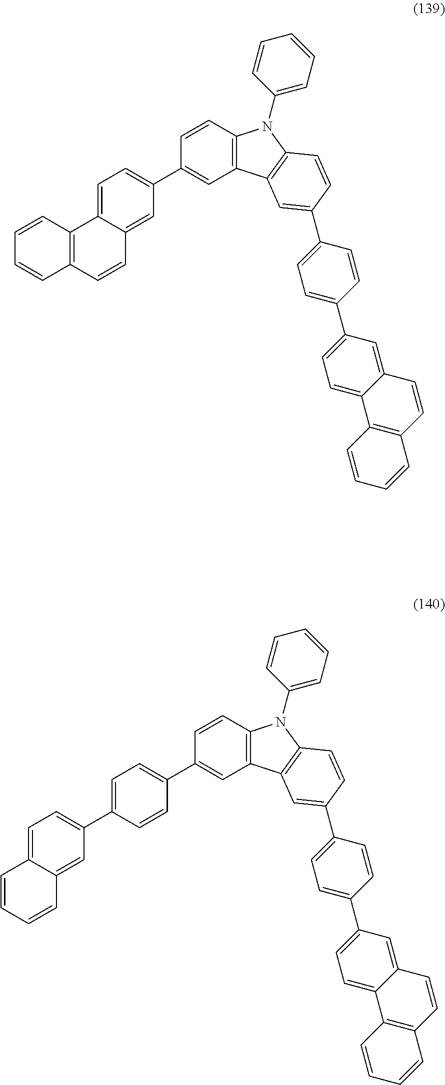

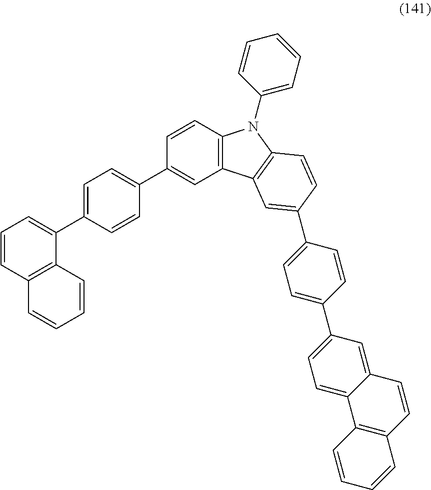

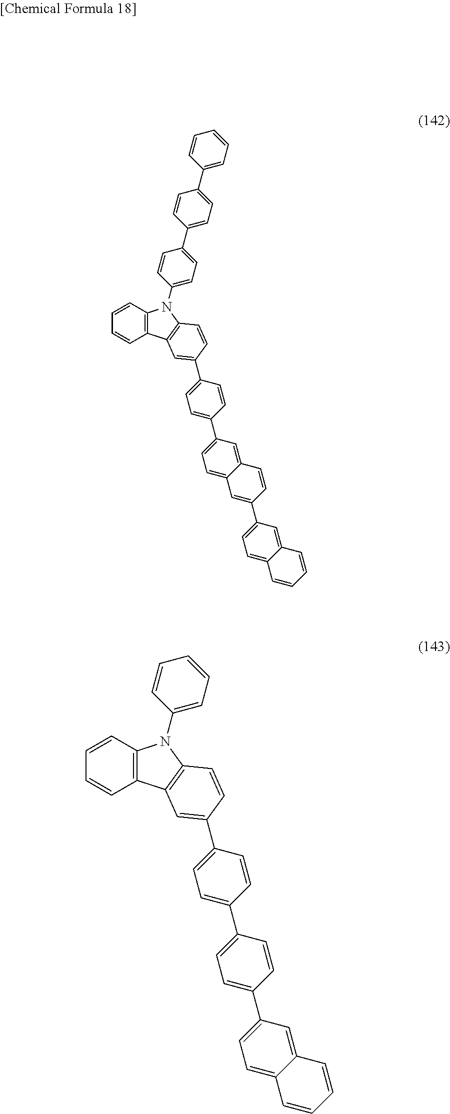

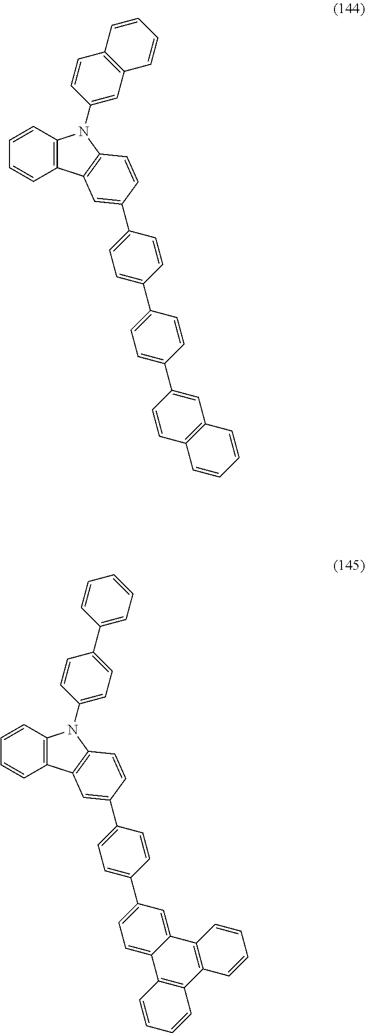

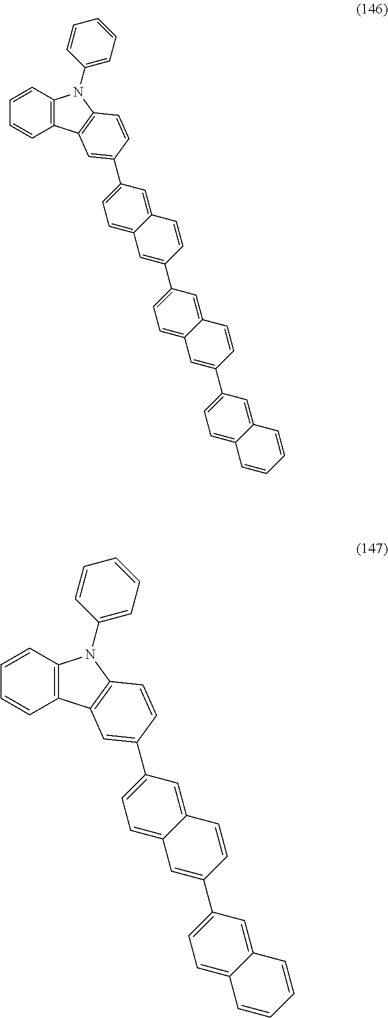

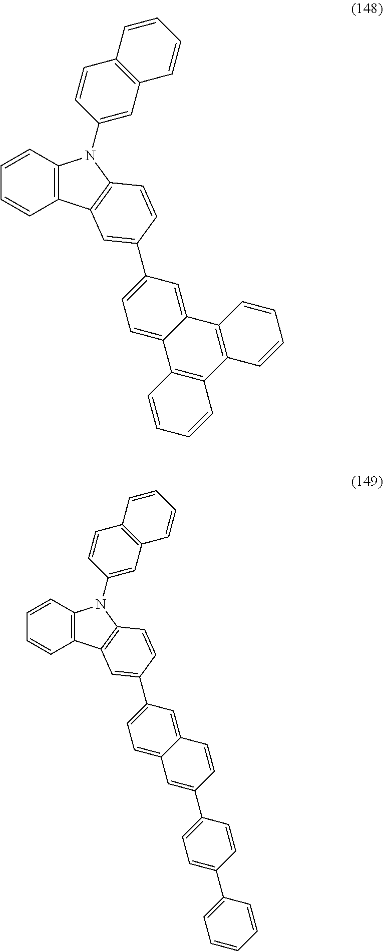

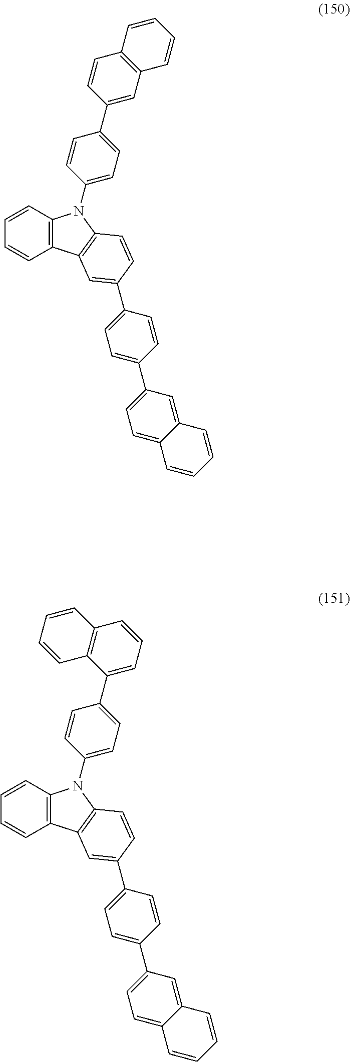

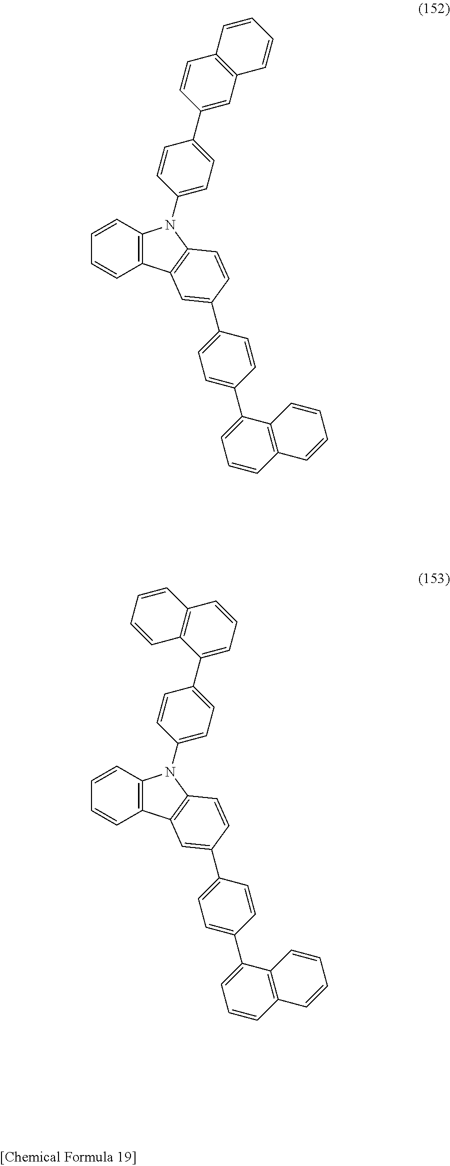

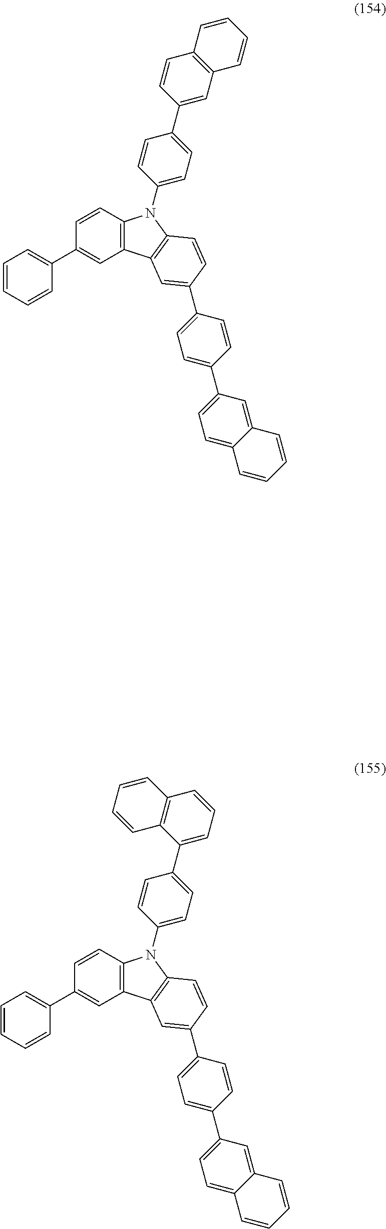

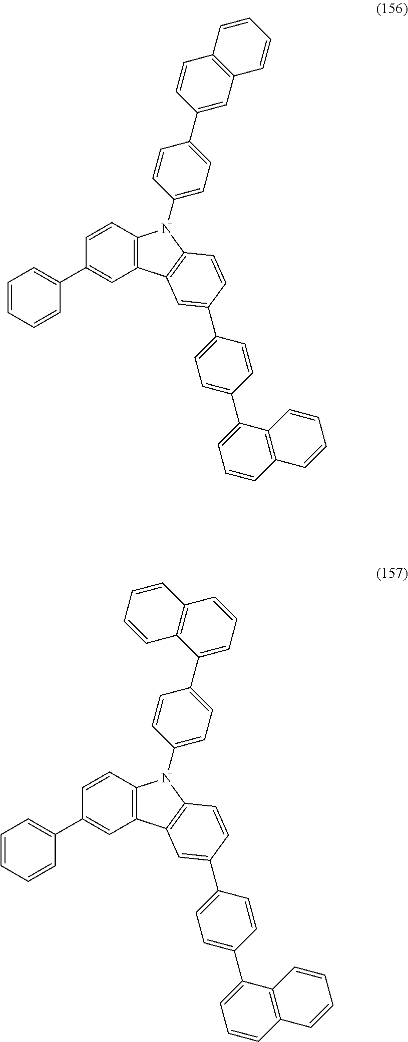

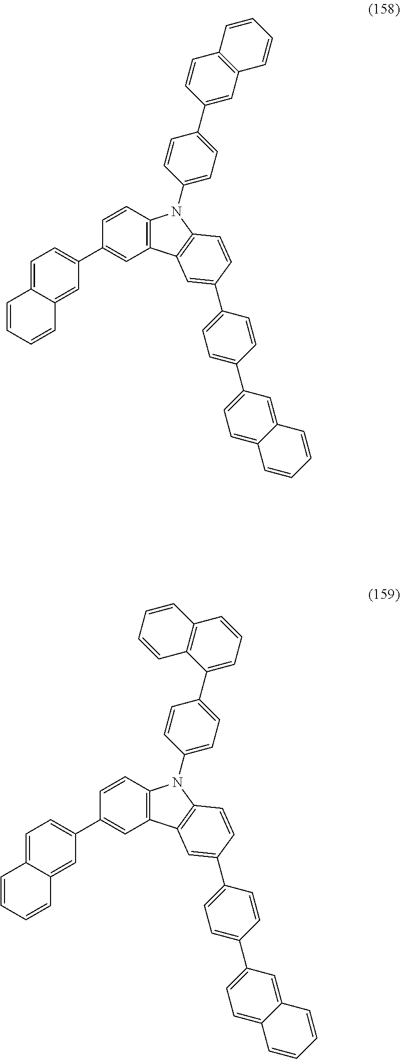

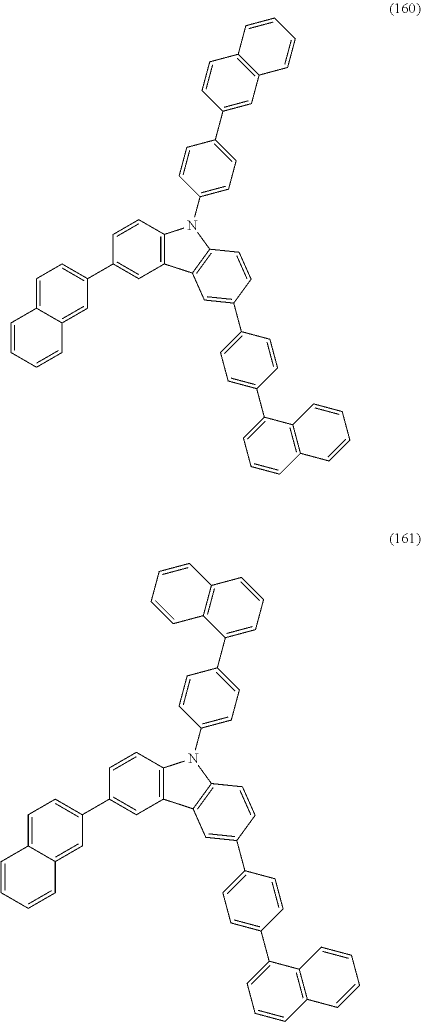

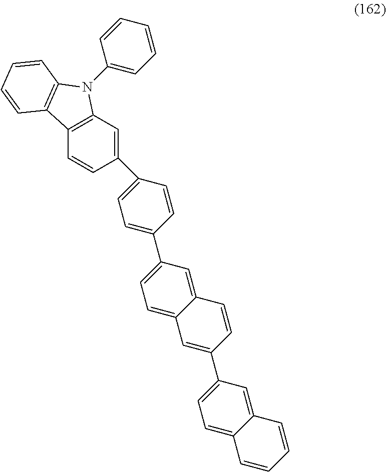

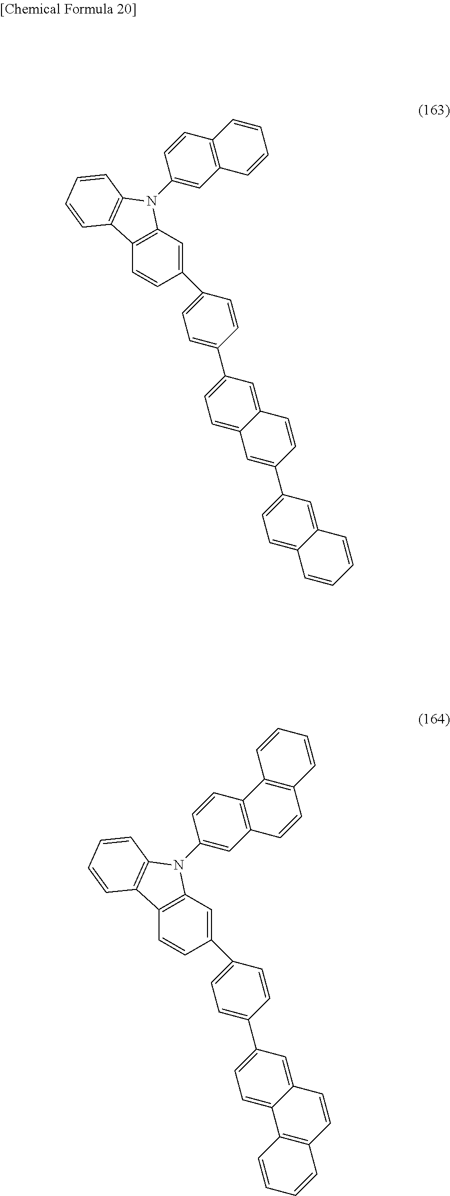

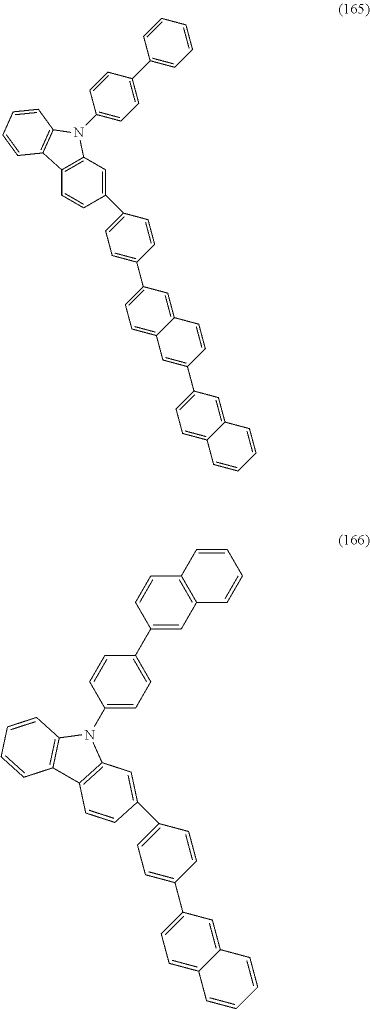

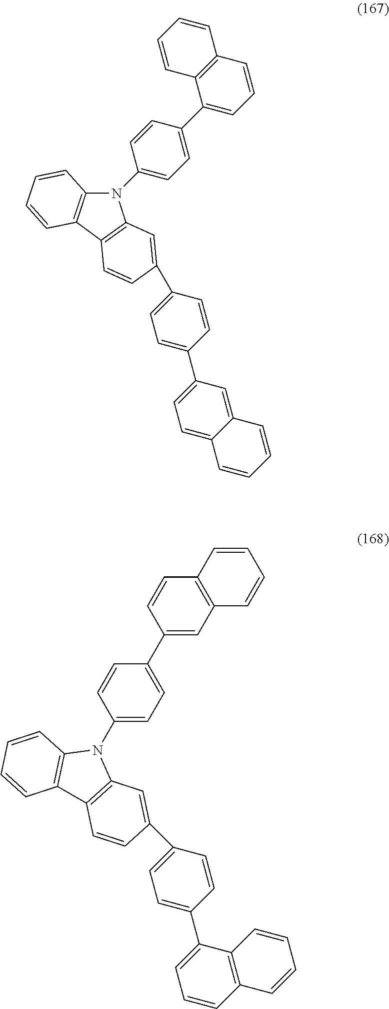

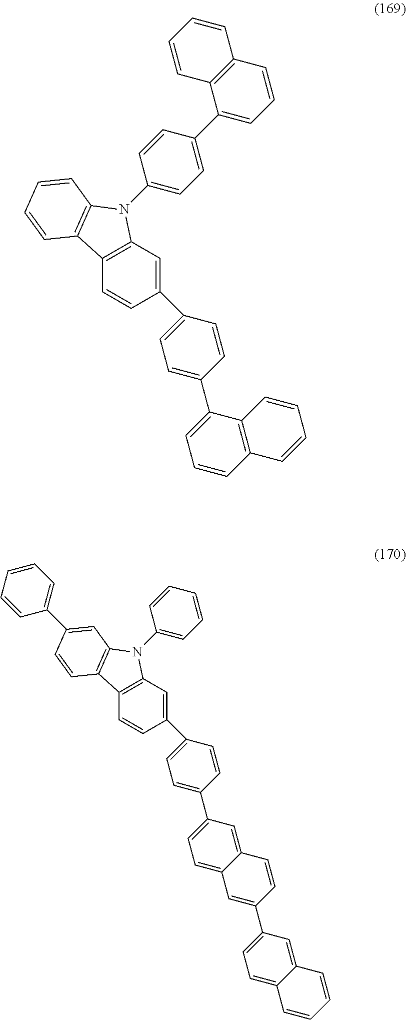

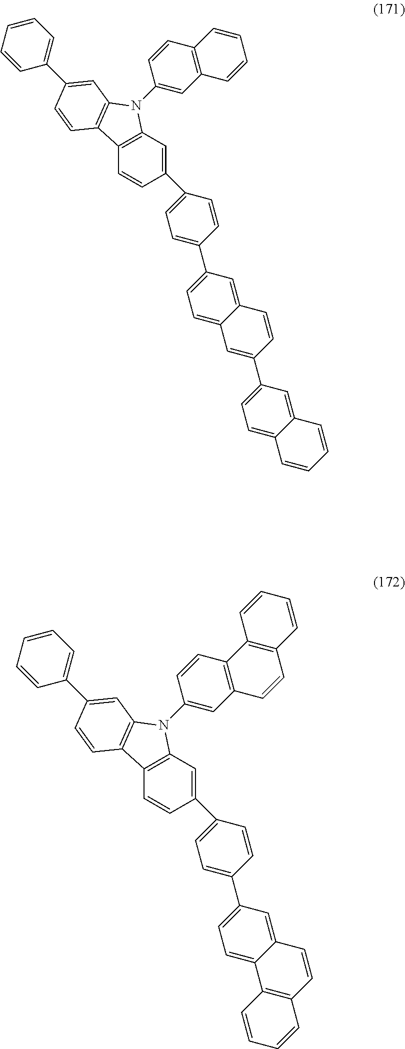

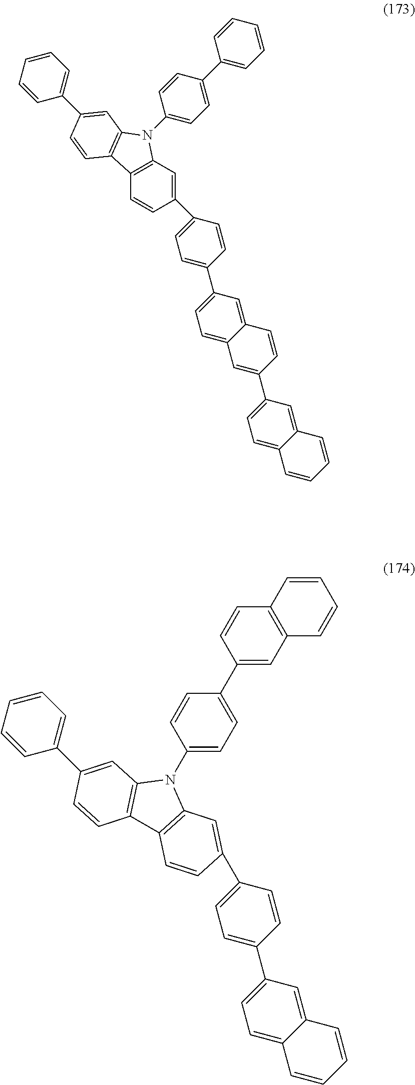

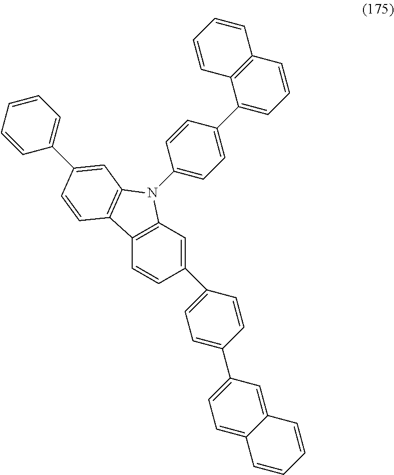

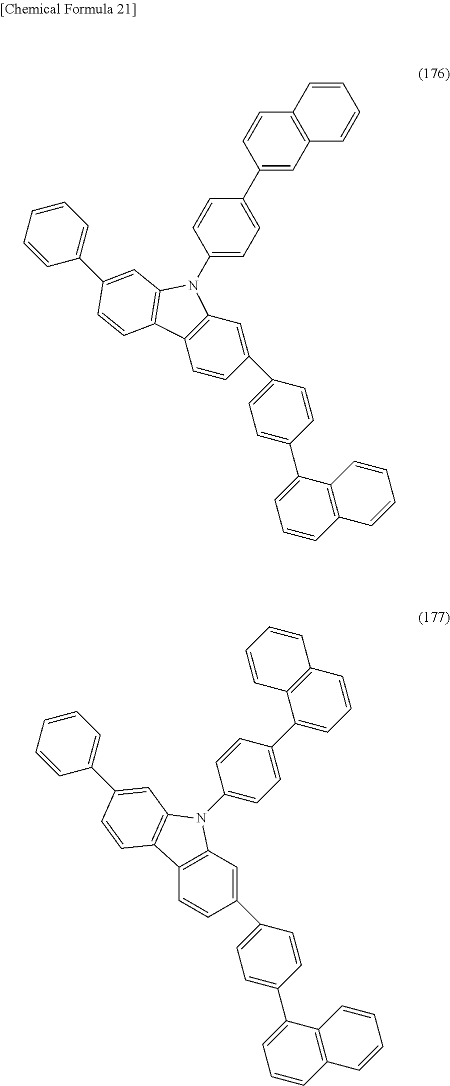

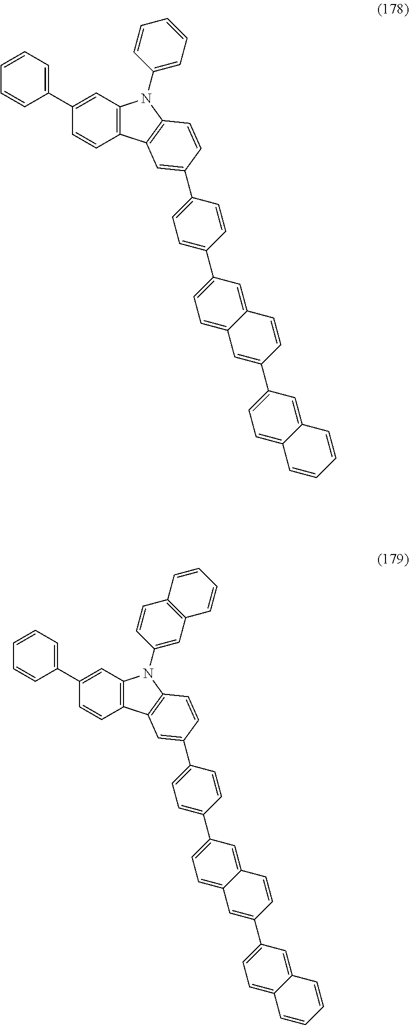

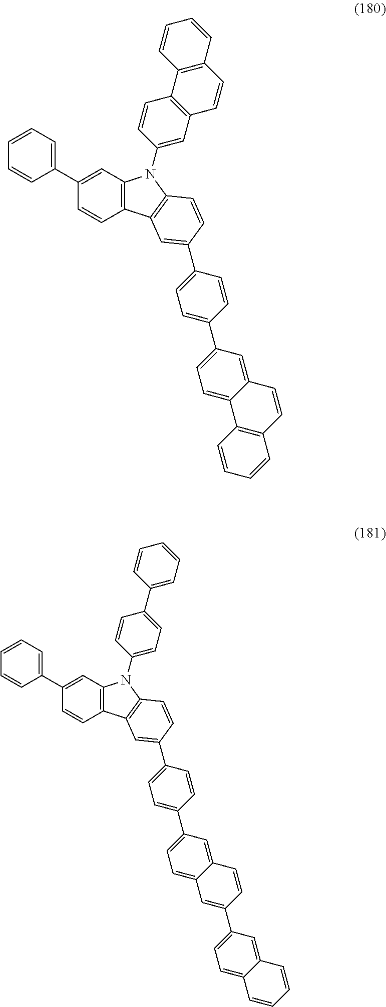

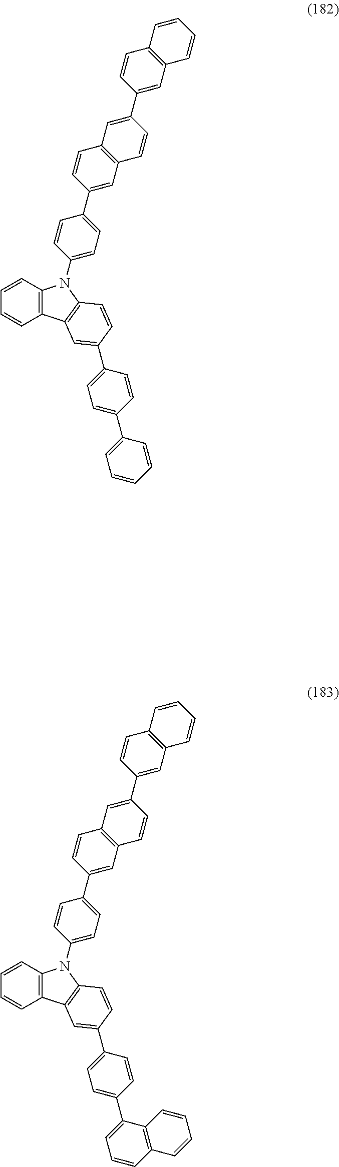

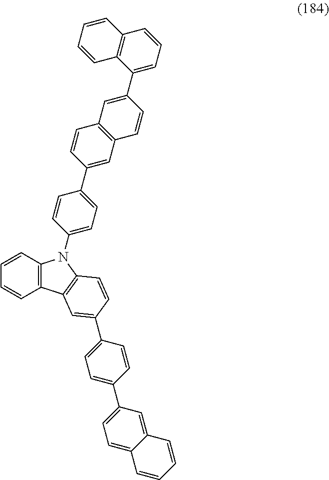

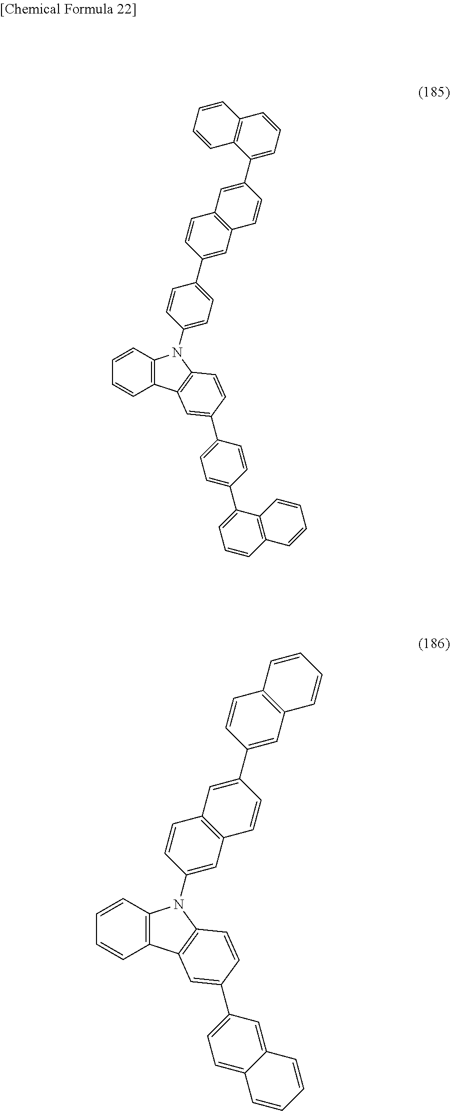

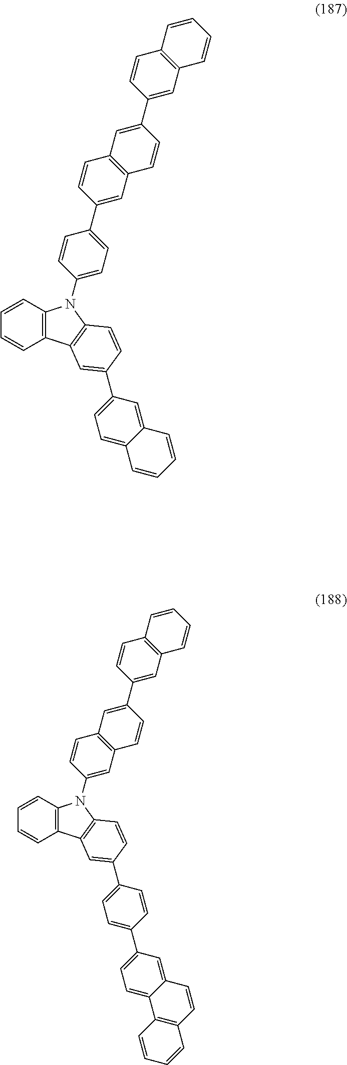

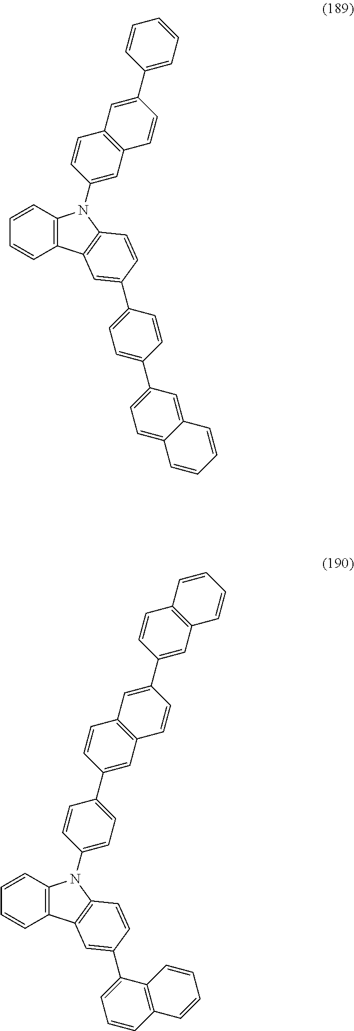

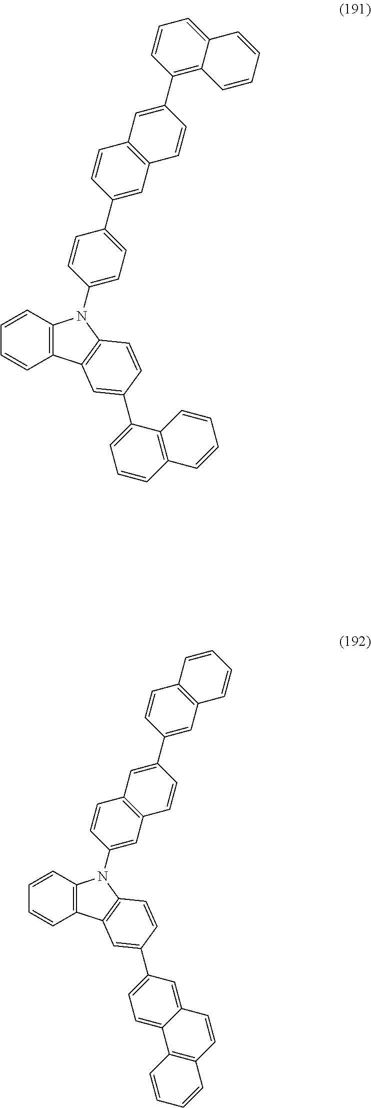

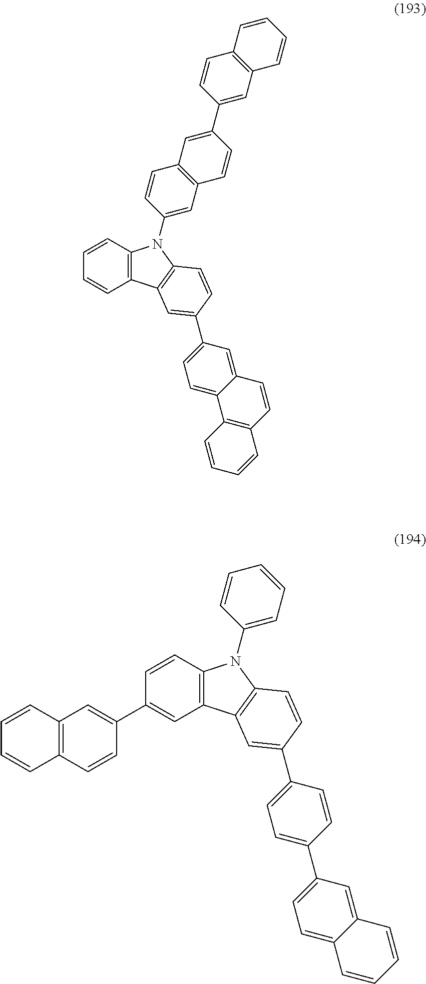

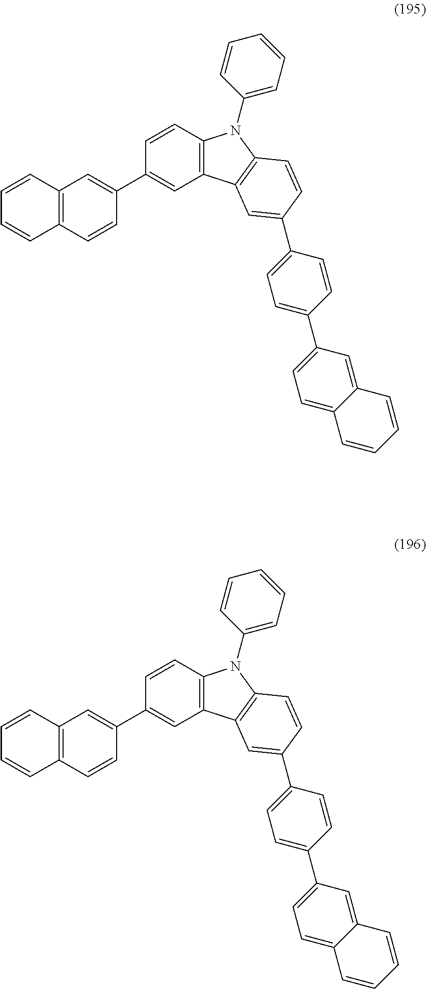

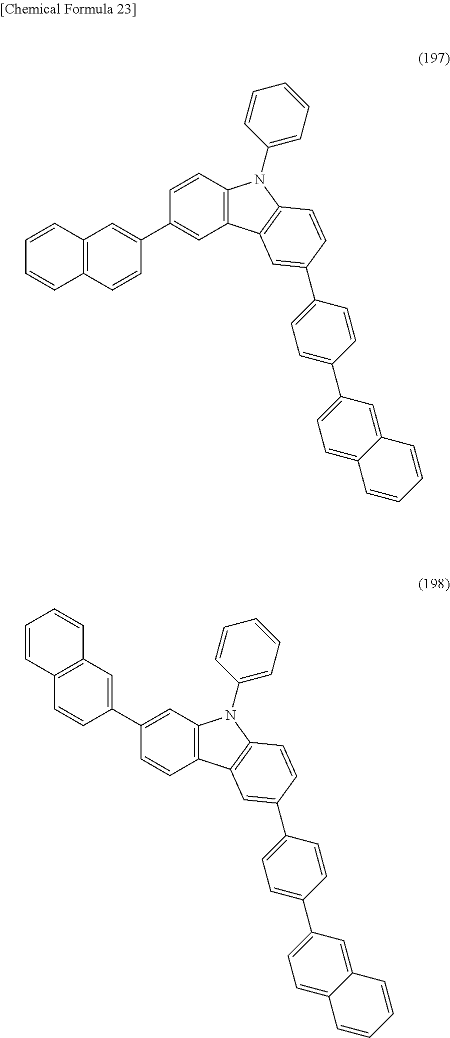

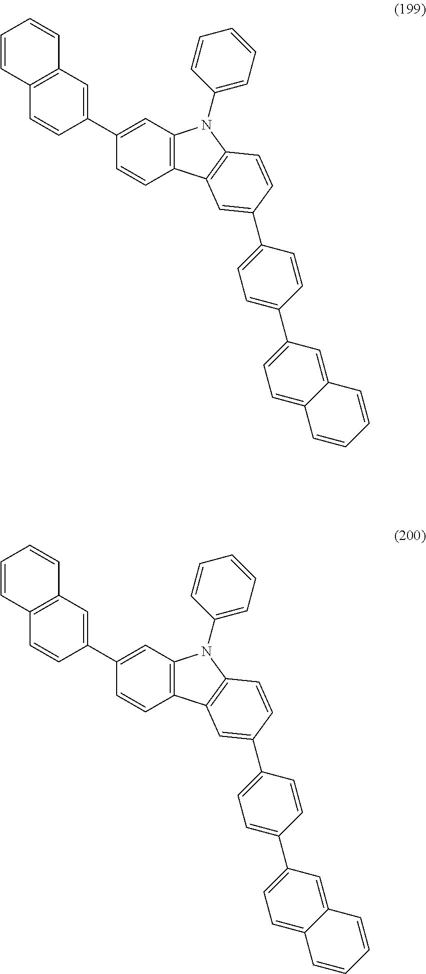

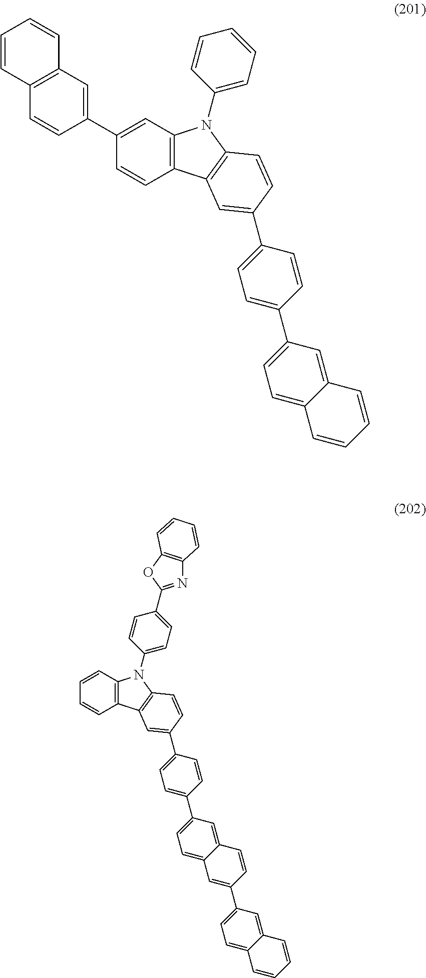

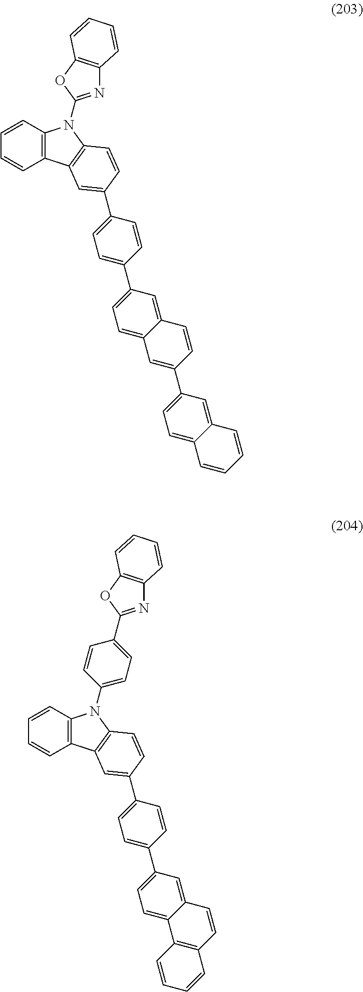

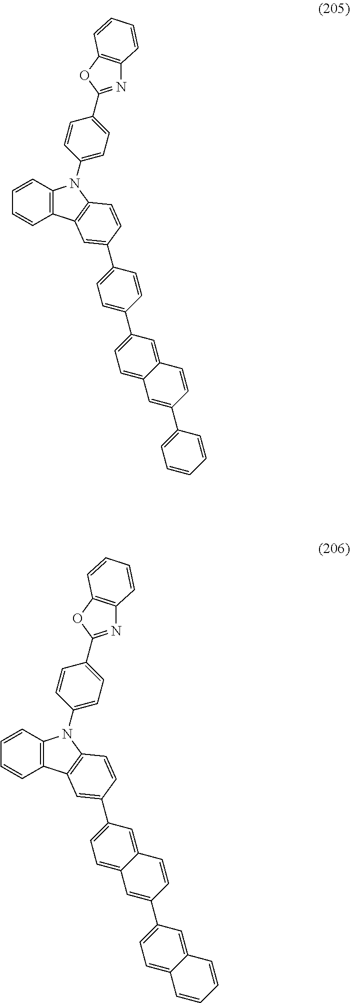

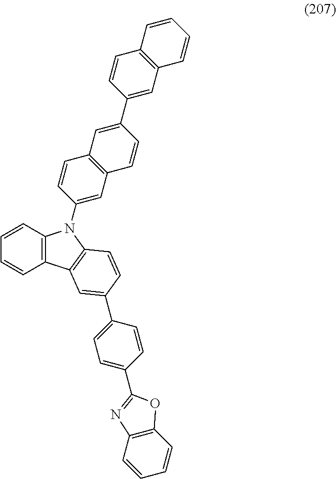

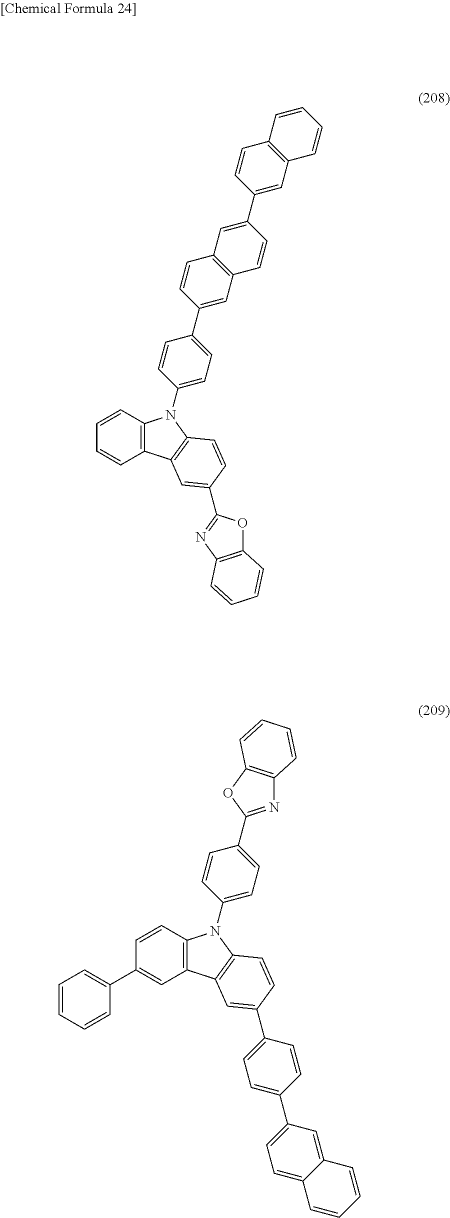

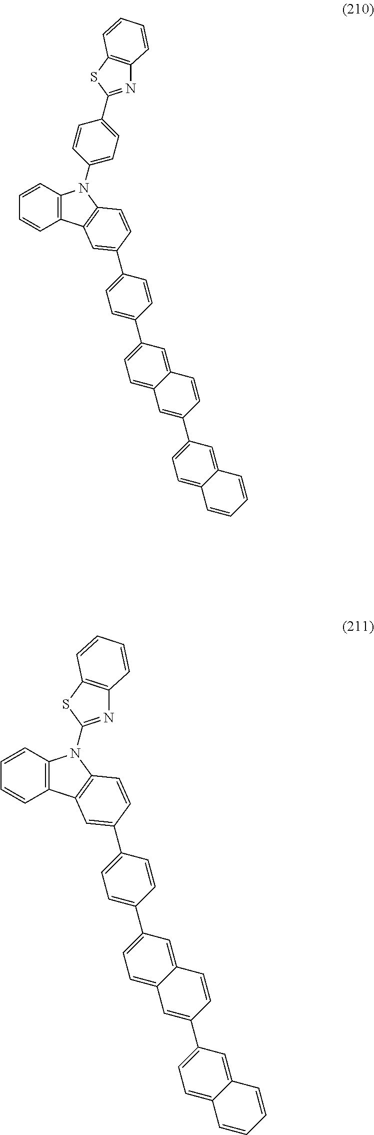

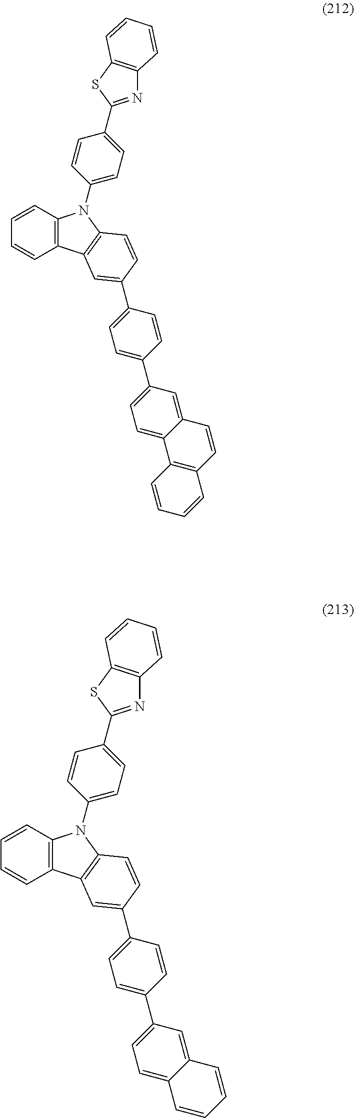

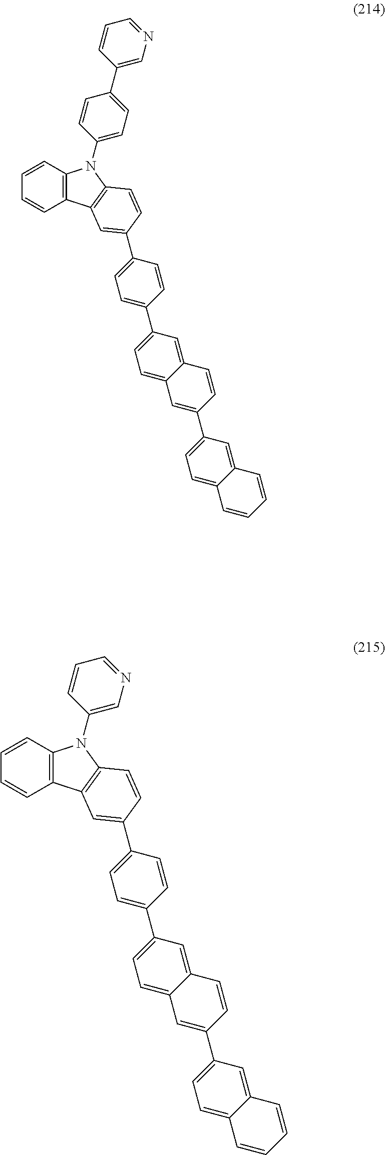

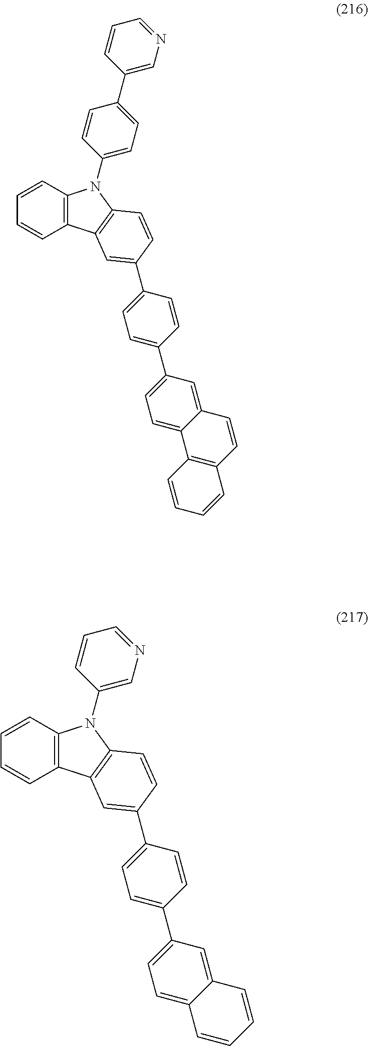

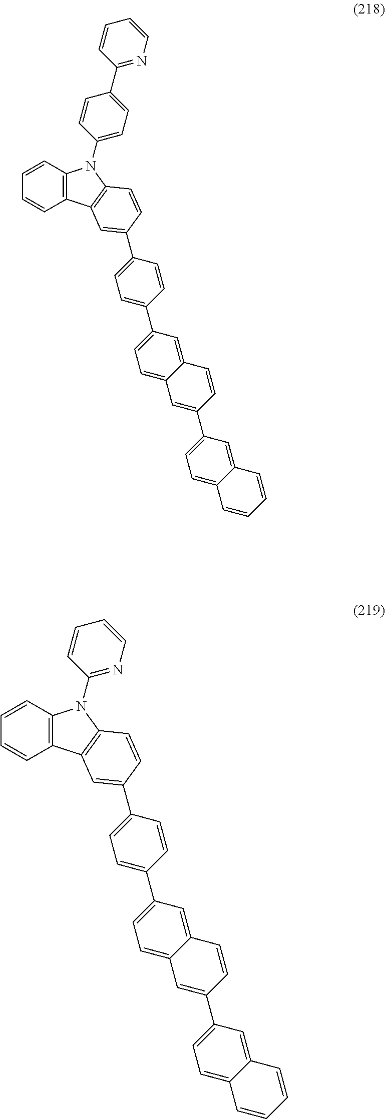

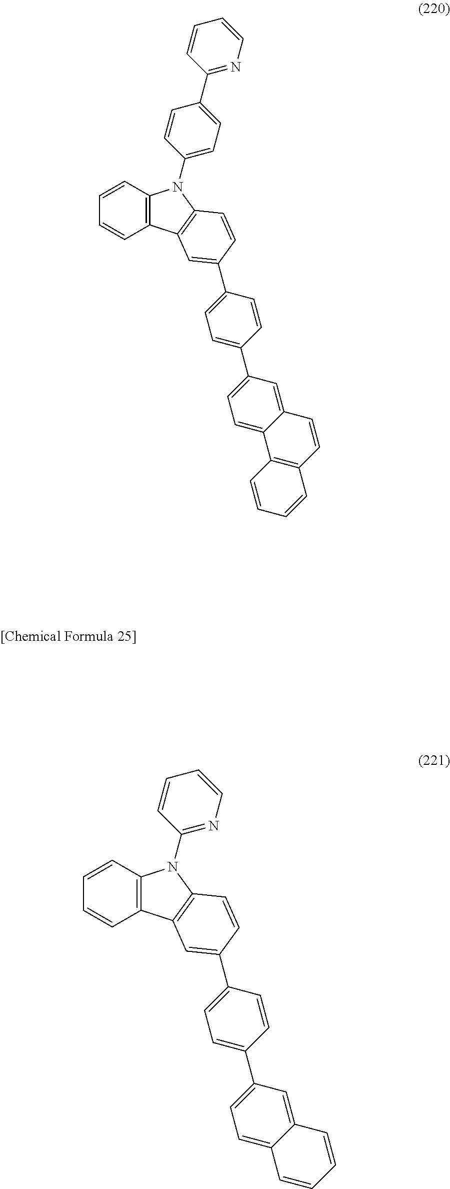

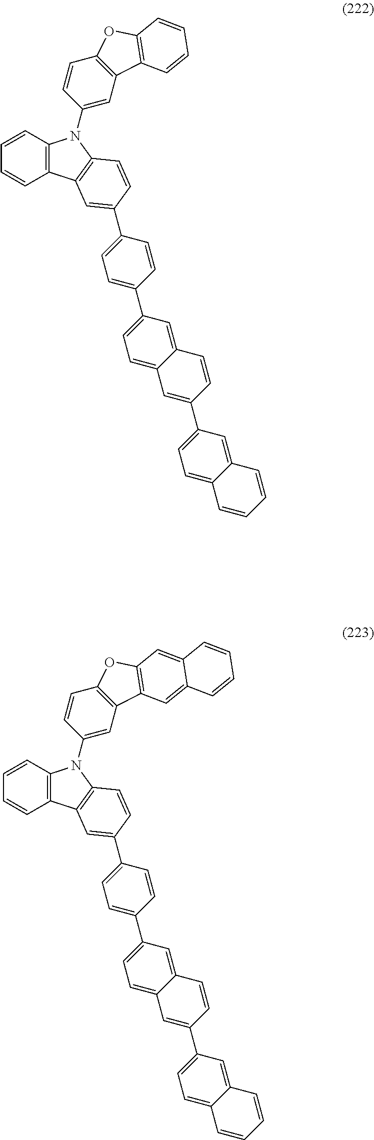

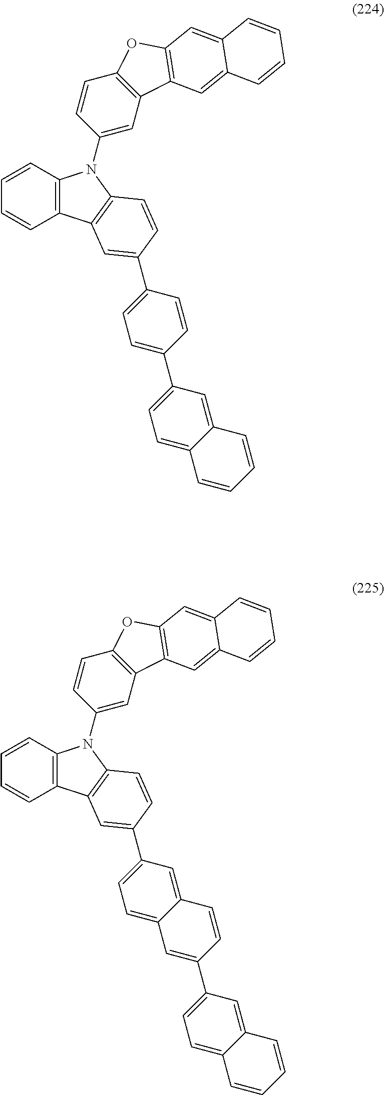

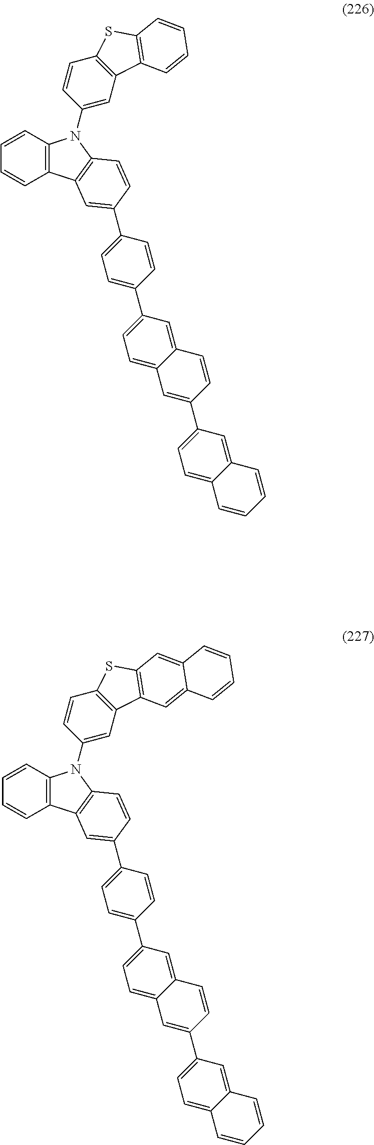

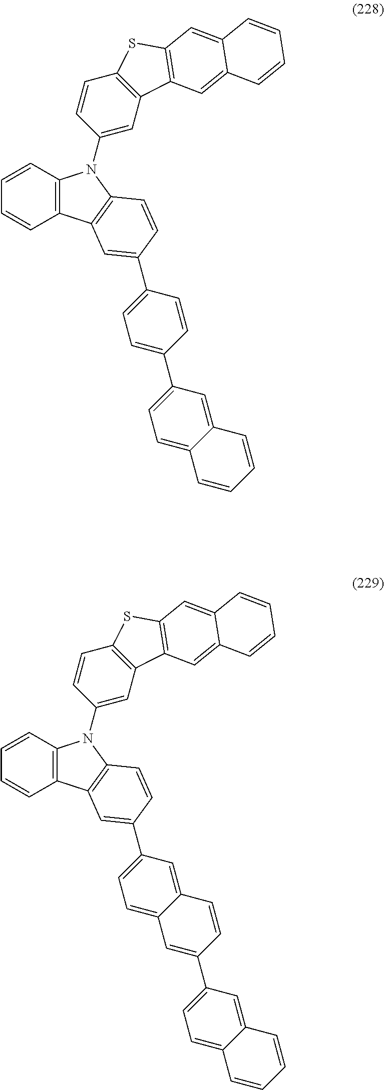

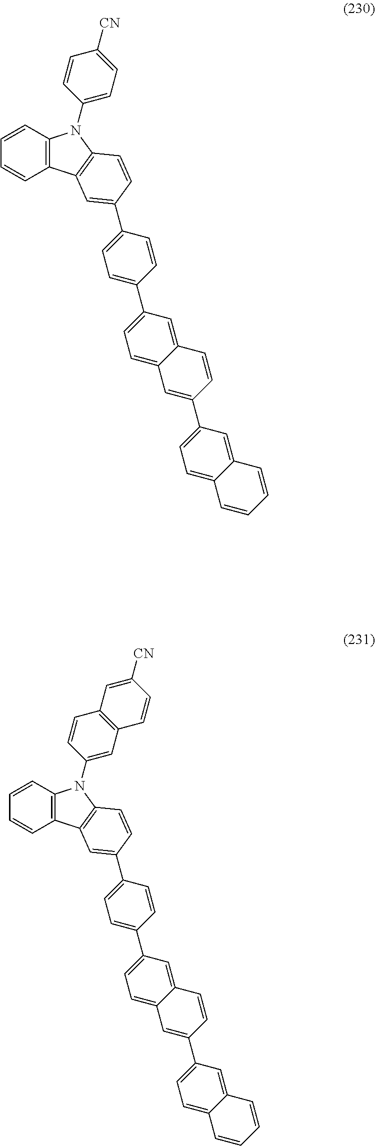

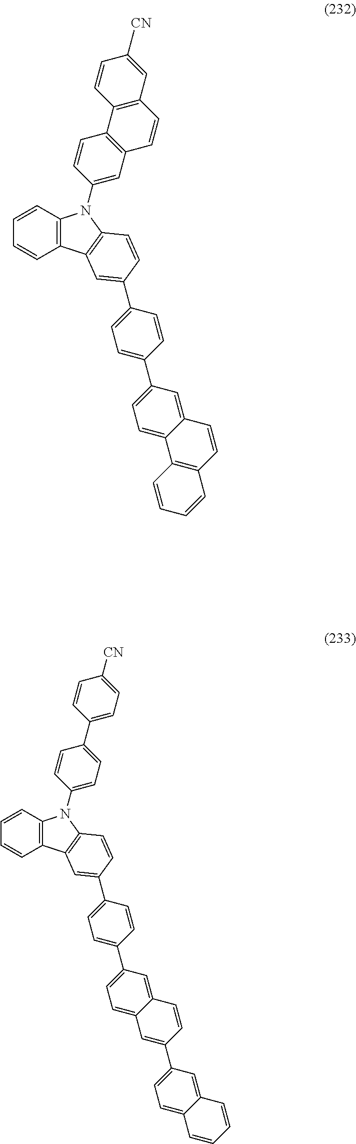

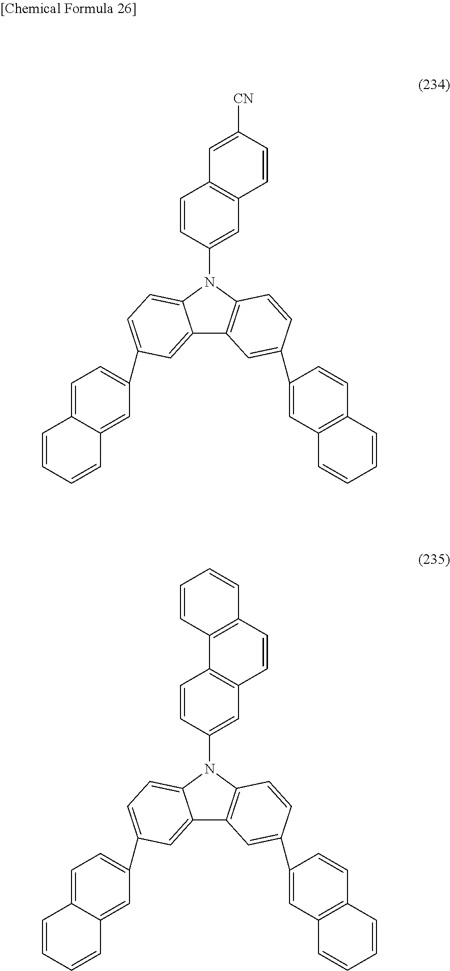

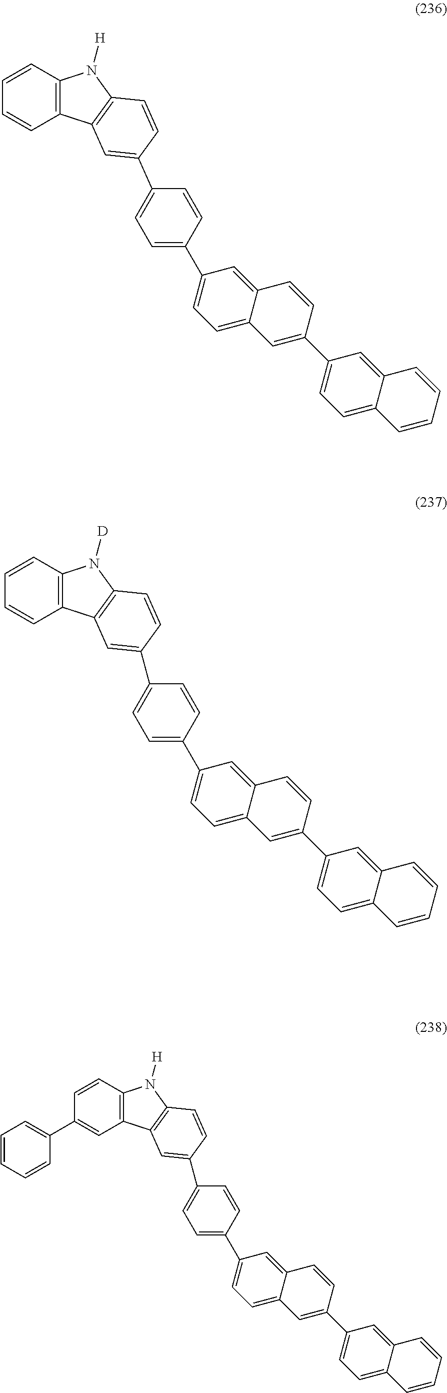

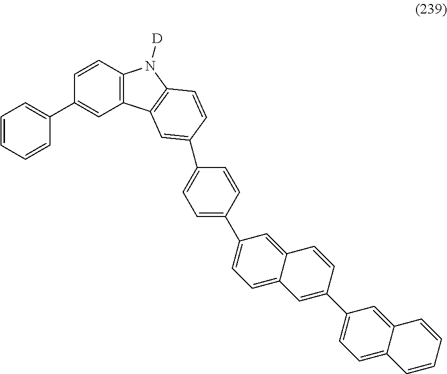

The organic compounds represented by General Formulae (G1), (G1-1), (G1-2), and (G1-3) and Structural Formulae (100) to (239) are examples, and the organic compound of one embodiment of the present invention is not limited thereto.

### <Method for Synthesizing Organic Compound>

A method for synthesizing the organic compound represented by General Formula (G1) described above in <Organic compound example 1> will be described. In General Formulae (G1), (G1-1), (G1-2), and (G1-3) shown below, the above-described structures can be employed as appropriate for R¹ to R⁸, Ar¹¹ to Ar¹⁷, and n11 to n17.

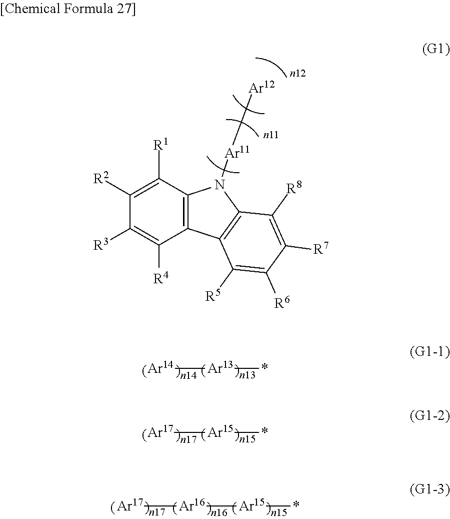

Here, General Formula (G1) can be represented by General Formula (G1a) or (G1b) shown below. General Formula (G1a) shows a structure in which General Formula (G1-1) is bonded to any one of R¹ to R⁴ in General Formula (G1), R¹¹ to R¹³ are substituted for another one of R¹ to R⁴, General Formula (G1-2) is bonded to any one of R⁵ to R⁸, and R¹⁴ to R¹⁶ are substituted for another one of R⁵ to R⁸. Similarly, General Formula (G1b) shows a structure in which General Formulae (G1-1) and (G1-3) are bonded to General Formula (G1) and R¹¹ to R¹⁶ are substituted. For R¹¹ to R¹⁶, R¹ to R⁸ described above can be appropriately employed. Although General Formula (G1-1) is bonded to any one of R¹ to R⁴ in the examples, all of R¹ to R⁴ may be hydrogen (including deuterium).

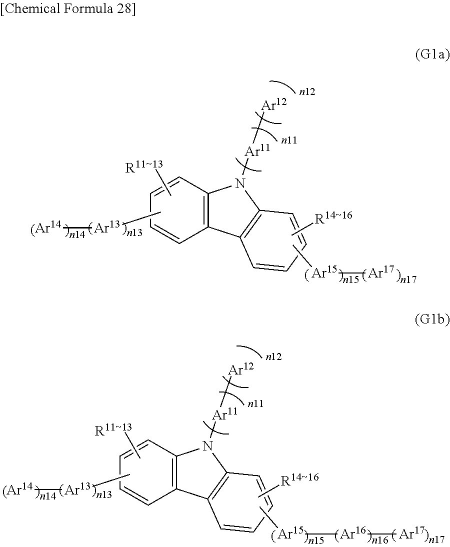

### <<Method for Synthesizing Organic Compound Represented by General Formula (G1a)>>

A variety of reactions can be used for the synthesis of the organic compound represented by General Formula (G1a) above. The organic compound represented by General Formula (G1a) can be synthesized by a simple method shown by the synthesis scheme below, for example.

As shown in Scheme (Sa-1) below, Compound 1 that is a compound including a carbazole skeleton (also referred to as a carbazole compound) and Compound 2 are coupled, whereby a carbazole compound represented by General Formula (G1a) is obtained. In Compound 1, B¹ is bonded to a carbon atom of a benzene ring and represents a boronic acid, a boronic ester, a cyclic-triolborate salt, or the like (also referred to as a boronic acid or the like). As the cyclic-triolborate salt, a lithium salt, a potassium salt, or a sodium salt may be used. In Compound 2, X¹ represents a halogen. The Suzuki-Miyaura reaction using a palladium catalyst can be used as the coupling reaction, for example.

Note that B¹ in Compound 1 may be X¹; in that case, X¹ in Compound 2 is preferably B¹. Two positions of R¹⁴ to R¹⁶ may have a boronic acid or the like. In that case, Compound 2 can be bonded to the two positions.

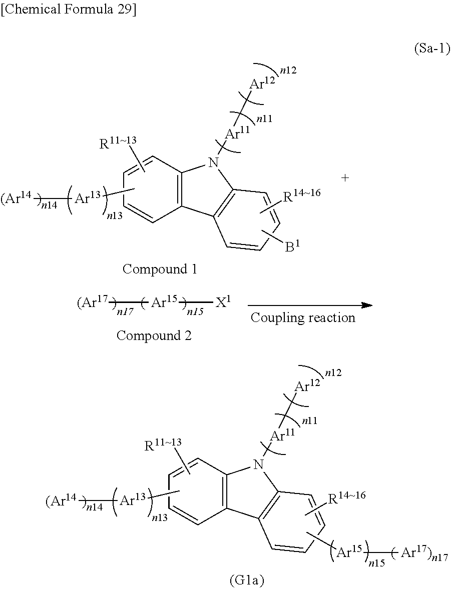

### <<Method for Synthesizing Organic Compound Represented by General Formula (G1b)>>

An example of a method for synthesizing the organic compound represented by General Formula (G1b) above is described.

First, as shown in Scheme (Sb-1) below, Compound 3 and Compound 4 are coupled, whereby Compound 5 is obtained. In Compound 3, B2 represents a boronic acid or the like. In Compound 4 and Compound 5, X² and X³ each represent a halogen. Note that X² and X³ may represent the same halogen element or different halogen elements. Different halogen elements (e.g., X² represents iodine and X³ represents bromine) are preferably used in order to make a coupling reaction based on one of the halogen elements selectively proceed.

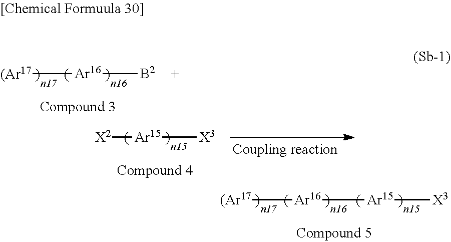

The obtained Compound 5 can be used instead of Compound 2 in Scheme (Sa-1) above. In that case, the organic compound represented by General Formula (G1b) can be obtained by a coupling reaction between Compound 1 and Compound 5.

### <<Another Method for Synthesizing Organic Compound Represented by General Formula (G1a) or (G1b)>>

An example of a method for synthesizing the organic compound represented by General Formula (G1a) or (G1b), which is different from the examples described above is described below.

The organic compound represented by General Formula (G1a) can be synthesized by Synthesis Schemes (Sa-2) and (Sa-3) shown below.

Compound 6 is a 9H-carbazole compound in which nitrogen and hydrogen are bonded. As shown in Synthesis Scheme (Sa-2), Compound 6 and Compound 2 are coupled, whereby Compound 7 is obtained.

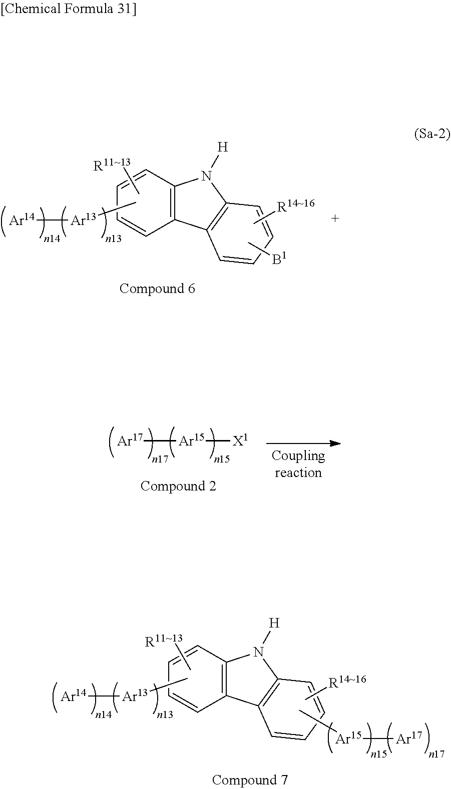

Next, as shown in Synthesis Scheme (Sa-3), Compound 7 and Compound 8 are coupled, whereby the organic compound represented by General Formula (G1a) can be obtained. As the coupling reaction, a Buchwald-Hartwig reaction using a palladium catalyst can be used, for example.

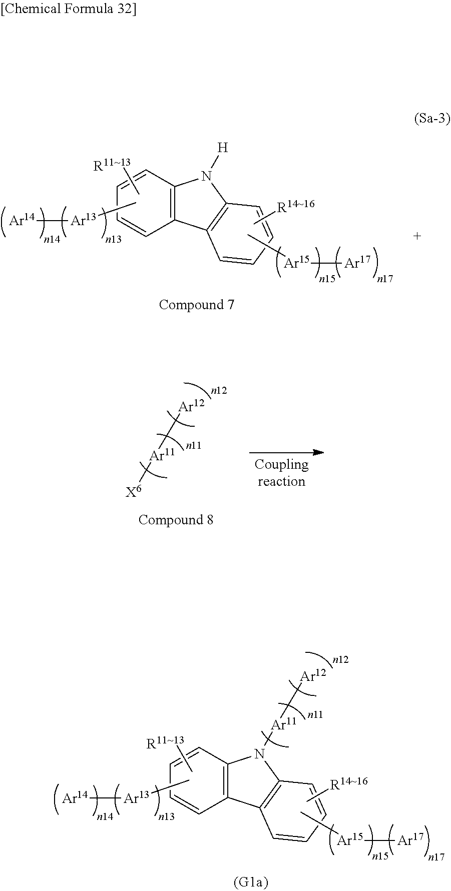

Although an example in which Compound 7 is used as an intermediate is described above, a device may be fabricated using Compound 7 as a function different from the intermediate. In addition, in Compound 6 and Compound 7, hydrogen includes deuterium.

When Compound 5 is used instead of Compound 2 in Synthesis Scheme (Sa-2), the organic compound represented by General Formula (G1b) can be obtained.

Compound 2 and Compound 8 may be the same compound. The same compound is preferably used, in which case Synthesis Scheme (Sa-2) and Synthesis Scheme (Sa-3) can be performed at the same time in some cases and the number of steps can be reduced.

The structure described above in this embodiment can be used in combination with any of the structures described in the other embodiments as appropriate.

### Embodiment 2

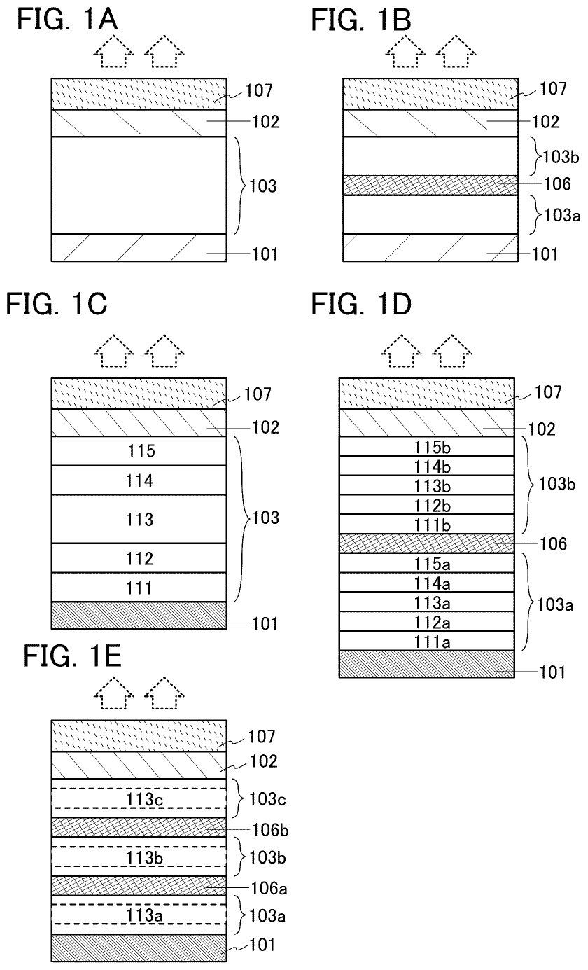

In this embodiment, structures of a light-emitting device including the organic compound described in Embodiment 1 will be described with reference to FIGS. 1A to 1E.

### <Basic Structure of Light-Emitting Device>

A basic structure of a light-emitting device is described. FIG. 1A illustrates a light-emitting device having a structure (single structure) in which an organic compound layer including a light-emitting layer is provided between a pair of electrodes. Specifically, the light-emitting device illustrated in FIG. 1A includes a first electrode 101, a second electrode 102, an organic compound layer 103, and a cap layer 107.

Specifically, the organic compound layer 103 is provided over the first electrode 101, the second electrode 102 is provided over the organic compound layer 103, and the cap layer 107 is provided over the second electrode 102. Here, the second electrode 102 is a light-transmitting electrode, and the light-emitting device emits light from the second electrode 102 side.

FIG. 1B illustrates a light-emitting device that has a stacked-layer structure (tandem structure) in which a plurality of organic compound layers 103 (two organic compound layers 103a and 103b in FIG. 1B) each including a layer containing a light-emitting substance are provided between a pair of electrodes and a charge-generation layer 106 is provided between the organic compound layers 103. A light-emitting device having a tandem structure enables manufacturing a light-emitting apparatus that increases efficiency without changing the amount of current.

The charge-generation layer 106 has a function of injecting electrons into one of the organic compound layers (the organic compound layer 103a or 103b) and injecting holes into the other of the organic compound layers (the organic compound layer 103b or 103a) when a potential difference is caused between the first electrode 101 and the second electrode 102. Thus, in FIG. 1i, when a voltage is applied so that the potential of the first electrode 101 is higher than that of the second electrode 102, the charge-generation layer 106 injects electrons into the organic compound layer 103a and injects holes into the organic compound layer 103b.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions as a charge-generation layer even if it has lower conductivity than the first electrode 101 or the second electrode 102.

FIG. 1C illustrates a stacked-layer structure of the organic compound layer 103 in the light-emitting device of one embodiment of the present invention. In this case, the first electrode 101 is regarded as functioning as an anode, and the second electrode 102 is regarded as functioning as a cathode. The organic compound layer 103 has a structure in which a hole-injection layer 111, a hole-transport layer 112, a light-emitting layer 113, an electron-transport layer 114, and an electron-injection layer 115 are stacked in this order over the first electrode 101. Note that the light-emitting layer 113 may have a stacked-layer structure of a plurality of light-emitting layers that emit light of different colors. For example, a light-emitting layer including a light-emitting substance that emits red light, a light-emitting layer including a light-emitting substance that emits green light, and a light-emitting layer including a light-emitting substance that emits blue light may be stacked with or without a layer including a carrier-transport material therebetween. Alternatively, a light-emitting layer including a light-emitting substance that emits yellow light and a light-emitting layer including a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer including a light-emitting substance that emits blue light and a second light-emitting layer including a light-emitting substance that emits blue light may be stacked with or without a layer including a carrier-transport material therebetween. The structure in which a plurality of light-emitting layers that emit light of the same color are stacked can sometimes achieve higher reliability than a single-layer structure. Also in the case where a plurality of EL layers are provided as in the tandem structure illustrated in FIG. 1B, the layers in each EL layer are sequentially stacked from the anode side as described above. When the first electrode 101 is the cathode and the second electrode 102 is the anode, the stacking order of the layers in the organic compound layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the organic compound layers (the organic compound layers 103, 103a, and 103b) includes an appropriate combination of a light-emitting substance and a plurality of substances, so that fluorescent light of a desired color or phosphorescent light of a desired color can be obtained. The light-emitting layer 113 may have a stacked-layer structure having different emission colors. In that case, a light-emitting substance and other substances are different between the stacked light-emitting layers. Furthermore, the plurality of organic compound layers (the organic compound layers 103a and 103b) in FIG. 1B may exhibit emission colors different from each other. In that case, the light-emitting substance and the other substances can differ between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 1C. Thus, light from the light-emitting layer 113 in the organic compound layer 103 can be resonated between the electrodes and light emitted through the second electrode 102 can be intensified. Thus, high resolution can be easily achieved. In addition, emission intensity at a predetermined wavelength in the front direction can be increased, whereby power consumption can be reduced.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer greater than or equal to 1) or close to mλ/2.

To amplify desired light (wavelength: λ) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer greater than or equal to 1) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

The light-emitting device illustrated in FIG. 1D is a light-emitting device having a tandem structure. The tandem structure enables a light-emitting device to emit light with high luminance. Furthermore, the amount of current needed for obtaining a predetermined luminance can be smaller in the tandem structure than in the single structure; thus, the tandem structure enables higher reliability. In addition, power consumption can be reduced.

The light-emitting device illustrated in FIG. 1E is an example of the light-emitting device having the tandem structure illustrated in FIG. 1B, and includes three organic compound layers (the organic compound layers 103a and 103b and an organic compound layer 103c) stacked with charge-generation layers (charge-generation layers 106a and 106b) positioned therebetween, as illustrated in FIG. 1E. The three organic compound layers (the organic compound layers 103a, 103b, and 103c) include respective light-emitting layers (light-emitting layers 113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light; alternatively, the light-emitting layer 113a can emit red light, the light-emitting layer 113b can emit blue light, green light, or yellow light, and the light-emitting layer 113c can emit red light.

In the above light-emitting device of one embodiment of the present invention, the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity lower than or equal to 1×10⁻² Ωcm.

When the first electrode 101 is a reflective electrode in the above light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity lower than or equal to 1×10⁻² Ωcm.

### <Specific Structure of Light-Emitting Device>

Next, a specific structure of the light-emitting device of one embodiment of the present invention will be described. Here, the description is given using FIG. 1D illustrating the tandem structure. Note that the structure of the organic compound layer applies also to the structure of the light-emitting devices having the single structure in FIGS. 1A and 1C. When the light-emitting device illustrated in FIG. 1D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the organic compound layer 103b, with the use of a material selected as appropriate.

### <Materials of Light-Emitting Device>

The components of the light-emitting device are described below.

### <<Cap Layer>>

The organic compound used in the cap layer 107 preferably has an ordinary refractive index (nₒ) at a wavelength of 450 nm of higher than or equal to 1.90, preferably higher than or equal to 2.00 in a deposited film state. Furthermore, the organic compound preferably has an ordinary refractive index (nₒ) at a wavelength of 520 nm of higher than or equal to 1.80, preferably higher than or equal to 1.9. Furthermore, the organic compound preferably has an ordinary refractive index (nₒ) at a wavelength of 630 nm of higher than or equal to 1.75, preferably higher than or equal to 1.85. By increasing the ordinary refractive index (n), light from the organic compound layer 103 can be inhibited from being totally reflected by the cap layer 107, leading to an improvement in light extraction efficiency.

Moreover, the organic compound preferably has an extraordinary refractive index (nₑ) at a wavelength of 450 nm of lower than or equal to 1.80, preferably lower than or equal to 1.70. Furthermore, the organic compound preferably has an extraordinary refractive index (nₑ) at a wavelength of 520 nm of lower than or equal to 1.70, preferably lower than or equal to 1.60. Furthermore, the organic compound preferably has an extraordinary refractive index (nₑ) at a wavelength of 630 nm of lower than or equal to 1.70, preferably lower than or equal to 1.60. When the extraordinary refractive index (nₑ) is low, the refractive index anisotropy tends to be large.

When the difference between the ordinary refractive index (nₒ) and the extraordinary refractive index (nₑ) (Δn=|nₒ−nₑ|) at any of the wavelengths higher than or equal to 360 nm and lower than or equal to 830 nm (e.g., three wavelengths of 450 nm, 520 nm, and 630 nm), preferably at each of the three wavelengths is greater than or equal to 0.1 and less than or equal to 0.4, the refractive index anisotropy can be much larger. In addition, Δn=|nₒ−nₑ| is preferably greater than or equal to 0.2 and less than or equal to 0.4, further preferably greater than or equal to 0.3 and less than or equal to 0.4.

Here, the difference between the ordinary refractive index (nₒ) and the extraordinary refractive index (nₑ) (Δn=|nₒ−nₑ|) can be used as an indicator of anisotropy in the refractive index. In the visible light range (higher than or equal to 360 nm and lower than or equal to 830 nm), the refractive index anisotropy tends to be larger with a larger Δn. In order to increase Δn, the ordinary refractive index (nₒ) is increased or the extraordinary refractive index (nₑ) is decreased.

With large refractive index anisotropy in the cap layer 107, light emitted from the organic compound layer 103 can be highly efficiently extracted.

In addition, the ordinary extinction coefficient (kₒ) of the cap layer 107 is preferably small. Specifically, the ordinary extinction coefficient (kₒ) in the visible range (greater than or equal to 450 nm and less than or equal to 630 nm) is preferably lower than or equal to 0.2, further preferably lower than or equal to 1×10⁻², still further preferably lower than or equal to 1×10⁻⁴. In the case where the ordinary extinction coefficient is low, light emitted from the organic compound layer 103 can be extracted to the outside without absorption of light; thus, a highly efficient element can be provided.

Note that in consideration of the extraction efficiency, the ordinary extinction coefficient in the above-described visible range is desirably lower than or equal to the lower detection limit of a measurement apparatus. Meanwhile, in the case where slight absorption occurs (the ordinary extinction coefficient is not 0) at a wavelength lower than or equal to 400 nm, ultraviolet rays (light having a shorter wavelength than visible light) from the outside of the light-emitting device may be absorbed by the cap layer, so that damage to the underlayer structure of the cap layer due to ultraviolet rays can be reduced. In consideration of such ultraviolet absorption, the ordinary extinction coefficient at a wavelength lower than or equal to 400 nm is preferably higher than or equal to the lower detection limit. For example, the ordinary extinction coefficient at 365 nm is preferably higher than or equal to 0.1, further preferably higher than or equal to 0.2.

Examples of the organic compound that can be used for the cap layer 107 are described below.

A compound having a heterocycle can be used as the organic compound. Examples of heteroatoms included in the heterocycle include nitrogen, oxygen, and sulfur. When the heteroatoms are included, the ordinary refractive index nₒ can be increased or the glass transition temperature T_{g} can be increased in some cases. In order to improve the refractive index, the heterocycle preferably has a five-membered heteroaromatic ring skeleton including nitrogen, oxygen, and sulfur and preferably has a pyrrole skeleton, a furan skeleton, a thiophene skeleton, an azole (e.g., imidazole, oxazole, thiazole, oxadiazole, or triazole) skeleton, or the like, for example. In particular, a compound having a molecular structure including an atom with a large atomic radius, like a sulfur atom, is expected to have a high refractive index. The condensed ring preferably has a structure including heteroatoms in order that the condensed ring can have an effect of increasing T_{g} and the heteroatoms can have an effect of increasing the refractive index at the same time. That is, a five-membered condensed heteroaromatic ring including nitrogen, oxygen, and sulfur is preferable as the heterocycle; examples include a carbazole ring, a dibenzofuran ring, a benzonaphthofuran ring, a dibenzothiophene ring, a benzonaphthothiophene ring, a benzoxazole ring, and a benzothiazole ring. From the above description, an organic compound having the above-described heterocycle and a phenanthrene ring is suitable for the cap layer 107 in one embodiment of the present invention. Furthermore, an organic compound having the above-described heterocycle and two to four naphthalene rings is particularly suitable for the cap layer 107.

Moreover, the organic compound that can be used is a compound having a carbazole ring (also referred to as a carbazole compound), particularly an organic compound having a carbazole ring and a phenanthrene ring, and further particularly an organic compound having a carbazole ring and two to four naphthalene rings.

A deposited film of the carbazole compound can have a high refractive index. First, the organic compound having a carbazole skeleton has a higher refractive index and larger anisotropy than the organic compound not having the skeleton.

When the organic compound suitable for the cap layer 107 includes a condensed ring in the molecule, the refractive index can be further increased. For example, a phenanthrene ring or a naphthalene ring is preferably included. These rings are preferably bonded to the carbazole ring as a substituent. It is preferable that one phenanthrene ring or naphthalene ring be included in the molecule, and when two or more phenanthrene or naphthalene rings are included, the refractive index can be further increased. For example, a binaphthalene structure in which two naphthalene rings are directly connected to each other is suitable. In the case where the substituent has a binaphthalene structure, the glass transition temperature T_{g} of the compound can be increased. Furthermore, when a structure in which the 2-position of naphthalene is bonded to another group (i.e., a 2-naphthyl group) is included, the refractive index can be further increased. This is because the density of the organic compound in the deposited film is increased. In particular, when a 2,2′-binaphthalene structure in which the 2-positions of naphthalenes are bonded to each other is included in a partial structure, the refractive index can be further increased. However, without limitation to this, T_{g} can be improved when a structure in which the 1-position of naphthalene is bonded to another group (i.e., a 1-naphthyl group) is included, for example.

An increase in the number of condensed rings may increase the evaporation temperature; thus, the number of naphthalene rings included in the molecule is preferably less than or equal to four, further preferably less than or equal to three. In terms of increasing the refractive index, the number of naphthalene rings is preferably two or more.

When the compound has benzene as a substituent, the compound preferably has paraphenylene (also referred to as p-phenylene). A compound having paraphenylene can have higher molecular orientation when deposited and thus have higher ordinary refractive index than a compound having metaphenylene (or orthophenylene). However, metaphenylene or orthophenylene can also be used, in which case an effect of decreasing the sublimation temperature can be expected.

The organic compound preferably has a high refractive index and a low sublimation temperature (or a low evaporation temperature) and is unlikely to be thermally decomposed at the time of sublimation by having one to three phenanthrene rings or two to four naphthalene rings as a substituent on the carbazole ring. In terms of the sublimation temperature, the number of naphthalene rings that the organic compound has is preferably greater than or equal to two and less than or equal to four, further preferably greater than or equal to two and less than or equal to three.

An organic compound with a low evaporation temperature is helpful when being used in an industrial product because the organic compound can be deposited at a low temperature and thus is less thermally affected during the deposition and decomposition due to heat can be reduced.

In particular, in the mass production process, the same material is heated continuously for a long time; an organic compound having an excessively high evaporation temperature is easily decomposed by the heating. When the material is decomposed, the evaporation temperature is further increased, for example, whereby a stable mass production system cannot be established. Thus, the organic compound material that can be deposited at a low temperature can be deposited without decomposition of the material, resulting in stable mass production.

Specifically, the evaporation temperature of the organic compound in an evaporation apparatus is preferably lower than or equal to 350° C., further preferably lower than or equal to 330° C., still further preferably lower than or equal to 300° C., yet still further preferably lower than or equal to 270° C. The sublimation temperature of the organic compound is preferably lower than or equal to 350° C., further preferably lower than or equal to 300° C., still further preferably lower than or equal to 290° C., yet still further preferably lower than or equal to 260° C. It is also preferable that the 50% weight loss temperature be low in the measurement with a TG-DTA apparatus. The 50% weight loss temperature being low means that even when the sublimation weight increases, sublimation can be performed stably at a low temperature. The 50% weight loss temperature is preferably lower than or equal to 390° C., further preferably lower than or equal to 350° C., still further preferably lower than or equal to 320° C., yet still further preferably lower than or equal to 300° C.

Note that the number of carbazole rings in the organic compound is preferably only one. When the number of carbazole rings in the organic compound increases, the sublimation temperature increases, so that the evaporation temperature also increases and the yield in the manufacturing process decreases in some cases.

As the organic compound used for the cap layer 107, an organic compound represented by General Formula (G1) below is preferably used.

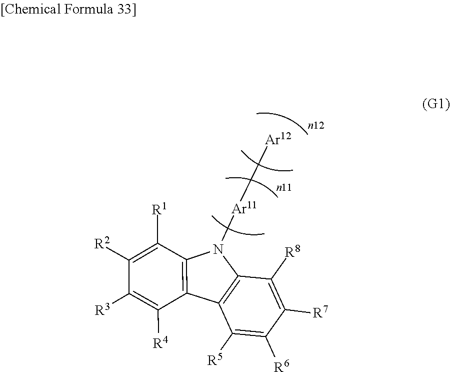

In General Formula (G1), each of R¹ to R⁴ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1). Each of R⁵ to R⁸ independently represents any of hydrogen (including deuterium), a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3). At least one of R⁵ to R⁸ represents the substituent represented by General Formula (G1-3). Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms. Ar¹² represents a substituted or unsubstituted monovalent aromatic hydrocarbon group having 6 to 30 carbon atoms or a substituted or unsubstituted monovalent aromatic heterocyclic group having 3 to 30 carbon atoms. In addition, n11 represents an integer greater than or equal to zero and less than or equal to three and n12 represents an integer greater than or equal to one and less than or equal to three.

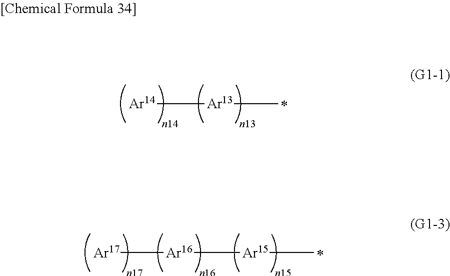

In General Formulae (G1-1) and (G1-3), each of Ar¹³, Ar¹⁵, and Ar¹⁶ independently represents a substituted or unsubstituted divalent aromatic hydrocarbon group having 6 to 30 carbon atoms or a substituted or unsubstituted divalent aromatic heterocyclic group having 3 to 30 carbon atoms. Each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms. At least two of Ar¹⁵, Ar¹⁶, and Ar¹⁷ represent a monovalent or divalent bicyclic or tricyclic aromatic hydrocarbon group or a monovalent or divalent aromatic heterocyclic group. Each of n13, n15, and n16 independently represents an integer greater than or equal to zero and less than or equal to three and each of n14 and n17 independently represents an integer greater than or equal to one and less than or equal to three. Furthermore, n15+n16+n17≥n13+n14 is satisfied.

In General Formulae (G1), (G1-1) and (G1-3), each of Ar¹¹, Ar¹³, Ar¹⁵, and Ar¹⁶ represents an arylene group, and each of Ar¹², Ar¹⁴, and Ar¹⁷ represents an aryl group. When n11 is greater than or equal to two, a plurality of Ar¹¹s may be the same or different substituents. The same applies to Ar¹² to Ar¹⁷. Each of Ar¹¹ to Ar¹⁷ preferably represents a substituent including carbon and hydrogen (including deuterium). Note that Ar¹², Ar¹⁴, or Ar¹⁷ may represent hydrogen (deuterium).

Here, in General Formula (G1) above, for R¹ to R⁸ and Ar¹¹ to Ar¹⁷, the descriptions of the substituents represented by R^{m} (m is an arbitrary number) or Ar^{m} (m is an arbitrary number) described in Embodiment 1 can be referred to. Similarly, R^{m} and Ar^{m} described in Embodiment 2 can be used in Embodiment 1.

Specific examples of the above-described organic compound represented by General Formula (G1) are shown below. Note that the above-described organic compound represented by General Formula (G1) includes the organic compounds described in Embodiment 1.

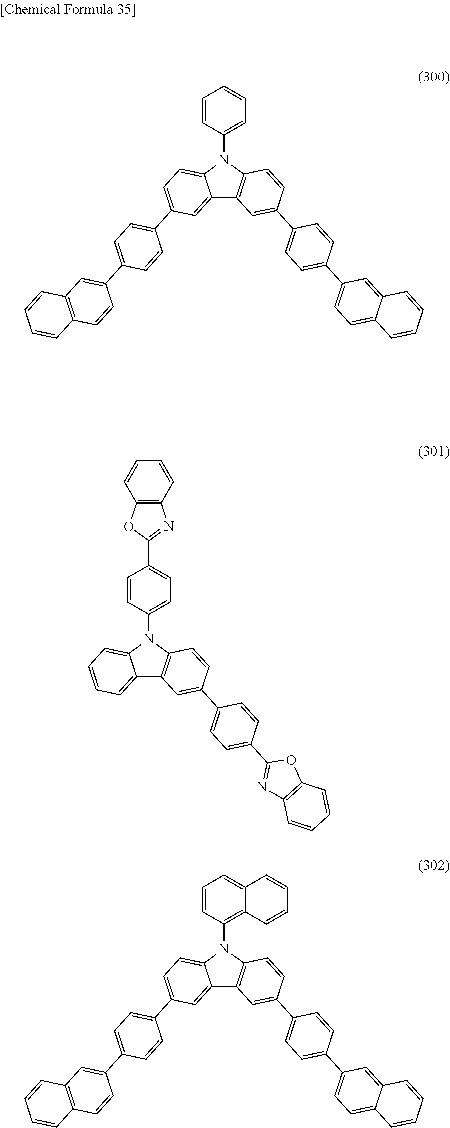

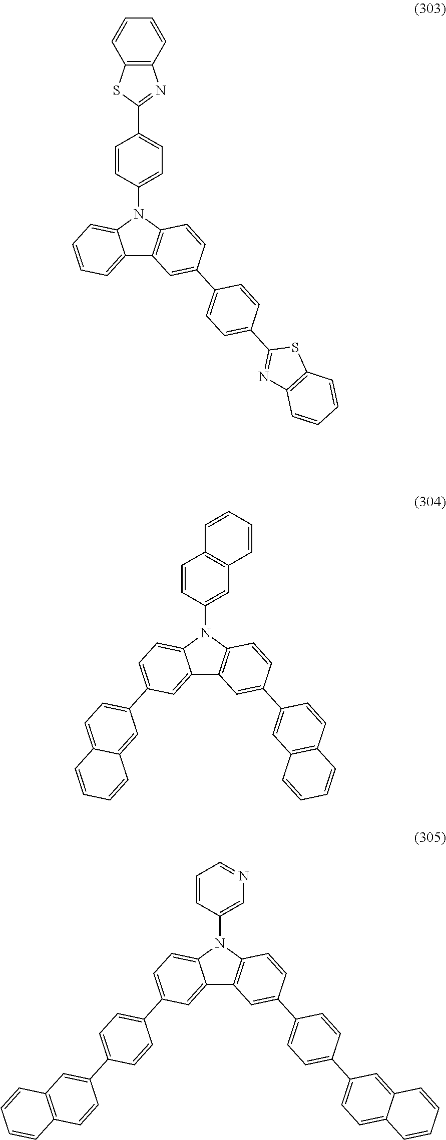

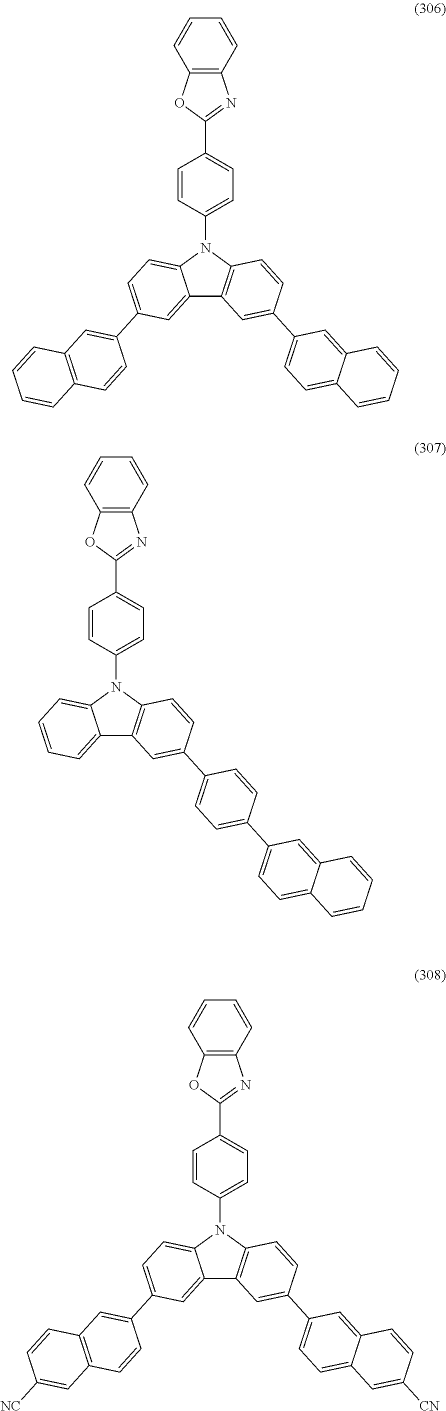

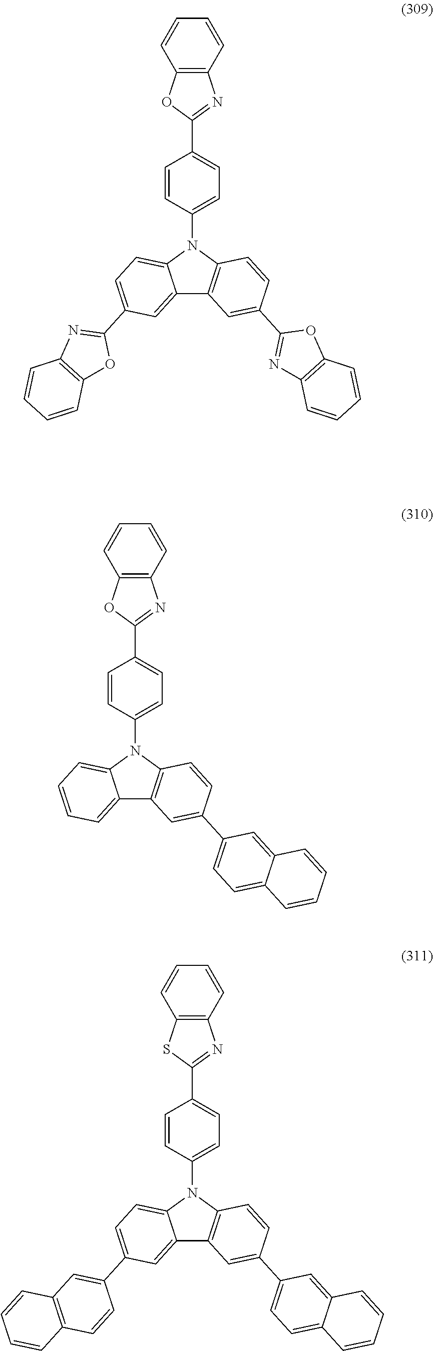

In particular, any of the organic compounds described in Embodiment 1 or any of the above-described organic compound is preferably used for the cap layer 107. The organic compounds described in Embodiment 1 each have a low sublimation temperature and thus can be deposited at a low evaporation temperature. In the mass production process, the same material is heated continuously for a long time; an organic compound having an excessively high evaporation temperature is easily decomposed by the heating. When the material is decomposed, the evaporation temperature is further increased, for example, whereby a stable mass production system cannot be established. Thus, the cap layer material that can be deposited at a low temperature can be deposited without decomposition of the material, resulting in stable mass production. The above-described organic compounds each have a high refractive index or a high ordinary refractive index in the visible range and can increase light extraction efficiency and external quantum efficiency. The above-described organic compounds each have a low extinction coefficient or a low ordinary extinction coefficient in the visible range and can increase light extraction efficiency and external quantum efficiency.

Furthermore, a structure body on which the compound is deposited can be inhibited from being affected by heat during the evaporation. In particular, a thermal budget in formation of the cap layer in manufacturing a device causes a change in quality and deterioration of all the materials used in the structure body to which the compound is deposited. The deterioration of the materials used in the structure body to which the compound is deposited causes an increase in variation in device characteristics, for example, which directly leads to a decrease in manufacturing yield. Accordingly, when the cap layer is deposited by evaporation at a low temperature, a change in quality and deterioration of the materials used in the structure body to which the compound is deposited can be inhibited and manufacturing with high yield is possible.

Specific examples of the material that can be used for the cap layer also include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II).

Note that in the case where the cap layer is formed in contact with the electrode, the use of a π-electron deficient heteroaromatic compound may cause an interaction (formation of a coordination bond) with a metal such as silver (Ag), gold (Au), or aluminum (Al) used for the electrode, so that light absorption is observed in the visible range and the near-infrared range in some cases. In that case, light generated in the light-emitting layer might be absorbed and the outcoupling efficiency might be lowered. In order to inhibit a decrease in the outcoupling efficiency, it is preferable that the cap layer should not include a π-electron deficient heteroaromatic compound. Specifically, the organic compound represented by General Formula (G1) of one embodiment of the present invention which does not have a π-electron deficient heteroaromatic ring as the substituent is preferably used.

The compound used for the cap layer preferably has a high lowest unoccupied molecular orbital (LUMO) level (shallow LUMO level). When a compound having a somewhat high LUMO level is used, the above-described light absorption due to the interaction with the metal might be small. Thus, as long as having a somewhat high LUMO level, a compound having a π-electron deficient heteroaromatic ring can be used for the cap layer. For example, a compound having a LUMO level higher than or equal to −2.7 eV, preferably higher than or equal to −2.6 eV, further preferably higher than or equal to −2.5 eV can be used for the cap layer. The LUMO level of the compound used for the cap layer is preferably higher than the LUMO level of a compound used for the electron-transport layer (e.g., the electron-transport layer in contact with the electrode), for example. In the case where the light-emitting device includes a plurality of (e.g., two or three) electron-transport layers, a compound having a higher LUMO level than compounds included in the plurality of electron-transport layers can be used for the cap layer.

Note that optical interference can be utilized by adjusting the thickness of the cap layer. For example, the organic compound represented by General Formula (G1) of one embodiment of the present invention has a high ordinary refractive index; thus, the thickness of the organic compound at which the light extraction efficiency is maximized can be small and the usage amount of the material can be reduced. Specifically, in the case of a blue light-emitting element, the maximum emission efficiency can be obtained with a cap layer thickness of around 60 nm or in the range from 50 nm to 70 nm.

### “Light-Emitting Layer”

The light-emitting layers (113, 113a, and 113b) include a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables exhibiting different emission colors (e.g., white light emission obtained by a combination of complementary emission colors). Furthermore, one light-emitting layer may have a stacked-layer structure including different light-emitting substances.

The light-emitting layers (113, 113a, and 113b) may each include one or more kinds of organic compounds (e.g., a host material) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (Si level) of the second host material is higher than that of the first host material, and the lowest triplet excitation energy level (T1 level) of the second host material is higher than that of the guest material. Preferably, the lowest triplet excitation energy level (T1 level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With this structure, high efficiency, low voltage, and a long lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the S1 level and the T1 level and functions as a TADF material capable of converting triplet excitation energy into singlet excitation energy. In an example of a preferable combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound of the combination for forming an exciplex. The organic compound described in Embodiment 1 has an electron-transport property and thus can be efficiently used as the first host material. Furthermore, since the organic compound has a hole-transport property, it can be used as the second host material.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

[Light-Emitting Substance that Converts Singlet Excitation Energy into Light]

The following substances that emit fluorescent light (fluorescent substances) can be given as examples of the light-emitting substance that converts singlet excitation energy into light and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-diphenyl-N,N-bis[4-(9-phenyl-9H-fluoren-9-yl)phenyl]pyrene-1,6-diamine (abbreviation: 1,6FLPAPrn), N,N-bis(dibenzofuran-2-yl)-N,N-diphenylpyrene-1,6-diamine (abbreviation: 1,6FrAPrn), N,N-bis(dibenzothiophen-2-yl)-N,N-diphenylpyrene-1,6-diamine (abbreviation: 1,6ThAPrn), N,N-(pyrene-1,6-diyl)bis[(N-phenylbenzo[b]naphtho[1,2-d]furan)-6-amine](abbreviation: 1,6BnfAPrn), N,N-(pyrene-1,6-diyl)bis[(N-phenylbenzo[b]naphtho[1,2-d]furan)-8-amine](abbreviation: 1,6BnfAPrn-02), and N,N-(pyrene-1,6-diyl)bis[(6,N-diphenylbenzo[b]naphtho[1,2-d]furan)-8-amine](abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N,N-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), N-[9,10-bis(biphenyl-2-yl)-2-anthryl]-N,N,N-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, N,N-diphenyl-N,N-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, a pyrenediamine compound such as 1,6FLPAPrn, 1,6mMemFLPAPrn, or 1,6BnfAPrn-03 can be used, for example.

[Light-Emitting Substance that Converts Triplet Excitation Energy into Light]

Examples of the light-emitting substance that converts triplet excitation energy into light and can be used in the light-emitting layer 113 include substances that emit phosphorescent light (phosphorescent substances) and thermally activated delayed fluorescent (TADF) materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), or a rare earth metal complex, for example. Specifically, the phosphorescent substance preferably contains a transition metal element. It is preferable that the phosphorescent substance contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

[Phosphorescent Substance (from 450 nm to 570 nm: Blue or Green)]

As examples of a phosphorescent substance which emits blue or green light and whose emission spectrum has a peak wavelength higher than or equal to 450 nm and lower than or equal to 570 nm, the following substances can be given.

Examples of the phosphorescent substance include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Jr(mpptz-dmp)₃]), tris(5-methyl-3,4-diphenyl-4H-1,2,4-triazolato)iridium(III) (abbreviation: [Jr(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Jr(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Jr(Mptz1-mp)₃]) and tris(1-methyl-5-phenyl-3-propyl-1H-1,2,4-triazolato)iridium(III) (abbreviation: [Jr(Prptz1-Me)₃]); organometallic complexes having an imidazole ring, such as fac-tris[1-(2,6-diisopropylphenyl)-2-phenyl-1H-imidazole]iridium(III) (abbreviation: [Jr(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

[Phosphorescent Substance (from 495 nm to 590 nm: Green or Yellow)]

As examples of a phosphorescent substance which emits green or yellow light and whose emission spectrum has a peak wavelength higher than or equal to 495 nm and lower than or equal to 590 nm, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(JJ) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Jr(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²)iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: [Ir(5mppy-d3)₂(mbfpypy-d3)]), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d6)₂(mbfpypy-iPr-d4)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C²′}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato)(monophenanthroline)terbium(III) (abbreviation: [Tb(acac)₃(Phen)]).

[Phosphorescent Substance (from 570 nm to 750 nm: Yellow or Red)]

As examples of a phosphorescent substance which emits yellow or red light and whose emission spectrum has a peak wavelength higher than or equal to 570 nm and lower than or equal to 750 nm, the following substances can be given.

Examples of the phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-k²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N,C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C²′)iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### [Tadf Material]

Any of materials described below can be used as the TADF material. The TADF material is a material that has a small difference between its S1 and T1 levels (preferably less than or equal to 0.2 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0 eV and less than or equal to 0.2 eV, preferably greater than or equal to 0 eV and less than or equal to 0.1 eV. Delayed fluorescent light by the TADF material refers to light emission having a spectrum similar to that of normal fluorescent light and an extremely long lifetime. The lifetime is longer than or equal to 1×10⁻⁶ seconds, or longer than or equal to 1×10⁻³ seconds.

Note that the TADF material can be also used as an electron-transport material, a hole-transport material, or a host material.

Examples of the TADF material include fullerene, a derivative thereof, an acridine derivative such as proflavine, and eosin. Other examples thereof include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

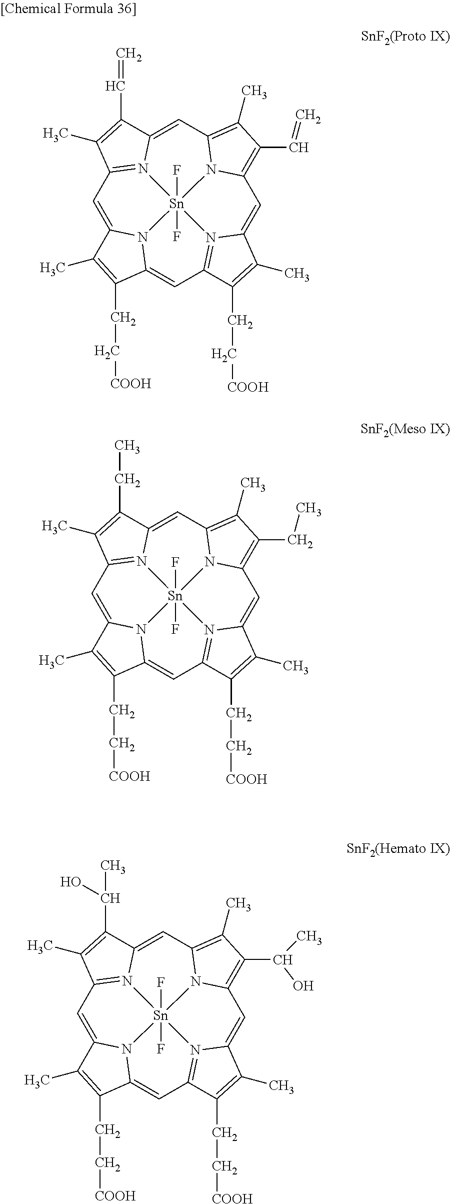

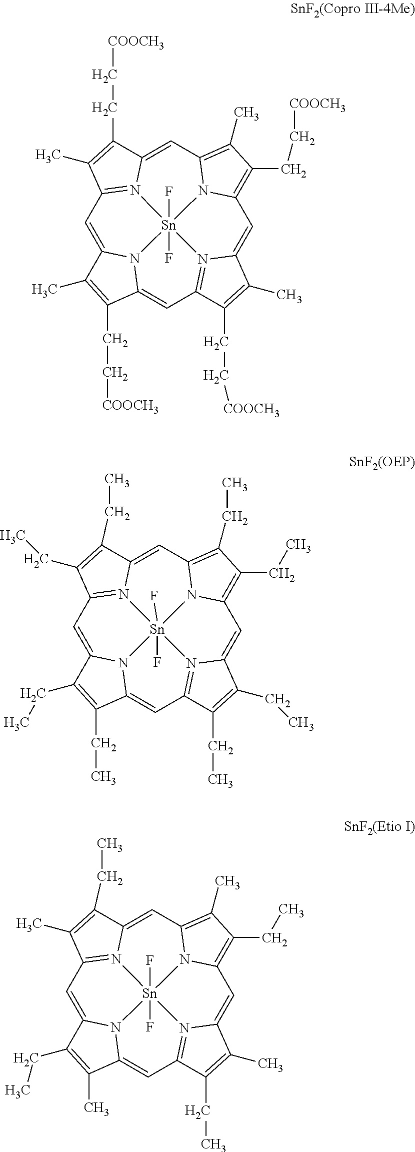

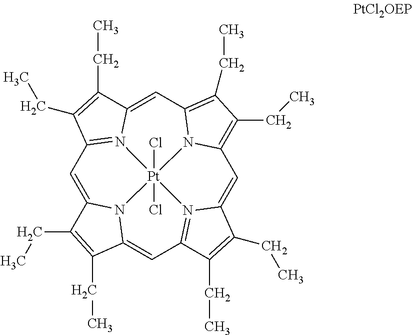

Additionally, a heteroaromatic compound having a p-electron rich heteroaromatic compound and a p-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are enhanced and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

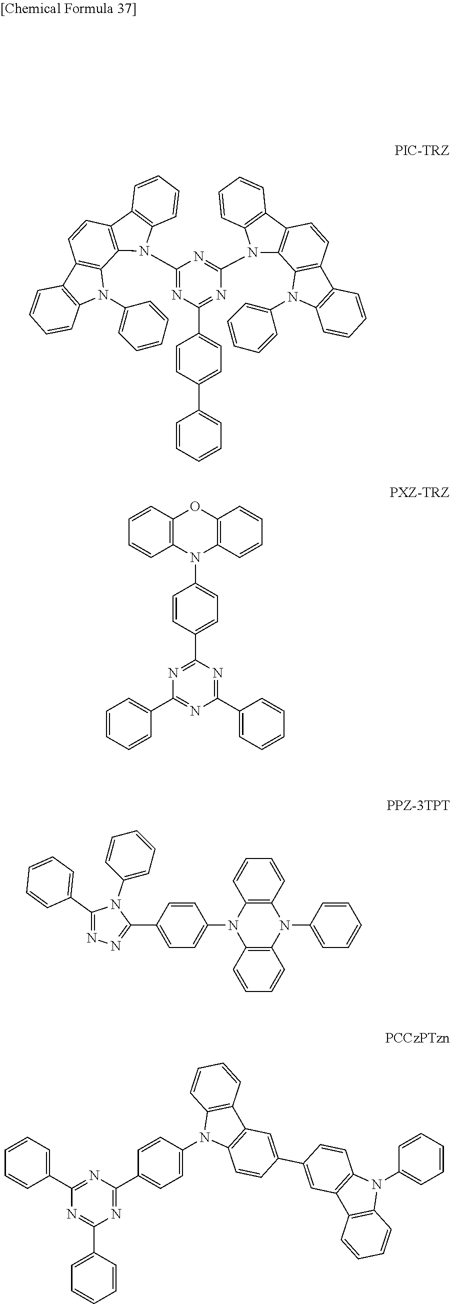 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

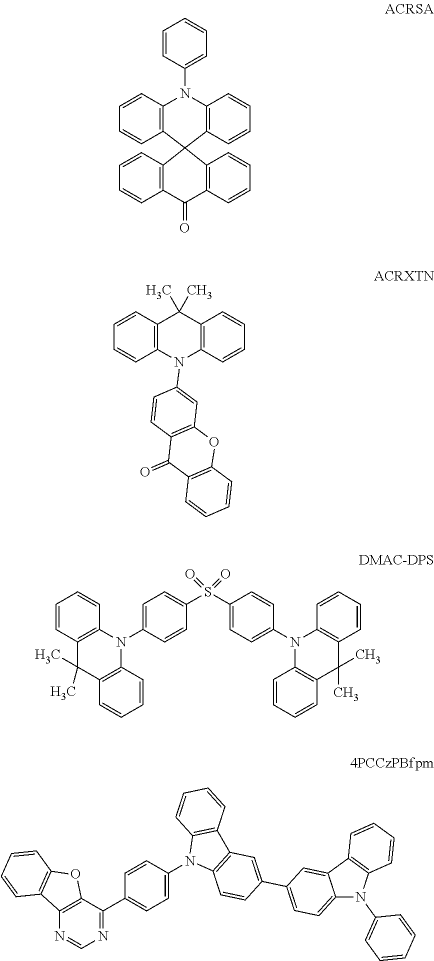 [SMILES: O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; c1ccc(-n2c3ccccc3c3cc(-c4ccc5c(c4)c4ccccc4n5-c4ccc(-c5ncnc6c5oc5ccccc56)cc4)ccc32)cc1]

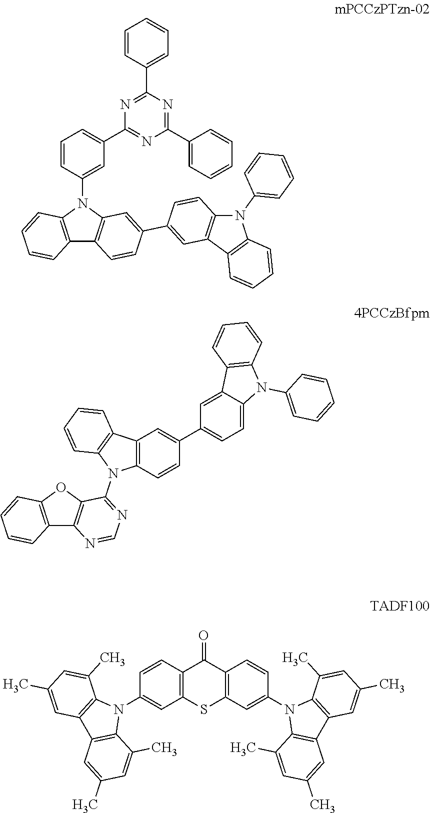

In addition to the above, another example of a material having a function of converting triplet excitation energy into light is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, 113b, and 113c), one or more selected from substances having a larger energy gap than the light-emitting substance (guest material) can be used.

### [Host Material for Fluorescence]

In the case where the light-emitting substance used in the light-emitting layers (113, 113a, 113b, and 113c) is a fluorescent substance, an organic compound (host material) used in combination with the fluorescent substance is preferably an organic compound that has a high energy level in a singlet excited state and has a low energy level in a triplet excited state or an organic compound having a high fluorescence quantum yield. Therefore, the hole-transport material (described above) and the electron-transport material (described below) shown in this embodiment, for example, can be used as long as they are organic compounds that satisfy such a condition. In addition, the organic compound described in Embodiment 1 can be used.

In terms of a preferable combination with the light-emitting substance (fluorescent substance), examples of the organic compound (host material), some of which are mentioned in the above specific examples, include condensed polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N′,N′,N′″,N′″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNβAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: PN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mQNβAnth), 1-{4-[10-(biphenyl-4-yl)-9-anthracenyl]phenyl}-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### [Host Material for Phosphorescence]

In the case where the light-emitting substance used in the light-emitting layers (113, 113a, 113b, and 113c) is a phosphorescent substance, an organic compound having triplet excitation energy (an energy difference between a ground state and a triplet excited state) which is higher than that of the light-emitting substance may be selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance. In addition, the organic compound described in Embodiment 1 can be used.

As the host material, a compound having a naphthalene ring may be used. With a naphthalene ring, the electron-transport property or T_{g} can be increased. Alternatively, a compound having an anthracene ring and a naphthalene ring may be used. The number of naphthalene rings that the host material has is preferably equal to, further preferably less than the number of naphthalene rings that the cap layer material has.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferable, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compounds (the host material and the assist material), some of which are mentioned in the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Specific examples of the aromatic amine and the carbazole derivative, which are organic compounds having a high hole-transport property among the above-described organic compounds, are the same as the specific examples of the hole-transport materials described above, and those materials are preferable as the host material.

Specific examples of the dibenzothiophene derivative and the dibenzofuran derivative, which are organic compounds having a high hole-transport property among the above-described organic compounds, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Other examples of preferable host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property among the above-described organic compounds, include: an organic compound including a heteroaromatic ring having a polyazole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound having a heteroaromatic ring having a pyridine ring such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2,2′-biphenyl-4,4′-diylbis(9-phenyl-1,10-phenanthroline) (abbreviation: PPhen2BP); 2-[3-(dibenzothiophen-4-yl)phenyl]dibenzo[f,h]quinoxaline (abbreviation: 2mDBTPDBq-II); 2-[3′-(dibenzothiophen-4-yl)biphenyl-3-yl]dibenzo[f,h]quinoxaline (abbreviation: 2mDBTBPDBq-II); 2-[3′-(9H-carbazol-9-yl)biphenyl-3-yl]dibenzo[f,h]quinoxaline (abbreviation: 2mCzBPDBq); 2-[4-(3,6-diphenyl-9H-carbazol-9-yl)phenyl]dibenzo[f,h]quinoxaline (abbreviation: 2CzPDBq-III); 7-[3-(dibenzothiophen-4-yl)phenyl]dibenzo[f;h]quinoxaline (abbreviation: 7mDBTPDBq-II); 6-[3-(dibenzothiophen-4-yl)phenyl]dibenzo[f,h]quinoxaline (abbreviation: 6mDBTPDBq-II); 2-{4-[9,10-di(2-naphthyl)-2-anthryl]phenyl}-1-phenyl-1H-benzimidazole (abbreviation: ZADN); and 2-[4′-(9-phenyl-9H-carbazol-3-yl)-3,1′-biphenyl-1-yl]dibenzo[f,h]quinoxaline (abbreviation: 2mpPCBPDBq). Such organic compounds are preferred as the host material.

Specific examples of the pyridine derivative, the diazine derivative (e.g., the pyrimidine derivative, the pyrazine derivative, and the pyridazine derivative), the triazine derivative, and the furodiazine derivative, which are organic compounds having a high electron-transport property among the above organic compounds, include organic compounds including a heteroaromatic ring having a diazine ring such as 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′: 4,5]furo[2,3-b]pyrazine (abbreviation: 9mDBtBPNfpr), 9-[3′-(dibenzothiophen-4-yl)biphenyl-4-yl]naphtho[1′,2′: 4,5]furo[2,3-b]pyrazine (abbreviation: 9pmDBtBPNfpr), 11-[3′-(dibenzothiophen-4-yl)biphenyl-3-yl]phenanthro[9′,10′: 4,5]furo[2,3-b]pyrazine (abbreviation: 11mDBtBPPnfpr), 11-[3′-(dibenzothiophen-4-yl)biphenyl-4-yl]phenanthro[9′,10′: 4,5]furo[2,3-b]pyrazine, 11-[3′-(9H-carbazol-9-yl)biphenyl-3-yl]phenanthro[9′,10′: 4,5]furo[2,3-b]pyrazine, 12-(9′-phenyl-3,3′-bi-9H-carbazol-9-yl)phenanthro[9′,10′: 4,5]furo[2,3-b]pyrazine (abbreviation: 12PCCzPnfpr), 9-[3′-(9-phenyl-9H-carbazol-3-yl)biphenyl-4-yl]naphtho[1′,2′: 4,5]furo[2,3-b]pyrazine (abbreviation: 9pmPCBPNfpr), 9-(9′-phenyl-3,3′-bi-9H-carbazol-9-yl)naphtho[1′,2′: 4,5]furo[2,3-b]pyrazine (abbreviation: 9PCCzNfpr), 10-(9′-phenyl-3,3′-bi-9H-carbazol-9-yl)naphtho[1′,2′: 4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′: 4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′: 4,5]furo[2,3-b]pyrazine (abbreviation: 9mFDBtPNfpr), 9-[3′-(6-phenyldibenzothiophen-4-yl)biphenyl-3-yl]naphtho[1′,2′: 4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′: 4,5]furo[2,3-b]pyrazine (abbreviation: 9mPCCzPNfpr), 9-[3′-(2,8-diphenyldibenzothiophen-4-yl)biphenyl-3-yl]naphtho[1′,2′: 4,5]furo[2,3-b]pyrazine, 11-[3′-(2,8-diphenyldibenzothiophen-4-yl)biphenyl-3-yl]phenanthro[9′,10′: 4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′: 4′,1″-terphenyl)-4-yl]-1-dibenzofuranyl}-1,3,5-triazine (abbreviation: mBP-TPDBfTzn), 6-(biphenyl-3-yl)-4-[3,5-bis(9H-carbazol-9-yl)phenyl)-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm), and those materials are preferable as the host material.

Specific examples of the metal complex, which is an organic compound having a high electron-transport property among the above organic compounds, include zinc- or aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. These metal complexes are preferable as the host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) are preferable as the host material.

Furthermore, any of the following organic compounds having a diazine ring and bipolar properties, which have a high hole-transport property and a high electron-transport property, can be used as the host material: 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <<Hole-Injection Layer>>

The hole-injection layers (111, 111a, and 111b) inject holes from the first electrode 101 serving as the anode and the charge-generation layers (106, 106a, and 106b) to the organic compound layers (103, 103a, and 103b) and include an organic acceptor material or a material having a high hole-injection property.

Note that in the hole-injection layers (111, 111a, and 111b), it is further preferable that an organic compound having a hole-transport property used for a composite material have a relatively low highest occupied molecular orbital (HOMO) level of higher than or equal to −5.7 eV and lower than or equal to −5.4 eV. When the organic compound having a hole-transport property that is used in the composite material has a relatively low HOMO level, holes can be easily injected into the hole-transport layer to easily provide a light-emitting device having a long lifetime. In addition, when the organic compound having a hole-transport property that is used in the composite material has a relatively low HOMO level, induction of holes can be inhibited properly, so that the light-emitting device can have a longer lifetime.

The hole-injection layers (111, 111a, and 111b) have a function of lowering a barrier for hole injection from one of the pair of electrodes (the first electrode 101 or the second electrode 102) to promote hole injection and is formed using a transition metal oxide, a phthalocyanine derivative, or an aromatic amine, for example. As examples of the transition metal oxide, molybdenum oxide, vanadium oxide, ruthenium oxide, tungsten oxide, and manganese oxide can be given. As examples of the phthalocyanine derivative, phthalocyanine and metal phthalocyanine can be given. As examples of the aromatic amine, a benzidine derivative and a phenylenediamine derivative can be given. It is also possible to use a high molecular compound such as polythiophene or polyaniline; a typical example thereof is poly(ethylenedioxythiophene)/poly(styrenesulfonic acid), which is self-doped polythiophene.

As each of the hole-injection layers (111, 111a, and 111b), a layer containing a composite material of a hole-transport material and a material having a property of accepting electrons from the hole-transport material can also be used. Alternatively, a stack of a layer containing a material having an electron-accepting property and a layer containing a hole-transport material may also be used. In a steady state or in the presence of an electric field, electric charge can be transferred between these materials. As examples of the material having an electron-accepting property, organic acceptors such as a quinodimethane derivative, a chloranil derivative, and a hexaazatriphenylene derivative can be given. A specific example is a compound having an electron-withdrawing group (a halogen group or a cyano group), such as 7,7,8,8-tetracyano-2,3,5,6-tetrafluoroquinodimethane (abbreviation: F₄-TCNQ), chloranil, or 2,3,6,7,10,11-hexacyano-1,4,5,8,9,12-hexaazatriphenylene (abbreviation: HAT-CN). Alternatively, a transition metal oxide such as an oxide of a metal from Group 4 to Group 8 can be used. Specifically, vanadium oxide, niobium oxide, tantalum oxide, chromium oxide, molybdenum oxide, tungsten oxide, manganese oxide, rhenium oxide, or the like can be used. In particular, molybdenum oxide is preferable because it is stable in the air, has a low hygroscopic property, and is easily handled.

A material having a hole-transport property higher than an electron-transport property can be used as a hole-transport material, and a material having a hole mobility higher than or equal to 1×10⁻⁶ cm²/Vs is preferably used. Specifically, any of the aromatic amine, carbazole derivative, aromatic hydrocarbon, stilbene derivative, and the like described as examples of the hole-transport material that can be used in the light-emitting layer 113 can be used. The organic compound represented by General Formula (G1) of one embodiment of the present invention is also a carbazole derivative. Furthermore, the hole-transport material may be a high molecular compound.

### <<Hole-Transport Layer>>

The hole-transport layers (112, 112a, and 112b) contain a hole-transport material and can be formed using any of the hole-transport materials given as examples of the material of the hole-injection layers (111, 111a, and 111b). In order that the hole-transport layers (112, 112a, and 112b) can have a function of transporting holes injected into the hole-injection layers (111, 111a, and 111b) to the light-emitting layers (113, 113a, and 113b), the HOMO level of the hole-transport layers (112, 112a, and 112b) is preferably equal or close to the HOMO level of the hole-injection layers (111, 111a, and 111b).

As the hole-transport material, a substance having a hole mobility higher than or equal to 1×10⁻⁶ cm²/Vs is preferably used. Note that other substances may also be used as long as their hole-transport properties are higher than their electron-transport properties. The layer including a substance having a high hole-transport property is not limited to a single layer and may be a stack of two or more layers each containing any of the above substances.

The ordinary refractive index of the hole-transport layer or the hole-injection layer is preferably lower than that of the cap layer. The ordinary refractive index of the hole-transport layer or the hole-injection layer is preferably lower than that of the light-emitting layer. Specifically, the ordinary refractive indices of the deposited films of the hole-transport material, the host material of the light-emitting layer, and the material of the cap layer are compared with each other. The ordinary refractive indices of the films preferably satisfy the relation: the hole-transport material<the host material<the material of the cap layer, in which case the extraction efficiency can be increased.

Note that the total thickness (thickness m) of the layers sandwiched between the reflective electrode and the light-emitting layer (e.g., the transparent electrode, the hole-injection layer, the hole-transport layer, and the electron-blocking layer) is preferably a thickness enabling optical interference (microcavity), in which case light extraction efficiency can be increased. In this case, it is further preferable that the layer with the thickness m have a stacked-layer structure of a layer with a low ordinary refractive index (a layer L) and a layer with a higher ordinary refractive index (a layer H) than the layer L, and that light incident from the light-emitting layer be reflected at the interface between the layer L and the layer H and amplified by optical interference. In this case, the difference in ordinary refractive index between the layer L and the layer H is preferably greater than or equal to 0.1, further preferably greater than or equal to 0.2, still further preferably greater than or equal to 0.3 at the emission wavelength of the light-emitting element, in which case the interface reflection is increased. Note that the organic compound represented by General Formula (G1) of one embodiment of the present invention has a high refractive index and thus can be used for the layer H.

### <<Electron-Transport Layer>>

The electron-transport layers (114, 114a, and 114b) have a function of transporting, to the light-emitting layer 113, electrons injected from the other of the pair of electrodes (the first electrode 101 or the second electrode 102) through the electron-injection layers (115, 115a, and 115b). As the electron-transport material, a material having an electron-transport property higher than a hole-transport property can be used, and a material having an electron mobility higher than or equal to 1×10⁻⁶ cm²/Vs is preferable. As the compound which easily accepts electrons (the material having an electron-transport property), a compound having a π-electron deficient heteroaromatic ring skeleton such as a nitrogen-containing heteroaromatic compound, an organometallic complex, or the like can be used. Specific examples include an organometallic complex having a quinoline ligand, a benzoquinoline ligand, an oxazole ligand, or a thiazole ligand, which is described as the electron-transport material usable for the light-emitting layer 113. In addition, an oxadiazole derivative, a triazole derivative, a benzimidazole derivative, a quinoxaline derivative, a dibenzoquinoxaline derivative, a phenanthroline derivative, a pyridine derivative, a bipyridine derivative, a pyrimidine derivative, a triazine derivative, or the like can be used. As the electron-transport material, a substance having an electron mobility higher than or equal to 1×10⁻⁶ cm²/Vs is preferably used. Note that other substances may also be used for the electron-transport layer as long as their electron-transport properties are higher than their hole-transport properties. Each of the electron-transport layers (114, 114a, and 114b) is not limited to a single layer and may be a stack of two or more layers each containing any of the above substances.

A compound having a naphthalene ring may be used as the electron-transport material. For example, naphthalene has higher reactivity than benzene and is advantageous in terms of synthesis. The compound containing naphthalene can have a high electron-transport property or a high T_{g}. When the ordinary refractive index of the electron-transport layer is low, the light extraction efficiency can be high. Thus, the number of naphthalene rings that the electron-transport material has is preferably less than or equal to two, further preferably one. The electron-transport layer preferably has a lower ordinary refractive index than the cap layer. Thus, the number of naphthalene rings included in the electron-transport material is preferably equal to, further preferably less than the number of naphthalene rings included in the material of the cap layer. As the electron-transport material, an azine compound having a naphthalene ring (a compound having a triazine skeleton or a pyrimidine skeleton) can be used, for example. Note that as long as the light extraction efficiency is sufficient, the difference in ordinary refractive index between the electron-transport layer and the cap layer may be small. In that case, the same organic compound can also be used for the electron-transport layer and the cap layer.

A compound having a cyano group may be used as the electron-transport material. When a compound having a cyano group is used in the electron-transport layer, the driving voltage of the device is reduced, efficiency is improved, or the element lifetime is increased in some cases. However, a large number of cyano groups may disturb the carrier balance in the device; thus, the number of cyano groups is preferably less than or equal to two, further preferably less than or equal to one. The number of cyano groups included in the electron-transport material is equal to or preferably smaller than the number of cyano groups included in the material of the cap layer. As the electron-transport material, an azine compound including a cyano group or an azine compound including a cyano group and a naphthalene ring can be used, for example.

As the electron-transport material, a compound having an anthracene ring (also referred to as an anthracene compound) can be used. When a structure having a naphthyl ring is used, the electron-transport property or T_{g} can be improved. Therefore, a structure having an anthracene ring and a naphthalene ring may be used. Note that the number of naphthalene rings included in the electron-transport material is preferably smaller than the number of naphthalene rings included in the cap layer material.

The ordinary refractive index of the electron-transport layer or the electron-injection layer is preferably lower than that of the light-emitting layer. Taking the cap layer in consideration, the ordinary refractive indices of the films preferably satisfy the relation: the electron-transport material<the host material<the material of the cap layer, in which case the extraction efficiency can be increased.

Between the electron-transport layer (114, 114a, or 114b) and the light-emitting layer (113, 113a, or 113b), a layer that controls transfer of electron carriers may be provided. This is a layer formed by addition of a small amount of a substance having a high electron-trapping property to a material having a high electron-transport property as described above, and the layer is capable of adjusting carrier balance by suppressing transport of electron carriers. Such a structure is very effective in inhibiting a problem (such as a reduction in element lifetime) caused when electrons pass through the light-emitting layer.

### <<Electron-Injection Layer>>

The electron-injection layers (115, 115a, and 115b) have a function of reducing a barrier for electron injection from the second electrode 102 to promote electron injection and can be formed using a Group 1 metal or a Group 2 metal, or an oxide, a halide, or a carbonate of any of these metals, for example. Alternatively, a composite material containing an electron-transport material described above and a material having a property of donating electrons to the electron-transport material can also be used. As examples of the material having an electron-donating property, a Group 1 metal, a Group 2 metal, an oxide of any of these metals, and the like can be given. Specifically, an alkali metal, an alkaline earth metal, or a compound thereof, such as lithium fluoride (LiF), sodium fluoride (NaF), cesium fluoride (CsF), calcium fluoride (CaF₂), or lithium oxide (LiOₓ), can be used. Alternatively, a rare earth metal compound like erbium fluoride (ErF₃) can be used. Electride may also be used for the electron-injection layer 115. Examples of the electride include a substance in which electrons are added at high concentration to calcium oxide-aluminum oxide. The electron-injection layers (115, 115a, and 115b) can be formed using the substance that can be used for the electron-transport layers (114, 114a, and 114b).

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. In this case, the organic compound is preferably a material that is excellent in transporting the generated electrons. Specifically, the above-described substances for forming the electron-transport layer 114 (e.g., an organometallic complex or a heteroaromatic compound) can be used, for example. As the electron donor, a substance showing an electron-donating property with respect to an organic compound can be used. Specifically, it is preferable to use an alkali metal, an alkaline earth metal, or a rare earth metal, such as lithium, sodium, cesium, magnesium, calcium, erbium, or ytterbium. It is also preferable to use an alkali metal oxide or an alkaline earth metal oxide, such as lithium oxide, calcium oxide, or barium oxide. Alternatively, a Lewis base such as magnesium oxide can be used. Further alternatively, an organic compound such as tetrathiafulvalene (abbreviation: TTF) can be used.

Note that the light-emitting layer, the hole-injection layer, the hole-transport layer, the electron-transport layer, and the electron-injection layer described above can each be formed by an evaporation method (including a vacuum evaporation method), an ink-jet method, a coating method, a gravure printing method, or the like. Besides the above-mentioned materials, an inorganic compound such as a quantum dot or a high molecular compound (e.g., an oligomer, a dendrimer, or a polymer) may be used in the light-emitting layer, the hole-injection layer, the hole-transport layer, the electron-transport layer, and the electron-injection layer.

The quantum dot may be a colloidal quantum dot, an alloyed quantum dot, a core-shell quantum dot, or a core quantum dot, for example. The quantum dot containing elements belonging to Groups 2 and 16, elements belonging to Groups 13 and 15, elements belonging to Groups 13 and 17, elements belonging to Groups 11 and 17, or elements belonging to Groups 14 and 15 may be used. Alternatively, the quantum dot containing an element such as cadmium (Cd), selenium (Se), zinc (Zn), sulfur (S), phosphorus (P), indium (In), tellurium (Te), lead (Pb), gallium (Ga), arsenic (As), or aluminum (Al) may be used.

### <<Pair of Electrodes>>

The first electrode 101 and the second electrode 102 function as an anode and a cathode of the light-emitting device. The first electrode 101 and the second electrode 102 can be formed using a metal, an alloy, or a conductive compound, a mixture or a stack thereof, or the like.

One of the first electrode 101 and the second electrode 102 is preferably formed using a conductive material having a function of reflecting light. Examples of the conductive material include aluminum (Al), an alloy containing Al, and the like. Examples of the alloy containing Al include an alloy containing Al and L (L represents one or more of titanium (Ti), neodymium (Nd), nickel (Ni), and lanthanum (La)), such as an alloy containing Al and Ti and an alloy containing Al, Ni, and La. Aluminum (Al) has low resistance and high light reflectivity. Aluminum (Al) is included in earth's crust in large amount and is inexpensive; therefore, it is possible to reduce costs for manufacturing a light-emitting element with aluminum. Alternatively, silver (Ag), an alloy of Ag and N(N represents one or more of yttrium (Y), Nd, magnesium (Mg), ytterbium (Yb), Al, Ti, gallium (Ga), zinc (Zn), indium (In), tungsten (W), manganese (Mn), tin (Sn), iron (Fe), Ni, copper (Cu), palladium (Pd), iridium (Ir), and gold (Au)), or the like may be used. Examples of the alloy containing silver (Ag) include an alloy containing silver, palladium, and copper, an alloy containing silver and copper, an alloy containing silver and magnesium, an alloy containing silver and nickel, an alloy containing silver and gold, an alloy containing silver and ytterbium, and the like. Besides, a transition metal such as tungsten, chromium (Cr), molybdenum (Mo), copper, or titanium can be used.

Light emitted from the light-emitting layer is extracted through the first electrode 101 and/or the second electrode 102. Thus, at least one of the first electrode 101 and the second electrode 102 is preferably formed using a conductive material having a function of transmitting light. As the conductive material, a conductive material having a visible light transmittance higher than or equal to 40% and lower than or equal to 100%, preferably higher than or equal to 60% and lower than or equal to 100%, and a resistivity lower than or equal to 1×10⁻² Ω·cm can be used.

The first electrode 101 and the second electrode 102 may each be formed using a conductive material having functions of transmitting light and reflecting light. As the conductive material, a conductive material having a visible light reflectivity higher than or equal to 20% and lower than or equal to 80%, preferably higher than or equal to 40% and lower than or equal to 70%, and a resistivity lower than or equal to 1×10⁻² Ω·cm can be used. For example, one or more kinds of conductive metals and alloys, conductive compounds, and the like can be used. Specifically, a metal oxide such as indium tin oxide (hereinafter, referred to as ITO), indium tin oxide containing silicon or silicon oxide (ITSO), indium oxide-zinc oxide (indium zinc oxide), indium oxide-tin oxide containing titanium, indium titanium oxide, or indium oxide containing tungsten oxide and zinc oxide can be used. A metal thin film having a thickness that allows transmission of light (preferably, a thickness greater than or equal to 1 nm and less than or equal to 30 nm) can also be used. As the metal, Ag, an alloy of Ag and Al, an alloy of Ag and Mg, an alloy of Ag and Au, an alloy of Ag and Yb, or the like can be used.

In this specification and the like, as the material having a function of transmitting light, a material having a function of transmitting visible light and having conductivity is used. Examples of the material include, in addition to the above-described oxide conductor typified by ITO, an oxide semiconductor and an organic conductor containing an organic substance. Examples of the organic conductor containing an organic substance include a composite material in which an organic compound and an electron donor (donor) are mixed and a composite material in which an organic compound and an electron acceptor (acceptor) are mixed. Alternatively, an inorganic carbon-based material such as graphene may be used. The resistivity of the material is preferably lower than or equal to 1×10⁵ Ω·cm, further preferably lower than or equal to 1×10₄ Ω·cm.

The first electrode 101 and/or the second electrode 102 may be formed by stacking two or more of the materials described above.

In order to improve the light extraction efficiency, a material whose refractive index is higher than that of an electrode having a function of transmitting light may be formed in contact with the electrode. The material may be electrically conductive or non-conductive as long as it has a function of transmitting visible light. In addition to the oxide conductors described above, an oxide semiconductor and an organic substance are given as the examples of the material. Examples of the organic substance include the materials for the light-emitting layer, the hole-injection layer, the hole-transport layer, the electron-transport layer, and the electron-injection layer. Alternatively, an inorganic carbon-based material or a metal film thin enough to transmit light can be used. Further alternatively, stacked layers with a thickness of several nanometers to several tens of nanometers may be used.

In the case where the first electrode 101 or the second electrode 102 functions as the cathode, the electrode preferably contains a material having a low work function (lower than or equal to 3.8 eV). For example, it is possible to use an element belonging to Group 1 or 2 of the periodic table (e.g., an alkali metal such as lithium, sodium, or cesium, an alkaline earth metal such as calcium or strontium, or magnesium), an alloy containing any of these elements (e.g., Ag—Mg or Al—Li), a rare earth metal such as europium (Eu) or Yb, an alloy containing any of these rare earth metals, an alloy containing aluminum or silver, or the like.

When the first electrode 101 or the second electrode 102 is used as an anode, a material with a high work function (4.0 eV or higher) is preferably used.

The first electrode 101 and the second electrode 102 may be a stacked layer of a conductive material having a function of reflecting light and a conductive material having a function of transmitting light. This structure is preferably employed, in which case the first electrode 101 and the second electrode 102 can have a function of adjusting the optical path length so that light of a desired wavelength emitted from each light-emitting layer resonates and is intensified.

As the method for forming the first electrodes 101 and 102, a sputtering method, an evaporation method, a printing method, a coating method, a molecular beam epitaxy (MBE) method, a chemical vapor deposition (CVD) method, a pulsed laser deposition method, an atomic layer deposition (ALD) method, or the like can be used as appropriate.

### <<Charge-Generation Layer>>

The charge-generation layer 106 has a function of injecting electrons into the organic compound layer 103a and injecting holes into the organic compound layer 103b when a voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these structures may be stacked. Furthermore, an electron-relay layer may be provided between the p-type layer and the electron-injection buffer layer. Note that forming the charge-generation layer 106 with the use of any of the above materials can inhibit an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films including the respective materials may be used.

In the case where the charge-generation layer 106 is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a p-type layer and an electron-injection buffer layer in the charge-generation layer 106, the electron-relay layer includes at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer is preferably higher than or equal to −5.00 eV, further preferably higher than or equal to −5.00 eV and lower than or equal to −3.00 eV, still further preferably higher than or equal to −4.30 eV and lower than or equal to −3.00 eV, yet still further preferably higher than or equal to −4.30 eV and lower than or equal to −3.30 eV. Note that as the substance having an electron-transport property in the electron-relay layer, a phthalocyanine-based material or an organometallic complex having a metal-oxygen bond and an aromatic ligand is preferably used.

Specifically, for the organic compound that can be used for the electron-relay layer, an alkylamine skeleton having 1 to 6 carbon atoms can be used. Specific examples of the organic compound include an organic compound having a basic skeleton such as an acetamidine skeleton, a guanidine skeleton, a pyrrolidine skeleton, or the like, represented by Structural Formulae (401) to (404). In particular, organic compounds each having a guanidine skeleton, which are represented by Structural Formulae (403) and (404), are preferable because of their high basicity. Furthermore, such an organic compound having an alkylamine skeleton preferably has an electron-transport property as a substituent and preferably has one or more of an aromatic hydrocarbon skeleton, a π-electron deficient heteroaromatic ring skeleton, and a nitrogen-containing heteroaromatic skeleton. Specific examples include a benzene skeleton, a fluorene skeleton, a naphthalene skeleton, an anthracene skeleton, a phenanthrene skeleton, a triphenylene skeleton, a pyrene skeleton, a polyazole skeleton, a pyridine skeleton, a pyrimidine skeleton, a pyrazine skeleton, a diazine skeleton, a triazine skeleton, a quinoline skeleton, a quinazoline skeleton, a quinoxaline skeleton, a phenanthroline skeleton, and a dibenzoquinoxaline skeleton.

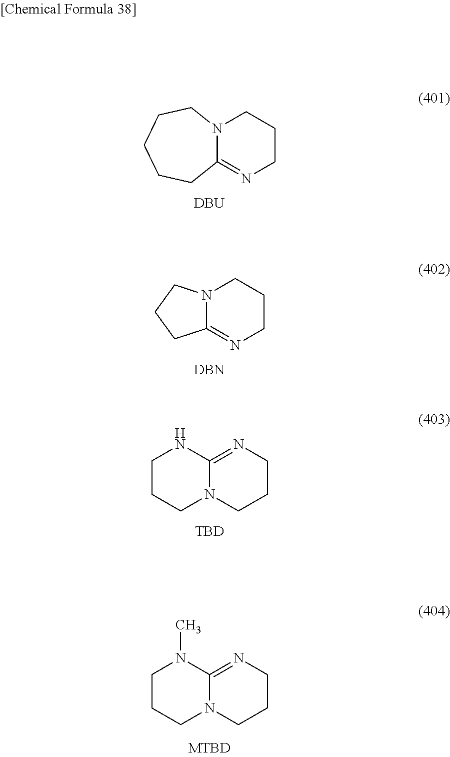

It is preferable that the organic compound be specifically an organic compound that has a bicyclo ring structure having 2 or more nitrogen atoms as some of the element atoms that constitute the ring and a heteroaromatic ring having 2 to 30 carbon atoms in the ring or an aromatic hydrocarbon ring having 6 to 30 carbon atoms in the ring, and more specifically be an organic compound that has a 1,3,4,6,7,8-hexahydro-2H-pyrimido[1,2-a]pyrimidine skeleton and a heteroaromatic ring having 2 to 30 carbon atoms in the ring or an aromatic hydrocarbon ring having 6 to 30 carbon atoms in the ring. An organic compound that has a bicyclo ring structure having 2 or more nitrogen atoms in the ring and a heteroaromatic ring having 2 to 30 carbon atoms in the ring, more specifically an organic compound that has a 1,3,4,6,7,8-hexahydro-2H-pyrimido[1,2-a]pyrimidine skeleton and a heteroaromatic ring having 2 to 30 carbon atoms in the ring is further preferable. An organic compound having a guanidine skeleton is preferable, and an organic compound in which the bicyclo ring structure having 2 or more nitrogen atoms in the ring is a molecular structure including a guanidine skeleton is further preferable.

Further specifically, the organic compound is preferably an organic compound represented by General Formula (R1) below.

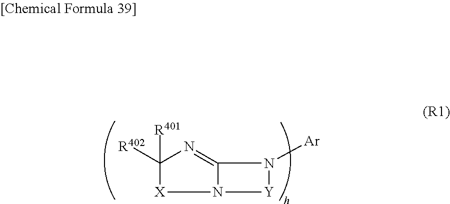

In the organic compound represented by General Formula (R1) above, X represents a group represented by General Formula (R1-1) below, and Y represents a group represented by General Formula (R1-2) below. Furthermore, R⁴⁰¹ and R⁴⁰² each independently represent hydrogen (including deuterium), h represents an integer of 1 to 6, and Ar represents a substituted or unsubstituted heteroaromatic ring having 2 to 30 carbon atoms in the ring or a substituted or unsubstituted aromatic hydrocarbon ring having 6 to 30 carbon atoms in the ring. Ar is preferably the substituted or unsubstituted heteroaromatic ring having 2 to 30 carbon atoms in the ring.

Furthermore, the organic compound can have a high sublimation property when h is an integer of 1 or 2.

Ar preferably has one or more of an aromatic ring skeleton, a π-electron deficient heteroaromatic ring skeleton, and a nitrogen-containing heteroaromatic skeleton. Specifically, a substituent having a benzene skeleton, a naphthalene skeleton, an anthracene skeleton, a phenanthrene skeleton, a polyazole skeleton, a pyridine skeleton, a pyrimidine skeleton, a pyrazine skeleton, a diazine skeleton, a triazine skeleton, a quinoline skeleton, a quinazoline skeleton, a quinoxaline skeleton, a phenanthroline skeleton, or a dibenzoquinoxaline skeleton is preferable.

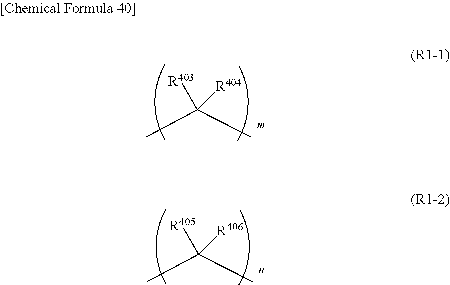

In General Formulae (R1-1) and (R1-2) above, R⁴⁰³ to R⁴⁰⁶ each independently represent hydrogen (including deuterium), m represents an integer of 0 to 4, n represents an integer of 1 to 5, and m+1≥n is satisfied. In the case where m or n is 2 or more, R⁴⁰³s may be the same as or different from each other, and the same applies to R⁴⁰⁴s, R⁴⁰⁵s, and R⁴⁰⁶s. In the case where m is 0, carbon (C) and nitrogen (N) are preferably bonded to each other in General Formula (R1) above.

The organic compound represented by General Formula (R1) above is preferably any one of compounds represented by General Formulae (R2-1) to (R2-6) below.

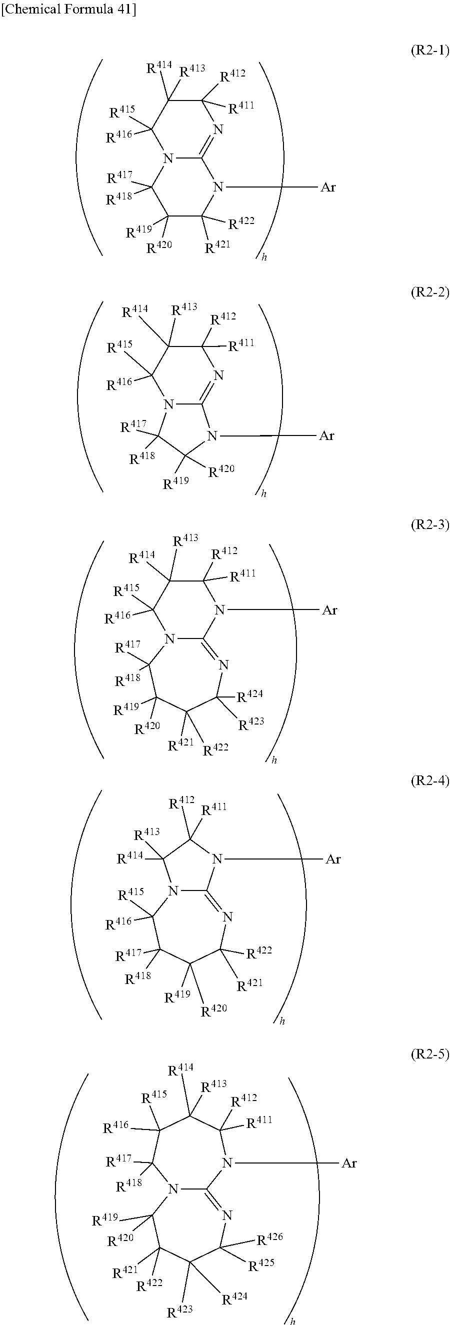

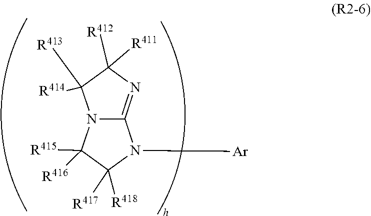

R⁴¹¹ to R⁴²⁶ each independently represent hydrogen (including deuterium), h represents an integer of 1 to 6, and Ar represents a substituted or unsubstituted heteroaromatic ring having 2 to 30 carbon atoms in the ring or a substituted or unsubstituted aromatic hydrocarbon ring having 6 to 30 carbon atoms in the ring. Ar is preferably the substituted or unsubstituted heteroaromatic ring having 2 to 30 carbon atoms in the ring.

In General Formulae (R1) and (R2-1) to (R2-6) above, the substituted or unsubstituted heteroaromatic ring having 2 to 30 carbon atoms in the ring or the substituted or unsubstituted aromatic hydrocarbon ring having 6 to 30 carbon atoms in the ring that is represented by Ar is specifically a pyridine ring, a bipyridine ring, a pyrimidine ring, a bipyrimidine ring, a pyrazine ring, a bipyrazine ring, a triazine ring, a quinoline ring, an isoquinoline ring, a benzoquinoline ring, a phenanthroline ring, a quinoxaline ring, a benzoquinoxaline ring, a dibenzoquinoxaline ring, an azofluorene ring, a diazofluorene ring, a carbazole ring, a benzocarbazole ring, a dibenzocarbazole ring, a dibenzofuran ring, a benzonaphthofuran ring, a dinaphthofuran ring, a dibenzothiophene ring, a benzonaphthothiophene ring, a dinaphthothiophene ring, a benzofuropyridine ring, a benzofuropyrimidine ring, a benzothiopyridine ring, a benzothiopyrimidine ring, a naphthofuropyridine ring, a naphthofuropyrimidine ring, a naphthothiopyridine ring, a naphthothiopyrimidine ring, an acridine ring, a xanthene ring, a phenothiazine ring, a phenoxazine ring, a phenazine ring, a triazole ring, an oxazole ring, an oxadiazole ring, a thiazole ring, a thiadiazole ring, an imidazole ring, a benzimidazole ring, a pyrazole ring, a pyrrole ring, or the like. In General Formulae (R1) and (R2-1) to (R2-6) above, the substituted or unsubstituted aromatic hydrocarbon ring having 6 to 30 carbon atoms in the ring that is represented by Ar is specifically a benzene ring, a naphthalene ring, a fluorene ring, a dimethylfluorene ring, a diphenylfluorene ring, a spirofluorene ring, an anthracene ring, a phenanthrene ring, a triphenylene ring, a pyrene ring, a tetracene ring, a chrysene ring, a benzo[a]anthracene ring, or the like, and is especially preferably the ring represented by any one of Structural Formulae (Ar-401) to (Ar-427) below.

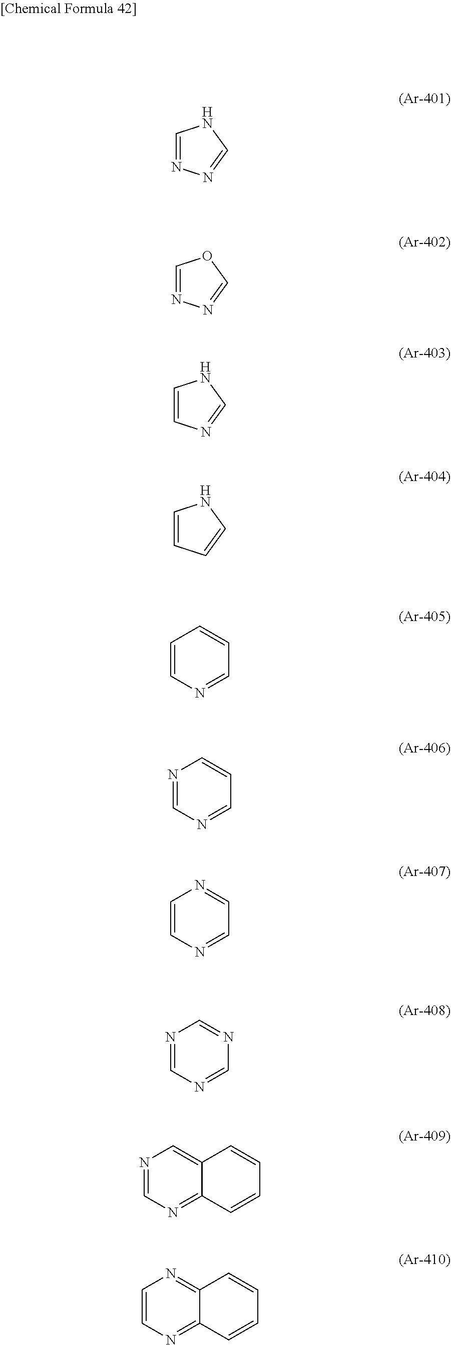

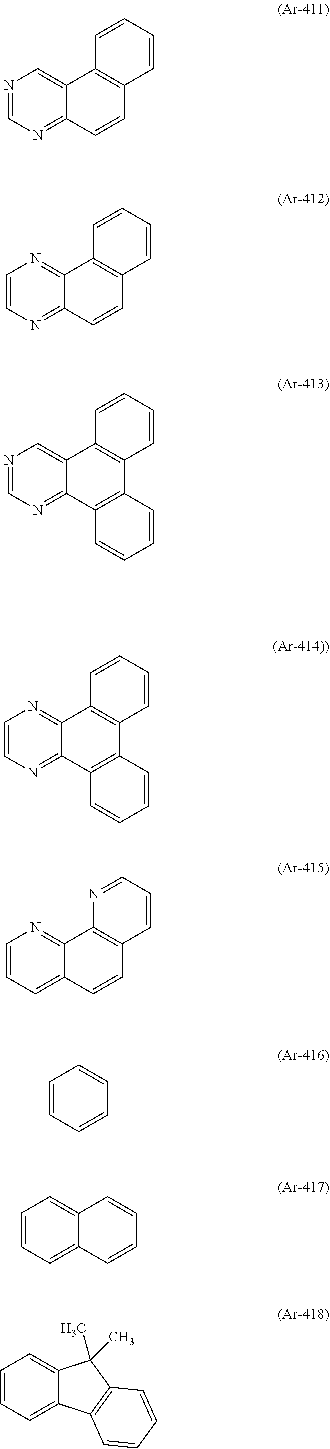

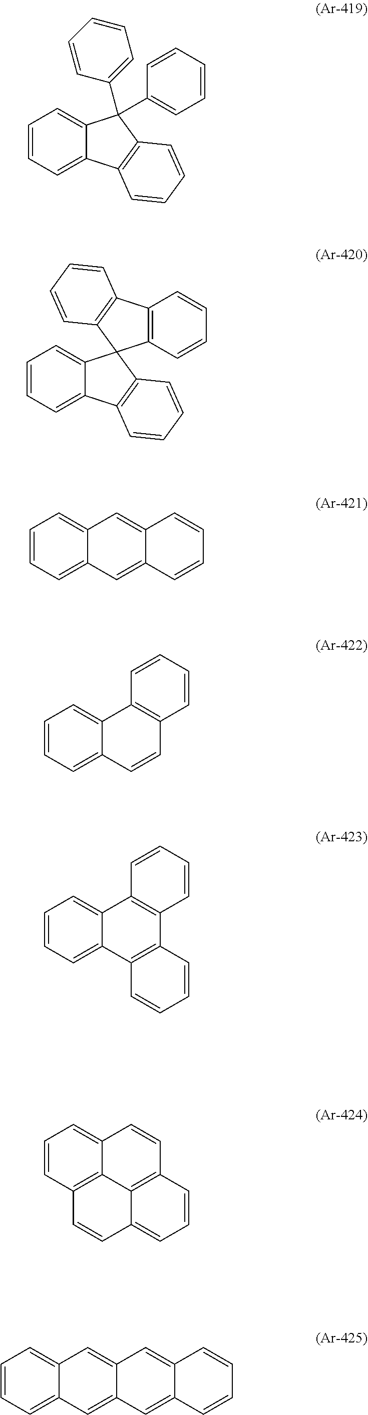

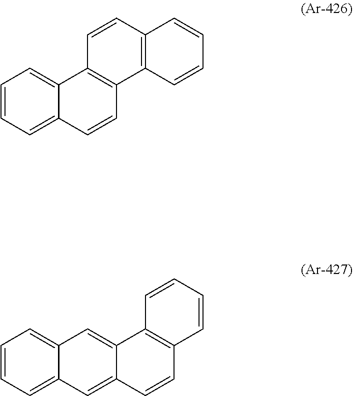

Note that Ar preferably has a nitrogen atom in its ring and is preferably bonded to the skeleton within parentheses in General Formula (R1) above by a bond of the nitrogen atom or a bond of a carbon atom adjacent to the nitrogen atom.

Specific examples of the organic compounds represented by General Formulae (R1) and (R2-1) to (R2-6) above include organic compounds represented by Structural Formulae (405) to (424) below.

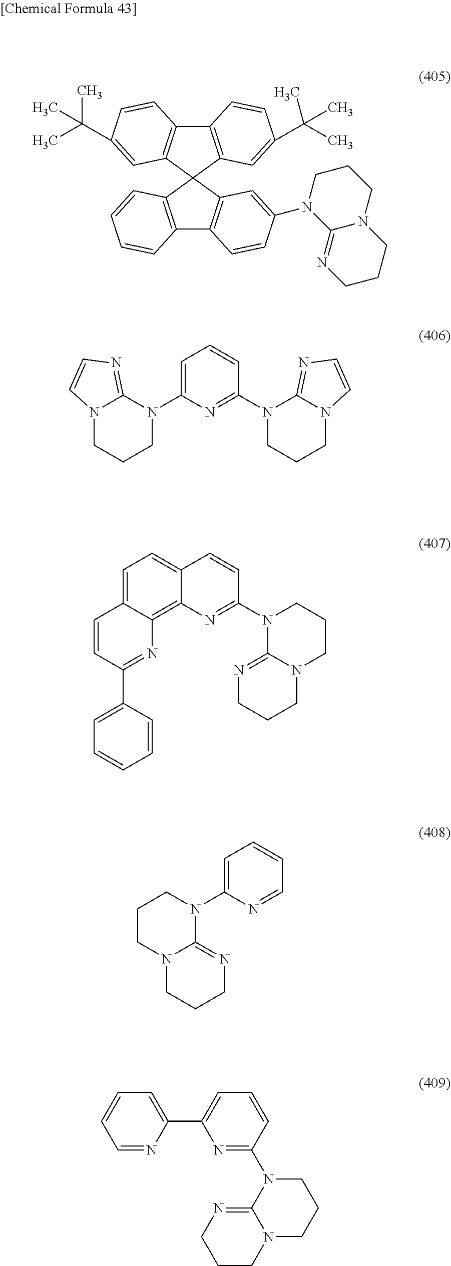

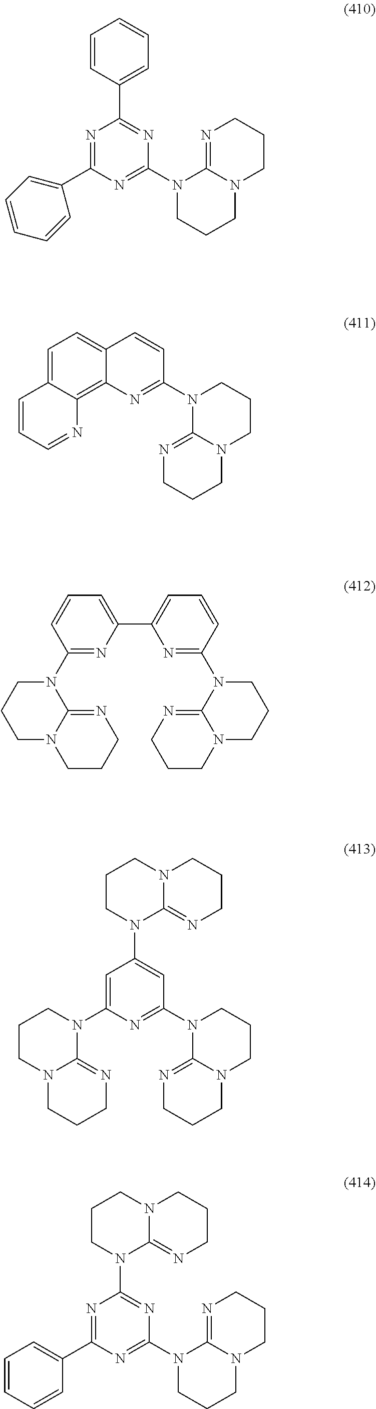

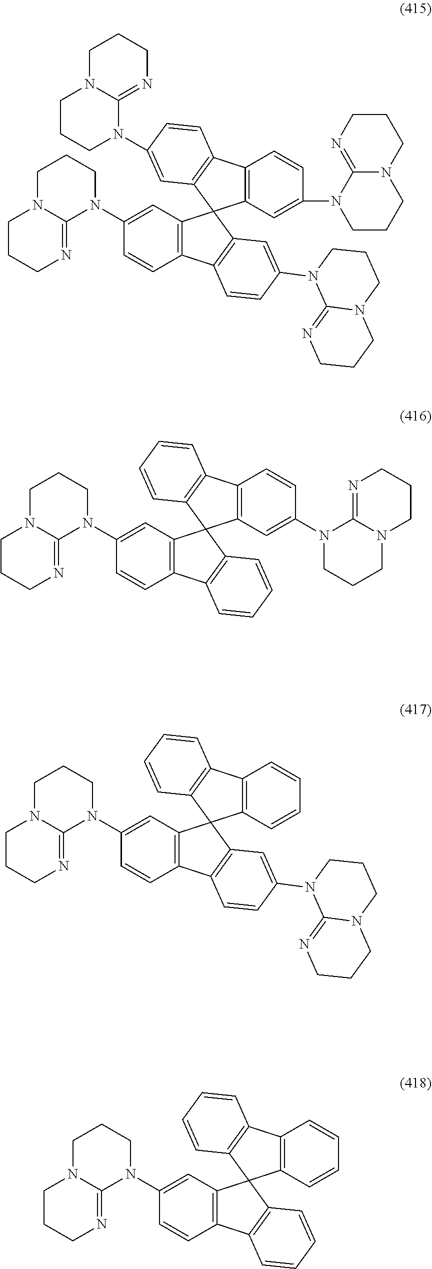

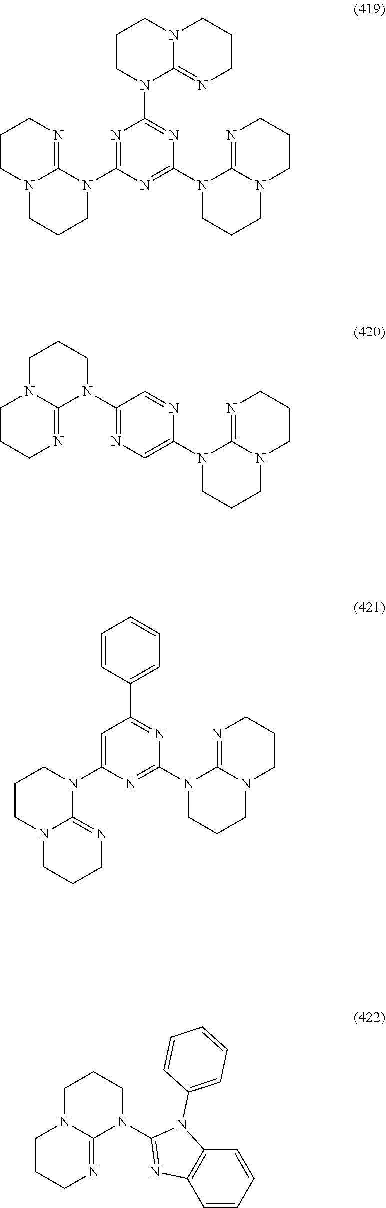

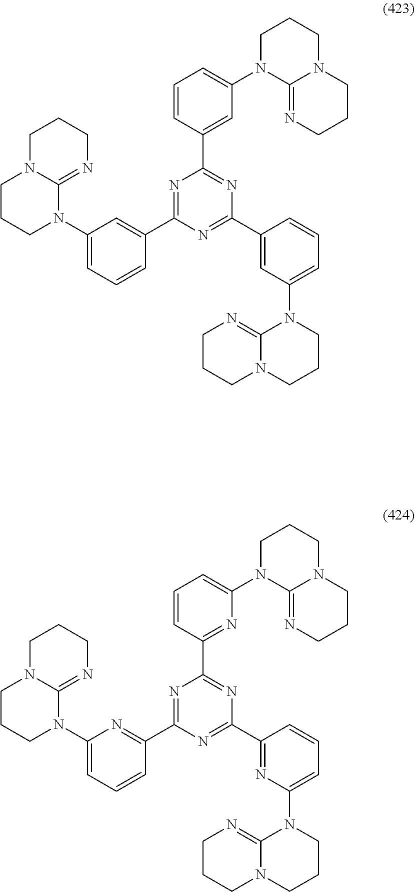

Although FIG. 1D illustrates the structure in which two of the organic compound layers 103 are stacked, three or more organic compound layers may be stacked with charge-generation layers each provided between two adjacent organic compound layers.

### <<Substrate>>

A light-emitting device of one embodiment of the present invention may be formed over a substrate of glass, plastic, or the like. As the way of stacking layers over the substrate, layers may be sequentially stacked from the first electrode 101 side or sequentially stacked from the second electrode 102 side.

For the substrate over which the light-emitting element of one embodiment of the present invention can be formed, glass, quartz, plastic, or the like can be used, for example. Alternatively, a flexible substrate may be used. The flexible substrate means a substrate that can be bent, such as a plastic substrate made of polycarbonate or polyarylate, for example. Alternatively, a film, an inorganic vapor deposition film, or the like can be used. Another material may be used as long as the substrate functions as a support in a manufacturing process of the light-emitting elements or the optical elements. Another material having a function of protecting the light-emitting elements or the optical elements may be used.

In this specification and the like, a light-emitting element can be formed using any of a variety of substrates, for example. There is no particular limitation on the type of the substrate. Examples of the substrate include a semiconductor substrate (e.g., a single crystal substrate or a silicon substrate); a silicon on insulator (SOI) substrate; a glass substrate; a quartz substrate; a plastic substrate; a metal substrate; a stainless steel substrate; a substrate including stainless steel foil; a tungsten substrate; a substrate including tungsten foil; a flexible substrate; an attachment film; and cellulose nanofiber (CNF), paper, and a base material film that include a fibrous material. Examples of a glass substrate include a barium borosilicate glass substrate, an aluminoborosilicate glass substrate, and a soda lime glass substrate. Examples of the flexible substrate, the attachment film, the base material film, and the like are substrates of plastics typified by polyethylene terephthalate (PET), polyethylene naphthalate (PEN), polyether sulfone (PES), and polytetrafluoroethylene (PTFE). Another example is an acrylic resin. Furthermore, polypropylene, polyester, polyvinyl fluoride, and polyvinyl chloride can be given as examples. Other examples include a resin such as a polyamide resin, a polyimide resin, an aramid resin, or an epoxy resin, an inorganic vapor deposition film, and paper.

Alternatively, a flexible substrate may be used as the substrate such that the light-emitting element is provided directly on the flexible substrate. Further alternatively, a separation layer may be provided between the substrate and the light-emitting element. The separation layer can be used when part or the whole of a light-emitting element formed over the separation layer is separated from the substrate and transferred onto another substrate. In such a case, the light-emitting element can be transferred to a substrate having low heat resistance or a flexible substrate as well. For the above separation layer, a stack including inorganic films, which are a tungsten film and a silicon oxide film, or a structure in which a resin film of polyimide or the like is formed over a substrate can be used, for example.

In other words, after the light-emitting element is formed using a substrate, the light-emitting element may be transferred to another substrate. Examples of the substrate to which the light-emitting element is transferred include, in addition to the above-described substrates, a cellophane substrate, a stone substrate, a wood substrate, a cloth substrate (including a natural fiber (e.g., silk, cotton, and hemp), a synthetic fiber (e.g., nylon, polyurethane, and polyester), a regenerated fiber (e.g., acetate, cupro, rayon, and regenerated polyester), and the like), a leather substrate, and a rubber substrate. With the use of such a substrate, a light-emitting element with high durability, high heat resistance, reduced weight, or reduced thickness can be formed.

The light-emitting device may be formed over an electrode electrically connected to a field-effect transistor (FET), for example, that is formed over any of the above-described substrates. Accordingly, an active matrix display apparatus in which the FET controls the driving of the light-emitting device can be manufactured.

In this embodiment, one embodiment of the present invention has been described. Other embodiments of the present invention are described in other embodiments. Note that one embodiment of the present invention is not limited thereto. In other words, various embodiments of the invention are described in this embodiment and the other embodiments, and one embodiment of the present invention is not limited to a particular embodiment. For example, although the example in which one embodiment of the present invention is applied to a light-emitting element is described, one embodiment of the present invention is not limited thereto. For example, depending on circumstances or conditions, one embodiment of the present invention is not necessarily used in a light-emitting element. One embodiment of the present invention shows, but is not limited to, an example of including a first organic compound, a second organic compound, and a guest material capable of converting triplet excitation energy into light emission, in which the LUMO level of the first organic compound is lower than that of the second organic compound and the HOMO level of the first organic compound is lower than that of the second organic compound. Depending on circumstances or conditions, in one embodiment of the present invention, for example, the LUMO level of the first organic compound is not necessarily lower than that of the second organic compound. Alternatively, the HOMO level of the first organic compound is not necessarily lower than that of the second organic compound. One embodiment of the present invention shows, but is not limited to, an example in which the first organic compound and the second organic compound form an exciplex. Depending on circumstances or conditions, in one embodiment of the present invention, for example, the first organic compound and the second organic compound do not necessarily form an exciplex. One embodiment of the present invention shows, but is not limited to, an example in which the LUMO level of the guest material is higher than that of the first organic compound and the HOMO level of the guest material is lower than that of the second organic compound. Depending on circumstances or conditions, in one embodiment of the present invention, for example, the LUMO level of the guest material is not necessarily higher than that of the first organic compound. Alternatively, the HOMO level of the guest material is not necessarily lower than that of the second organic compound.

The structure described above in this embodiment can be used in combination with any of the structures described in the other embodiments as appropriate.

### Embodiment 3

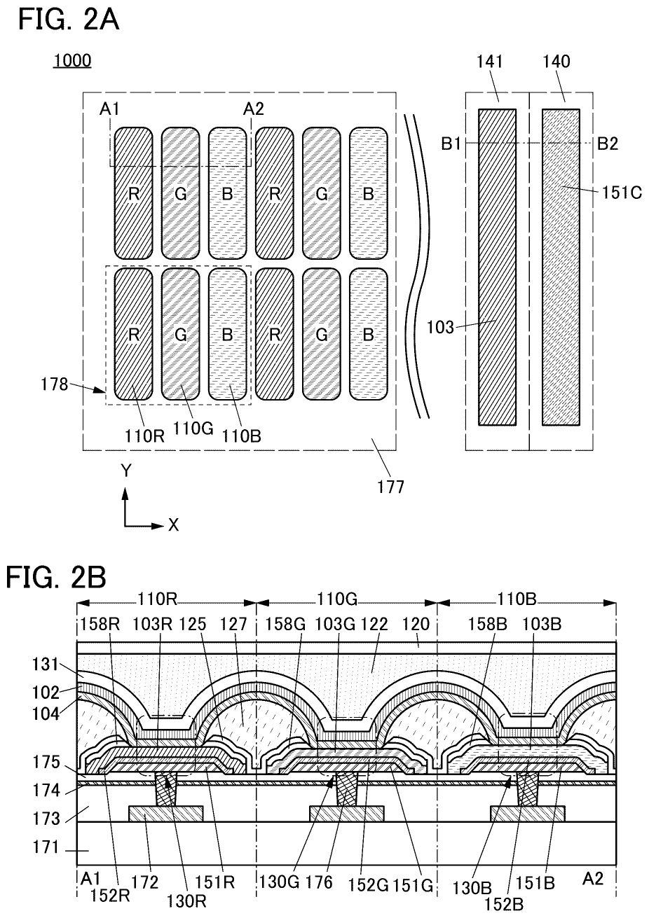

As illustrated as an example in FIGS. 2A and 2B, a plurality of light-emitting devices, which are described in the above embodiment, are formed over the insulating layer 175 to constitute part of a light-emitting apparatus. In this embodiment, the light-emitting apparatus of one embodiment of the present invention will be described in detail.

A light-emitting apparatus 1000 includes a pixel portion 177 in which a plurality of pixels 178 are arranged in matrix. The pixel 178 includes a subpixel 110R, a subpixel 110G, and a subpixel 110B.

In this specification and the like, for example, matters common to the subpixels 110R, 110G, and 110B are sometimes described using the collective term “subpixel 110”. As for components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by subpixels; however, the structure of the present invention is not limited to this structure. That is, subpixels of a different combination of colors may be employed. The number of subpixels is not limited to three, and four or more subpixels may be used, for example. Examples of four subpixels include subpixels emitting light of four colors of R, G, B, and white (W), subpixels emitting light of four colors of R, G, B, and yellow (Y), and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 2A illustrates an example where subpixels of different colors are arranged in the X direction and subpixels of the same color are arranged in the Y direction. Note that subpixels of different colors may be arranged in the Y direction, and subpixels of the same color may be arranged in the X direction.

A connection portion 140 and a region 141 may be provided outside the pixel portion 177. The region 141 is preferably positioned between the pixel portion 177 and the connection portion 140, for example. The organic compound layer 103 is provided in the region 141. A conductive layer 151C is provided in the connection portion 140.

Although FIG. 2A illustrates an example where the region 141 and the connection portion 140 are positioned on the right side of the pixel portion 177, the positions of the region 141 and the connection portion 140 are not particularly limited. The number of the regions 141 and the number of the connection portions 140 can each be one or more.

FIG. 2B is an example of a cross-sectional view along the dashed-dotted line A1-A2 in FIG. 2A. As illustrated in FIG. 2B, the light-emitting apparatus 1000 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is preferably provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A substrate 120 is bonded to the light-emitting device 130 with a resin layer 122. An inorganic insulating layer 125 and an insulating layer 127 over the inorganic insulating layer 125 may be provided between adjacent light-emitting devices 130.

Although FIG. 2B illustrates cross sections of a plurality of the inorganic insulating layers 125 and a plurality of the insulating layers 127, the inorganic insulating layers 125 are preferably connected to each other and the insulating layers 127 are preferably connected to each other when the light-emitting apparatus 1000 is seen from above. That is, the insulating layer 125 and the insulating layer 127 preferably have openings above first electrodes.

In FIG. 2B, a light-emitting device 130R, a light-emitting device 130G, and a light-emitting device 130B are each illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light.

Note that the organic compound layer 103 at least includes a light-emitting layer and can include other functional layers (a hole-injection layer, a hole-transport layer, a hole-blocking layer, an electron-blocking layer, an electron-transport layer, an electron-injection layer, and the like). The organic compound layer 103 and a common layer 104 may collectively include functional layers (a hole-injection layer, a hole-transport layer, a hole-blocking layer, a light-emitting layer, an electron-blocking layer, an electron-transport layer, an electron-injection layer, and the like) included in an EL layer that emits light.

The light-emitting apparatus of one embodiment of the present invention can be, for example, a top-emission light-emitting apparatus where light is emitted in the direction opposite to a substrate over which light-emitting devices are formed. Note that the light-emitting apparatus of one embodiment of the present invention may be of a bottom emission type.

Each of the light-emitting devices 130 (the light-emitting devices 130R, 130G, and 130B) has a structure as described in Embodiment 1 and includes the first electrode (pixel electrode) including a conductive layer 151 (conductive layers 151R, 151G, and 151B) and a conductive layer 152 (conductive layers 152R, 152G, and 152B), the organic compound layer 103 (the organic compound layers 103B, 103G, and 103B) over the first electrode, the common layer 104 over the organic compound layer 103, the second electrode (common electrode) 102 over the common layer 104, and the cap layer 131.

Note that the common layer 104 is not necessarily provided. The common layer 104 can reduce damage to the organic compound layer 103 caused in a later step. In the case where the common layer 104 is provided, the common layer 104 may function as an electron-injection layer. In the case where the common layer 104 functions as an electron-injection layer, a stack of the organic compound layer 103 and the common layer 104 corresponds to the organic compound layer 103 in Embodiment 1.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layer 103R, an organic compound layer 103G, and an organic compound layer 103B are island-shaped layers that are independent of each other. Alternatively, an organic compound layer of the light-emitting devices of one emission color may be independent of an organic compound layer of the light-emitting devices of another emission color. Providing the island-shaped organic compound layer 103 in each of the light-emitting devices 130 can suppress leakage current between the adjacent light-emitting devices 130 even in a high-resolution light-emitting apparatus. This can prevent crosstalk, so that a light-emitting apparatus with extremely high contrast can be obtained. Specifically, a light-emitting apparatus having high current efficiency at low luminance can be obtained.

The organic compound layer 103 may be provided to cover top and side surfaces of the first electrode (pixel electrode) of the light-emitting device 130. In that case, the aperture ratio of the light-emitting apparatus 1000 can be easily increased as compared to the structure where an edge portion of the organic compound layer 103 is positioned inward from an edge portion of the pixel electrode. Covering the side surface of the pixel electrode of the light-emitting device 130 with the organic compound layer 103 can inhibit the pixel electrode from being in contact with the second electrode 102; hence, a short circuit of the light-emitting device 130 can be inhibited. Furthermore, the distance between a light-emitting region (i.e., a region overlapping the pixel electrode) in the organic compound layer 103 and the edge portion of the organic compound layer 103 can be increased. Since the edge portion of the organic compound layer 103 might be damaged by processing, using a region that is away from the edge portion of the organic compound layer 103 as the light-emitting region can increase the reliability of the light-emitting device 130.

In the light-emitting apparatus of one embodiment of the present invention, the first electrode (pixel electrode) of the light-emitting device may have a stacked-layer structure. For example, in the example illustrated in FIG. 2B, the first electrode of the light-emitting device 130 is a stack of the conductive layer 151 and the conductive layer 152.

In the case where the light-emitting apparatus 1000 is a top-emission light-emitting apparatus, for example, in the pixel electrode of the light-emitting device 130, the conductive layer 151 preferably has high visible light reflectance and the conductive layer 152 preferably has a visible-light-transmitting property and a high work function. The higher the visible light reflectance of the pixel electrode is, the higher the efficiency of extraction of the light emitted by the organic compound layer 103 is. In the case where the pixel electrode functions as an anode, the higher the work function of the pixel electrode is, the easier it is to inject holes into the organic compound layer 103. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

Specifically, the visible light reflectance of the conductive layer 151 is preferably higher than or equal to 40% and lower than or equal to 100%, further preferably higher than or equal to 70% and lower than or equal to 100%, for example. When the conductive layer 152 is used as an electrode having a visible-light-transmitting property, the visible light transmittance is preferably higher than or equal to 40%, for example.

In the case where a film formed after the formation of the pixel electrode having a stacked-layer structure is removed by a wet etching method, for example, a stack including the pixel electrode might be impregnated with a chemical solution used for the etching. When the chemical solution reaches the pixel electrode, galvanic corrosion between a plurality of layers constituting the pixel electrode might occur, leading to deterioration of the pixel electrode.

In view of the above, the conductive layer 152 is preferably formed to cover the top and side surfaces of the conductive layer 151. When the conductive layer 151 is covered with the conductive layer 152, the chemical solution does not reach the conductive layer 151; thus, occurrence of galvanic corrosion in the pixel electrode can be inhibited. This allows the light-emitting apparatus 1000 to be fabricated by a high-yield method and to be accordingly inexpensive. In addition, generation of a defect in the light-emitting apparatus 1000 can be inhibited, which makes the light-emitting apparatus 1000 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a work function of higher than or equal to 4.0 eV, for example.

The conductive layer 151 and the conductive layer 152 may each be a stack of a plurality of layers containing different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can contain the same material as a layer of the conductive layer 152 in contact with the conductive layer 151.

The conductive layer 151 preferably has an end portion with a tapered shape. Specifically, the end portion of the conductive layer 151 preferably has a tapered shape with a taper angle of less than 90°. In that case, the conductive layer 152 provided along the side surface of the conductive layer 151 also has an end portion with a tapered shape. When the end portion of the conductive layer 152 has a tapered shape, coverage with the organic compound layer 103 provided along the side surface of the conductive layer 152 can be improved.

In the case where the conductive layer 151 or the conductive layer 152 has a stacked-layer structure, at least one of the stacked layers preferably has a tapered side surface. The stacked layers of the conductive layer(s) may have different tapered shapes.

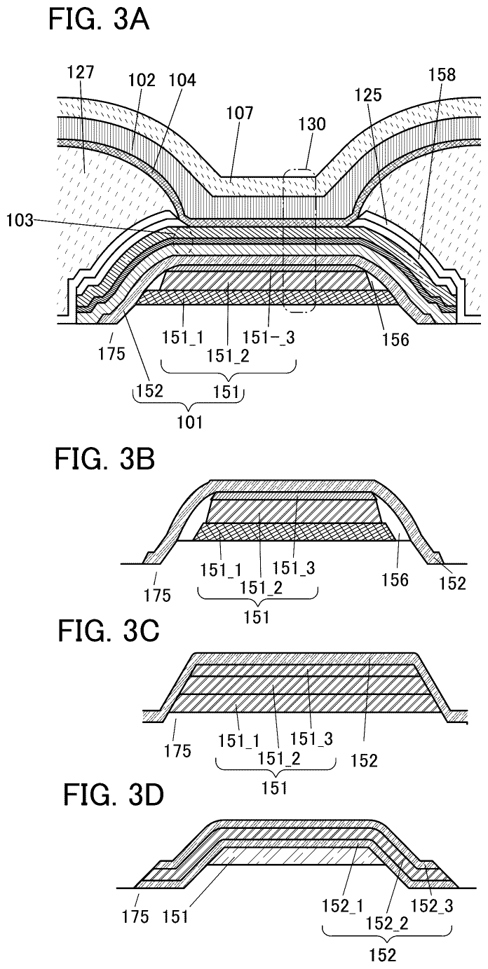

FIG. 3A illustrates the case where the conductive layer 151 has a stacked-layer structure of a plurality of layers containing different materials. As illustrated in FIG. 3A, the conductive layer 151 includes a conductive layer 151_1, a conductive layer 151_2 over the conductive layer 151_1, and a conductive layer 151_3 over the conductive layer 151_2. In other words, the conductive layer 151 illustrated in FIG. 3A has a three-layer structure. In the case where the conductive layer 151 is a stack of a plurality of layers as described above, the visible light reflectance of at least one of the layers included in the conductive layer 151 is made higher than that of the conductive layer 152.

In the example illustrated in FIG. 3A, the conductive layer 151_2 is interposed between the conductive layers 151_1 and 151_3. A material that is less likely to change in quality than the conductive layer 151_2 is preferably used for the conductive layers 151_1 and 151_3. The conductive layer 151_1 can be formed using, for example, a material that is less likely to migrate owing to contact with the insulating layer 175 than the material for the conductive layer 151_2. The conductive layer 151_3 can be formed using a material an oxide of which has lower electrical resistivity than an oxide of the material used for the conductive layer 151_2 and which is less likely to be oxidized than the conductive layer 151_2.

In this manner, the structure in which the conductive layer 151_2 is interposed between the conductive layers 151_1 and 151_3 can expand the range of choices for the material for the conductive layer 151_2. The conductive layer 1512, for example, can thus have higher visible light reflectance than at least one of the conductive layers 151_1 and 151_3. For example, aluminum can be used for the conductive layer 151_2. The conductive layer 151_2 may be formed using an alloy containing aluminum. The conductive layer 151_1 can be formed using titanium; titanium has lower visible light reflectance than aluminum but is less likely to migrate by contact with the insulating layer 175 than aluminum. Furthermore, the conductive layer 151_3 can be formed using titanium; titanium is less likely to be oxidized than aluminum and an oxide of titanium has lower electrical resistivity than aluminum oxide, although titanium has lower visible light reflectance than aluminum.

The conductive layer 151_3 may be formed using silver or an alloy containing silver. Silver is characterized by its visible light reflectance higher than that of titanium. In addition, silver is characterized by being less likely to be oxidized than aluminum, and silver oxide is characterized by its electrical resistivity lower than that of aluminum oxide. Thus, the conductive layer 151_3 formed using silver or an alloy containing silver can suitably increase the visible light reflectance of the conductive layer 151 and inhibit an increase in the electric resistance of the pixel electrode due to oxidation of the conductive layer 151_2. Here, as the alloy containing silver, an alloy of silver, palladium, and copper (also referred to as Ag—Pd—Cu or APC) can be used, for example. When the conductive layer 151_3 is formed using silver or an alloy containing silver and the conductive layer 151_2 is formed using aluminum, the visible light reflectance of the conductive layer 151_3 can be higher than that of the conductive layer 151_2. Here, the conductive layer 151_2 may be formed using silver or an alloy containing silver. The conductive layer 151_1 may be formed using silver or an alloy containing silver.

Meanwhile, a film formed using titanium has better processability in etching than a film formed using silver. Thus, use of titanium for the conductive layer 151_3 can facilitate formation of the conductive layer 151_3. Note that a film formed using aluminum also has better processability in etching than a film formed using silver.

The conductive layer 151 having a stacked-layer structure of a plurality of layers as described above can improve the characteristics of the light-emitting apparatus. For example, the light-emitting apparatus 1000 can have high light extraction efficiency and high reliability.

Here, in the case where the light-emitting device 130 has a microcavity structure, use of silver or an alloy containing silver, i.e., a material with high visible light reflectance, for the conductive layer 151_3 can favorably increase the light extraction efficiency of the light-emitting apparatus 1000.

Depending on the selected material or the processing method of the conductive layer 151, a side surface of the conductive layer 151_2 is positioned on an inner side than side surfaces of the conductive layer 151_1 and the conductive layer 151_3 and a protruding portion might be formed as illustrated in FIG. 3A. The protruding portion might impair coverage of the conductive layer 151 with the conductive layer 152 to cause a step-cut of the conductive layer 152.

Thus, an insulating layer 156 is preferably provided as illustrated in FIG. 3A. FIG. 3A illustrates an example in which the insulating layer 156 is provided over the conductive layer 151_1 to include a region overlapping with the side surface of the conductive layer 151_2. Such a structure can inhibit occurrence of the step-cut or a reduction in the thickness of the conductive layer 152 due to the protruding portion; thus, connection defects or an increase in driving voltage can be inhibited.

Although FIG. 3A illustrates the structure in which the side surface of the conductive layer 151_2 is entirely covered with the insulating layer 156, part of the side surface the conductive layer 151_2 is not necessarily covered with the insulating layer 156. Also in a pixel electrode with a later-described structure, part of the side surface of the conductive layer 151_2 is not necessarily covered with the insulating layer 156.

Here, the insulating layer 156 preferably has a curved surface as illustrated in FIG. 3A. In that case, a step-cut in the conductive layer 152 covering the insulating layer 156 is less likely to occur than in the case where the insulating layer 156 has a perpendicular side surface (a side surface parallel to the Z direction), for example. In addition, a step-cut in the conductive layer 152 covering the insulating layer 156 is less likely to occur also in the case where the side surface of the insulating layer 156 has a tapered shape, or specifically, a tapered shape with a taper angle of less than 90°, than in the case where the insulating layer 156 has a perpendicular side surface, for example. As described above, the light-emitting apparatus 1000 can be fabricated by a high-yield method. Moreover, the light-emitting apparatus 1000 can have high reliability since generation of defects is inhibited therein.

Note that one embodiment of the present invention is not limited thereto. FIGS. 3B to 3D illustrate other examples of the structure of the first electrode 101.

FIG. 3B illustrates a variation structure of the first electrode 101 in FIGS. 1A to 1E, in which the insulating layer 156 covers the side surfaces of the conductive layers 151_1, 151_2, and 151_3 instead of covering only the side surface of the conductive layer 151_2.

FIG. 3C illustrates a variation structure of the first electrode 101 in FIGS. 1A to 1E, in which the insulating layer 156 is not provided.

FIG. 3D illustrates a variation structure of the first electrode 101 in FIGS. 1A to 1E, in which the conductive layer 151 does not have a stacked-layer structure but the conductive layer 152 has a stacked-layer structure.

A conductive layer 152_1 has higher adhesion to a conductive layer 152_2 than the insulating layer 175 does, for example. For the conductive layer 152_1, an oxide containing one or more selected from indium, tin, zinc, gallium, titanium, aluminum, and silicon, for example, can be used. For example, it is preferable to use a conductive oxide containing one or more of indium oxide, an indium tin oxide, an indium zinc oxide, zinc oxide, zinc oxide containing gallium, titanium oxide, an indium titanium oxide, zinc titanate, an aluminum zinc oxide, an indium zinc oxide containing gallium, an indium zinc oxide containing aluminum, an indium tin oxide containing silicon, an indium zinc oxide containing silicon, and the like. Accordingly, peeling of the conductive layer 152_2 can be inhibited. The conductive layer 1522 is not in contact with the insulating layer 175.

The conductive layer 152_2 is a layer whose visible light reflectance (e.g., reflectance with respect to light with a predetermined wavelength in a range greater than or equal to 400 nm and less than 750 nm) is higher than that of the conductive layers 151, 152_1, and 152_3. The visible light reflectance of the conductive layer 152_2 can be, for example, higher than or equal to 70% and lower than or equal to 100%, and is preferably higher than or equal to 80% and lower than or equal to 100%, further preferably higher than or equal to 90% and lower than or equal to 100%. For the conductive layer 152_2, silver or an alloy containing silver can be used, for example. An example of the alloy containing silver is an alloy of silver, palladium, and copper (APC). In the above manner, the light-emitting apparatus 1000 can have high light extraction efficiency. Note that a metal other than silver may be used for the conductive layer 152_2.

When the conductive layers 151 and 152 serve as the anode, a layer having a high work function is preferably used as the conductive layer 1523. The conductive layer 152_3 has a higher work function than the conductive layer 1522, for example. For the conductive layer 1523, a material similar to the material usable for the conductive layer 152_1 can be used, for example. For example, the conductive layers 152_1 and 152_3 can be formed using the same kind of material.

When the conductive layers 151 and 152 serve as the cathode, a layer having a low work function is preferably used as the conductive layer 1523. The conductive layer 152_3 has a lower work function than the conductive layer 1522, for example.

The conductive layer 152_3 is preferably a layer having high visible light transmittance (e.g., transmittance with respect to light with a predetermined wavelength in a range greater than or equal to 400 nm and less than 750 nm). For example, the visible light transmittance of the conductive layer 152_3 is preferably higher than that of the conductive layers 151 and 152_2. The visible light transmittance of the conductive layer 1523 can be, for example, higher than or equal to 60% and lower than or equal to 100%, and is preferably higher than or equal to 70% and lower than or equal to 100%, further preferably higher than or equal to 80% and lower than or equal to 100%. Accordingly, the amount of light absorbed by the conductive layer 152_3 among light emitted from the organic compound layer 103 can be reduced. As described above, the conductive layer 152_2 under the conductive layer 152_3 can be a layer having high visible light reflectance. Thus, the light-emitting apparatus 1000 can have high light extraction efficiency.

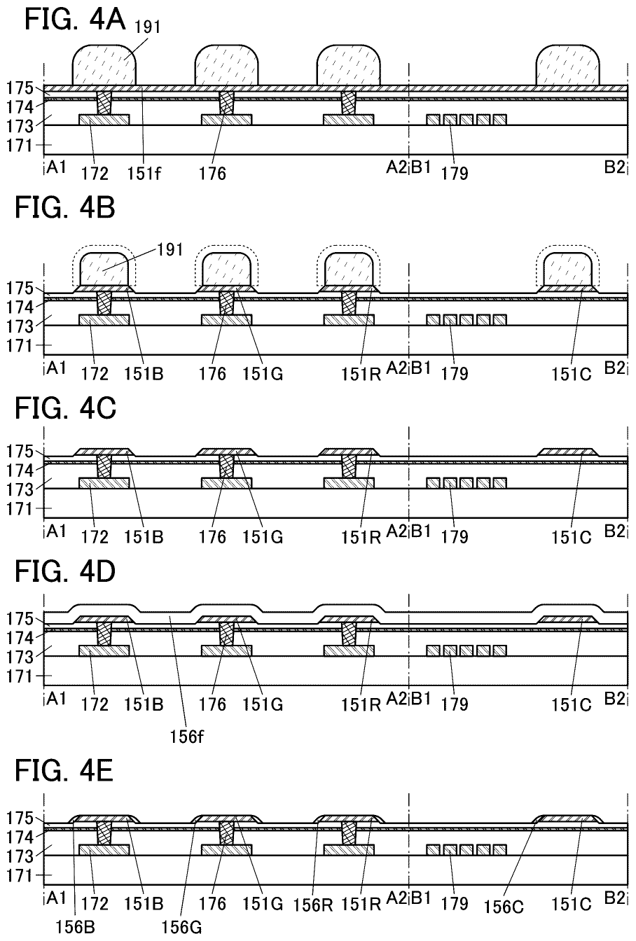

Next, an exemplary method for fabricating the light-emitting apparatus 1000 having the structure illustrated in FIGS. 2A and 2B is described with reference to FIGS. 4A to 4E, FIGS. 5A to 5E, FIGS. 6A to 6C, FIGS. 7A to 7C, FIGS. 8A to 8C, FIGS. 9A to 9C, and FIGS. 10A to 10C.

### Fabrication Method Example 1

Thin films included in the light-emitting apparatus (e.g., insulating films, semiconductor films, and conductive films) can be formed by a sputtering method, a chemical vapor deposition method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the light-emitting apparatus (e.g., insulating films, semiconductor films, and conductive films) can also be formed by a wet process such as spin coating, dipping, spray coating, ink-jetting, dispensing, screen printing, offset printing, doctor blade coating, slit coating, roll coating, curtain coating, or knife coating.

Specifically, for fabrication of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the light-emitting apparatus can be processed by a photolithography method, for example. Alternatively, a nanoimprinting method, a sandblasting method, a lift-off method, or the like may be used to process thin films. Alternatively, island-shaped thin films may be directly formed by a film formation method using a shielding mask such as a metal mask.

There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

For etching of thin films, a dry etching method, a wet etching method, a sandblast method, or the like can be used.

First, as illustrated in FIG. 4A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate that has heat resistance high enough to withstand at least heat treatment performed later can be used. When an insulating substrate is used, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like; a compound semiconductor substrate of silicon germanium or the like; or an SOI substrate.

Next, as illustrated in FIG. 4A, openings reaching the conductive layer 172 are formed in the insulating layers 175, 174, and 173. Then, the plugs 176 are formed to fill the openings.

Next, as illustrated in FIG. 4A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Subsequently, a resist mask 191 is formed over the conductive film 151f, for example, as illustrated in FIG. 4A. The resist mask 191 can be formed by application of a photosensitive material (photoresist), light exposure, and development.

Subsequently, as illustrated in FIG. 4B, the conductive film 151f in a region that is not overlapped by the resist mask 191, for example, is removed by an etching method, specifically, a dry etching method, for instance. Note that in the case where the conductive film 151f includes a layer formed using a conductive oxide such as an indium tin oxide, for example, the layer may be removed by a wet etching method. In this manner, the conductive layer 151 is formed. In the case where part of the conductive film 151f is removed by a dry etching method, for example, a recessed portion (also referred to as a depression) may be formed in a region of the insulating layer 175 that is not overlapped by the conductive layer 151.

Next, the resist mask 191 is removed as illustrated in FIG. 4C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 4D, an insulating film 156f to be an insulating layer 156R, an insulating layer 156G, an insulating layer 156B, and an insulating layer 156C is formed over the conductive layer 151R, the conductive layer 151G, the conductive layer 151B, the conductive layer 151C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film containing silicon, a nitride insulating film containing silicon, an oxynitride insulating film containing silicon, a nitride oxide insulating film containing silicon, or the like can be used as the insulating film 156f. For the insulating film 156f, silicon oxynitride can be used, for example.

Subsequently, as illustrated in FIG. 4E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a photolithography method.

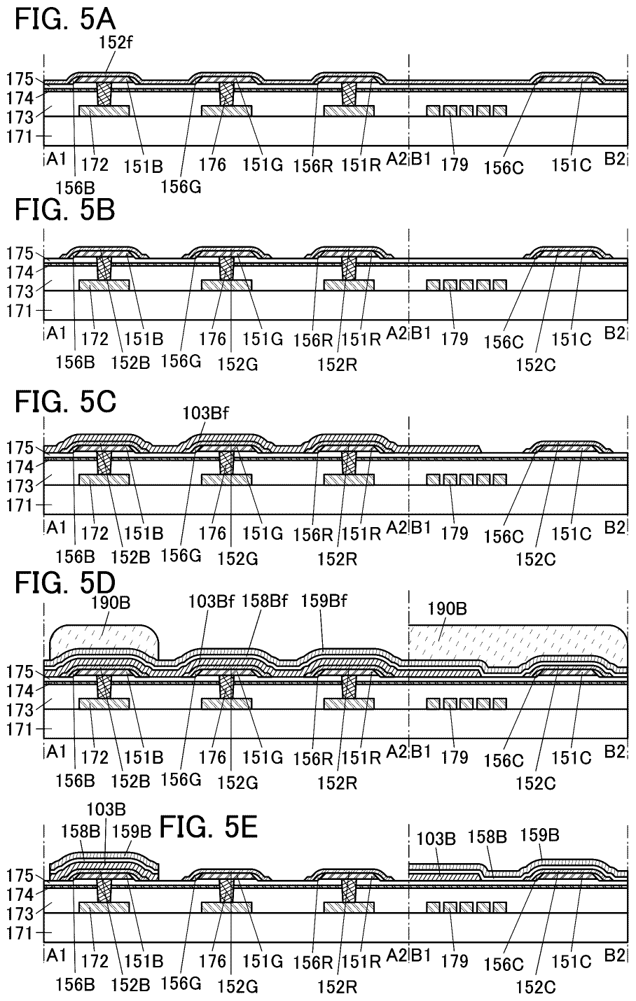

Then, as illustrated in FIG. 5A, a conductive film 152f to be the conductive layers 152R, 152G, and 152B and a conductive layer 152C is formed over the conductive layers 151R, 151G, 151B, and 151C and the insulating layers 156R, 156G, 156B, 156C, and 175. Specifically, the conductive film 152f is formed to cover the conductive layers 151R, 151G, 151B, and 151C and the insulating layers 156R, 156G, 156B, and 156C, for example.

The conductive film 152f can be formed by a sputtering method or a vacuum evaporation method, for example. The conductive film 152f can be formed by an ALD method. A conductive oxide can be used for the conductive film 152f, for example. The conductive film 152f can be a stack of a film formed using a metal material and a film formed thereover using a conductive oxide. For example, the conductive film 152f can be a stack of a film formed using titanium, silver, or an alloy containing silver and a film formed thereover using a conductive oxide.

Then, as illustrated in FIG. 5B, the conductive film 152f is processed by a photolithography method, for example, whereby the conductive layers 152R, 152G, 152B, and 152C are formed. Specifically, after a resist mask is formed, part of the conductive film 152f is removed by an etching method, for example. The conductive film 152f can be removed by a wet etching method, for example. The conductive film 152f may be removed by a dry etching method. Through the above steps, the pixel electrode including the conductive layer 151 and the conductive layer 152 is formed.

Next, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step and suppress film peeling. Note that the hydrophobization treatment is not necessarily performed.

Next, as illustrated in FIG. 5C, an organic compound film 103Bf to be the organic compound layer 103B is formed over the conductive layers 152B, 152G, and 152R and the insulating layer 175.

Note that in one embodiment of the present invention, the organic compound film 103Bf includes a plurality of organic compound layers including at least one light-emitting layer. The structure of the light-emitting device 130 described in Embodiment 1 can be referred to for the specific structure. The organic compound film 103Bf may have a structure in which the plurality of organic compound layers including at least one light-emitting layer are stacked with an intermediate layer positioned therebetween.

As illustrated in FIG. 5C, the organic compound film 103Bf is not formed over the conductive layer 152C. For example, a mask for specifying a film formation area (also referred to as an area mask, a rough metal mask, or the like to distinguish from a fine metal mask) is used, so that the organic compound film 103Bf can be formed only in a desired region. Employing a film formation step using an area mask and a processing step using a resist mask enables a light-emitting device to be fabricated by a relatively easy process.

The organic compound film 103Bf can be formed by an evaporation method, specifically a vacuum evaporation method, for example. The organic compound film 103Bf may be formed by a transfer method, a printing method, an ink-jet method, a coating method, or the like.

Next, as illustrated in FIG. 5D, a sacrificial film 158Bf to be a sacrificial layer 158B and a mask film 159Bf to be a mask layer 159B are sequentially formed over the organic compound film 103Bf.

The sacrificial film 158Bf and the mask film 159Bf can be formed by a sputtering method, an ALD method (including a thermal ALD method or a PEALD method), a CVD method, or a vacuum evaporation method, for example. Alternatively, the sacrificial film 158Bf and the mask film 159Bf may be formed by the above-described wet process.

The sacrificial film 158Bf and the mask film 159Bf are formed at a temperature lower than the upper temperature limit of the organic compound film 103Bf. The typical substrate temperatures in formation of the sacrificial film 158Bf and the mask film 159Bf are each lower than or equal to 200° C., preferably lower than or equal to 150° C., further preferably lower than or equal to 120° C., still further preferably lower than or equal to 100° C., yet still further preferably lower than or equal to 80° C.

Although this embodiment shows an example where a mask film having a two-layer structure of the sacrificial film 158Bf and the mask film 159Bf is formed, a mask film may have a single-layer structure or a stacked-layer structure of three or more layers.

Providing the sacrificial layer over the organic compound film 103Bf can reduce damage to the organic compound film 103Bf in the fabrication process of the light-emitting apparatus, resulting in an increase in reliability of the light-emitting device.

As the sacrificial film 158Bf, a film that is highly resistant to the process conditions for the organic compound film 103Bf, specifically, a film having high etching selectivity with respect to the organic compound film 103Bf is used. For the mask film 159Bf, a film having high etching selectivity with respect to the sacrificial film 158Bf is used.

The sacrificial film 158Bf and the mask film 159Bf are preferably films that can be removed by a wet etching method. The use of a wet etching method can reduce damage to the organic compound film 103Bf in processing of the sacrificial film 158Bf and the mask film 159Bf, as compared to the case of using a dry etching method.

In the case where a wet etching method is employed, it is particularly preferable to use an acidic chemical solution. As an acidic chemical solution, a chemical solution containing one of phosphoric acid, hydrofluoric acid, nitric acid, acetic acid, oxalic acid, sulfuric acid, and the like or a mixed chemical solution (also referred to as a mixed acid) that contains two or more of these acids is preferably used.

As each of the sacrificial film 158Bf and the mask film 159Bf, one or more of a metal film, an alloy film, a metal oxide film, a semiconductor film, an organic insulating film, and an inorganic insulating film, for example, can be used.

When a film containing a material having a property of blocking ultraviolet rays is used as each of the sacrificial film 158Bf and the mask film 159Bf, the organic compound layer can be inhibited from being irradiated with ultraviolet rays in a light exposure step, for example. The organic compound layer is inhibited from being damaged by ultraviolet rays, so that the reliability of the light-emitting device can be improved.

Note that the same effect is obtained when a film containing a material having a property of blocking ultraviolet rays is used for an after-mentioned inorganic insulating film 125f.

For each of the sacrificial film 158Bf and the mask film 159Bf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver.

The sacrificial film 158Bf and the mask film 159Bf can each be formed using a metal oxide such as an In—Ga—Zn oxide, an indium oxide, an In—Zn oxide, an In—Sn oxide, an indium titanium oxide (In—Ti oxide), an indium tin zinc oxide (In—Sn—Zn oxide), an indium titanium zinc oxide (In—Ti—Zn oxide), an indium gallium tin zinc oxide (In—Ga—Sn—Zn oxide), or an indium tin oxide containing silicon.

In addition, in place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

The sacrificial film 158Bf and the mask film 159Bf are preferably formed using a semiconductor material such as silicon or germanium, for example, for excellent compatibility with a semiconductor manufacturing process. An oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

As each of the sacrificial film 158Bf and the mask film 159Bf, any of a variety of inorganic insulating films can be used. In particular, an oxide insulating film is preferable because its adhesion to the organic compound film 103Bf is higher than that of a nitride insulating film. For example, an inorganic insulating material such as aluminum oxide, hafnium oxide, or silicon oxide can be used for the sacrificial film 158Bf and the mask film 159Bf. As the sacrificial film 158Bf and the mask film 159Bf, aluminum oxide films can be formed by an ALD method, for example. An ALD method is preferably used, in which case damage to a base (in particular, the organic compound layer) can be reduced.

One or both of the sacrificial film 158Bf and the mask film 159Bf may be formed using an organic material. For example, as the organic material, a material that can be dissolved in a solvent chemically stable with respect to at least the uppermost film of the organic compound film 103Bf may be used. Specifically, a material that will be dissolved in water or an alcohol can be suitably used. In forming a film of such a material, it is preferable to apply the material dissolved in a solvent such as water or an alcohol by a wet process and then perform heat treatment for evaporating the solvent. At this time, the heat treatment is preferably performed in a reduced-pressure atmosphere, in which case the solvent can be removed at a low temperature in a short time and thermal damage to the organic compound film 103Bf can be reduced accordingly.

The sacrificial film 158Bf and the mask film 159Bf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or any of the above wet processes can be used as the sacrificial film 158Bf, and an inorganic film (e.g., a silicon nitride film) formed by a sputtering method can be used as the mask film 159Bf.

Subsequently, a resist mask 190B is formed over the mask film 159Bf as illustrated in FIG. 5D. The resist mask 190B can be formed by application of a photosensitive material (photoresist), light exposure, and development.

The resist mask 190B may be formed using either a positive resist material or a negative resist material.

The resist mask 190B is provided at a position overlapping the conductive layer 152B. The resist mask 190B is preferably provided also at a position overlapping the conductive layer 152C. This can inhibit the conductive layer 152C from being damaged during the fabrication process of the light-emitting apparatus. Note that the resist mask 190B is not necessarily provided over the conductive layer 152C. The resist mask 190B is preferably provided to cover the area from the edge portion of the organic compound film 103Bf to the edge portion of the conductive layer 152C (the edge portion closer to the organic compound film 103Bf), as illustrated in the cross-sectional view along the line B1-B2 in FIG. 5C.

Next, as illustrated in FIG. 5E, part of the mask film 159Bf is removed using the resist mask 190B, whereby the mask layer 159B is formed. The mask layer 159B remains over the conductive layers 152B and 152C. After that, the resist mask 190B is removed. Then, part of the sacrificial film 158Bf is removed using the mask layer 159B as a mask (also referred to as a hard mask), whereby the sacrificial layer 158B is formed.

Each of the sacrificial film 158Bf and the mask film 159Bf can be processed by a wet etching method or a dry etching method. The sacrificial film 158Bf and the mask film 159Bf are preferably processed by wet etching.

The use of a wet etching method can reduce damage to the organic compound film 103Bf in processing of the sacrificial film 158Bf and the mask film 159Bf, as compared to the case of using a dry etching method. In the case of using a wet etching method, it is preferable to use a developer, an aqueous solution of tetramethylammonium hydroxide (TMAH), dilute hydrofluoric acid, oxalic acid, phosphoric acid, acetic acid, nitric acid, or a chemical solution containing a mixed solution of any of these acids, for example.

Since the organic compound film 103Bf is not exposed in the processing of the mask film 159Bf, the range of choice for a processing method for the mask film 159Bf is wider than that for the sacrificial film 158Bf. Specifically, even in the case where a gas containing oxygen is used as the etching gas in the processing of the mask film 159Bf, deterioration of the organic compound film 103Bf can be suppressed.

In the case where a wet etching method is employed, it is particularly preferable to use an acidic chemical solution. As an acidic chemical solution, a chemical solution containing one of phosphoric acid, hydrofluoric acid, nitric acid, acetic acid, oxalic acid, sulfuric acid, and the like or a mixed chemical solution (also referred to as a mixed acid) that contains two or more of these acids is preferably used.

In the case of using a dry etching method to process the sacrificial film 158Bf, deterioration of the organic compound film 103Bf can be suppressed by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

The resist mask 190B can be removed by a method similar to that for the resist mask 191. At this time, the sacrificial film 158Bf is positioned on the outermost surface, and the organic compound film 103Bf is not exposed; thus, the organic compound film 103Bf can be inhibited from being damaged in the step of removing the resist mask 190B. In addition, the range of choice of the method for removing the resist mask 190B can be widened.

Next, as illustrated in FIG. 5E, the organic compound film 103Bf is processed, so that the organic compound layer 103B is formed. For example, part of the organic compound film 103Bf is removed using the mask layer 159B and the sacrificial layer 158B as a hard mask, whereby the organic compound layer 103B is formed.

Accordingly, as illustrated in FIG. 5E, the stacked-layer structure of the organic compound layer 103B, the sacrificial layer 158B, and the mask layer 159B remains over the conductive layer 152B. The conductive layers 152G and 152R are exposed.

The organic compound film 103Bf can be processed by dry etching or wet etching. In the case where the processing is performed by dry etching, for example, an etching gas containing oxygen can be used. When the etching gas contains oxygen, the etching rate can be increased. Thus, the etching can be performed under a low-power condition while an adequately high etching rate is maintained. Accordingly, damage to the organic compound film 103Bf can be inhibited. Furthermore, a defect such as attachment of a reaction product generated during the etching can be inhibited.

An etching gas that does not contain oxygen may be used. In that case, deterioration of the organic compound film 103Bf can be inhibited, for example.

As described above, in one embodiment of the present invention, the mask layer 159B is formed in the following manner: the resist mask 190B is formed over the mask film 159Bf and part of the mask film 159Bf is removed using the resist mask 190B. After that, part of the organic compound film 103Bf is removed using the mask layer 159B as a hard mask, so that the organic compound layer 103B is formed. In other words, the organic compound layer 103B is formed by processing the organic compound film 103Bf by a photolithography method. Note that part of the organic compound film 103Bf may be removed using the resist mask 190B. Then, the resist mask 190B may be removed.

Here, hydrophobization treatment for the conductive layer 152G may be performed as necessary. At the time of processing the organic compound film 103Bf, a surface of the conductive layer 152G changes to have hydrophilic properties in some cases, for example. The hydrophobization treatment for the conductive layer 152G, for example, can increase the adhesion between the conductive layer 152G and a layer to be formed in a later step (which is the organic compound layer 103G here) and inhibit film peeling.

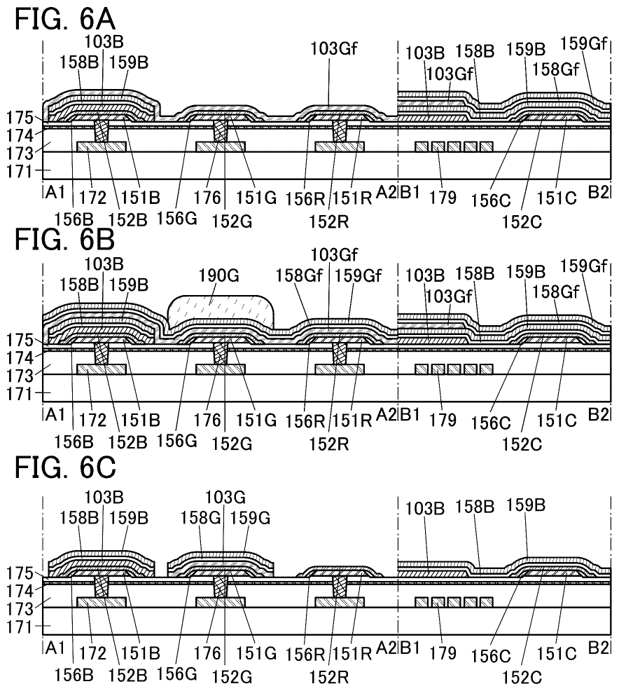

Next, as illustrated in FIG. 6A, an organic compound film 103Gf to be the organic compound layer 103G is formed over the conductive layer 152G, the conductive layer 152R, the mask layer 159B, and the insulating layer 175.

The organic compound film 103Gf can be formed by a method similar to that for forming the organic compound film 103Bf. The organic compound film 103Gf can have a structure similar to that of the organic compound film 103Bf.

Then, as illustrated in FIG. 6B, a sacrificial film 158Gf to be a sacrificial layer 158G and a mask film 159Gf to be a mask layer 159G are sequentially formed over the organic compound film 103Gf and the mask layer 159B. After that, a resist mask 190G is formed. The materials and the formation methods of the sacrificial film 158Gf and the mask film 159Gf are similar to those for the sacrificial film 158Bf and the mask film 159Bf. The material and the formation method of the resist mask 190G are similar to those for the resist mask 190B.

The resist mask 190G is provided at a position overlapping the conductive layer 152G.

Subsequently, as illustrated in FIG. 6C, part of the mask film 159Gf is removed using the resist mask 190G, whereby the mask layer 159G is formed. The mask layer 159G remains over the conductive layer 152G. After that, the resist mask 190G is removed. Then, part of the sacrificial film 158Gf is removed using the mask layer 159G as a mask, whereby the sacrificial layer 158G is formed. Next, the organic compound film 103Gf is processed to form the organic compound layer 103G. For example, part of the organic compound film 103Gf is removed using the mask layer 159G and the sacrificial layer 158G as a hard mask to form the organic compound layer 103G.

Accordingly, as illustrated in FIG. 6C, the stacked-layer structure of the organic compound layer 103G, the sacrificial layer 158G, and the mask layer 159G remains over the conductive layer 152G. The mask layer 159B and the conductive layer 152R are exposed.

Hydrophobization treatment for the conductive layer 152R may be performed, for example.

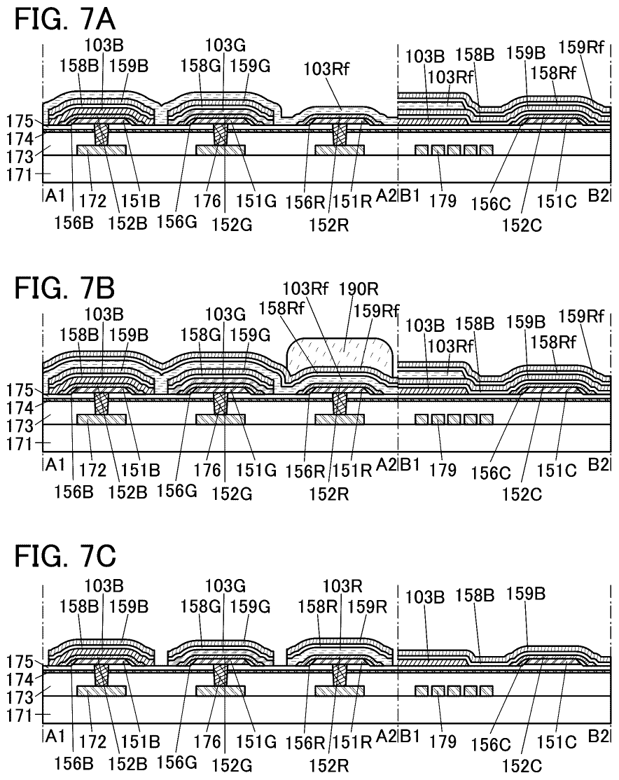

Next, as illustrated in FIG. 7A, an organic compound film 103Rf to be the organic compound layer 103R is formed over the conductive layer 152R, the mask layer 159G, the mask layer 159B, and the insulating layer 175.

The organic compound film 103Rf can be formed by a method similar to that for forming the organic compound film 103Gf. The organic compound film 103Rf can have a structure similar to that of the organic compound film 103Gf.

Subsequently, as illustrated in FIGS. 7B and 7C, a sacrificial layer 158R, a mask layer 159R, and the organic compound layer 103R are formed from a sacrificial film 158Rf, a mask film 159Rf, and the organic compound film 103Rf, respectively, using a resist mask 190R. For the formation methods of the sacrificial layer 158R, the mask layer 159R, and the organic compound layer 103R, the description for the organic compound layer 103G can be referred to.

Note that the side surfaces of the organic compound layers 103B, 103G, and 103R are preferably perpendicular or substantially perpendicular to their formation surfaces. For example, the angle between the formation surfaces and these side surfaces is preferably greater than or equal to 600 and less than or equal to 90°.

The distance between two adjacent layers among the organic compound layers 103B, 103G, and 103R, which are formed by a photolithography method as described above, can be reduced to less than or equal to 8 μm, less than or equal to 5 μm, less than or equal to 3 μm, less than or equal to 2 μm, or less than or equal to 1 μm. Here, the distance can be specified, for example, by a distance between opposite edge portions of two adjacent layers among the organic compound layers 103B, 103G, and 103R. Reducing the distance between the island-shaped organic compound layers can provide a light-emitting apparatus having high resolution and a high aperture ratio. In addition, the distance between the first electrodes of adjacent light-emitting devices can also be shortened to be, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

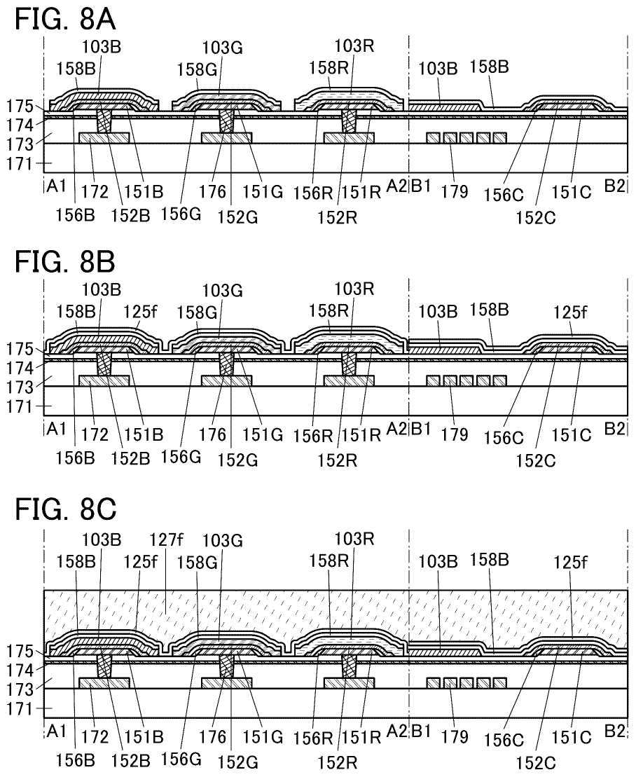

Next, as illustrated in FIG. 8A, the mask layers 159B, 159G, and 159R are removed.

This embodiment shows an example where the mask layers 159B, 159G, and 159R are removed; however, it is possible that the mask layers 159B, 159G, and 159R are not removed. For example, in the case where the mask layers 159B, 159G, and 159R contain the above-described material having a property of blocking ultraviolet rays, the procedure preferably proceeds to the next step without removing the mask layers 159B, 159G, and 159R, in which case the organic compound layer can be protected from light irradiation (including lighting).

The step of removing the mask layers can be performed by a method similar to that for the step of processing the mask films. Specifically, by using a wet etching method, damage applied to the organic compound layers 103B, 103G, and 103R at the time of removing the mask layers can be reduced as compared to the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layers 103B, 103G, and 103R and water adsorbed on the surfaces of the organic compound layers 103B, 103G, and 103R. For example, heat treatment in an inert atmosphere or a reduced-pressure atmosphere can be performed. The heat treatment can be performed at a substrate temperature of higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 8B, the inorganic insulating film 125f to be the inorganic insulating layer 125 is formed to cover the organic compound layers 103B, 103G, and 103R and the sacrificial layers 158B, 158G, and 158R.

As described later, an insulating film to be the insulating layer 127 is to be formed in contact with the top surface of the inorganic insulating film 125f. Thus, the top surface of the inorganic insulating film 125f preferably has a high affinity for the material used for the insulating film to be the insulating layer 127 (e.g., a photosensitive resin composition containing an acrylic resin). To improve the affinity, surface treatment may be performed on the top surface of the inorganic insulating film 125f. Specifically, the surface of the inorganic insulating film 125f is preferably made hydrophobic (or its hydrophobic property is preferably improved). For example, it is preferable to perform the treatment using a silylation agent such as hexamethyldisilazane (HMDS). By making the top surface of the inorganic insulating film 125f hydrophobic in such a manner, an insulating film 127f can be formed with favorable adhesion.

Then, as illustrated in FIG. 8C, an insulating film 127f to be the insulating layer 127 is formed over the inorganic insulating film 125f.

The inorganic insulating film 125f and the insulating film 127f are preferably formed by a formation method by which the organic compound layers 103B, 103G, and 103R are less damaged. The inorganic insulating film 125f, which is formed in contact with the side surfaces of the organic compound layers 103B, 103G, and 103R, is particularly preferably formed by a formation method that causes less damage to the organic compound layers 103B, 103G, and 103R than the method of forming the insulating film 127f.

Each of the insulating films 125f and 127f is formed at a temperature lower than the upper temperature limit of the organic compound layers 103B, 103G, and 103R. When the insulating film 125f is formed at a high substrate temperature, the formed insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness of greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case deposition damage is reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher deposition rate than an ALD method. In that case, a highly reliable light-emitting apparatus can be fabricated with high productivity.

The insulating film 127f is preferably formed by the aforementioned wet process. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limit of the organic compound layers 103B, 103G, and 103R. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent contained in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are sandwiched between any two of the conductive layers 152B, 152G, and 152R and around the conductive layer 152C. Thus, the top surfaces of the conductive layers 152B, 152G, 152R, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 formed later can be controlled in accordance with the exposed region of the insulating film 127f. In this embodiment, processing is performed such that the insulating layer 127 includes a portion overlapping the top surface of the conductive layer 151.

Here, when a barrier insulating layer against oxygen (e.g., an aluminum oxide film) is provided as one or both of the sacrificial layer 158 (the sacrificial layers 158B, 158G, and 158R) and the inorganic insulating film 125f, diffusion of oxygen to the organic compound layers 103B, 103G, and 103R can be suppressed. When the organic compound layer is irradiated with light (visible light or ultraviolet rays), the organic compound contained in the organic compound layer is brought into an excited state and a reaction between the organic compound and oxygen in the atmosphere is promoted in some cases. Specifically, when the organic compound layer is irradiated with light (visible light or ultraviolet rays) in an atmosphere including oxygen, oxygen might be bonded to the organic compound contained in the organic compound layer. By providing the sacrificial layer 158 and the inorganic insulating film 125f over the island-shaped organic compound layer, bonding of oxygen in the atmosphere to the organic compound contained in the organic compound layer can be suppressed.

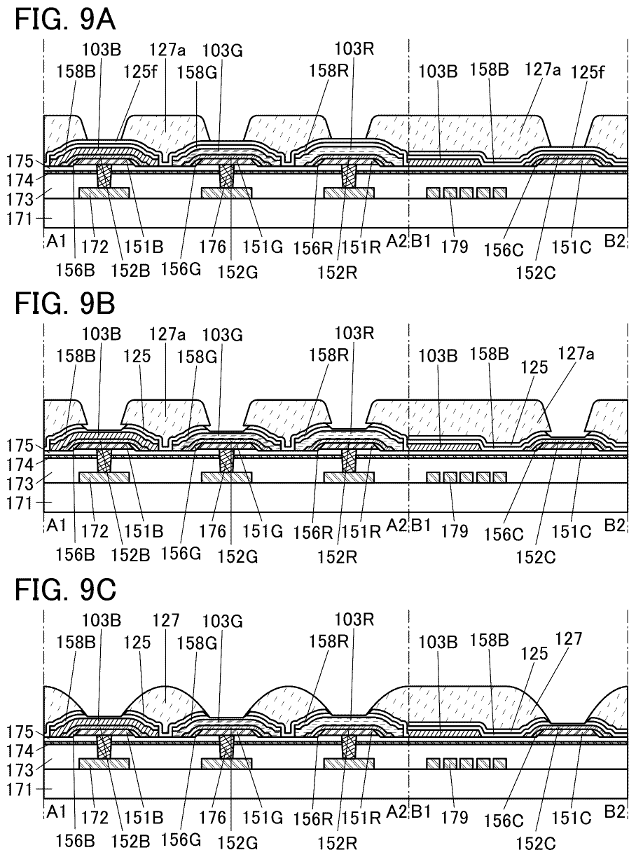

Next, as illustrated in FIG. 9A, development is performed to remove the exposed region of the insulating film 127f, whereby an insulating layer 127a is formed. The insulating layer 127a is formed in regions that are sandwiched between any two of the conductive layers 152B, 152G, and 152R and a region surrounding the conductive layer 152C. Here, when an acrylic resin is used for the insulating film 127f, an alkaline solution, such as TMAH, can be used as a developer.

Next, as illustrated in FIG. 9B, etching treatment is performed with the insulating layer 127a as a mask to remove part of the inorganic insulating film 125f and reduce the thickness of part of the sacrificial layers 158B, 158G, and 158R. Thus, the inorganic insulating layer 125 is formed under the insulating layer 127a. Note that the etching treatment for processing the inorganic insulating film 125 fusing the insulating layer 127a as a mask may be hereinafter referred to as first etching treatment.

In other words, the sacrificial layers 158B, 158G, and 158R are not removed completely by the first etching treatment, and the etching treatment is stopped when the thicknesses of the sacrificial layers 158B, 158G, and 158R are reduced. The corresponding sacrificial layers 158B, 158G, and 158R remain over the organic compound layers 103B, 103G, and 103R in this manner, whereby the organic compound layers 103B, 103G, and 103R can be prevented from being damaged by treatment in a later step.

The first etching treatment can be performed by dry etching or wet etching. Note that the inorganic insulating film 125f is preferably formed using a material similar to that of the sacrificial layers 158B, 158G, and 158R, in which case the processing of the inorganic insulating film 125f and thinning of the exposed part of the sacrificial layer 158 can be concurrently performed by the first etching treatment.

By etching using the insulating layer 127a with a tapered side surface as a mask, the side surface of the inorganic insulating layer 125 and upper edge portions of the side surfaces of the sacrificial layers 158B, 158G, and 158R can be made to have a tapered shape relatively easily.

In the case where the first etching treatment is performed by dry etching, for example, a chlorine-based gas can be used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158B, 158G, and 158R can be formed with favorable in-plane uniformity.

The first etching treatment can be performed by wet etching, for example. The use of wet etching can reduce damage to the organic compound layers 103B, 103G, and 103R, as compared to the case of using dry etching.

The wet etching is preferably performed using an acidic chemical solution. As an acidic chemical solution, a chemical solution containing one of phosphoric acid, hydrofluoric acid, nitric acid, acetic acid, oxalic acid, sulfuric acid, and the like or a mixed chemical solution (also referred to as a mixed acid) that contains two or more of these acids is preferably used.

The wet etching can be performed using an alkaline solution. For instance, TMAH, which is an alkaline solution, can be used for the wet etching of an aluminum oxide film. In that case, puddle wet etching can be performed.

Then, heat treatment (also referred to as post-baking) is performed. The heat treatment can change the insulating layer 127a into the insulating layer 127 having a tapered side surface (see FIG. 9C). The heat treatment is conducted at a temperature lower than the upper temperature limit of the organic compound layer. The heat treatment can be performed at a substrate temperature of higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f.

The heat treatment can improve adhesion between the insulating layer 127 and the inorganic insulating layer 125 and increase corrosion resistance of the insulating layer 127. Furthermore, owing to the change in shape of the insulating layer 127a, an end portion of the inorganic insulating layer 125 can be covered with the insulating layer 127.

When the sacrificial layers 158B, 158G, and 158R are not completely removed by the first etching treatment and the thinned sacrificial layers 158B, 158G, and 158R are left, the organic compound layers 103B, 103G, and 103R can be prevented from being damaged and deteriorating in the heat treatment. This increases the reliability of the light-emitting device.

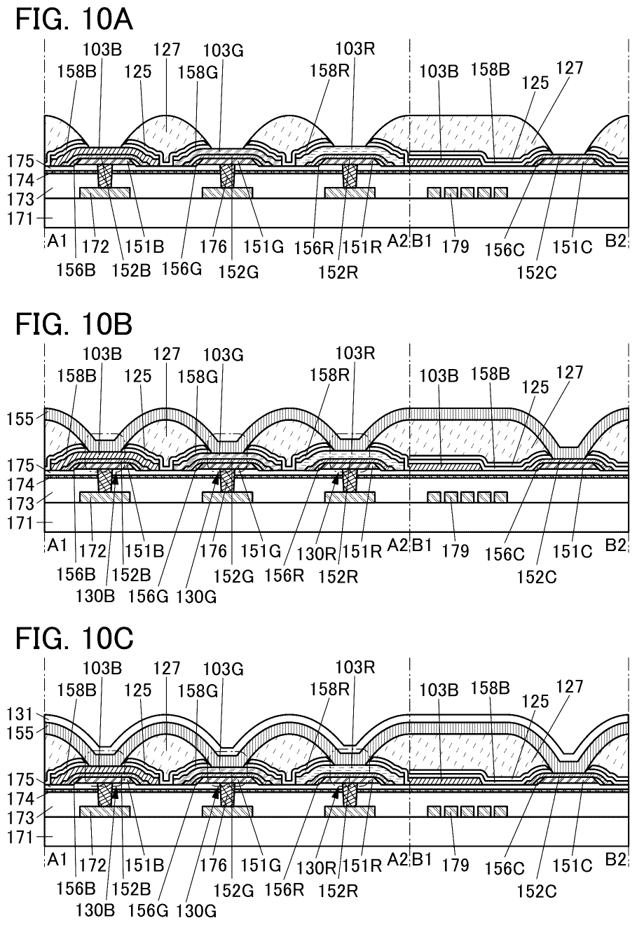

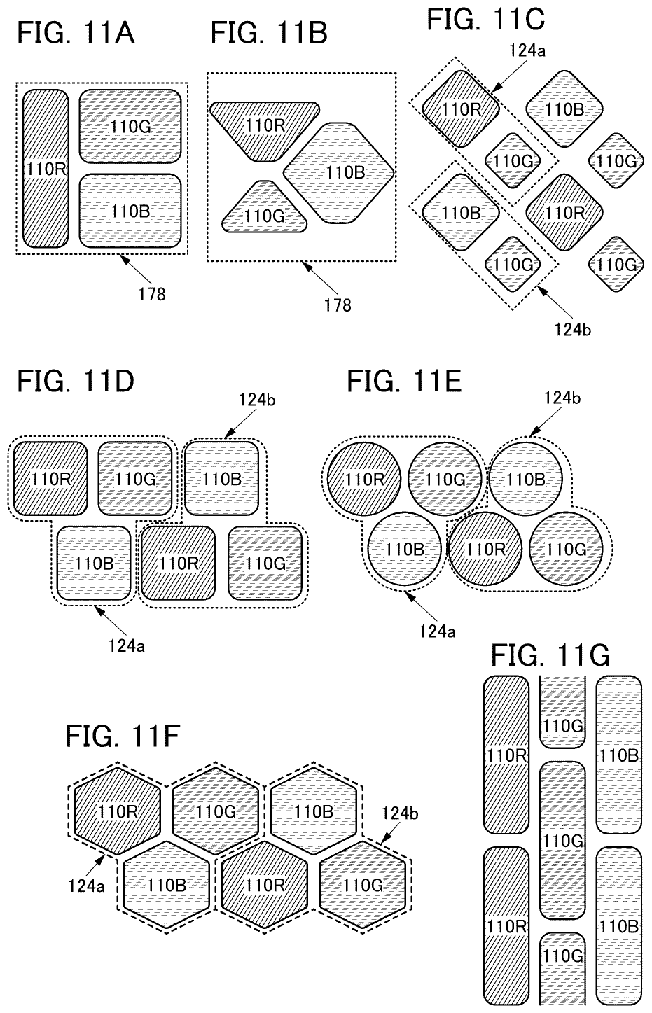

Next, as illustrated in FIG. 10A, etching treatment is performed with the insulating layer 127 as a mask to remove parts of the sacrificial layers 158B, 158G, and 158R. At this time, part of the inorganic insulating layer 125 is also removed in some cases. By the etching treatment, openings are formed in the sacrificial layers 158B, 158G, and 158R, and the top surfaces of the organic compound layers 103B, 103G, and 103R and the conductive layer 152C are exposed in the openings. Note that the etching treatment for exposing the organic compound layers 103B, 103G, and 103R using the insulating layer 127 as a mask may be hereinafter referred to as second etching treatment.

The second etching treatment is performed by wet etching. The use of a wet etching method can reduce damage to the organic compound layers 103B, 103G, and 103R, as compared to the case of using a dry etching method. The wet etching can be performed using an acidic chemical solution or an alkaline solution as in the case of the first etching treatment.

Heat treatment may be performed after the organic compound layers 103B, 103G, and 103R are partly exposed. By the heat treatment, water included in the organic compound layer and water adsorbed on the surface of the organic compound layer, for example, can be removed. The shape of the insulating layer 127 may be changed by the heat treatment. Specifically, the insulating layer 127 may be widened to cover at least one of the edge portion of the inorganic insulating layer 125, the edge portions of the sacrificial layers 158B, 158G, and 158R, and the top surfaces of the organic compound layers 103B, 103G, and 103R.

FIG. 10A illustrates an example in which part of the edge portion of the sacrificial layer 158G (specifically a tapered portion formed by the first etching treatment) is covered with the insulating layer 127 and a tapered portion formed by the second etching treatment is exposed (see FIG. 3A).

The insulating layer 127 may cover the entire edge portion of the sacrificial layer 158G. For example, the edge portion of the insulating layer 127 may droop to cover the edge portion of the sacrificial layer 158G. As another example, the edge portion of the insulating layer 127 may be in contact with the top surface of at least one of the organic compound layers 103B, 103G, and 103R.

Next, as illustrated in FIG. 10B, the common electrode 155 is formed over the organic compound layers 103B, 103G, and 103R, the conductive layer 152C, and the insulating layer 127. The common electrode 155 can be formed by a sputtering method, a vacuum evaporation method, or the like. Alternatively, the common electrode 155 may be formed by stacking a film formed by an evaporation method and a film formed by a sputtering method.

Next, as illustrated in FIG. 10C, the cap layer 131 is formed over the common electrode 155. The cap layer 131 can be formed by a vacuum evaporation method, a sputtering method, a CVD method, an ALD method, or the like.

Then, the substrate 120 is bonded over the cap layer 131 using the resin layer 122, whereby the light-emitting apparatus can be fabricated. In the method for fabricating the light-emitting apparatus of one embodiment of the present invention, the insulating layer 156 is formed to include a region overlapping the side surface of the conductive layer 151 and the conductive layer 152 is formed to cover the conductive layer 151 and the insulating layer 156 as described above. This can increase the yield of the light-emitting apparatus and inhibit generation of defects.

As described above, in the method for fabricating the light-emitting apparatus of one embodiment of the present invention, the island-shaped organic compound layers 103B, 103G, and 103R are formed not by using a fine metal mask but by processing a film formed on the entire surface; thus, the island-shaped layers can be formed to have a uniform thickness. Consequently, a high-resolution light-emitting apparatus or a light-emitting apparatus with a high aperture ratio can be obtained. Furthermore, even when the resolution or the aperture ratio is high and the distance between the subpixels is extremely short, the organic compound layers 103B, 103G, and 103R can be inhibited from being in contact with each other in the adjacent subpixels. As a result, generation of a leakage current between the subpixels can be inhibited. This can prevent crosstalk, so that a light-emitting apparatus with extremely high contrast can be obtained. Moreover, even a light-emitting apparatus that includes tandem light-emitting devices formed by a photolithography method can have favorable characteristics.

The structure described above in this embodiment can be combined as appropriate with any of the structures described in the other embodiments.

### Embodiment 4

In this embodiment, the light-emitting apparatus of one embodiment of the present invention will be described with reference to FIGS. 11A to 11G and FIGS. 12A to 121.

### [Pixel Layout]

In this embodiment, pixel layouts different from that in FIGS. 2A and 2B will be mainly described. There is no particular limitation on the arrangement of subpixels, and a variety of methods can be employed. Examples of the arrangement of subpixels include stripe arrangement, S-stripe arrangement, matrix arrangement, delta arrangement, Bayer arrangement, and PenTile arrangement.

In this embodiment, the top surface shapes of the subpixels shown in the diagrams correspond to top surface shapes of light-emitting regions.

Examples of a top surface shape of the subpixel include polygons such as a triangle, a tetragon (including a rectangle and a square), and a pentagon; polygons with rounded corners; an ellipse; and a circle.

The circuit constituting the subpixel is not necessarily placed within the dimensions of the subpixel illustrated in the diagrams and may be placed outside the subpixel.

The pixel 178 illustrated in FIG. 11A employs S-stripe arrangement. The pixel 178 illustrated in FIG. 11A includes three subpixels, the subpixel 110R, the subpixel 110G, and the subpixel 110B.

The pixel 178 illustrated in FIG. 11B includes the subpixel 110R whose top surface has a rough trapezoidal or rough triangle shape with rounded corners, the subpixel 110G whose top surface has a rough trapezoidal or rough triangle shape with rounded corners, and the subpixel 110B whose top surface has a rough tetragonal or rough hexagonal shape with rounded corners. The subpixel 110R has a larger light-emitting area than the subpixel 110G. In this manner, the shapes and sizes of the subpixels can be determined independently. For example, the size of a subpixel including a light-emitting device with higher reliability can be smaller.

Pixels 124a and 124b illustrated in FIG. 11C employ PenTile arrangement. FIG. 11C shows an example in which the pixels 124a including the subpixels 110R and 110G and the pixels 124b including the subpixels 110G and 110B are alternately arranged.

The pixels 124a and 124b illustrated in FIGS. 11D to 11F employ delta arrangement. The pixel 124a includes two subpixels (the subpixels 110R and 110G) in the upper row (first row) and one subpixel (the subpixel 110B) in the lower row (second row). The pixel 124b includes one subpixel (the subpixel 110B) in the upper row (first row) and two subpixels (the subpixels 110R and 110G) in the lower row (second row).

FIG. 11D illustrates an example where each subpixel has a rough tetragonal top surface with rounded corners. FIG. 11E illustrates an example where each subpixel has a circular top surface. FIG. 11F illustrates an example where each subpixel has a rough hexagonal top surface with rounded corners.

In FIG. 11F, subpixels are placed in respective hexagonal regions that are arranged densely. Focusing on one of the subpixels, the subpixel is placed so as to be surrounded by six subpixels. The subpixels are arranged such that subpixels that emit light of the same color are not adjacent to each other. For example, focusing on the subpixel 110R, the subpixel 110R is surrounded by three subpixels 110G and three subpixels 110B that are alternately arranged.

FIG. 11G shows an example where subpixels of different colors are arranged in a zigzag manner. Specifically, the positions of the top sides of two subpixels arranged in the row direction (e.g., the subpixels 110R and 110G or the subpixels 110G and 110B) are not aligned in the top view.

In the pixels illustrated in FIGS. 11A to 11G, for example, it is preferred that the subpixel 110R be a subpixel R that emits red light, the subpixel 110G be a subpixel G that emits green light, and the subpixel 110B be a subpixel B that emits blue light. Note that the structures of the subpixels are not limited thereto, and the colors and the order of the subpixels can be determined as appropriate. For example, the subpixel 110G may be the subpixel R that emits red light, and the subpixel 110R may be the subpixel G that emits green light.

In a photolithography method, as a pattern to be formed by processing becomes finer, the influence of light diffraction becomes more difficult to ignore; therefore, the fidelity in transferring a photomask pattern by light exposure is degraded, and it becomes difficult to process a resist mask into a desired shape. Thus, a pattern with rounded corners is likely to be formed even with a rectangular photomask pattern. Consequently, the top surface of a subpixel may have a polygonal shape with rounded corners, an elliptical shape, a circular shape, or the like.

Furthermore, in the method for fabricating the light-emitting apparatus of one embodiment of the present invention, the organic compound layer is processed into an island shape with the use of a resist mask. A resist film formed over the organic compound layer needs to be cured at a temperature lower than the upper temperature limit of the organic compound layer. Therefore, the resist film is insufficiently cured in some cases depending on the upper temperature limit of the material of the organic compound layer and the curing temperature of the resist material. An insufficiently cured resist film may have a shape different from a desired shape by processing. As a result, the top surface of the organic compound layer may have a polygonal shape with rounded corners, an elliptical shape, a circular shape, or the like. For example, when a resist mask with a square top surface is intended to be formed, a resist mask with a circular top surface may be formed, and the top surface of the organic compound layer may be circular.

To obtain a desired top surface shape of the organic compound layer, a technique of correcting a mask pattern in advance so that a transferred pattern agrees with a design pattern (an optical proximity correction (OPC) technique) may be used. Specifically, with the OPC technique, a pattern for correction is added to a corner portion of a figure on a mask pattern, for example.

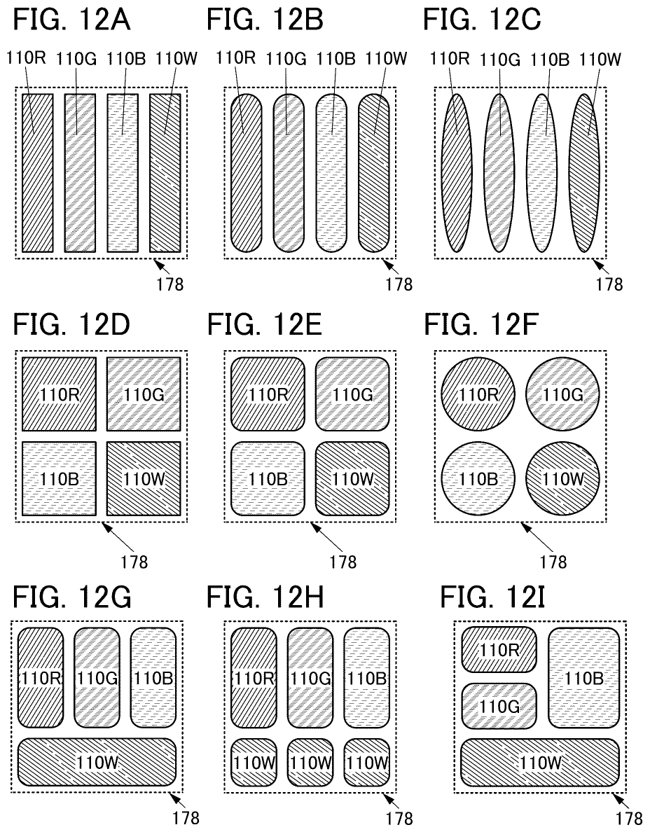

As illustrated in FIGS. 12A to 121, the pixel can include four types of subpixels.

The pixels 178 illustrated in FIGS. 12A to 12C employ stripe arrangement.

FIG. 12A illustrates an example where each subpixel has a rectangular top surface. FIG. 12B illustrates an example where each subpixel has a top surface shape formed by combining two half circles and a rectangle. FIG. 12C illustrates an example where each subpixel has an elliptical top surface.

The pixels 178 illustrated in FIGS. 12D to 12F employ matrix arrangement.

FIG. 12D illustrates an example where each subpixel has a square top surface. FIG. 12E illustrates an example where each subpixel has a substantially square top surface with rounded corners. FIG. 12F illustrates an example where each subpixel has a circular top surface.

FIGS. 12G and 12H each illustrate an example where one pixel 178 is composed of two rows and three columns.

The pixel 178 illustrated in FIG. 12G includes three subpixels (the subpixels 110R, 110G, and 110B) in the upper row (first row) and one subpixel (a subpixel 110W) in the lower row (second row). In other words, the pixel 178 includes the subpixel 110R in the left column (first column), the subpixel 110G in the middle column (second column), the subpixel 110B in the right column (third column), and the subpixel 110W across these three columns.

The pixel 178 illustrated in FIG. 12H includes three subpixels (the subpixels 110R, 110G, and 110B) in the upper row (first row) and three of the subpixels 110W in the lower row (second row). In other words, the pixel 178 includes the subpixels 110R and 110W in the left column (first column), the subpixels 110G and 110W in the middle column (second column), and the subpixels 110B and 110W in the right column (third column). Matching the positions of the subpixels in the upper row and the lower row as illustrated in FIG. 12H enables dust that would be produced in the fabrication process, for example, to be removed efficiently. Thus, a light-emitting apparatus having high display quality can be provided.

In the pixel 178 illustrated in FIGS. 12G and 12H, the subpixels 110R, 110G, and 110B are arranged in a stripe pattern, whereby the display quality can be improved.

FIG. 12I illustrates an example where one pixel 178 is composed of three rows and two columns.

The pixel 178 illustrated in FIG. 12I includes the subpixel 110R in the upper row (first row), the subpixel 110G in the middle row (second row), the subpixel 110B across the first row and the second row, and one subpixel (the subpixel 110W) in the lower row (third row). In other words, the pixel 178 includes the subpixels 110R and 110G in the left column (first column), the subpixel 110B in the right column (second column), and the subpixel 110W across these two columns.

In the pixel 178 illustrated in FIG. 12I, the subpixels 110R, 110G, and 110B are arranged in what is called an S-stripe pattern, whereby the display quality can be improved.

The pixel 178 illustrated in each of FIGS. 12A to 12I is composed of four subpixels, which are the subpixels 110R, 110G, 110B, and 110W. For example, the subpixel 110R can be a subpixel that emits red light, the subpixel 110G can be a subpixel that emits green light, the subpixel 110B can be a subpixel that emits blue light, and the subpixel 110W can be a subpixel that emits white light. Note that at least one of the subpixels 110R, 110G, 110B, and 110W may be a subpixel that emits cyan light, magenta light, yellow light, or near-infrared light.

As described above, the pixel composed of the subpixels each including the light-emitting device can employ any of a variety of layouts in the light-emitting apparatus of one embodiment of the present invention.

This embodiment can be combined as appropriate with the other embodiments or an example. In this specification, in the case where a plurality of structure examples are shown in one embodiment, the structure examples can be combined as appropriate.

### Embodiment 5

In this embodiment, a light-emitting apparatus of one embodiment of the present invention will be described.

The light-emitting apparatus in this embodiment can be a high-resolution light-emitting apparatus. Thus, the light-emitting apparatus in this embodiment can be used for display portions of information terminals (wearable devices) such as watch-type and bracelet-type information terminals and display portions of wearable devices capable of being worn on a head, such as a VR device like a head mounted display (HIMID) and a glasses-type AR device.

The light-emitting apparatus in this embodiment can be a high-definition light-emitting apparatus or a large-sized light-emitting apparatus. Accordingly, the light-emitting apparatus in this embodiment can be used for display portions of a digital camera, a digital video camera, a digital photo frame, a mobile phone, a portable game console, a portable information terminal, and an audio reproducing device, in addition to display portions of electronic apparatuses with a relatively large screen, such as a television device, desktop and notebook personal computers, a monitor of a computer and the like, digital signage, and a large game machine such as a pachinko machine.

### [Display Module]

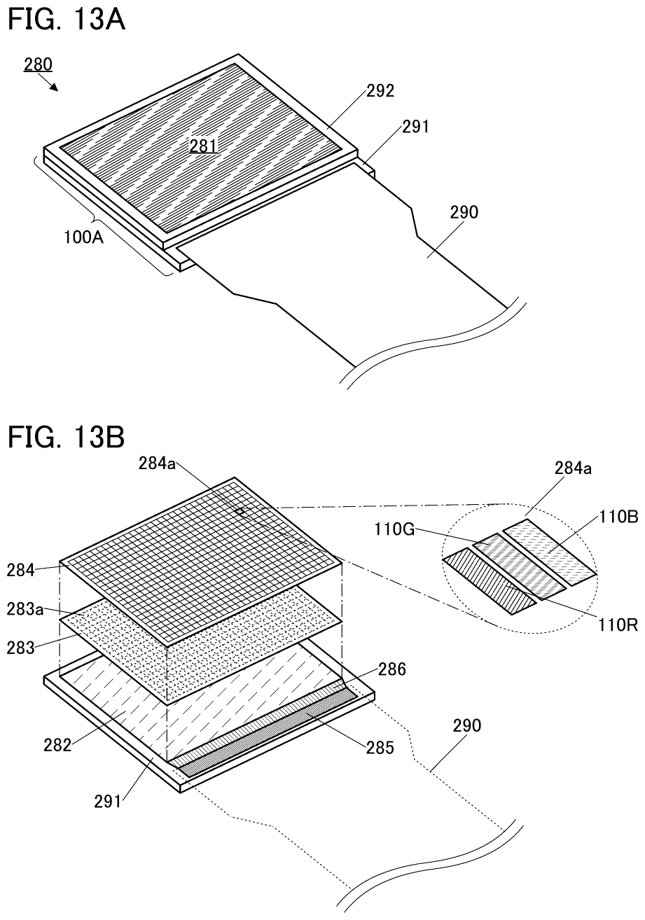

FIG. 13A is a perspective view of a display module 280. The display module 280 includes a light-emitting apparatus 100A and an FPC 290. Note that the light-emitting apparatus included in the display module 280 is not limited to the light-emitting apparatus 100A and may be either of light-emitting apparatuses 100B and 100C described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 13B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapped by the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 13B. The pixels 284a can employ any of the structures described in the above embodiments.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor for one light-emitting device. A gate signal is input to a gate of the selection transistor, and a video signal is input to a source or a drain of the selection transistor. With such a structure, an active-matrix light-emitting apparatus is achieved.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be greater than or equal to 40% and less than 100%, preferably greater than or equal to 50% and less than or equal to 95%, further preferably greater than or equal to 60% and less than or equal to 95%. Furthermore, the pixels 284a can be arranged extremely densely and thus the display portion 281 can have significantly high resolution. For example, the pixels 284a are preferably arranged in the display portion 281 with a resolution of greater than or equal to 2000 ppi, further preferably greater than or equal to 3000 ppi, still further preferably greater than or equal to 5000 ppi, yet still further preferably greater than or equal to 6000 ppi, and less than or equal to 20000 ppi or less than or equal to 30000 ppi.

Such a display module 280 has extremely high resolution, and thus can be suitably used for a VR device such as a HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic apparatuses including a relatively small display portion. For example, the display module 280 can be favorably used in a display portion of a wearable electronic apparatus, such as a wrist watch.

### [Light-Emitting Apparatus 100A]

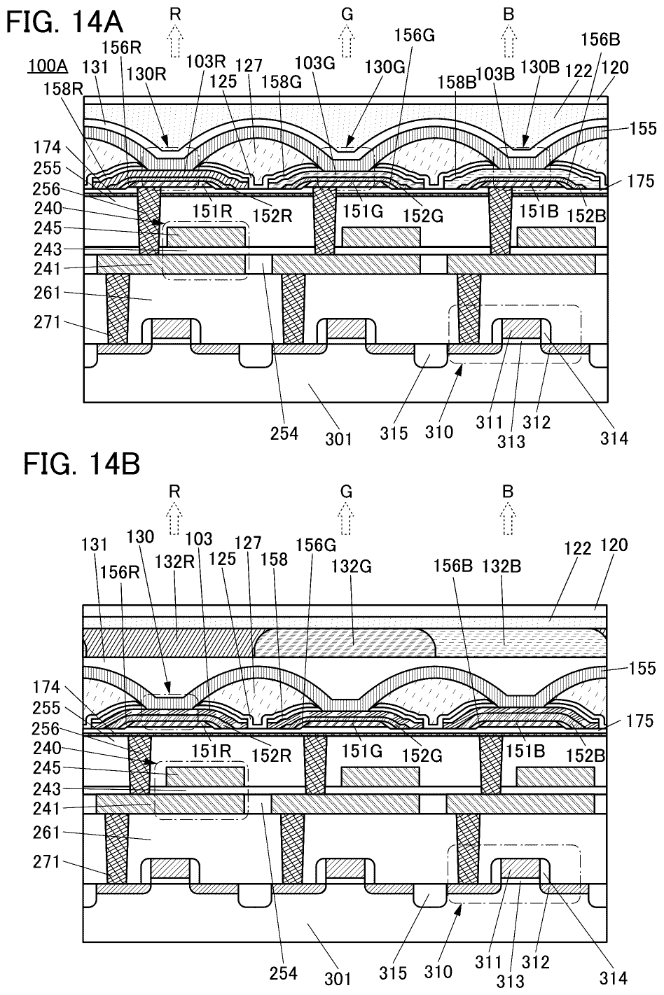

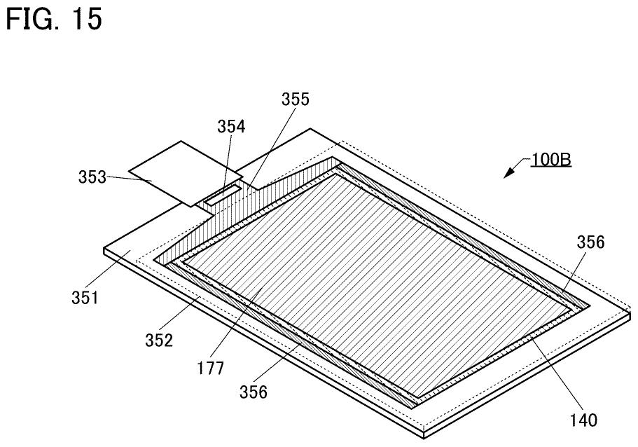

The light-emitting apparatus 100A illustrated in FIG. 14A includes a substrate 301, the light-emitting devices 130R, 130G, and 130B, a capacitor 240, and a transistor 310.

The substrate 301 corresponds to the substrate 291 in FIGS. 13A and 13B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. FIG. 14A illustrates an example in which the light-emitting devices 130R, 130G, and 130B each have the stacked-layer structure illustrated in FIG. 6A. An insulator is provided in regions between adjacent light-emitting devices. For example, in FIG. 14A, the inorganic insulating layer 125 and the insulating layer 127 over the inorganic insulating layer 125 are provided in those regions.

The insulating layer 156R is provided to include a region overlapping the side surface of the conductive layer 151R of the light-emitting device 130R. The insulating layer 156G is provided to include a region overlapping the side surface of the conductive layer 151G of the light-emitting device 130G. The insulating layer 156B is provided to include a region overlapping the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R of the light-emitting device 130R. The sacrificial layer 158G is positioned over the organic compound layer 103G of the light-emitting device 130G. The sacrificial layer 158B is positioned over the organic compound layer 103B of the light-emitting device 130B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The substrate 120 is bonded to the cap layer 131 of each light-emitting device with the resin layer 122. Embodiment 2 can be referred to for the details of the light-emitting device 130 and the components thereover up to the substrate 120. The substrate 120 corresponds to the substrate 292 in FIG. 13A.

FIG. 14B illustrates a variation example of the light-emitting apparatus 100A illustrated in FIG. 14A. The light-emitting apparatus illustrated in FIG. 14B includes the coloring layers 132R, 132G, and 132B, and each of the light-emitting devices 130 includes a region overlapped by one of the coloring layers 132R, 132G, and 132B. In the light-emitting apparatus illustrated in FIG. 14B, the light-emitting device 130 can emit white light, for example. For example, the coloring layer 132R, the coloring layer 132G, and the coloring layer 132B can transmit red light, green light, and blue light, respectively.

### [Light-Emitting Apparatus 100B]

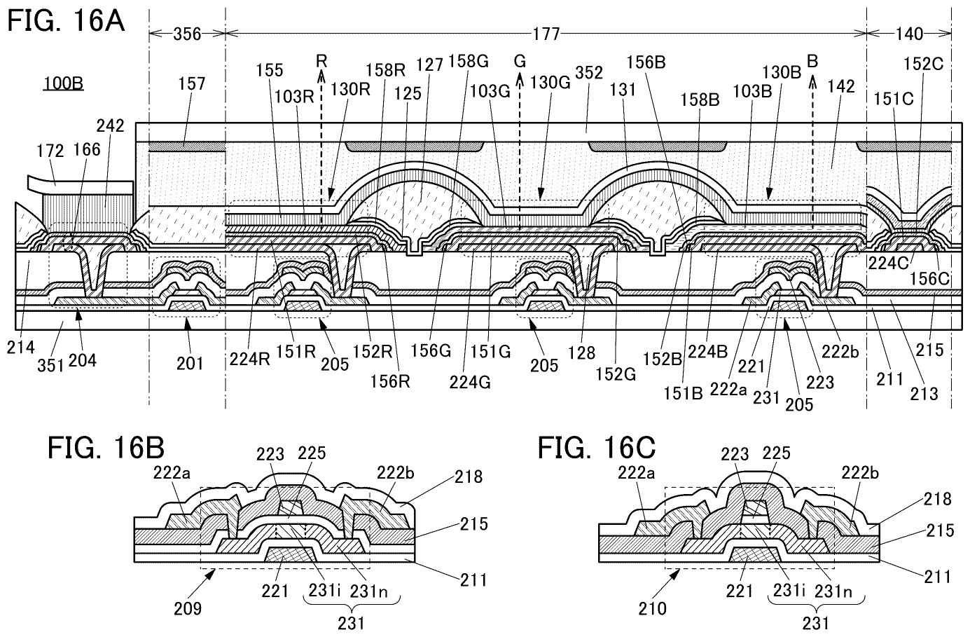

FIG. 15 is a perspective view of the light-emitting apparatus 100B, and FIG. 16A is a cross-sectional view of the light-emitting apparatus 100B.

In the light-emitting apparatus 100B, a substrate 352 and a substrate 351 are bonded to each other. In FIG. 15, the substrate 352 is denoted by a dashed line.

The light-emitting apparatus 100B includes the pixel portion 177, the connection portion 140, a circuit 356, a wiring 355, and the like. FIG. 15 illustrates an example in which an IC 354 and an FPC 353 are mounted on the light-emitting apparatus 100B. Thus, the structure illustrated in FIG. 15 can be regarded as a display module including the light-emitting apparatus 100B, the integrated circuit (IC), and the FPC. Here, a light-emitting apparatus in which a substrate is equipped with a connector such as an FPC or mounted with an IC is referred to as a display module.

The connection portion 140 is provided outside the pixel portion 177. The connection portion 140 can be provided along one side or a plurality of sides of the pixel portion 177. The number of connection portions 140 may be one or more. FIG. 15 illustrates an example in which the connection portion 140 is provided to surround the four sides of the pixel portion 177. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 15 illustrates an example in which the IC 354 is provided over the substrate 351 by a chip on glass (COG) method, a chip on film (COF) method, or the like. An IC including a scan line driver circuit, a signal line driver circuit, or the like can be used as the IC 354, for example. Note that the light-emitting apparatus 100B and the display module are not necessarily provided with an IC. Alternatively, the IC may be mounted on the FPC by a COF method, for example.

FIG. 16A illustrates an example of cross sections of part of a region including the FPC 353, part of the circuit 356, part of the pixel portion 177, part of the connection portion 140, and part of a region including an edge portion of the light-emitting apparatus 100B.

The light-emitting apparatus 100B illustrated in FIG. 16A includes a transistor 201, a transistor 205, the light-emitting device 130R that emits red light, the light-emitting device 130G that emits green light, the light-emitting device 130B that emits blue light, and the like between the substrate 351 and the substrate 352.

The stacked-layer structure of each of the light-emitting devices 130R, 130G, and 130B is the same as that illustrated in FIG. 6A except for the structure of the pixel electrode. Embodiments 1 and 2 can be referred to for the details of the light-emitting devices.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B. Here, the conductive layers 224R, 151R, and 152R can be collectively referred to as the pixel electrode of the light-emitting device 130R; the conductive layers 151R and 152R excluding the conductive layer 224R can also be referred to as the pixel electrode of the light-emitting device 130R.

Similarly, the conductive layers 224G, 151G, and 152G can be collectively referred to as the pixel electrode of the light-emitting device 130G; the conductive layers 151G and 152G excluding the conductive layer 224G can also be referred to as the pixel electrode of the light-emitting device 130G. The conductive layers 224B, 151B, and 152B can be collectively referred to as the pixel electrode of the light-emitting device 130B; the conductive layers 151B and 152B excluding the conductive layer 224B can also be referred to as the pixel electrode of the light-emitting device 130B.

The conductive layer 224R is connected to a conductive layer 222b included in the transistor 205 through the opening provided in an insulating layer 214. The edge portion of the conductive layer 151R is positioned outward from the edge portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G and the insulating layer 156G in the light-emitting device 130G are not described in detail because they are respectively similar to the conductive layers 224R, 151R, and 152R and the insulating layer 156R in the light-emitting device 130R; the same applies to the conductive layers 224B, 151B, and 152B and the insulating layer 156B in the light-emitting device 130B.

The conductive layers 224R, 224G, and 224B each have a depression portion covering an opening provided in the insulating layer 214. A layer 128 is embedded in the depression portion.

The layer 128 has a function of filling the depression portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping the depression portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The cap layer 131 is provided over the common electrode 155. The cap layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 16A, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided not to overlap the light-emitting device. Alternatively, the space may be filled with a resin other than the frame-like adhesive layer 142.

As the solid sealing structure, an inorganic insulating film such as a film containing nitrogen (e.g., a silicon nitride film) is provided. The cap layer 131 and the inorganic insulating film are preferably in contact with each other. The thickness of the inorganic insulating film is preferably larger than that of the cap layer, and the ordinary refractive index nₒ of the inorganic insulating film is preferably higher than that of the cap layer. This structure is expected to increase the light extraction efficiency.

FIG. 16A illustrates an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and the conductive layer 152C obtained by processing the same conductive film as the conductive layers 152R, 152G, and 152B. In the example illustrated in FIG. 16A, the insulating layer 156C is provided to include a region overlapping the side surface of the conductive layer 151C.

The light-emitting apparatus 100B has a top-emission structure. Light from the light-emitting device is emitted toward the substrate 352. For the substrate 352, a material having a high visible-light-transmitting property is preferably used. The pixel electrode contains a material that reflects visible light, and the counter electrode (the common electrode 155) contains a material that transmits visible light.

The transistor 201 and the transistor 205 are formed over the substrate 351. These transistors can be fabricated using the same materials in the same steps.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

A material through which impurities such as water and hydrogen do not easily diffuse is preferably used for at least one of the insulating layers covering the transistors. This is because such an insulating layer can function as a barrier layer. Such a structure can effectively inhibit diffusion of impurities to the transistors from the outside and increase the reliability of the light-emitting apparatus.

An inorganic insulating film is preferably used as each of the insulating layers 211, 213, and 215. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may be used. A stack including two or more of the above insulating films may also be used.

An organic insulating layer is suitable as the insulating layer 214 functioning as a planarization layer. Examples of materials that can be used for the organic insulating layer include an acrylic resin, a polyimide resin, an epoxy resin, a polyamide resin, a polyimide-amide resin, a siloxane resin, a benzocyclobutene-based resin, a phenol resin, and precursors of these resins. The insulating layer 214 may have a stacked-layer structure of an organic insulating layer and an inorganic insulating layer. The outermost layer of the insulating layer 214 preferably functions as an etching protective layer. This can inhibit formation of a recessed portion in the insulating layer 214 at the time of processing of the conductive layer 224R, 151R, or 152R or the like. Alternatively, a recessed portion may be provided in the insulating layer 214 at the time of processing of the conductive layer 224R, 151R, or 152R or the like.

Each of the transistors 201 and 205 includes a conductive layer 221 functioning as a gate, the insulating layer 211 functioning as a gate insulating layer, a conductive layer 222a and a conductive layer 222b functioning as a source and a drain, a semiconductor layer 231, the insulating layer 213 functioning as a gate insulating layer, and a conductive layer 223 functioning as a gate. Here, a plurality of layers obtained by processing the same conductive film are shown with the same hatching pattern. The insulating layer 211 is positioned between the conductive layer 221 and the semiconductor layer 231. The insulating layer 213 is positioned between the conductive layer 223 and the semiconductor layer 231.

There is no particular limitation on the structure of the transistors included in the light-emitting apparatus of this embodiment. For example, a planar transistor, a staggered transistor, or an inverted staggered transistor can be used. A top-gate transistor or a bottom-gate transistor can be used. Alternatively, gates may be provided above and below a semiconductor layer where a channel is formed.

The structure in which the semiconductor layer where a channel is formed is provided between two gates is used for the transistors 201 and 205. The two gates may be connected to each other and supplied with the same signal to operate the transistor. Alternatively, the threshold voltage of the transistor may be controlled by applying a potential for controlling the threshold voltage to one of the two gates and a potential for driving to the other of the two gates.

There is no particular limitation on the crystallinity of a semiconductor material used for the transistors, and either an amorphous semiconductor or a semiconductor having crystallinity (a microcrystalline semiconductor, a polycrystalline semiconductor, a single crystal semiconductor, or a semiconductor partly including crystal regions) can be used. A semiconductor having crystallinity is preferably used, in which case deterioration of transistor characteristics can be suppressed.

The semiconductor layer of the transistor preferably includes a metal oxide. That is, a transistor including a metal oxide in its channel formation region (hereinafter, also referred to as an OS transistor) is preferably used in the light-emitting apparatus of this embodiment.

Examples of an oxide semiconductor having crystallinity include a c-axis-aligned crystalline oxide semiconductor (CAAC-OS) and a nanocrystalline oxide semiconductor (nc-OS).

Alternatively, a transistor including silicon in its channel formation region (a Si transistor) may be used. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and excellent frequency characteristics.

With the use of Si transistors such as LTPS transistors, a circuit required to be driven at a high frequency (e.g., a source driver circuit) can be formed on the same substrate as the display portion. This allows for simplification of an external circuit mounted on the light-emitting apparatus and a reduction in costs of parts and mounting costs.

An OS transistor has much higher field-effect mobility than a transistor containing amorphous silicon. In addition, the OS transistor has an extremely low leakage current between a source and a drain in an off state (hereinafter also referred to as an off-state current), and charge accumulated in a capacitor that is connected in series to the transistor can be held for a long period. Furthermore, the power consumption of the light-emitting apparatus can be reduced with the OS transistor.

To increase the luminance of the light-emitting device included in the pixel circuit, the amount of current fed through the light-emitting device needs to be increased. To increase the current amount, the source-drain voltage of a driving transistor included in the pixel circuit needs to be increased. An OS transistor has a higher breakdown voltage between a source and a drain than a Si transistor; hence, a high voltage can be applied between the source and the drain of the OS transistor. Therefore, when an OS transistor is used as the driving transistor in the pixel circuit, the amount of current flowing through the light-emitting device can be increased, so that the luminance of the light-emitting device can be increased.

When transistors operate in a saturation region, a change in a source-drain current relative to a change in a gate-source voltage can be smaller in an OS transistor than in a Si transistor. Accordingly, when an OS transistor is used as the driving transistor in the pixel circuit, a current flowing between the source and the drain can be set minutely by a change in a gate-source voltage; hence, the amount of current flowing through the light-emitting device can be controlled. Consequently, the number of gray levels expressed by the pixel circuit can be increased.

Regarding saturation characteristics of a current flowing when transistors operate in a saturation region, even in the case where the source-drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source-drain current hardly changes with an increase in the source-drain voltage; hence, the luminance of the light-emitting device can be stable.

As described above, by using OS transistors as the driving transistors included in the pixel circuits, it is possible to inhibit black-level degradation, increase the luminance, increase the number of gray levels, and suppress variations in light-emitting devices, for example.

The semiconductor layer preferably contains indium, an element M1 (the element M1 is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, the element M1 is preferably one or more of aluminum, gallium, yttrium, and tin.

It is particularly preferable that an oxide containing indium (In), gallium (Ga), and zinc (Zn) (also referred to as IGZO) be used for the semiconductor layer. It is preferable to use an oxide containing indium, tin, and zinc. It is preferable to use an oxide containing indium, gallium, tin, and zinc. It is preferable to use an oxide containing indium (In), aluminum (Al), and zinc (Zn) (also referred to as IAZO). It is preferable to use an oxide containing indium (In), aluminum (Al), gallium (Ga), and zinc (Zn) (also referred to as IAGZO).

When the semiconductor layer is an In-M-Zn oxide, the atomic ratio of In is preferably greater than or equal to the atomic ratio of Min the In-M-Zn oxide. Examples of the atomic ratio of the metal elements in such an In-M-Zn oxide are In:M:Zn=1:1:1, 1:1:1.2, 2:1:3, 3:1:2, 4:2:3, 4:2:4.1, 5:1:3, 5:1:6, 5:1:7, 5:1:8, 6:1:6, and 5:2:5 and a composition in the vicinity of any of the above atomic ratios. Note that the vicinity of an atomic ratio includes ±30% of the intended atomic ratio.

For example, in the case of describing an atomic ratio of In:Ga:Zn=4:2:3 or a composition in the vicinity thereof, the case is included in which with the atomic proportion of In being 4, the atomic proportion of Ga is greater than or equal to 1 and less than or equal to 3 and the atomic proportion of Zn is greater than or equal to 2 and less than or equal to 4. In the case of describing an atomic ratio of In:Ga:Zn=5:1:6 or a composition in the vicinity thereof, the case is included in which with the atomic proportion of In being 5, the atomic proportion of Ga is greater than 0.1 and less than or equal to 2 and the atomic proportion of Zn is greater than or equal to 5 and less than or equal to 7. In the case of describing an atomic ratio of In:Ga:Zn=1:1:1 or a composition in the vicinity thereof, the case is included in which with the atomic proportion of In being 1, the atomic proportion of Ga is greater than 0.1 and less than or equal to 2 and the atomic proportion of Zn is greater than 0.1 and less than or equal to 2.

The transistors included in the circuit 356 and the transistors included in the pixel portion 177 may have the same structure or different structures. One structure or two or more kinds of structures may be employed for a plurality of transistors included in the circuit 356. Similarly, one structure or two or more kinds of structures may be employed for a plurality of transistors included in the pixel portion 177.

All transistors included in the pixel portion 177 may be OS transistors, or all transistors included in the pixel portion 177 may be Si transistors. Alternatively, some of the transistors included in the pixel portion 177 may be OS transistors and the others may be Si transistors.

For example, when both an LTPS transistor and an OS transistor are used in the pixel portion 177, the light-emitting apparatus can have low power consumption and high driving capability. Note that a structure in which an LTPS transistor and an OS transistor are used in combination is referred to as LTPO in some cases. For example, it is preferable that an OS transistor be used as a transistor functioning as a switch for controlling electrical continuity between wirings and an LTPS transistor be used as a transistor for controlling a current.

For example, one transistor included in the pixel portion 177 functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. In that case, the amount of current flowing through the light-emitting device can be increased in the pixel circuit.

Another transistor included in the pixel portion 177 functions as a switch for controlling selection or non-selection of a pixel and can be referred to as a selection transistor. A gate of the selection transistor is electrically connected to a gate line, and one of a source and a drain thereof is electrically connected to a source line (signal line). An OS transistor is preferably used as the selection transistor. In that case, the gray level of the pixel can be maintained even with an extremely low frame frequency (e.g., lower than or equal to 1 fps); thus, power consumption can be reduced by stopping the driver in displaying a still image.

As described above, the light-emitting apparatus of one embodiment of the present invention can have all of a high aperture ratio, high resolution, high display quality, and low power consumption.

Note that the light-emitting apparatus of one embodiment of the present invention has a structure including the OS transistor and the light-emitting device having a metal maskless (MML) structure. This structure can significantly reduce a leakage current that would flow through a transistor and a leakage current that would flow between adjacent light-emitting devices (sometimes referred to as a horizontal leakage current or a lateral leakage current). Displaying images on the light-emitting apparatus having this structure can bring one or more of image crispness, image sharpness, high color saturation, and a high contrast ratio to the viewer. When a leakage current that would flow through the transistor and a lateral leakage current that would flow between the light-emitting devices are extremely low, leakage of light at the time of black display (black-level degradation) or the like can be minimized.

In particular, in the case where a light-emitting device having an MML structure employs the above-described side-by-side (SBS) structure, a layer provided between light-emitting devices (for example, also referred to as an organic layer or a common layer which is shared by the light-emitting devices) is disconnected; accordingly, a leakage current can be prevented or be made extremely low.

FIGS. 16B and 16C illustrate other structure examples of transistors.

Transistors 209 and 210 each include the conductive layer 221 functioning as a gate, the insulating layer 211 functioning as a gate insulating layer, the semiconductor layer 231 including a channel formation region 231i and a pair of low-resistance regions 231n, the conductive layer 222a connected to one of the pair of low-resistance regions 231n, the conductive layer 222b connected to the other of the pair of low-resistance regions 231n, an insulating layer 225 functioning as a gate insulating layer, the conductive layer 223 functioning as a gate, and the insulating layer 215 covering the conductive layer 223. The insulating layer 211 is positioned between the conductive layer 221 and the channel formation region 231i. The insulating layer 225 is positioned at least between the conductive layer 223 and the channel formation region 231i. Furthermore, an insulating layer 218 covering the transistor may be provided.

FIG. 16B illustrates an example of the transistor 209 in which the insulating layer 225 covers the top and side surfaces of the semiconductor layer 231. The conductive layer 222a and the conductive layer 222b are connected to the corresponding low-resistance regions 231n through openings provided in the insulating layer 225 and the insulating layer 215. One of the conductive layers 222a and 222b functions as a source, and the other functions as a drain.

In the transistor 210 illustrated in FIG. 16C, the insulating layer 225 overlaps the channel formation region 231i of the semiconductor layer 231 and does not overlap the low-resistance regions 231n. The structure illustrated in FIG. 16C is obtained by processing the insulating layer 225 with the conductive layer 223 as a mask, for example. In FIG. 16C, the insulating layer 215 is provided to cover the insulating layer 225 and the conductive layer 223, and the conductive layer 222a and the conductive layer 222b are connected to the corresponding low-resistance regions 231n through openings in the insulating layer 215.

A connection portion 204 is provided in a region of the substrate 351 where the substrate 352 does not overlap. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

A light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

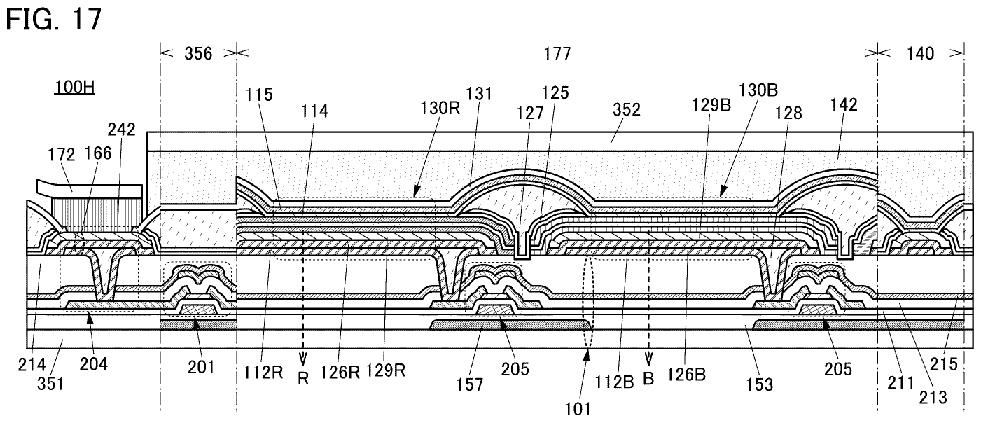

As the connection layer 242, an anisotropic conductive film (ACF), an anisotropic conductive paste (ACP), or the like can be used.

[Light-emitting apparatus 100H]A light-emitting apparatus 100H illustrated in FIG. 17 differs from the light-emitting apparatus 100B illustrated in FIG. 16A mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

The light-blocking layer 157 is preferably formed between the substrate 351 and the transistor 201 and between the substrate 351 and the transistor 205. FIG. 17 illustrates an example in which the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material having a high visible-light-transmitting property is used for each of the conductive layers 112R, 112B, 126R, 126B, 129R, and 129B. A material that reflects visible light is preferably used for the common electrode 155.

Although not illustrated in FIG. 17, the light-emitting device 130G is also provided in the light-emitting apparatus 100H.

Although FIG. 17 and the like illustrate an example in which the top surface of the layer 128 includes a flat portion, the shape of the layer 128 is not particularly limited.

### [Light-Emitting Apparatus 100C]

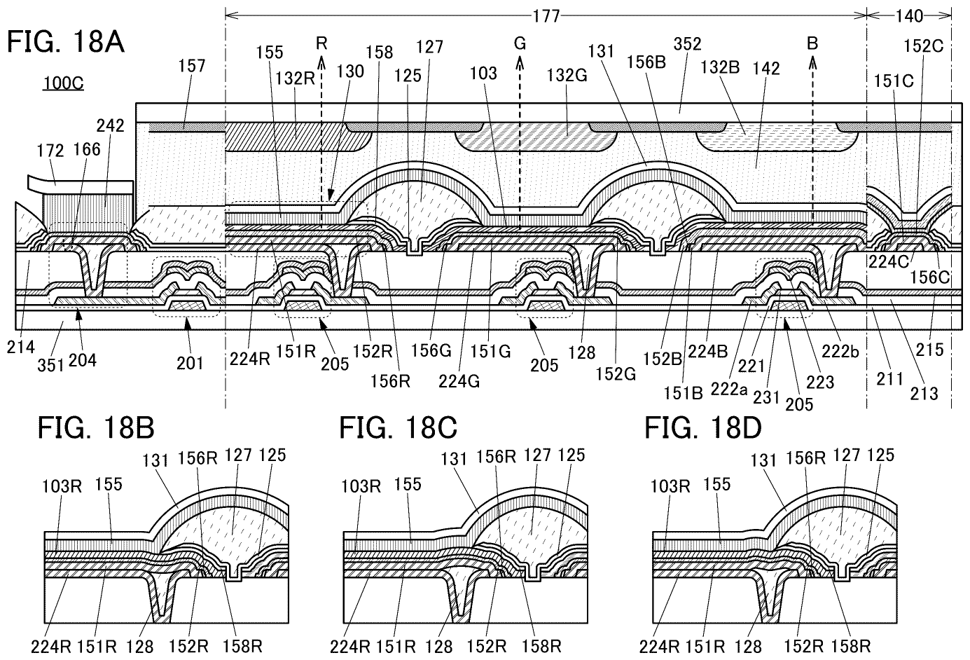

The light-emitting apparatus 100C illustrated in FIG. 18A is a variation example of the light-emitting apparatus 100B illustrated in FIG. 16A and differs from the light-emitting apparatus 100B mainly in including the coloring layers 132R, 132G, and 132B.

In the light-emitting apparatus 100C, the light-emitting device 130 includes a region overlapped by one of the coloring layers 132R, 132G, and 132B. The coloring layers 132R, 132G, and 132B can be provided on a surface of the substrate 352 on the substrate 351 side. The edge portions of the coloring layers 132R, 132G, and 132B can overlap the light-blocking layer 157.

In the light-emitting apparatus 100C, the light-emitting device 130 can emit white light, for example. The coloring layer 132R, the coloring layer 132G, and the coloring layer 132B can transmit red light, green light, and blue light, respectively, for example. Note that in the light-emitting apparatus 100C, the coloring layers 132R, 132G, and 132B may be provided between the cap layer 131 and the adhesive layer 142.

Although FIG. 16A, FIG. 18A, and the like illustrate an example in which the top surface of the layer 128 includes a flat portion, the shape of the layer 128 is not particularly limited. FIGS. 18B to 18D illustrate variation examples of the layer 128.

As illustrated in FIGS. 18B and 18D, the top surface of the layer 128 can have a shape such that its middle and the vicinity thereof are depressed (i.e., a shape including a concave surface) in the cross section.

As illustrated in FIG. 18C, the top surface of the layer 128 can have a shape in which its center and vicinity thereof bulge, i.e., a shape including a convex surface, in the cross section.

The top surface of the layer 128 may include one or both of a convex surface and a concave surface. The number of convex surfaces and the number of concave surfaces included in the top surface of the layer 128 are not limited and can each be one or more.

The level of the top surface of the layer 128 and the level of the top surface of the conductive layer 224R may be the same or substantially the same, or may be different from each other. For example, the level of the top surface of the layer 128 may be either lower or higher than the level of the top surface of the conductive layer 224R.

FIG. 18B can be regarded as illustrating an example in which the layer 128 fits in the depression portion of the conductive layer 224R. By contrast, as illustrated in FIG. 18D, the layer 128 may exist also outside the depression portion of the conductive layer 224R, i.e., the top surface of the layer 128 may extend beyond the depression portion.

This embodiment can be combined as appropriate with the other embodiments or examples. In this specification, in the case where a plurality of structure examples are shown in one embodiment, the structure examples can be combined as appropriate.

### Embodiment 6

In this embodiment, electronic apparatuses of embodiments of the present invention will be described.

Electronic apparatuses in this embodiment include the light-emitting apparatus of one embodiment of the present invention in their display portions. The light-emitting apparatus of one embodiment of the present invention is highly reliable and can be easily increased in resolution and definition. Thus, the light-emitting apparatus of one embodiment of the present invention can be used for display portions of a variety of electronic apparatuses.

Examples of the electronic apparatuses include a digital camera, a digital video camera, a digital photo frame, a mobile phone, a portable game console, a portable information terminal, and an audio reproducing device, in addition to electronic apparatuses with a relatively large screen, such as a television device, desktop and notebook personal computers, a monitor of a computer and the like, digital signage, and a large game machine such as a pachinko machine.

In particular, the light-emitting apparatus of one embodiment of the present invention can have high resolution, and thus can be favorably used for an electronic apparatus having a relatively small display portion. Examples of such an electronic apparatus include watch-type and bracelet-type information terminal devices (wearable devices) and wearable devices worn on the head, such as a VR device like a head-mounted display, a glasses-type AR device, and an MR device.

The definition of the light-emitting apparatus of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the light-emitting apparatus of one embodiment of the present invention is preferably higher than or equal to 100 ppi, further preferably higher than or equal to 300 ppi, further preferably higher than or equal to 500 ppi, further preferably higher than or equal to 1000 ppi, still further preferably higher than or equal to 2000 ppi, still further preferably higher than or equal to 3000 ppi, still further preferably higher than or equal to 5000 ppi, yet further preferably higher than or equal to 7000 ppi. With such a light-emitting apparatus having one or both of high definition and high resolution, the electronic apparatus can provide higher realistic sensation, sense of depth, and the like in personal use such as portable use or home use. There is no particular limitation on the screen ratio (aspect ratio) of the light-emitting apparatus of one embodiment of the present invention. For example, the light-emitting apparatus is compatible with a variety of screen ratios such as 1:1 (a square), 4:3, 16:9, and 16:10.

The electronic apparatus in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic apparatus in this embodiment can have a variety of functions. For example, the electronic apparatus in this embodiment can have a function of displaying a variety of data (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

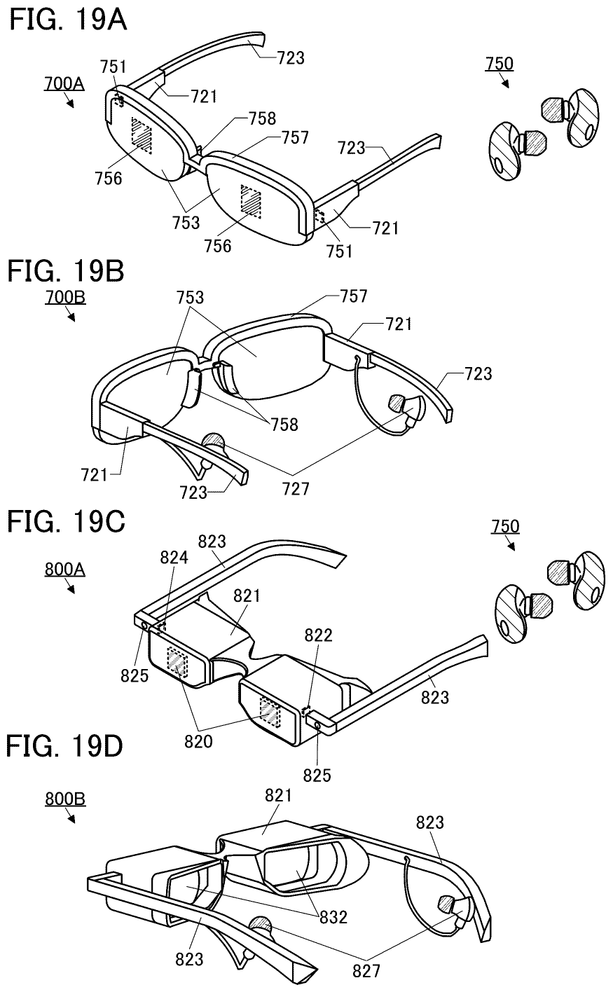

Examples of head-mounted wearable devices are described with reference to FIGS. 19A to 19D. These wearable devices have at least one of a function of displaying AR contents, a function of displaying VR contents, a function of displaying SR contents, and a function of displaying MR contents. The electronic apparatus having a function of displaying contents of at least one of AR, VR, SR, MR, and the like enables the user to feel a higher level of immersion.

An electronic apparatus 700A illustrated in FIG. 19A and an electronic apparatus 700B illustrated in FIG. 19B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The light-emitting apparatus of one embodiment of the present invention can be used for the display panels 751. Thus, a highly reliable electronic apparatus is obtained.

The electronic apparatuses 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic apparatuses 700A and 700B are electronic apparatuses capable of AR display.

In the electronic apparatuses 700A and 700B, a camera capable of capturing images of the front side may be provided as the image capturing portion. Furthermore, when the electronic apparatuses 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic apparatuses 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a video can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic apparatus 800A illustrated in FIG. 19C and an electronic apparatus 800B illustrated in FIG. 19D each include a pair of display portions 820, a housing 821, a communication portion 822, a pair of wearing portions 823, a control portion 824, a pair of image capturing portions 825, and a pair of lenses 832.

The light-emitting apparatus of one embodiment of the present invention can be used in the display portions 820. Thus, a highly reliable electronic apparatus is obtained.

The display portions 820 are positioned inside the housing 821 so as to be seen through the lenses 832. When the pair of display portions 820 display different images, three-dimensional display using parallax can be performed.

The electronic apparatuses 800A and 800B can be regarded as electronic apparatuses for VR. The user who wears the electronic apparatus 800A or the electronic apparatus 800B can see images displayed on the display portions 820 through the lenses 832.

The electronic apparatuses 800A and 800B preferably include a mechanism for adjusting the lateral positions of the lenses 832 and the display portions 820 so that the lenses 832 and the display portions 820 are positioned optimally in accordance with the positions of the user's eyes. Moreover, the electronic apparatuses 800A and 800B preferably include a mechanism for adjusting focus by changing the distance between the lenses 832 and the display portions 820.

The electronic apparatus 800A or the electronic apparatus 800B can be mounted on the user's head with the wearing portions 823. FIG. 19C, for instance, shows an example where the wearing portion 823 has a shape like a temple (also referred to as a joint or the like) of glasses; however, one embodiment of the present invention is not limited thereto. The wearing portion 823 can have any shape with which the user can wear the electronic apparatus, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is shown here, a range sensor (hereinafter also referred to as a sensing portion) capable of measuring a distance between the user and an object just needs to be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic apparatus 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic apparatus 800A.

The electronic apparatuses 800A and 800B may each include an input terminal. To the input terminal, a cable for supplying a video signal from a video output device or the like, power for charging a battery provided in the electronic apparatus, and the like can be connected.

The electronic apparatus of one embodiment of the present invention may have a function of performing wireless communication with earphones 750. The earphones 750 include a communication portion (not illustrated) and have a wireless communication function. The earphones 750 can receive information (e.g., audio data) from the electronic apparatus with the wireless communication function. For example, the electronic apparatus 700A in FIG. 19A has a function of transmitting information to the earphones 750 with the wireless communication function. As another example, the electronic apparatus 800A in FIG. 19C has a function of transmitting information to the earphones 750 with the wireless communication function.

The electronic apparatus may include an earphone portion. The electronic apparatus 700B in FIG. 19B includes earphone portions 727. For example, the earphone portion 727 can be connected to the control portion by wire. Part of a wiring that connects the earphone portion 727 and the control portion may be positioned inside the housing 721 or the mounting portion 723.

Similarly, the electronic apparatus 800B in FIG. 19D includes earphone portions 827. For example, the earphone portion 827 can be connected to the control portion 824 by wire. Part of a wiring that connects the earphone portion 827 and the control portion 824 may be positioned inside the housing 821 or the mounting portion 823. Alternatively, the earphone portions 827 and the mounting portions 823 may include magnets. This is preferred because the earphone portions 827 can be fixed to the mounting portions 823 with magnetic force and thus can be easily housed.

The electronic apparatus may include an audio output terminal to which earphones, headphones, or the like can be connected. The electronic apparatus may include one or both of an audio input terminal and an audio input mechanism. As the audio input mechanism, a sound collecting device such as a microphone can be used, for example. The electronic apparatus may have a function of a headset by including the audio input mechanism.

As described above, both the glasses-type device (e.g., the electronic apparatuses 700A and 700B) and the goggles-type device (e.g., the electronic apparatuses 800A and 800B) are preferable as the electronic apparatus of one embodiment of the present invention.

The electronic apparatus of one embodiment of the present invention can transmit information to earphones by wire or wirelessly.

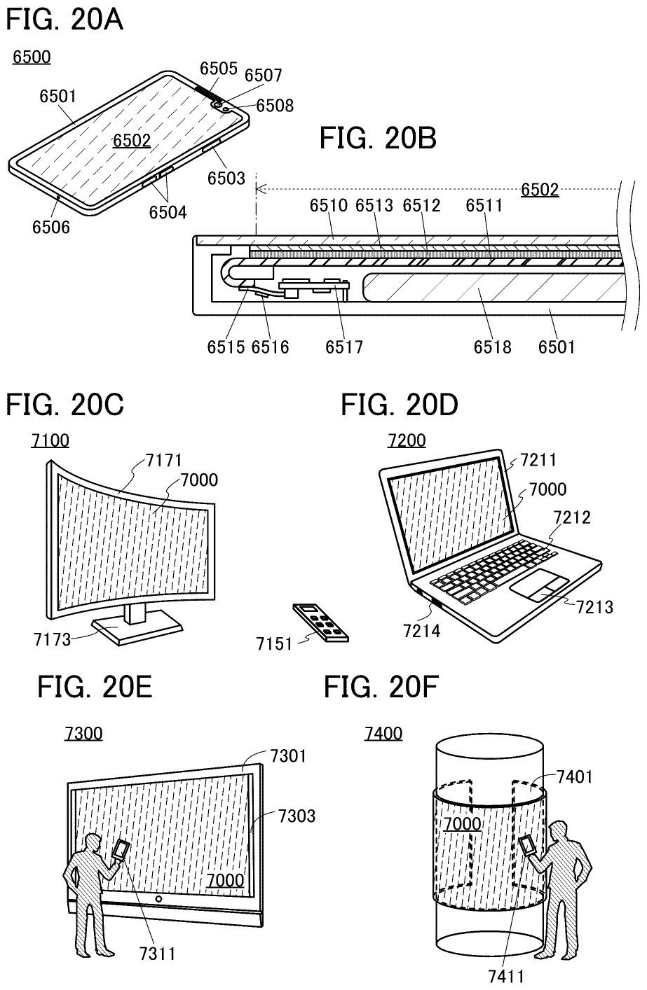

An electronic apparatus 6500 illustrated in FIG. 20A is a portable information terminal that can be used as a smartphone.

The electronic apparatus 6500 includes a housing 6501, a display portion 6502, a power button 6503, buttons 6504, a speaker 6505, a microphone 6506, a camera 6507, a light source 6508, and the like. The display portion 6502 has a touch panel function.

The light-emitting apparatus of one embodiment of the present invention can be used in the display portion 6502. Thus, a highly reliable electronic apparatus is obtained.

FIG. 20B is a schematic cross-sectional view including an edge portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

A flexible display using the organic compound of one embodiment of the present invention can be used as the display panel 6511. Thus, an extremely lightweight electronic apparatus can be achieved. Since the display panel 6511 is extremely thin, the battery 6518 with high capacity can be mounted without an increase in the thickness of the electronic apparatus. Moreover, part of the display panel 6511 is folded back so that a connection portion with the FPC 6515 is provided on the back side of the pixel portion, whereby an electronic apparatus with a narrow bezel can be achieved.

The organic compound of one embodiment of the present invention can be used in a display apparatus (e.g., a mobile phone or a tablet device) whose display panel 6511 is foldable or rollable. Light extraction efficiency might be decreased in a bent portion, such as a folded portion or a rolled portion, of the display panel. Using the organic compound of one embodiment of the present invention not only in a flat portion but also in a bent portion holds promise of inhibiting a decrease in light extraction efficiency.

FIG. 20C illustrates an example of a television device. In a television device 7100, a display portion 7000 is incorporated in a housing 7171. Here, the housing 7171 is supported by a stand 7173.

The light-emitting apparatus of one embodiment of the present invention can be used in the display portion 7000. Thus, a highly reliable electronic apparatus is obtained.

Operation of the television device 7100 illustrated in FIG. 20C can be performed with an operation switch provided in the housing 7171 and a separate remote controller 7151. Alternatively, the display portion 7000 may include a touch sensor, and the television device 7100 may be operated by touch on the display portion 7000 with a finger or the like. The remote controller 7151 may be provided with a display portion for displaying information output from the remote controller 7151. With operation keys or a touch panel of the remote controller 7151, channels and volume can be controlled and images displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 20D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The light-emitting apparatus of one embodiment of the present invention can be used in the display portion 7000. Thus, a highly reliable electronic apparatus is obtained.

FIGS. 20E and 20F illustrate examples of digital signage.

Digital signage 7300 illustrated in FIG. 20E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 20F shows digital signage 7400 attached to a cylindrical pillar 7401. The digital signage 7400 includes the display portion 7000 provided along a curved surface of the pillar 7401.

In FIGS. 20E and 20F, the light-emitting apparatus of one embodiment of the present invention can be used in the display portion 7000. Thus, a highly reliable electronic apparatus is obtained.

A larger area of the display portion 7000 can increase the amount of information that can be provided at a time. The display portion 7000 having a larger area attracts more attention, so that the effectiveness of the advertisement can be increased, for example.

The touch panel is preferably used in the display portion 7000, in which case in addition to display of still or moving images on the display portion 7000, intuitive operation by a user is possible. Moreover, in the case of an application for providing information such as route information or traffic information, usability can be enhanced by intuitive operation.

As illustrated in FIGS. 20E and 20F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

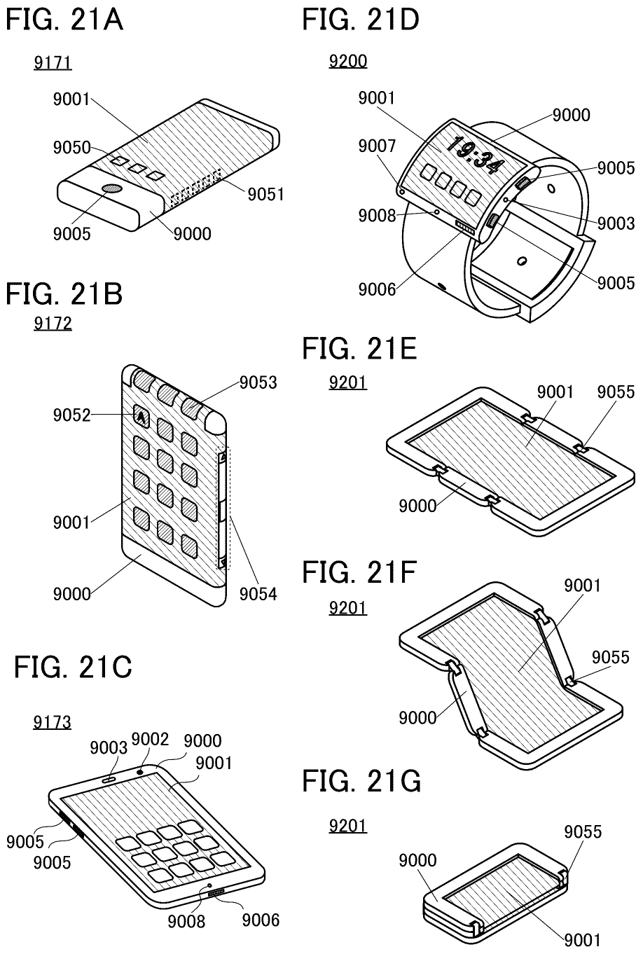

Electronic apparatuses illustrated in FIGS. 21A to 21G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic apparatuses illustrated in FIGS. 21A to 21G have a variety of functions. For example, the electronic apparatuses can have a function of displaying a variety of information (e.g., a still image, a moving image, and a text image) on the display portion, a touch panel function, a function of displaying a calendar, date, time, and the like, a function of controlling processing with the use of a variety of software (programs), a wireless communication function, and a function of reading out and processing a program or data stored in a recording medium. Note that the functions of the electronic apparatuses are not limited thereto, and the electronic apparatuses can have a variety of functions. The electronic apparatuses may include a plurality of display portions. The electronic apparatuses may be provided with a camera or the like and have a function of taking a still image or a moving image, a function of storing the taken image in a storage medium (an external storage medium or a storage medium incorporated in the camera), a function of displaying the taken image on the display portion, and the like.

The electronic apparatuses in FIGS. 21A to 21G are described in detail below.

FIG. 21A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 21A illustrates an example in which three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 21B is a perspective view of a portable information terminal 9172. The portable information terminal 9172 has a function of displaying information on three or more surfaces of the display portion 9001. Here, information 9052, information 9053, and information 9054 are displayed on different surfaces. For example, the user of the portable information terminal 9172 can check the information 9053 displayed such that it can be seen from above the portable information terminal 9172, with the portable information terminal 9172 put in a breast pocket of his/her clothes. Thus, the user can see the display without taking out the portable information terminal 9172 from the pocket and decide whether to answer the call, for example.

FIG. 21C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, the camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 21D is a perspective view of a watch-type portable information terminal 9200. The portable information terminal 9200 can be used as a Smartwatch (registered trademark), for example. The display surface of the display portion 9001 is curved, and an image can be displayed on the curved display surface. Furthermore, for example, mutual communication between the portable information terminal 9200 and a headset capable of wireless communication can be performed, and thus hands-free calling is possible. With the connection terminal 9006, the portable information terminal 9200 can perform mutual data transmission with another information terminal and charging. Note that the charging operation may be performed by wireless power feeding.

FIGS. 21E to 21G are perspective views of a foldable portable information terminal 9201. FIG. 21E is a perspective view showing the portable information terminal 9201 that is opened. FIG. 21G is a perspective view showing the portable information terminal 9201 that is folded. FIG. 21F is a perspective view showing the portable information terminal 9201 that is shifted from one of the states in FIGS. 21E and 21G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature of greater than or equal to 0.1 mm and less than or equal to 150 mm, for example.

This embodiment can be combined as appropriate with the other embodiments or an example. In this specification, in the case where a plurality of structure examples are shown in one embodiment, the structure examples can be combined as appropriate.

### Example 1

### Synthesis Example 1

In this synthesis example, a method for synthesizing 3-[4-(2,2′-binaphthalen-6-yl)phenyl]-9-phenyl-9H-carbazole (abbreviation: PCP(βN2)), which is the organic compound represented by Structural Formula (100) shown in Embodiment 1, is specifically described.

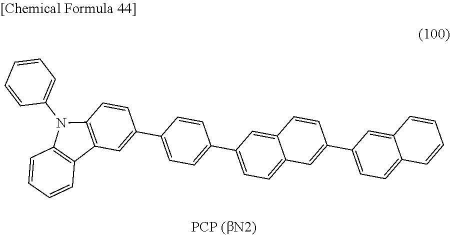 [SMILES: c1ccc(-n2c3ccccc3c3cc(-c4ccc(-c5ccc6cc(-c7ccc8ccccc8c7)ccc6c5)cc4)ccc32)cc1]

### Step 1: Synthesis of 6-(4-bromophenyl)-2,2′-binaphthalene

Step 1 was performed in the following procedure.

Into a 200-mL flask were put 2.97 g (10.5 mmol) of 4-bromoiodobenzene, 3.99 g (10.5 mmol) of 2-[(2,2′-binaphthalene)-6-yl]-4,4,5,5-tetramethyl-1,3,2-dioxaborolane, 192 mg (0.63 mmol) of tris(2-methylphenyl)phosphine (abbreviation: P(o-tolyl)₃), 4.35 g (31.5 mmol) of potassium carbonate (K₂CO₃), 53 mL of toluene, 11 mL of ethanol, and 16 mL of water. Then, the mixture was degassed while the pressure in the flask was reduced, and the air in the flask was replaced with nitrogen. Next, the mixture in the flask was heated at 40° C. under a nitrogen stream, and 71 mg (0.32 mmol) of palladium acetate (abbreviation: Pd(OAc)₂) was added thereto. Then, the temperature of the mixture was raised to 90° C. and the mixture was stirred for 7 hours while being heated. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene, so that 3.59 g of a white solid was obtained in a yield of 84%. A synthesis scheme of Step 1 is shown in (a-1) below.

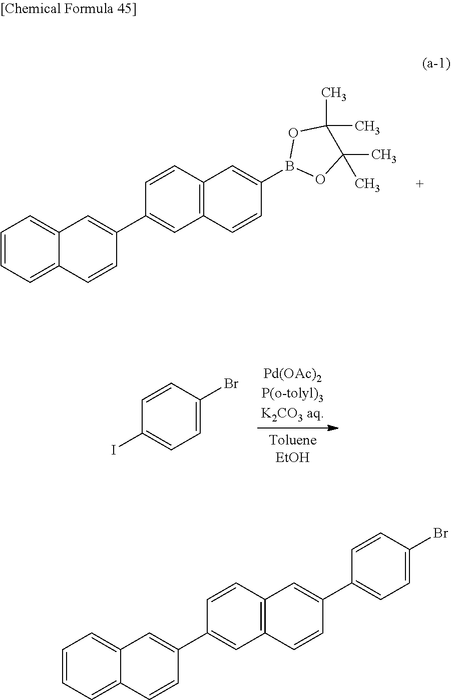 [SMILES: CC1(C)OB(c2ccc3cc(-c4ccc5ccccc5c4)ccc3c2)OC1(C)C ; Brc1ccc(I)cc1 ; Brc1ccc(-c2ccc3cc(-c4ccc5ccccc5c4)ccc3c2)cc1]

### Step 2: Synthesis of PCP(βN2)

Step 2 was performed in the following procedure.

Into a 200-mL three-neck flask were put 1.64 g (4.0 mmol) of 6-(4-bromophenyl)-2,2′-binaphthalene obtained in Step 1, 1.38 g (4.8 mmol) of 9-phenyl-9H-carbazol-3-boronic acid, 2.55 g (12.0 mmol) of tripotassium phosphate (abbreviation: K₃PO₄), and 20 mL of xylene. Then, the mixture was degassed while the pressure in the flask was reduced, and the air in the flask was replaced with nitrogen. Next, the mixture in the flask was heated at 40° C. under a nitrogen stream, and 86 mg (0.24 mmol) of di(1-adamantyl)-n-butylphosphine (abbreviation: cataCXium (registered trademark) A) and 27 mg (0.12 mmol) of palladium acetate were added thereto. Then, the temperature of the mixture was raised to 140° C. and the mixture was stirred for 16 hours. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene, so that 1.16 g of a white solid was obtained in a yield of 51%.

With a train sublimation method, 1.14 g of the obtained white solid was purified by heating at 310° C. for 27 hours under a pressure of 3.00 Pa with an argon flow rate of 6.5 mL/min to give 1.0 g of a white solid at a collection rate of 87%. As the result of mass spectrometry, it was confirmed that the target substance PCP(βN2) was obtained. A synthesis scheme of Step 2 is shown in (a-2) below.

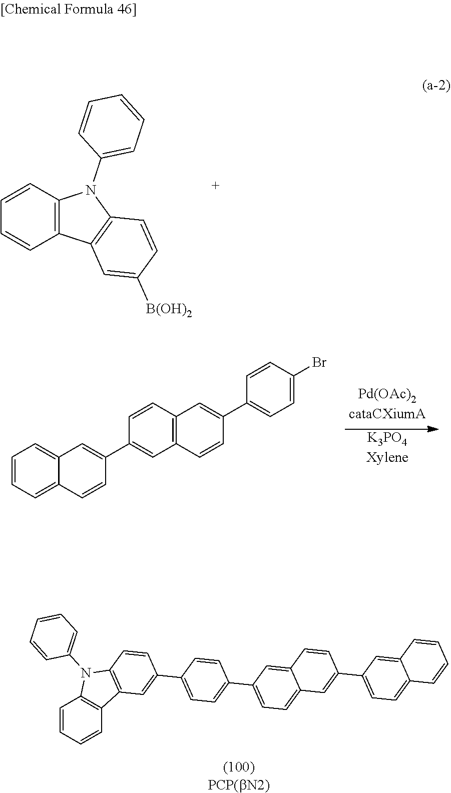 [SMILES: OB(O)c1ccc2c(c1)c1ccccc1n2-c1ccccc1 ; Brc1ccc(-c2ccc3cc(-c4ccc5ccccc5c4)ccc3c2)cc1 ; "K3PO4Xylene" ; c1ccc(-n2c3ccccc3c3cc(-c4ccc(-c5ccc6cc(-c7ccc8ccccc8c7)ccc6c5)cc4)ccc32)cc1]

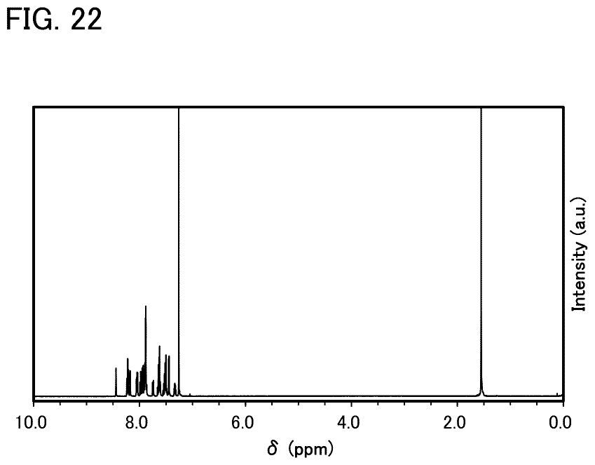

FIG. 22 shows a nuclear magnetic resonance spectroscopy (¹H-NMR) chart of PCP(βN2) after purification by sublimation in a deuterated chloroform (abbreviation: CDCl₃) solution. The results also show that PCP(βN2) was obtained.

¹H NMR (CDCl₃, 500 MHz): δ=7.32-7.36 (m, 1H), 7.45 (d, 2H), 7.49-7.56 (m, 4H), 7.61-7.66 (m, 4H), 7.75 (d, 1H), 7.86-7.99 (m, 10H), 8.05 (d, 2H), 8.18 (s, 1H), 8.21-8.24 (m, 3H), 8.44 (s, 1H).

### <Measurement of Emission and Absorption Spectra>

The absorption spectrum and the emission spectrum of PCP(βN2) were measured. The absorption spectrum was measured with an ultraviolet-visible spectrophotometer (V-770, manufactured by JASCO Corporation). The emission spectrum was measured with a fluorescence spectrophotometer (FP-8600, manufactured by JASCO Corporation).

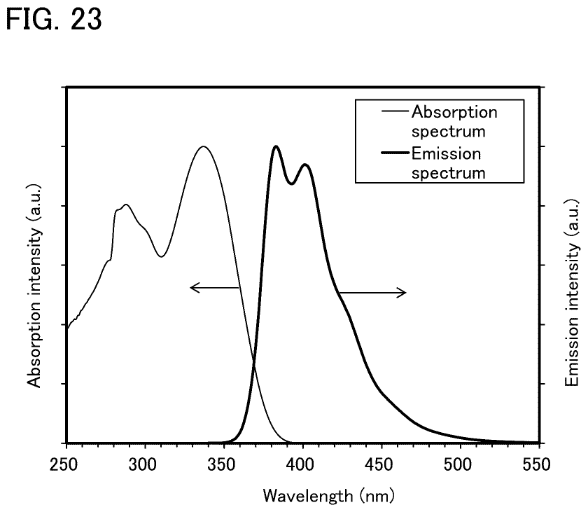

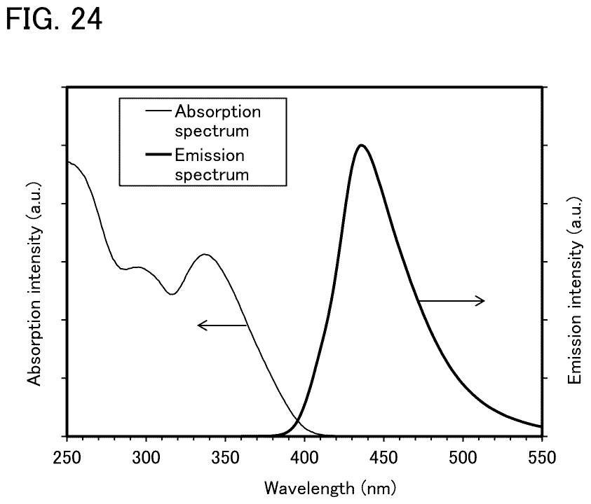

FIG. 23 shows an absorption spectrum and an emission spectrum of PCP(βN2) in a toluene solution. The absorption spectrum in the solution state was obtained by subtraction of a measured absorption spectrum of a solvent (toluene) alone in a quartz cell from a measured absorption spectrum of the toluene solution of PCP(βN2) in a quartz cell.

FIG. 24 shows an absorption spectrum and an emission spectrum of a thin film of PCP(βN2). The solid thin film was formed over a quartz substrate by a vacuum evaporation method and another quartz substrate as a counter substrate was used to perform sealing; thus, a measurement sample was fabricated. Note that the emission spectrum was obtained by measuring the sealed sample, and the absorption spectrum was obtained by measuring the sample from which the sealing was removed and the counter substrate was detached. The absorption spectrum of the thin film was obtained by subtraction of an absorption spectrum of the quartz substrate from an absorption spectrum of a PCP(βN2) film formed over the quartz substrate. Even after the removal of the sealing at room temperature, any change in film quality was not visually observed from the formed thin film and the stable amorphous film was found to be maintained. This revealed that the compound of the present application was able to form a thin film with excellent stability and the organic device fabricated with the compound of the present application exhibited excellent stability.

As shown in FIG. 23, PCP(βN2) in the toluene solution had absorption peaks at around 288 nm and 337 nm and emission peaks at around 383 nm and 401 nm (excitation wavelength: 330 nm). Furthermore, as shown in FIG. 24, PCP(βN2) in the thin film had absorption peaks at around 299 nm, 340 nm, and 370 nm, and an emission peak at around 436 nm (excitation wavelength: 336 nm).

These absorption characteristics indicate that no absorption was observed in the visible light range (wavelength range higher than or equal to 430 nm) needed for displays. Thus, PCP(βN2) can be suitably used for the cap film in a wavelength range needed for displays without reducing the emission efficiency of a light-emitting element. Furthermore, the obtained emission characteristics show that in the case where PCP(βN2) is used as a host in a light-emitting layer, energy can be efficiently transferred to a light-emitting material that emits light with a wavelength higher than or equal to 440 nm and lower than or equal to 700 nm and thus PCP(βN2) is suitable for a light-emitting element as well.

### <Measurement of Glass Transition Temperature (T_{g})>

The glass transition temperature (T_{g}) of PCP(βN2) was measured. The T_{g} was measured with a differential scanning calorimeter (PYRIS 1 DSC manufactured by PerkinElmer Japan Co., Ltd.) in a state where a powder was put on an aluminum sample pan.

The results showed that the T_{g} of PCP(βN2) was 126° C. The T_{g} available for an organic device is higher than or equal to 100° C., preferably higher than or equal to 110° C., further preferably higher than or equal to 120° C., still further preferably higher than or equal to 130° C. Therefore, the measurement results revealed that the compound of the present invention has an excellent thermal property and a thin film formed using the compound is expected to have stable film quality. The use of the compound capable of forming a thin film with stable film quality allows a highly heat-resistant organic device to be provided.

### <Calculation of HOMO and LUMO Levels>

The HOMO level and the LUMO level of PCP(βN2) were obtained through cyclic voltammetry (CV) measurement. The calculation method is shown below.

An electrochemical analyzer (ALS model 600A or 600C, manufactured by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (n-Bu₄NClO₄; produced by Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, produced by BAS Inc.) was used as a working electrode, a platinum electrode (Pt counter electrode for VC-3 (5 cm), produced by BAS Inc.) was used as an auxiliary electrode, and an Ag/Ag⁺ electrode (RE7 reference electrode for nonaqueous solvent, produced by BAS Inc.) was used as a reference electrode. Note that the measurement was performed at room temperature (20° C. to 25° C.).

The scan speed in the CV measurement was fixed to 0.1 V/see, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]=−4.94−Ec.

The CV measurement was repeated 100 times, and the oxidation-reduction wave in the hundredth cycle was compared with the oxidation-reduction wave in the first cycle to examine the electrical stability of the compound.

According to the measurement results of the oxidation potential Ea [V] of PCP(βN2), the HOMO level was found to be around −5.76 eV. According to the measurement results of the reduction potential Ec [V] of PCP(βN2), the LUMO level was found to be −2.49 eV. When the oxidation-reduction wave was repeatedly measured, in the Ea measurement, the peak intensity of the oxidation-reduction wave in the hundredth cycle was maintained to be 83% of that of the oxidation-reduction wave in the first cycle, and in the Ec measurement, the peak intensity of the oxidation-reduction wave in the hundredth cycle was maintained to be 93% of that of the oxidation-reduction wave in the first cycle; thus, resistance to oxidation and reduction of PCP(βN2) was found to be extremely high.

In consideration of the HOMO and LUMO levels, it is probable that holes and electrons can be favorably given and received, and PCP(βN2) can be suitably used for layers that need to transport carriers, such as a hole-transport layer, an electron-transport layer, a light-emitting layer, and a charge-generation layer of a tandem element in an organic device. In particular, since PCP(βN2) has a carbazole ring in its molecular structure, it can be suitably used for a hole-transport layer, a light-emitting layer, and a charge-generation layer of a tandem element which are responsible for hole transport.

### <Refractive Index Measurement>

The refractive index of PCP(βN2) was measured by a spectroscopic ellipsometer (M-2000U, manufactured by J. A. Woollam Japan). The PCP(βN2) film used for the measurement was formed to a thickness of approximately 50 nm over a quartz substrate by a vacuum evaporation method.

At a wavelength of 630 nm, n Ordinary (nₒ) that is the ordinary refractive index was 1.87, n Extra-ordinary (nₑ) that is the extraordinary refractive index was 1.65, and the difference between nₒ and nₑ was 0.21. At a wavelength of 520 nm, nₒ was 1.92, nₑ was 1.68, and the difference between nₒ and nₑ was 0.25. At a wavelength of 450 nm, nₒ was 2.01, nₑ was 1.71, and the difference between nₒ and nₑ was 0.30. Note that each measured value was expressed to three significant figures.

For the cap layer material, the difference between nₒ and nₑ is preferably greater than or equal to 0.1, further preferably greater than or equal to 0.2, still further preferably greater than or equal to 0.3 at any of three wavelengths of 450 nm, 520 nm, and 630 nm or at each of the three wavelengths. Therefore, the measurement results revealed that PCP(βN2) can be effectively used as a material of a cap layer provided over a cathode in a light-emitting apparatus.

Thus, it was found from the measurement results of the physical property values that PCP(βN2) can be effectively used for a cap layer used over a cathode. Furthermore, the results showed that PCP(βN2) can also be effectively used as a light-emitting substance or a host material used in combination with a substance that emits light in the visible range.

### Example 2

### Synthesis Example 2

In this synthesis example, a method for synthesizing 3-[4-(2,2′-binaphthalen-6-yl)phenyl]-9-(2-naphthyl)-9H-carbazole (abbreviation: βNCP(βN2)), which is the organic compound represented by Structural Formula (101) shown in Embodiment 1, is specifically described.

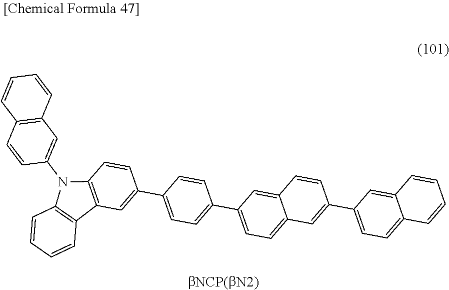 [SMILES: c1ccc2cc(-c3ccc4cc(-c5ccc(-c6ccc7c(c6)c6ccccc6n7-c6ccc7ccccc7c6)cc5)ccc4c3)ccc2c1]

### Step 1: Synthesis of βNCP(βN2)

Step 1 was performed in the following procedure.

Into a 200-mL three-neck flask were put 1.63 g (4.0 mmol) of 6-(4-bromophenyl)-2,2′-binaphthalene obtained in Step 1 of the above synthesis example 1, 1.62 g (4.8 mmol) of 9-(2-naphthalenyl)-9H-carbazol-3-boronic acid, 2.55 g (12.0 mmol) of tripotassium phosphate (abbreviation: K₃PO₄), and 20 mL of xylene. Then, the mixture was degassed while the pressure in the flask was reduced, and the air in the flask was replaced with nitrogen. Next, the mixture in the flask was heated at 40° C. under a nitrogen stream, and 86 mg (0.24 mmol) of di(1-adamantyl)-n-butylphosphine (abbreviation: cataCXium (registered trademark) A) and 27 mg (0.12 mmol) of palladium acetate were added thereto. Then, the temperature of the mixture was raised to 140° C. and the mixture was stirred for 12 hours. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene, so that 1.19 g of a white solid was obtained in a yield of 48%.

With a train sublimation method, 0.85 g of the obtained white solid was purified by heating at 325° C. for 18 hours under a pressure of 3.00 Pa with an argon flow rate of 6.5 mL/min to give 0.74 g of a white solid at a collection rate of 87%. As the result of mass spectrometry, it was confirmed that the target substance βNCP(βN2) was obtained. A synthesis scheme of Step 1 is shown in (b-1) below.

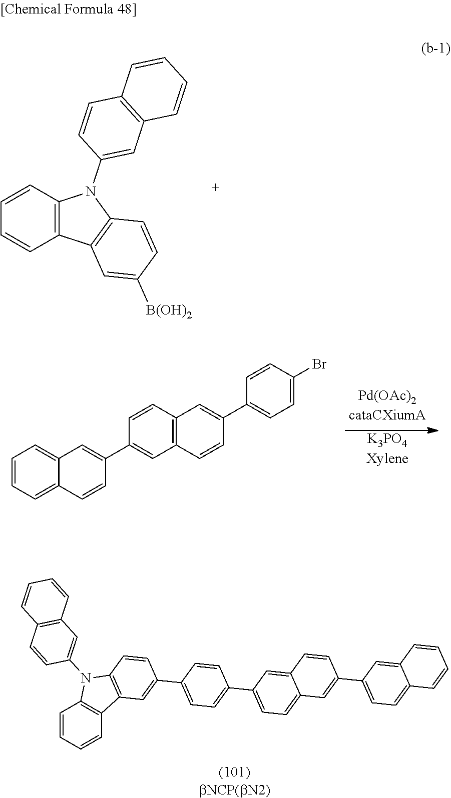 [SMILES: OB(O)c1ccc2c(c1)c1ccccc1n2-c1ccc2ccccc2c1 ; Brc1ccc(-c2ccc3cc(-c4ccc5ccccc5c4)ccc3c2)cc1 ; "K3PO4Xylene" ; c1ccc2cc(-c3ccc4cc(-c5ccc(-c6ccc7c(c6)c6ccccc6n7-c6ccc7ccccc7c6)cc5)ccc4c3)ccc2c1]

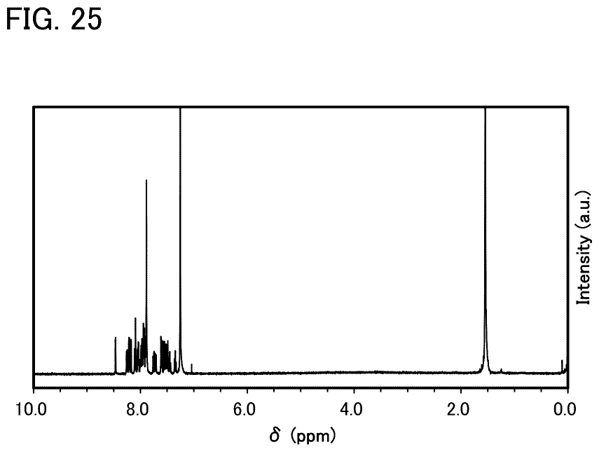

FIG. 25 shows a nuclear magnetic resonance spectroscopy (¹H-NMR) chart of βNCP(βN2) after purification by sublimation in a deuterated chloroform (abbreviation: CDCl₃) solution. The results also show that βNCP(βN2) was obtained.

¹H NMR (CDCl₃, 500 MHz): δ=7.35 (t, 1H), 7.44-7.56 (m, 5H), 7.60-7.62 (m, 2H), 7.72 (d, 1H), 7.76 (d, 1H), 7.89-8.01 (m, 12H), 8.05 (d, 2H), 8.09-8.11 (m, 2H), 8.18 (s, 1H), 8.21 (d, 2H), 8.25 (d, 1H), 8.47 (s, 1H).

### <Measurement of Emission and Absorption Spectra>

The absorption spectrum and the emission spectrum of βNCP(βN2) were measured. The absorption spectrum was measured with an ultraviolet-visible spectrophotometer (V-770, manufactured by JASCO Corporation). The emission spectrum was measured with a fluorescence spectrophotometer (FP-8600, manufactured by JASCO Corporation).

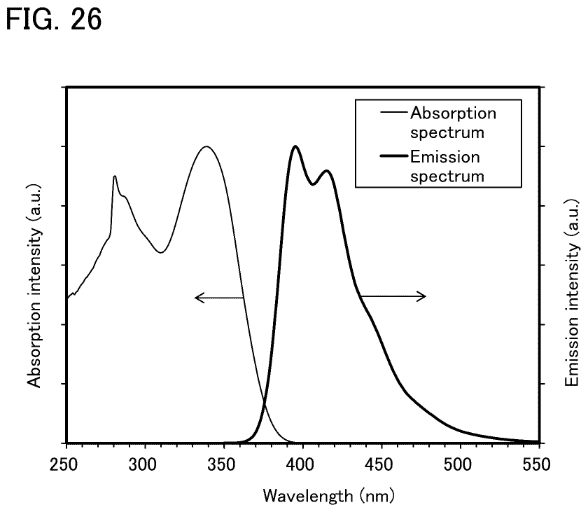

FIG. 26 shows an absorption spectrum and an emission spectrum of βNCP(βN2) in a toluene solution. The absorption spectrum in the solution state was obtained by subtraction of a measured absorption spectrum of a solvent (toluene) alone in a quartz cell from a measured absorption spectrum of the toluene solution of βNCP(βN2) in a quartz cell.

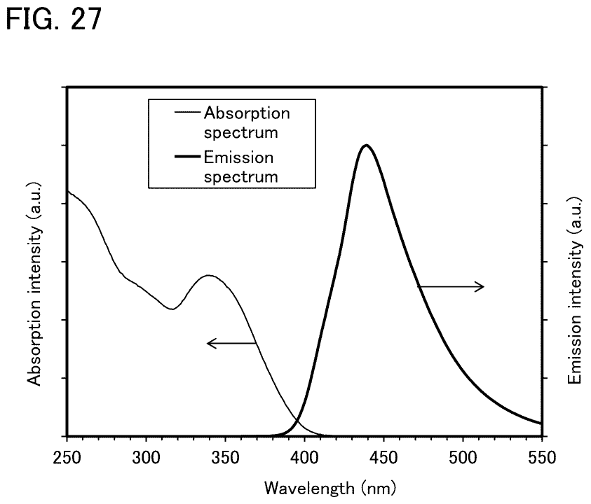

FIG. 27 shows an absorption spectrum and an emission spectrum of a thin film of βNCP(βN2). The solid thin film was formed over a quartz substrate by a vacuum evaporation method and another quartz substrate as a counter substrate was used to perform sealing; thus, a measurement sample was fabricated. Note that the emission spectrum was obtained by measuring the sealed sample, and the absorption spectrum was obtained by measuring the sample from which the sealing was removed and the counter substrate was detached. The absorption spectrum of the thin film was obtained by subtraction of an absorption spectrum of the quartz substrate from an absorption spectrum of a βNCP(βN2) film formed over the quartz substrate. Even after the removal of the sealing at room temperature, any change in film quality was not visually observed from the formed thin film and the stable amorphous film was found to be maintained. This revealed that the compound of the present application was able to form a thin film with excellent stability and the organic device fabricated with the compound of the present application exhibited excellent stability.

As shown in FIG. 26, βNCP(βN2) in the toluene solution had absorption peaks at around 281 nm and 339 nm and emission peaks at around 395 nm and 415 nm (excitation wavelength: 340 nm). Furthermore, as shown in FIG. 27, βNCP(βN2) in the thin film had absorption peaks at around 262 nm, 295 nm, and 345 nm, and an emission peak at around 439 nm (excitation wavelength: 341 nm).

These absorption characteristics indicate that no absorption was observed in the visible light range (wavelength range higher than or equal to 430 nm) needed for displays. Thus, βNCP(βN2) can be suitably used for the cap film in a wavelength range needed for displays without reducing the emission efficiency of a light-emitting element. Furthermore, the obtained emission characteristics show that in the case where βNCP(βN2) is used as a host in a light-emitting layer, energy can be efficiently transferred to a light-emitting material that emits light with a wavelength higher than or equal to 440 nm and lower than or equal to 700 nm and thus βNCP(βN2) is suitable for a light-emitting element as well.

### <Measurement of Glass Transition Temperature (T_{g})>

The glass transition temperature (T_{g}) of βNCP(βN2) was measured. The T_{g} was measured with a differential scanning calorimeter (PYRIS 1 DSC manufactured by PerkinElmer Japan Co., Ltd.) in a state where a powder was put on an aluminum sample pan.

The results showed that the T_{g} of βNCP(βN2) was 126° C. The T_{g} available for an organic device is higher than or equal to 100° C., preferably higher than or equal to 110° C., further preferably higher than or equal to 120° C., still further preferably higher than or equal to 130° C. Therefore, the measurement results revealed that the compound of the present invention has an excellent thermal property and a thin film formed using the compound is expected to have stable film quality. The use of the compound capable of forming a thin film with stable film quality allows a highly heat-resistant organic device to be provided.

### <Calculation of HOMO and LUMO Levels>

The HOMO level and the LUMO level of βNCP(βN2) were obtained through cyclic voltammetry (CV) measurement. The calculation method is shown below.

An electrochemical analyzer (ALS model 600A or 600C, manufactured by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (n-Bu₄NClO₄; produced by Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, produced by BAS Inc.) was used as a working electrode, a platinum electrode (Pt counter electrode for VC-3 (5 cm), produced by BAS Inc.) was used as an auxiliary electrode, and an Ag/Ag⁺ electrode (RE7 reference electrode for nonaqueous solvent, produced by BAS Inc.) was used as a reference electrode. Note that the measurement was performed at room temperature (20° C. to 25° C.).

The scan speed in the CV measurement was fixed to 0.1 V/see, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]=−4.94−Ec.

The CV measurement was repeated 100 times, and the oxidation-reduction wave in the hundredth cycle was compared with the oxidation-reduction wave in the first cycle to examine the electrical stability of the compound.

According to the measurement results of the oxidation potential Ea [V] of βNCP(βN2), the HOMO level was found to be around −5.74 eV. According to the measurement results of the reduction potential Ec [V] of βNCP(βN2), the LUMO level was found to be −2.48 eV. When the oxidation-reduction wave was repeatedly measured, in the Ea measurement, the peak intensity of the oxidation-reduction wave in the hundredth cycle was maintained to be 76% of that of the oxidation-reduction wave in the first cycle, and in the Ec measurement, the peak intensity of the oxidation-reduction wave in the hundredth cycle was maintained to be 91% of that of the oxidation-reduction wave in the first cycle; thus, resistance to oxidation and reduction of βNCP(βN2) was found to be extremely high.

In consideration of the HOMO and LUMO levels, it is probable that holes and electrons can be favorably given and received, and βNCP(βN2) can be suitably used for layers that need to transport carriers, such as a hole-transport layer, an electron-transport layer, a light-emitting layer, and a charge-generation layer of a tandem element in an organic device. In particular, since βNCP(βN2) has a carbazole ring in its molecular structure, it can be suitably used for a hole-transport layer, a light-emitting layer, and a charge-generation layer of a tandem element which are responsible for hole transport.

### <Refractive Index Measurement>

The refractive index of βNCP(βN2) was measured by a spectroscopic ellipsometer (M-2000U, manufactured by J. A. Woollam Japan). The βNCP(βN2) film used for the measurement was formed to a thickness of approximately 51 nm over a quartz substrate by a vacuum evaporation method.

At a wavelength of 630 nm, n Ordinary (nₒ) that is the ordinary refractive index was 1.87, n Extra-ordinary (nₑ) that is the extraordinary refractive index was 1.66, and the difference between nₒ and nₑ was 0.21. At a wavelength of 520 nm, nₒ was 1.93, nₑ was 1.69, and the difference between nₒ and nₑ was 0.24. At a wavelength of 450 nm, nₒ was 2.03, nₑ was 1.73, and the difference between nₒ and nₑ was 0.30. Note that each measured value was expressed to three significant figures.

For the cap layer material, the difference between nₒ and nₑ is preferably greater than or equal to 0.1, further preferably greater than or equal to 0.2, still further preferably greater than or equal to 0.3 at any of three wavelengths of 450 nm, 520 nm, and 630 nm or at each of the three wavelengths. Therefore, the measurement results revealed that βNCP(βN2) can be effectively used as a material of a cap layer provided over a cathode in a light-emitting apparatus.

Thus, it was found from the measurement results of the physical property values that βNCP(βN2) can be effectively used for a cap layer used over a cathode. Furthermore, the results showed that βNCP(βN2) can also be effectively used as a light-emitting substance or a host material used in combination with a substance that emits light in the visible range.

### Example 3

### Synthesis Example 3

In this synthesis example, a method for synthesizing 3-[4-(2-phenanthlenyl)phenyl]-9-(2-phenanthryl)-9H-carbazole (abbreviation: PnCPPn), which is the organic compound represented by Structural Formula (102) shown in Embodiment 1, is specifically described.

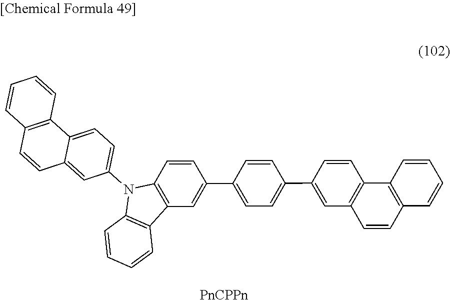 [SMILES: c1ccc2c(c1)ccc1cc(-c3ccc(-c4ccc5c(c4)c4ccccc4n5-c4ccc5c(ccc6ccccc65)c4)cc3)ccc12]

### Step 1: Synthesis of4,4,5,5-tetramethyl-2-(phenanthren-2-yl)-1,3,2-dioxaborolane

Step 1 was performed in the following procedure.

Into a 500-mL flask were put 10.3 g (40.0 mmol) of 2-bromophenanthrene, 12.2 g (48.0 mmol) of bis(pinacolato)diboron, 11.8 g (120 mmol) of potassium acetate (KOAc), and 200 mL of 1,4-dioxane. Then, the mixture was degassed while the pressure in the flask was reduced, and the air in the flask was replaced with nitrogen. Next, the mixture in the flask was heated at 40° C. under a nitrogen stream, and 980 mg (1.2 mmol) of [1,1′-bis(diphenylphosphino)ferrocene]palladium(II) dichloride dichloromethane adduct (abbreviation: PdCl₂(dppf)-CH₂Cl₂) was added thereto. Then, the temperature of the mixture was raised to 100° C. and the mixture was stirred for 7 hours while being heated. After the reaction, this mixture was suction-filtered, and the obtained filtrate was concentrated, and purification by silica gel column chromatography (toluene:hexane=1:1) was performed. The fraction was concentrated and dried to give 11.8 g of a white solid in a yield of 97%. A synthesis scheme of Step 1 is shown in (c-1) below.

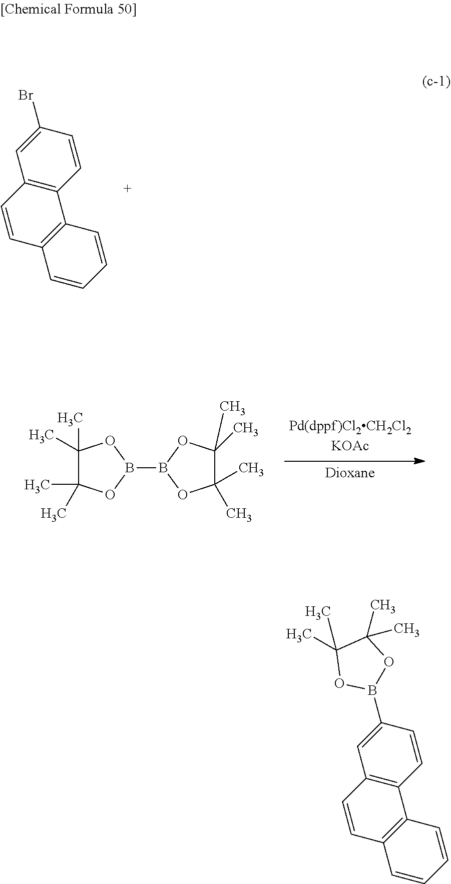

### Step 2: Synthesis of 2-(4-bromophenyl)-phenanthrene

Step 2 was performed in the following procedure.

Into a 500-mL three-neck flask were put 11.8 g (38.8 mmol) of 4,4,5,5-tetramethyl-2-(phenanthren-2-yl)-1,3,2-dioxaborolane obtained in Step 1, 11.8 g (38.8 mmol) of 4-bromoiodobenzene, 14.7 mg (106 mmol) of potassium carbonate (abbreviation: K₂CO₃), 177 mL of toluene, 35 mL of ethanol, and 53 mL of water. Then, the mixture was degassed while the pressure in the flask was reduced, and the air in the flask was replaced with nitrogen. Next, the mixture in the flask was heated at 40° C. under a nitrogen stream, and 645 mg (2.12 mmol) of tris(2-methylphenyl)phosphine (abbreviation: P(o-tolyl)₃) and 238 mg (1.06 mmol) of palladium acetate were added thereto. Then, the temperature of the mixture was raised to 90° C. and the mixture was stirred for 6 hours while being heated. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene and ethanol, so that 11.1 g of a white solid was obtained in a yield of 86%. A synthesis scheme of Step 2 is shown in (c-2) below.

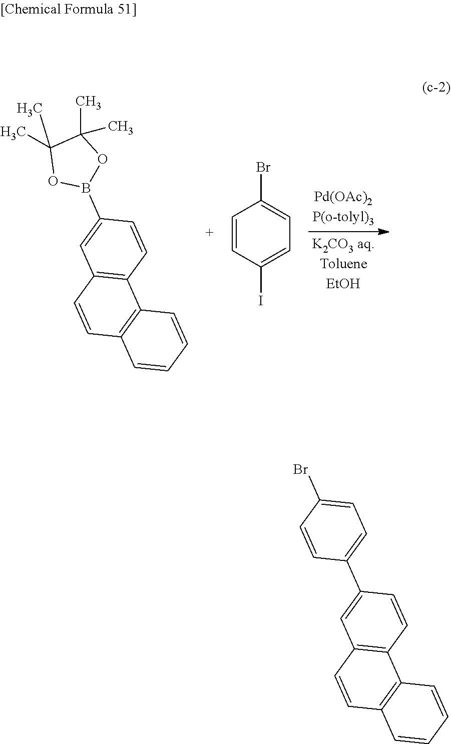 [SMILES: CC1(C)OB(c2ccc3c(ccc4ccccc43)c2)OC1(C)C ; Brc1ccc(I)cc1 ; Brc1ccc(-c2ccc3c(ccc4ccccc43)c2)cc1]

### Step 3: Synthesis of 4,4,5,5-tetramethyl-2-[4-(phenanthren-2-yl)phenyl]-1,3,2-dioxaborolane

Step 3 was performed in the following procedure.

Into a 500-mL flask were put 6.00 g (18.0 mmol) of 2-(4-bromophenyl)-phenanthrene obtained in Step 2, 5.49 g (21.6 mmol) of bis(pinacolato)diboron, 5.30 g (54.0 mmol) of potassium acetate (KOAc), and 90 mL of 1,4-dioxane. Then, the mixture was degassed while the pressure in the flask was reduced, and the air in the flask was replaced with nitrogen. Next, the mixture in the flask was heated at 40° C. under a nitrogen stream, and 441 mg (0.54 mmol) of [1,1′-bis(diphenylphosphino)ferrocene]palladium(II) dichloride dichloromethane adduct (abbreviation: PdCl₂(dppf)-CH₂Cl₂) was added thereto. Then, the temperature of the mixture was raised to 100° C. and the mixture was stirred for 7 hours while being heated. After the reaction, water was added to the mixture, and extraction with toluene was performed. The obtained organic layer was washed with water and saturated brine, and dried with magnesium sulfate. The obtained filtrate was concentrated, and purification by silica gel column chromatography (toluene:hexane=1:1) was performed. The fraction was concentrated and dried to give 4.59 g of a white solid in a yield of 67%. A synthesis scheme of Step 3 is shown in (c-1) below.

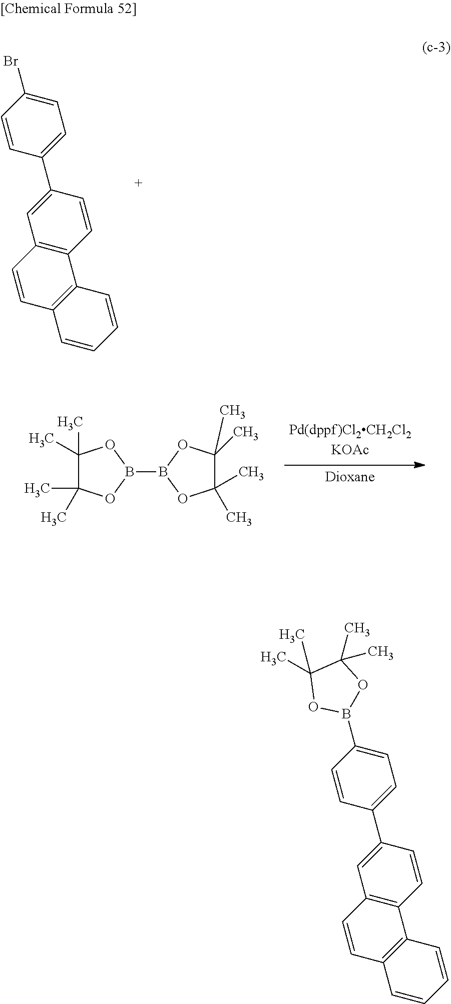

### Step 4: Synthesis of 3-[4-(phenanthren-2-yl)phenyl]-9H-carbazole

Step 4 was performed in the following procedure.

Into a 200-mL three-neck flask were put 3.19 g (8.4 mmol) of 4,4,5,5-tetramethyl-2-[4-(phenanthren-2-yl)phenyl]-1,3,2-dioxaborolane obtained in Step 3, 1.72 g (7.0 mmol) of 3-bromocarbazole, 2.9 mg (21.0 mmol) of potassium carbonate (abbreviation: K₂CO₃), 35 mL of toluene, 7 mL of ethanol, and 10 mL of water. Then, the mixture was degassed while the pressure in the flask was reduced, and the air in the flask was replaced with nitrogen. Next, the mixture in the flask was heated at 40° C. under a nitrogen stream, and 128 mg (0.42 mmol) of tris(2-methylphenyl)phosphine (abbreviation: P(o-tolyl)₃) and 47 mg (0.21 mmol) of palladium acetate were added thereto. Then, the temperature of the mixture was raised to 90° C. and the mixture was stirred for 7 hours while being heated. After the reaction, this mixture was suction-filtered, and the obtained residue was washed with water and ethanol. The resulting solid was dissolved in heated toluene, followed by filtration through Celite. The obtained filtrate was concentrated and recrystallized with toluene, so that 1.77 g of a brown solid was obtained in a yield of 60%. A synthesis scheme of Step 4 is shown in (c-4) below.

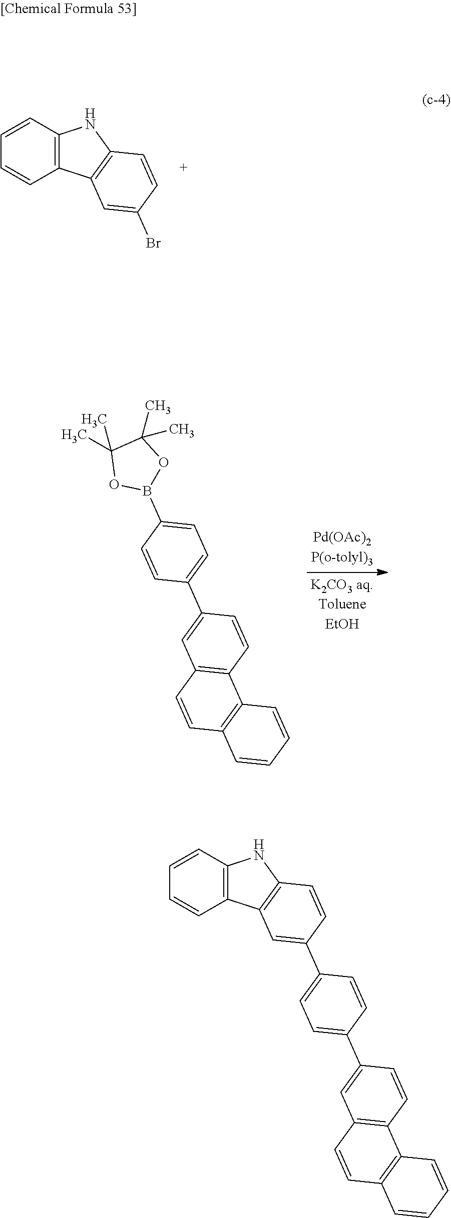 [SMILES: Brc1ccc2[nH]c3ccccc3c2c1 ; CC1(C)OB(c2ccc(-c3ccc4c(ccc5ccccc54)c3)cc2)OC1(C)C ; c1ccc2c(c1)ccc1cc(-c3ccc(-c4ccc5[nH]c6ccccc6c5c4)cc3)ccc12]

### Step 5: Synthesis of PnCPPn

Step 5 was performed in the following procedure.

Into a 200-mL three-neck flask were put 1.81 g (4.32 mmol) of 3-[4-(phenanthren-2-yl)phenyl]-9H-carbazole obtained in Step 4, 0.93 g (3.6 mmol) of 2-bromophenanthrene, 0.52 g (5.4 mmol) of sodium tert-butoxide, and 20 mL of toluene. Then, the mixture was degassed while the pressure in the flask was reduced, and the air in the flask was replaced with nitrogen. Next, the mixture in the flask was heated at 40° C. under a nitrogen stream, and 17 mg (0.09 mmol) of tri(tert-butyl)phosphine (a 10 wt % hexane solution) and 33 mg (0.04 mmol) of tris(dibenzylideneacetone)dipalladium(0) were added thereto. Then, the temperature of the mixture was raised to 90° C. and the mixture was stirred for 7 hours while being heated. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene, so that 1.75 g of a white solid was obtained in a yield of 82%.

With a train sublimation method, 1.74 g of the obtained white solid was purified by heating at 340° C. for 29 hours under a pressure of 2.50 Pa with an argon flow rate of 18 mL/min to give 1.33 g of a white solid at a collection rate of 76%. As the result of mass spectrometry, it was confirmed that the target substance PnCPPn was obtained. A synthesis scheme of Step 5 is shown in (c-5) below.

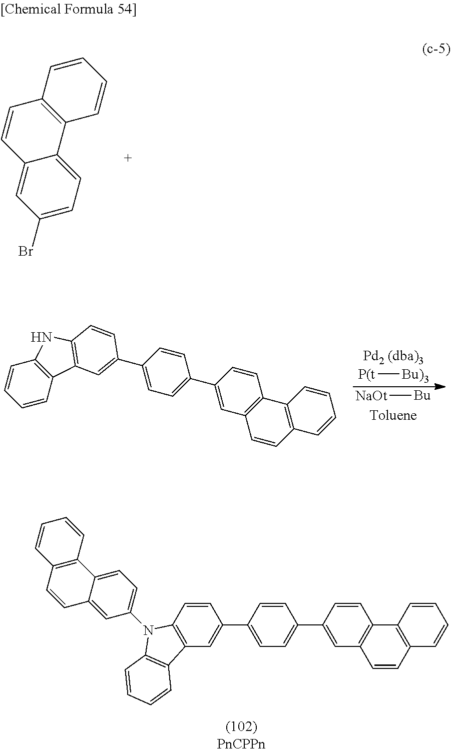

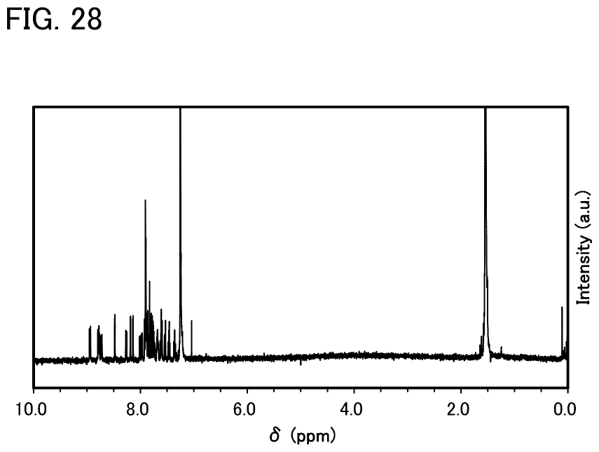

FIG. 28 shows a nuclear magnetic resonance spectroscopy (¹H-NMR) chart of PnCPPn after purification by sublimation in a deuterated chloroform (abbreviation: CDCl₃) solution. The results also show that PnCPPn was obtained.

¹H NMR (CDCl₃, 500 MHz): δ=7.37 (t, 1H), 7.45-7.49 (m, 1H), 7.55 (d, 1H), 7.60-7.64 (m, 2H), 7.68-7.69 (m, 2H), 7.74-7.93 (m, 12H), 7.98 (d, 1H), 8.01 (d, 1H), 8.15 (s, 1H), 8.19 (s, 1H), 8.28 (d, 1H), 8.49 (s, 1H), 8.74 (d, 1H), 8.73-8.80 (m, 2H), 8.95 (d, 1H).

### <Measurement of Emission and Absorption Spectra>

The absorption spectrum and the emission spectrum of PnCPPn were measured. The absorption spectrum was measured with an ultraviolet-visible spectrophotometer (V-770, manufactured by JASCO Corporation). The emission spectrum was measured with a fluorescence spectrophotometer (FP-8600, manufactured by JASCO Corporation).

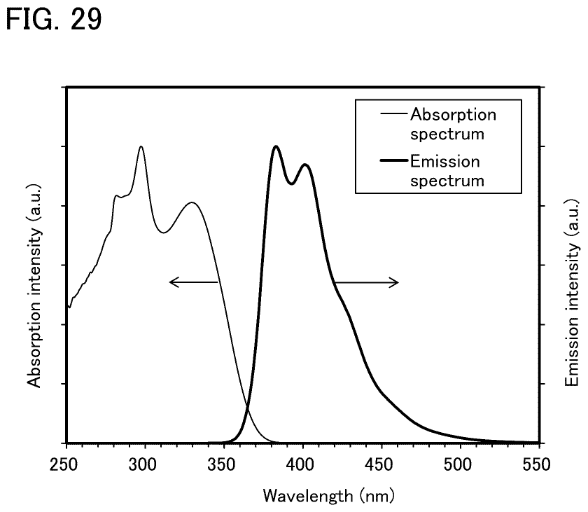

FIG. 29 shows an absorption spectrum and an emission spectrum of PnCPPn in a toluene solution. The absorption spectrum in the solution state was obtained by subtraction of a measured absorption spectrum of a solvent (toluene) alone in a quartz cell from a measured absorption spectrum of the toluene solution of PnCPPn in a quartz cell.

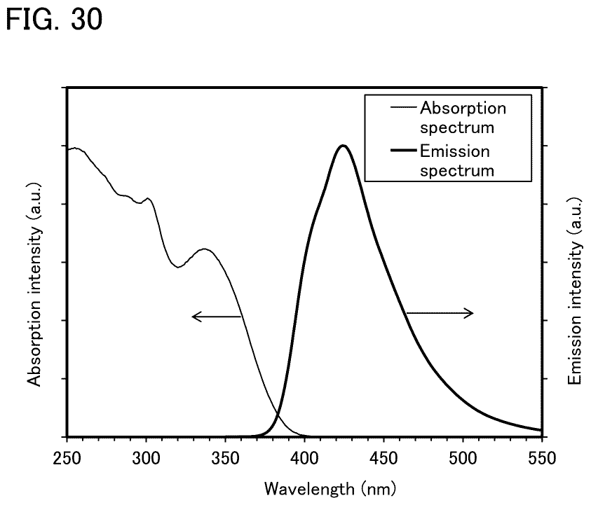

FIG. 30 shows an absorption spectrum and an emission spectrum of a thin film of PnCPPn. The solid thin film was formed over a quartz substrate by a vacuum evaporation method and another quartz substrate as a counter substrate was used to perform sealing; thus, a measurement sample was fabricated. Note that the emission spectrum was obtained by measuring the sealed sample, and the absorption spectrum was obtained by measuring the sample from which the sealing was removed and the counter substrate was detached. The absorption spectrum of the thin film was obtained by subtraction of an absorption spectrum of the quartz substrate from an absorption spectrum of a PnCPPn film formed over the quartz substrate. Even after the removal of the sealing at room temperature, any change in film quality was not visually observed from the formed thin film and the stable amorphous film was found to be maintained. This revealed that the compound of the present application was able to form a thin film with excellent stability and the organic device fabricated with the compound of the present application exhibited excellent stability.

As shown in FIG. 29, PnCPPn in the toluene solution had absorption peaks at around 297 nm and 329 nm and emission peaks at around 383 nm and 401 nm (excitation wavelength: 330 nm). Furthermore, as shown in FIG. 30, PnCPPn in the thin film had absorption peaks at around 290 nm, 304 nm, and 342 nm, and emission peaks at around 407 nm and 424 nm (excitation wavelength: 336 nm).

These absorption characteristics indicate that no absorption was observed in the visible light range (wavelength range higher than or equal to 430 nm) needed for displays. Thus, PnCPPn can be suitably used for the cap film in a wavelength range needed for displays without reducing the emission efficiency of a light-emitting element. Furthermore, the obtained emission characteristics show that in the case where PnCPPn is used as a host in a light-emitting layer, energy can be efficiently transferred to a light-emitting material that emits light with a wavelength higher than or equal to 440 nm and lower than or equal to 700 nm and thus PnCPPn is suitable for a light-emitting element as well.

### <Measurement of Glass Transition Temperature (T_{g})>

The glass transition temperature (T_{g}) of PnCPPn was measured. The T_{g} was measured with a differential scanning calorimeter (PYRIS 1 DSC manufactured by PerkinElmer Japan Co., Ltd.) in a state where a powder was put on an aluminum sample pan.

The results showed that the T_{g} of PnCPPn was 133° C. The T_{g} available for an organic device is higher than or equal to 100° C., preferably higher than or equal to 110° C., further preferably higher than or equal to 120° C., still further preferably higher than or equal to 130° C. Therefore, the measurement results revealed that the compound of the present invention has an excellent thermal property and a thin film formed using the compound is expected to have stable film quality. The use of the compound capable of forming a thin film with stable film quality allows a highly heat-resistant organic device to be provided.

### <Calculation of HOMO and LUMO Levels>

The HOMO level and the LUMO level of PnCPPn were obtained through cyclic voltammetry (CV) measurement. The calculation method is shown below.

An electrochemical analyzer (ALS model 600A or 600C, manufactured by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (n-Bu₄NClO₄; produced by Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, produced by BAS Inc.) was used as a working electrode, a platinum electrode (Pt counter electrode for VC-3 (5 cm), produced by BAS Inc.) was used as an auxiliary electrode, and an Ag/Ag⁺ electrode (RE7 reference electrode for nonaqueous solvent, produced by BAS Inc.) was used as a reference electrode. Note that the measurement was performed at room temperature (20° C. to 25° C.).

The scan speed in the CV measurement was fixed to 0.1 V/see, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]=−4.94−Ec.

The CV measurement was repeated 100 times, and the oxidation-reduction wave in the hundredth cycle was compared with the oxidation-reduction wave in the first cycle to examine the electrical stability of the compound.

According to the measurement results of the oxidation potential Ea [V] of PnCPPn, the HOMO level was found to be around −5.78 eV. According to the measurement results of the reduction potential Ec [V] of PnCPPn, the LUMO level was found to be −2.33 eV. When the oxidation-reduction wave was repeatedly measured, in the Ea measurement, the peak intensity of the oxidation-reduction wave in the hundredth cycle was maintained to be 74% of that of the oxidation-reduction wave in the first cycle, and in the Ec measurement, the peak intensity of the oxidation-reduction wave in the hundredth cycle was maintained to be 95% of that of the oxidation-reduction wave in the first cycle; thus, resistance to oxidation and reduction of PnCPPn was found to be extremely high.

In consideration of the HOMO and LUMO levels, it is probable that holes and electrons can be favorably given and received, and PnCPPn can be suitably used for layers that need to transport carriers, such as a hole-transport layer, an electron-transport layer, a light-emitting layer, and a charge-generation layer of a tandem element in an organic device. In particular, since PnCPPn has a carbazole ring in its molecular structure, it can be suitably used for a hole-transport layer, a light-emitting layer, and a charge-generation layer of a tandem element which are responsible for hole transport.

### <Refractive Index Measurement>

The refractive index of PnCPPn was measured by a spectroscopic ellipsometer (M-2000U, manufactured by J. A. Woollam Japan). The PnCPPn film used for the measurement was formed to a thickness of approximately 50 nm over a quartz substrate by a vacuum evaporation method.

At a wavelength of 630 nm, n Ordinary (nₒ) that is the ordinary refractive index was 1.89, n Extra-ordinary (nₑ) that is the extraordinary refractive index was 1.69, and the difference between nₒ and nₑ was 0.20. At a wavelength of 520 nm, nₒ was 1.94, nₑ was 1.72, and the difference between nₒ and nₑ was 0.23. At a wavelength of 450 nm, nₒ was 2.03, nₑ was 1.76, and the difference between nₒ and nₑ was 0.27.

For the cap layer material, the difference between nₒ and nₑ is preferably greater than or equal to 0.1, further preferably greater than or equal to 0.2, still further preferably greater than or equal to 0.3 at any of three wavelengths of 450 nm, 520 nm, and 630 nm or at each of the three wavelengths. Therefore, the measurement results revealed that PnCPPn can be effectively used as a material of a cap layer provided over a cathode in a light-emitting apparatus.

Thus, it was found from the measurement results of the physical property values that PnCPPn can be effectively used for a cap layer used over a cathode. Furthermore, the results showed that PnCPPn can also be effectively used as a light-emitting substance or a host material used in combination with a substance that emits light in the visible range.

### Example 4

In this example, the sublimation temperatures and the evaporation temperatures of the organic compounds of embodiments of the present invention were measured.

Specifically, the sublimation temperatures and the evaporation temperatures of 3-[4-(2,2′-binaphthalen-6-yl)phenyl]-9-phenyl-9H-carbazole (abbreviation: PCP(βN2)) represented by Structural Formula (100), 3-[4-(2,2′-binaphthalen-6-yl)phenyl]-9-(2-naphthyl)-9H-carbazole (abbreviation: βNCP(βN2)) represented by Structural Formula (101), and 3-[4-(2-phenanthlenyl)phenyl]-9-(2-phenanthryl)-9H-carbazole (abbreviation: PnCPPn) represented by Structural Formula (102) were measured. Moreover, the sublimation temperatures and the evaporation temperatures of 3-[4-(2-phenanthlenyl)phenyl]-9-(2-naphthyl)-9H-carbazole (abbreviation: βNCPPn-02) represented by Structural Formula (123) and 3-[4-(2-naphthalenyl)phenyl]-9-[4-(2-naphthalenyl)phenyl]-9H-carbazole (abbreviation: βNPCPβN) represented by Structural Formula (150) were measured.

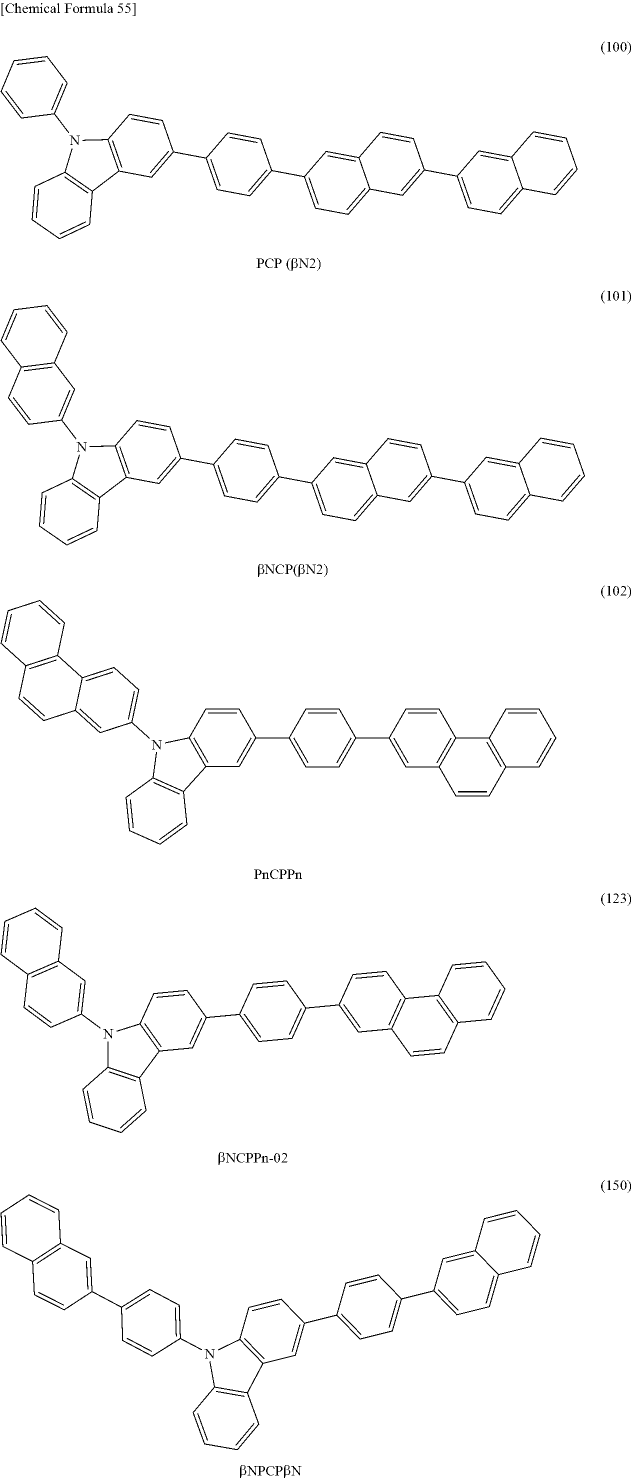 [SMILES: c1ccc(-n2c3ccccc3c3cc(-c4ccc(-c5ccc6cc(-c7ccc8ccccc8c7)ccc6c5)cc4)ccc32)cc1 ; c1ccc2cc(-c3ccc4cc(-c5ccc(-c6ccc7c(c6)c6ccccc6n7-c6ccc7ccccc7c6)cc5)ccc4c3)ccc2c1 ; c1ccc2c(c1)ccc1cc(-c3ccc(-c4ccc5c(c4)c4ccccc4n5-c4ccc5c(ccc6ccccc65)c4)cc3)ccc12 ; c1ccc2cc(-n3c4ccccc4c4cc(-c5ccc(-c6ccc7c(ccc8ccccc87)c6)cc5)ccc43)ccc2c1 ; c1ccc2cc(-c3ccc(-c4ccc5c(c4)c4ccccc4n5-c4ccc(-c5ccc6ccccc6c5)cc4)cc3)ccc2c1]

### <Thermogravimetry>

The thermogravimetry-differential thermal analysis (TG-DTA) was performed to find the sublimation temperatures. A high vacuum differential type differential thermal balance (STA-2500, manufactured by NETZSCH Japan K.K.) was used for the measurement. The measurement was performed at a temperature rising rate of 10° C./min under a nitrogen stream (flow rate: 1.2 mL/min to 1.7 mL/min) at a pressure of 10 Pa. The weight of the compound used for the measurement was 2 mg to 4 mg. Note that the 5% weight loss temperature was defined as the sublimation temperature for each organic compound.

### <Measurement of Evaporation Temperature>

The evaporation temperature was measured in an evaporation apparatus where deposition of an organic compound material is performed. Each organic compound weighing 100 mg was put into a Shapal-M soft (registered trademark), which is a ceramic crucible, and the crucible was set in the evaporation apparatus. After the evaporation apparatus was evacuated to a high vacuum below 1.0×10⁻⁴ Pa, the crucible was heated. The evaporation rate at each temperature was checked, whereby the evaporation temperature was obtained. Note that the temperature at which the evaporation rate was 1×10⁻¹ nm/s was defined as the evaporation temperature for each organic compound.

### <Each Measurement Result>

The results of the thermogravimetry and the evaporation temperature measurement are shown below. The results of the thermogravimetry of each organic compound are shown in the table below.

TABLE 1

| Structural | | 5% weight loss | 50% weight loss |
|---|---|---|---|
| Formula | Abbreviation | temperature (° C.) | temperature (° C.) |
| (100) | PCP(βN2) | 286 | 310 |
| (101) | βNCP(βN2) | 291 | 327 |
| (102) | PnCPPn | 289 | 343 |
| (123) | βNCPPn-02 | 256 | 289 |
| (150) | βNPCPβN | 260 | 294 |

The above table shows that PCP(βN2), βNCP(βN2), and PnCPPn, which are the organic compounds of embodiments of the present invention, each have a sublimation temperature below 300° C. and in particular PCP(βN2) and PnCPPn each have a sublimation temperature below 290° C. Accordingly, it was found that PCP(βN2), βNCP(βN2), and PnCPPn which are the organic compounds of the embodiments of the present invention can be favorably used in an evaporation process. In particular, a potential for PCP(βN2) and PnCPPn to enable faster deposition in a mass production process was demonstrated.

Furthermore, the above table shows that βNCPPn-02 and βNPCPβN, which are the organic compounds of embodiments of the present invention, each have a sublimation temperature below 260° C. and in particular βNCPPn-02 has a sublimation temperature of 256° C. Accordingly, it was found that βNCPPn-02 and βNPCPβN which are the organic compounds of the embodiments of the present invention can be favorably used in an evaporation process. A potential for βNCPPn-02 and βNPCPβN to enable faster deposition in a mass production process was demonstrated.

The results of the evaporation temperature measurement of each organic compound are shown in the table below.

TABLE 2

| Structural Formula | Abbreviation | Evaporation temperature (° C.) |
|---|---|---|
| (100) | PCP(βN2) | 296 |
| (101) | βNCP(βN2) | 323 |
| (102) | PnCPPn | 319 |
| (123) | βNCPPn-02 | 264 |
| (150) | βNPCPβN | 303 |

Thus, it was found that the evaporation temperatures of PCP(βN2), βNCP(βN2), and PnCPPn, which are the organic compounds of the embodiments of the present invention, were below 325° C., which is lower than 330° C. In particular, the evaporation temperature of PCP(βN2) was 296° C., which is even lower than 300° C.

Furthermore, the evaporation temperatures of βNCPPn-02 and βNPCPβN, which are the organic compounds of embodiments of the present invention, were found to be below 305° C. In particular, the evaporation temperature of βNCPPn-02 was 264° C.

As described above, the organic compounds of the embodiments of the present invention can be deposited at low temperatures and thus are less thermally affected during the deposition and decomposition due to heat can be reduced. In particular, in the mass production process, the same material is heated continuously for a long time; an organic compound having an excessively high evaporation temperature is easily decomposed by the heating. When the material is decomposed, the evaporation temperature is further increased, for example, whereby a stable mass production system cannot be established. Thus, a cap layer material that can be deposited at low temperature can be deposited without decomposition of the material, resulting in stable mass production.

Furthermore, a structure body to which the compound is deposited can be inhibited from being affected by heat. In particular, a thermal budget in formation of the cap layer in manufacturing a device can cause a change in quality and deterioration of all the materials used in the structure body to which the compound is deposited. The deterioration of the materials used in the structure body to which the compound is deposited causes an increase in variation in device characteristics, for example, which directly leads to a decrease in manufacturing yield. Accordingly, when the cap layer is deposited by evaporation at a low temperature, a change in quality and deterioration of the materials used in the structure body to which the compound is deposited can be inhibited and manufacturing with high yield is possible.

The structures, the compositions, the methods, and the like described in this embodiment can be combined as appropriate with any of the structures, the compositions, the methods, and the like described in the other embodiments and examples, for example.

### Example 5

In this example, the ordinary refractive indices and the extinction coefficients of organic compounds that can be used in a light-emitting device of one embodiment of the present invention were measured.

Specifically, the refractive indices and the extinction coefficients of 3-[4-(2,2′-binaphthalen-6-yl)phenyl]-9-phenyl-9H-carbazole (abbreviation: PCP(βN2)) represented by Structural Formula (100), 3-[4-(2,2′-binaphthalen-6-yl)phenyl]-9-(2-naphthyl)-9H-carbazole (abbreviation: βNCP(βN2)) represented by Structural Formula (101), 3-[4-(2-phenanthlenyl)phenyl]-9-(2-phenanthryl)-9H-carbazole (abbreviation: PnCPPn) represented by Structural Formula (102), 3-[4-(2-phenanthlenyl)phenyl]-9-(2-naphthyl)-9H-carbazole (abbreviation: βNCPPn-02) represented by Structural Formula (123), and 3-[4-(2-naphthalenyl)phenyl]-9-[4-(2-naphthalenyl)phenyl]-9H-carbazole (abbreviation: βNPCPβN) represented by Structural Formula (150) were measured.

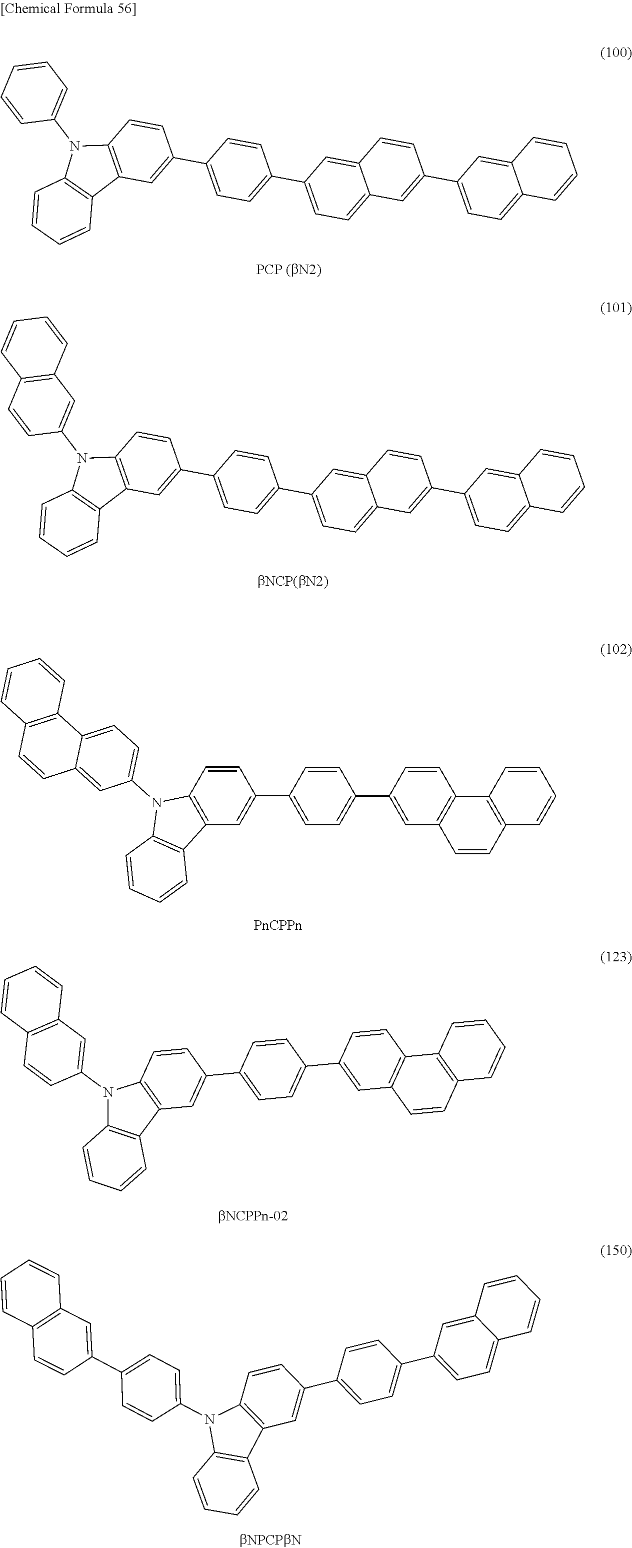 [SMILES: c1ccc(-n2c3ccccc3c3cc(-c4ccc(-c5ccc6cc(-c7ccc8ccccc8c7)ccc6c5)cc4)ccc32)cc1 ; c1ccc2cc(-c3ccc4cc(-c5ccc(-c6ccc7c(c6)c6ccccc6n7-c6ccc7ccccc7c6)cc5)ccc4c3)ccc2c1 ; c1ccc2c(c1)ccc1cc(-c3ccc(-c4ccc5c(c4)c4ccccc4n5-c4ccc5c(ccc6ccccc65)c4)cc3)ccc12 ; c1ccc2cc(-n3c4ccccc4c4cc(-c5ccc(-c6ccc7c(ccc8ccccc87)c6)cc5)ccc43)ccc2c1 ; c1ccc2cc(-c3ccc(-c4ccc5c(c4)c4ccccc4n5-c4ccc(-c5ccc6ccccc6c5)cc4)cc3)ccc2c1]

### <Measurement>

PCP(βN2), βNCP(βN2), PnCPPn, βNCPPn-02, and βNPCPβN were each deposited to a thickness of approximately 50 nm over a quartz substrate by a vacuum evaporation method to form measurement samples. The measurement was performed with a spectroscopic ellipsometer (M-2000U, manufactured by J. A. Woollam Japan Corp.).

### <Measurement Results>

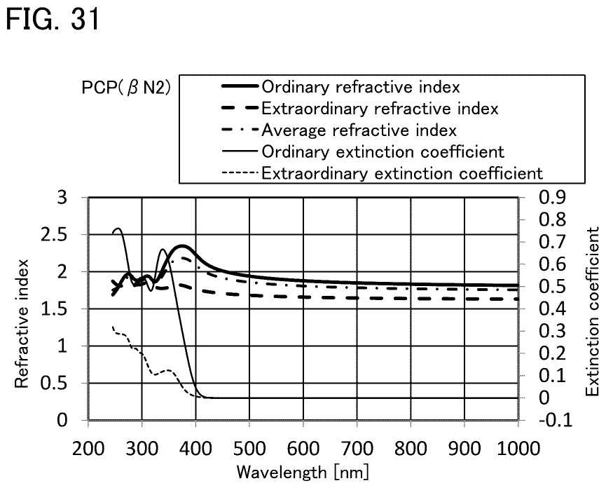

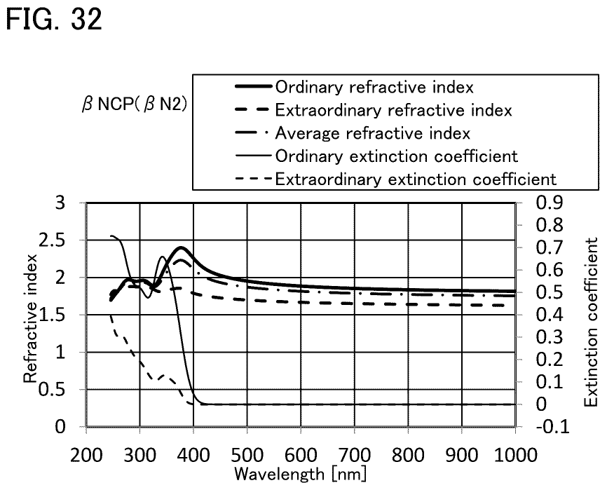

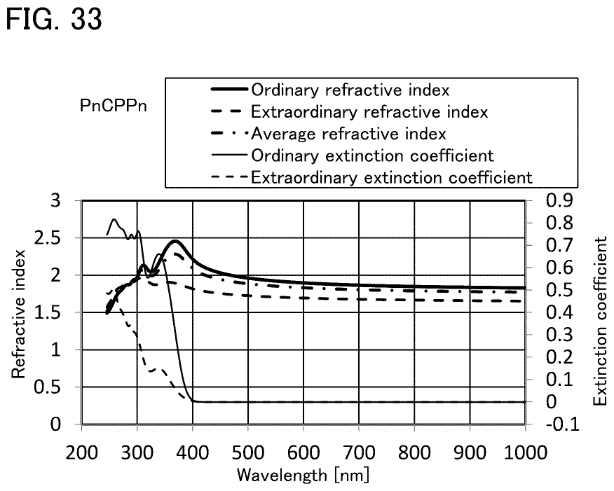

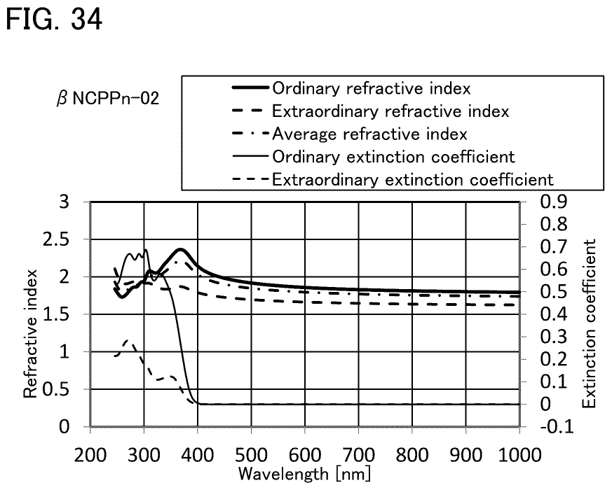

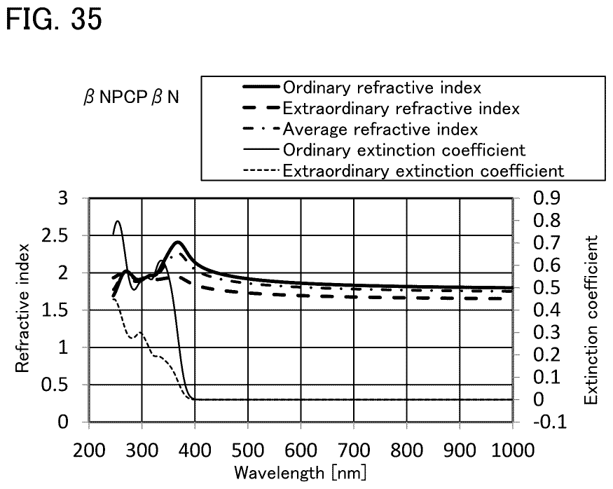

The measured ordinary refractive index (nₒ), extraordinary refractive index (nₑ), average refractive index (n), ordinary extinction coefficient (kₒ), and extraordinary extinction coefficient of PCP(βN2), βNCP(βN2), PnCPPn, βNCPPn-02, and βNPCPβN are shown in FIG. 31, FIG. 32, FIG. 33, FIG. 34, and FIG. 35. In addition, the values at a measurement wavelength of 450 nm and the difference between the ordinary refractive index (nₒ) and the extraordinary refractive index (nₑ) (Δn=|nₒ−nₑ|) are shown in the table below. Note that the difference between the ordinary refractive index (nₒ) and the extraordinary refractive index (nₑ) (Δn=|nₒ−nₑ|) can be used as an indicator of anisotropy in the refractive index. In the visible light range (higher than or equal to 360 nm and lower than or equal to 830 nm), the refractive index anisotropy tends to be larger with a larger Δn. The average refractive index (n) was obtained by calculating the square root of (2nₒ²+nₑ²)/3, where the refractive index in the optic axis direction (z direction) is the refractive index nₑ and the refractive indices in the directions perpendicular to the optic axis (x and y directions) are each the refractive index nₒ.

TABLE 3

| | | Ordinary extinction | Ordinary | Extraordinary | Average | |
|---|---|---|---|---|---|---|
| Structural | | coefficient | refractive index | refractive index | refractive index | Δn |
| Formula | Abbreviation | ko | no | ne | n | \|no − ne\| |
| (100) | PCP(βN2) | 0.0000 | 2.01 | 1.71 | 1.91 | 0.30 |
| (101) | βNCP(βN2) | 0.0000 | 2.03 | 1.73 | 1.93 | 0.30 |
| (102) | PnCPPn | 0.0000 | 2.03 | 1.76 | 1.94 | 0.27 |
| (123) | βNCPPn-02 | 0.0000 | 1.98 | 1.73 | 1.90 | 0.25 |
| (150) | βNPCPβN | 0.0000 | 1.98 | 1.76 | 1.91 | 0.22 |

It was found that PCP(βN2), βNCP(βN2), and PnCPPn are high refractive index materials each having an ordinary refractive index (nₒ) above 2.00 and an ordinary extinction coefficient (kₒ) below 1×10⁻⁴ at a measurement wavelength of 450 nm. It was also found that PCP(βN2), βNCP(βN2), and PnCPPn each have a difference between the ordinary refractive index (nₒ) and the extraordinary refractive index (nₑ) (Δn=|nₒ−nₑ|) of greater than or equal to 0.25 and have large refractive index anisotropy. The average refractive index (n) was higher than or equal to 1.9. Because the ordinary refractive index (nₒ) is high when the average refractive index (n) is high, a high average refractive index (n) is preferable. Note that the average refractive index (n) at a wavelength of 450 nm is preferably higher than or equal to 1.8.

It was found that βNCPPn-02 and βNPCPβN are high refractive index materials each having an ordinary refractive index (nₒ) of 1.98 and an ordinary extinction coefficient (kₒ) below 1×10⁻⁴ at a measurement wavelength of 450 nm. It was also found that βNCPPn-02 and βNPCPβN each have a difference between the ordinary refractive index (nₒ) and the extraordinary refractive index (nₑ) (Δn=|nₒ−nₑ|) of greater than or equal to 0.22 and have large refractive index anisotropy. The average refractive index (n) was higher than or equal to 1.9. Because the ordinary refractive index (nₒ) is high when the average refractive index (n) is high, a high average refractive index (n) is preferable.

As described above, PCP(βN2), βNCP(βN2), PnCPPn, βNCPPn-02, and βNPCPβN have high refractive indices and large refractive index anisotropy; therefore, when they are used as cap layers of light-emitting devices, light can be extracted with high efficiency. Since the ordinary extinction coefficients (kₒ) are low, light can be extracted from the light-emitting devices to the outside without absorption of light; thus, highly efficient elements can be provided.

The structures, the compositions, the methods, and the like described in this embodiment can be combined as appropriate with any of the structures, the compositions, the methods, and the like described in the other embodiments and examples, for example.

### Example 6

In this example, a light-emitting device 6A and a light-emitting device 6B of embodiments of the present invention were fabricated.

Specifically, the light-emitting devices in which 3-[4-(2,2′-binaphthalen-6-yl)phenyl]-9-phenyl-9H-carbazole (abbreviation: PCP(βN2)) represented by Structural Formula (100) and 3-[4-(2,2′-binaphthalen-6-yl)phenyl]-9-(2-naphthyl)-9H-carbazole (abbreviation: βNCP(βN2)) represented by Structural Formula (101) were used for cap layers were fabricated.

The structural formulae of organic compounds used in common in the light-emitting devices 6A and 6B are shown below.

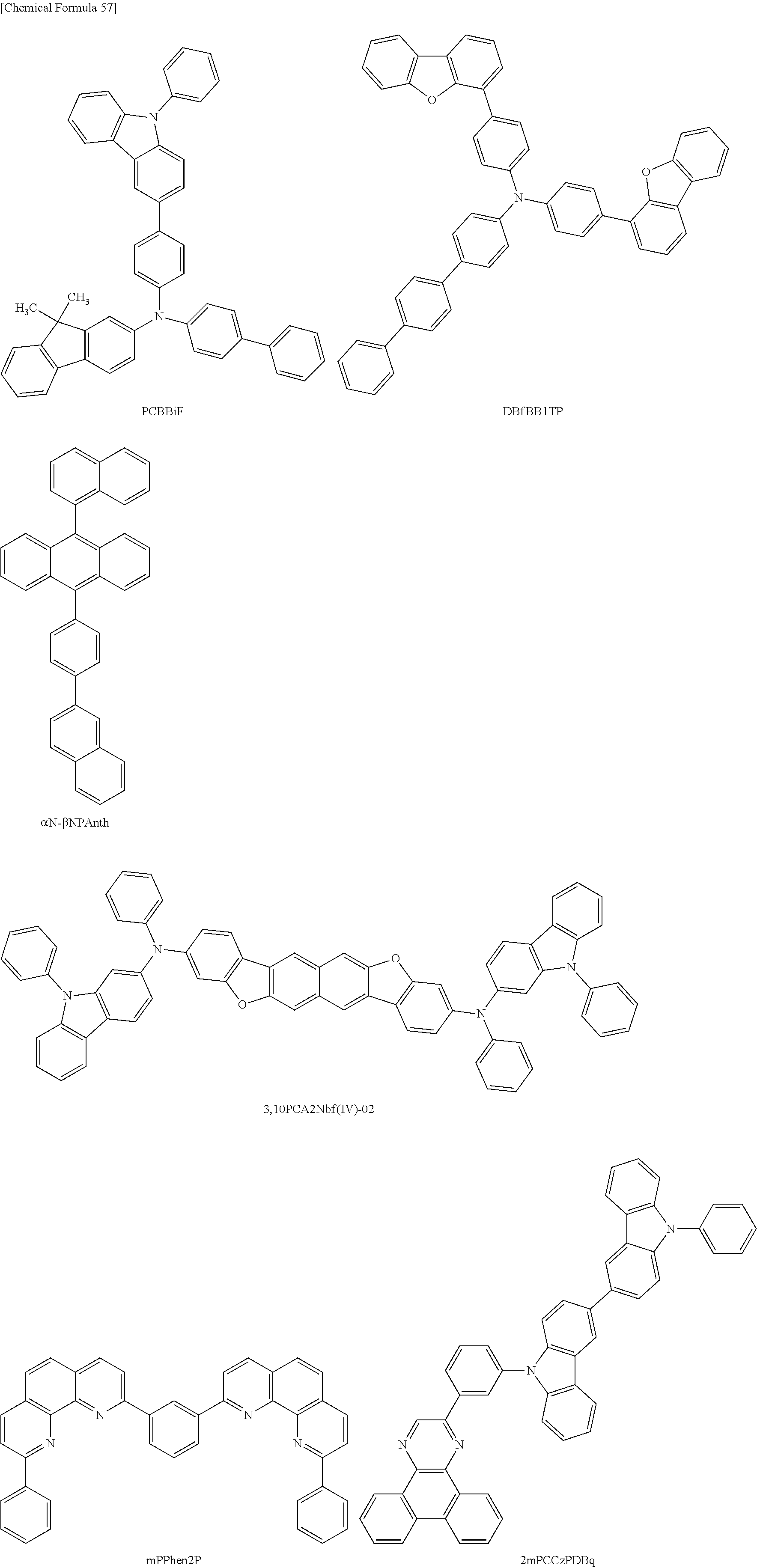

The structural formulae of the organic compound used in the light-emitting device 6A and the organic compound used in the light-emitting device 6B are shown below.

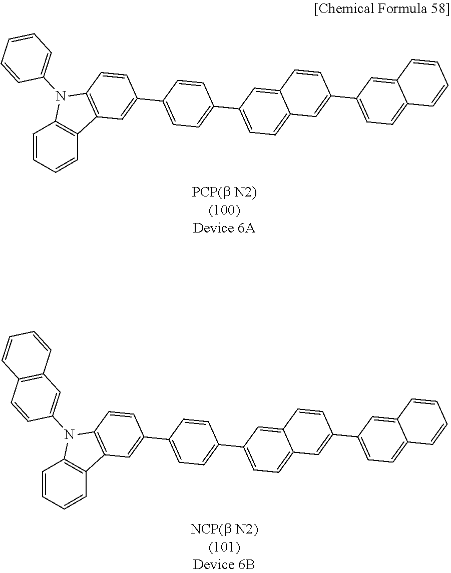 [SMILES: c1ccc(-n2c3ccccc3c3cc(-c4ccc(-c5ccc6cc(-c7ccc8ccccc8c7)ccc6c5)cc4)ccc32)cc1 ; c1ccc2cc(-c3ccc4cc(-c5ccc(-c6ccc7c(c6)c6ccccc6n7-c6ccc7ccccc7c6)cc5)ccc4c3)ccc2c1]

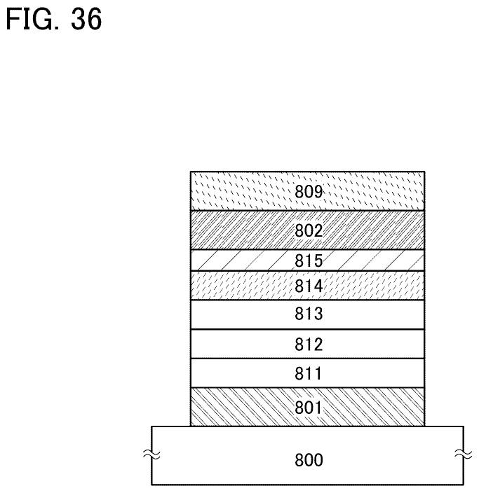

In each of the light-emitting devices, as illustrated in FIG. 36, a hole-injection layer 811, a hole-transport layer 812, a light-emitting layer 813, an electron-transport layer 814, and an electron-injection layer 815 are stacked in this order over a first electrode 801 formed over a glass substrate 800, and a second electrode 802 is stacked over the electron-injection layer 815. Furthermore, a cap layer 809 is provided over the second electrode 802.

### <Method for Fabricating Light-Emitting Device 6A>

Silver (Ag) was deposited over the glass substrate 800 to a thickness of 100 nm by a sputtering method, and then, as a transparent electrode, indium tin oxide containing silicon oxide (ITSO) was deposited to a thickness of 10 nm by a sputtering method, whereby the first electrode 801 was formed. The electrode area was set to 4 mm² (2 mm×2 mm).

Next, in pretreatment for forming the light-emitting device over the substrate, a surface of the substrate was washed with water, and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed to lower than or equal to 30° C.

Next, the substrate provided with the first electrode 801 was fixed to a substrate holder provided in the vacuum evaporation apparatus such that the surface on which the first electrode 801 was formed faced downward. Over the first electrode 801, N-(biphenyl-4-yl)-N-[4-(9-phenyl-9H-carbazol-3-yl)phenyl]-9,9-dimethyl-9H-fluoren-2-amine (abbreviation: PCBBiF) and an electron acceptor material containing fluorine and having a molecular weight of 672 (OCHD-003) were deposited to a thickness of 10 nm by co-evaporation at the weight ratio of 1:0.03, whereby the hole-injection layer 811 was formed.

Next, PCBBiF was deposited over the hole-injection layer 811 to a thickness of 102.5 nm by evaporation, and then N,N-bis[4-(dibenzofuran-4-yl)phenyl]-4-amino-p-terphenyl (abbreviation: DBfBB1TP) was deposited to a thickness of 10 nm by evaporation, whereby the hole-transport layer 812 was formed. As a material of the hole-transport layer, a material having a lower ordinary refractive index nₒ than the cap layer material of the present application is preferably used. When the material of the hole-transport layer has an ordinary refractive index nₒ at a wavelength of 630 nm of higher than or equal to 1.65 and lower than or equal to 1.85, efficiency can be increased. This can further increase the effect of increasing efficiency, together with the effect of the cap layer material of the present application. As a method for lowering the refractive index, an alkyl group is preferably included as a substituent of the organic compound, and in particular, a tertiary butyl group and an adamantyl group are preferably included.

Next, over the hole-transport layer 812, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited to a thickness of 25 nm by a co-evaporation method using resistance heating at the weight ratio of 1:0.015, whereby the light-emitting layer 813 was formed.

Next, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited to a thickness of 10 nm by evaporation, and then 2,2′-(1,3-phenylene)bis(9-phenyl-1,10-phenanthroline) (abbreviation: mPPhen2P) was deposited to a thickness of 20 nm by evaporation, whereby the electron-transport layer 814 was formed.

Next, lithium fluoride (LiF) was deposited to a thickness of 1 nm over the electron-transport layer 814 by evaporation, whereby the electron-injection layer 815 was formed.

Then, over the electron-injection layer 815, silver (Ag) and magnesium (Mg) were deposited to a thickness of 15 nm by co-evaporation such that the volume ratio of Ag to Mg was 1:0.1, whereby the second electrode 802 was formed.

Next, over the second electrode 802, PCP(βN2) was deposited to a thickness of 80 nm by an evaporation method using resistance heating, whereby the cap layer was formed.

### <Method for Fabricating Light-Emitting Device 6B>

Next, a method for fabricating the light-emitting device 6B is described.

The light-emitting device 6B is different from the light-emitting device 6A in the structure of the cap layer. In the light-emitting device 6B, the cap layer was formed by depositing βNCP(βN2) to a thickness of 80 nm by an evaporation method using resistance heating.

The other components were formed in a manner similar to that for the light-emitting device 6A.

The structures of the light-emitting devices 6A and 6B are listed in the following table.

TABLE 4

| | Film | | |
|---|---|---|---|
| | thickness | | |
| | [nm] | Device 6A | Device 6B |
| Cap layer | 80 | PCP(βN2) | βNCP(βN2) |

| | | |
|---|---|---|
| Second electrode | 15 | Ag:Mg (1:0.1) |
| Electron-injection | 1 | LiF |
| layer | | |
| Electron-transport | 20 | mPPhen2P |
| layer | 10 | 2mPCCzPDBq |
| Light-emitting | 25 | αN-βNPAnth:3,10PCA2Nbf(IV)-02 |
| layer | | (1:0.015) |
| Hole-transport | 10 | DBfBB1TP |
| layer | 102.5 | PCBBiF |
| Hole-injection | 10 | PCBBiF:OCHD-003 (1:0.03) |
| layer | | |
| First electrode | 10 | ITSO |
| | 100 | Ag |

### <Light-Emitting Device Characteristics>

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

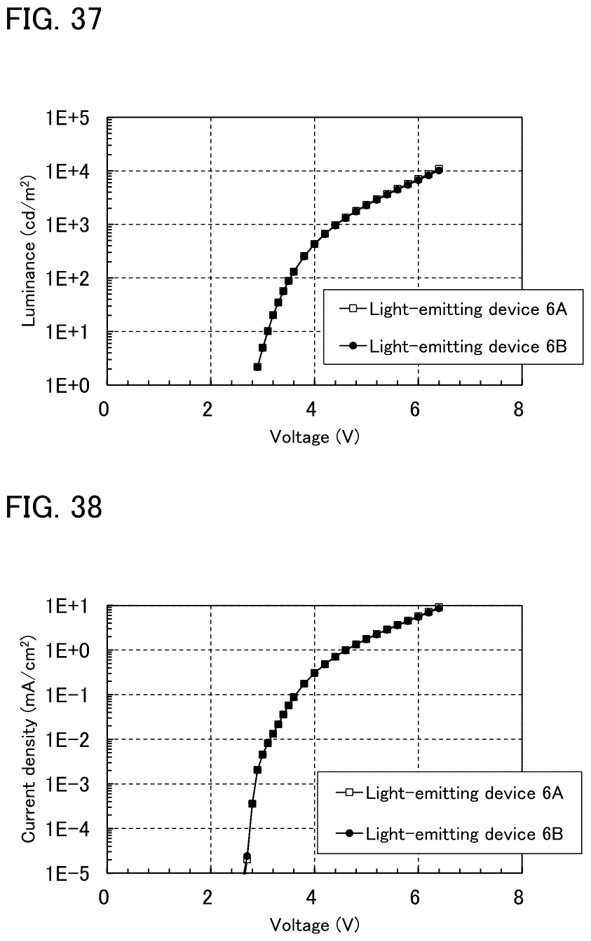

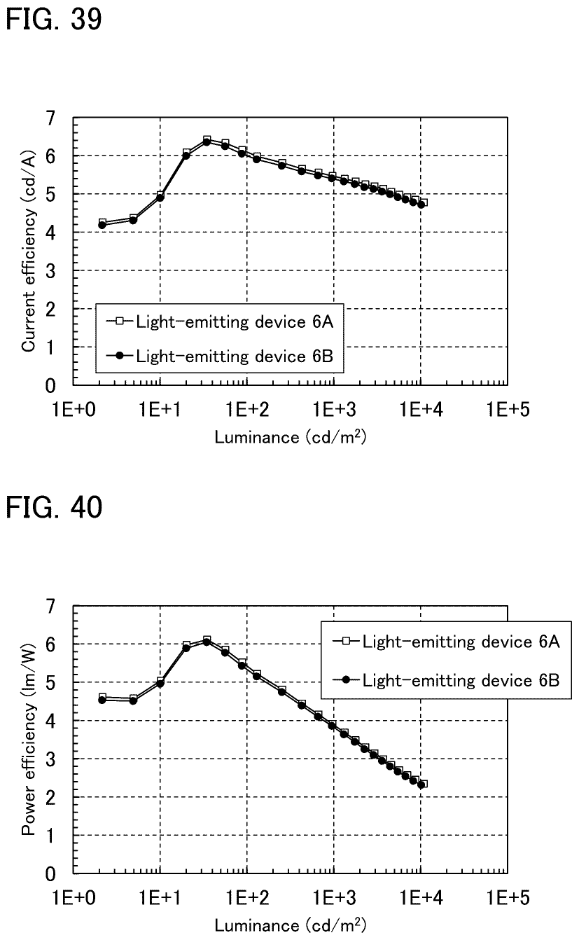

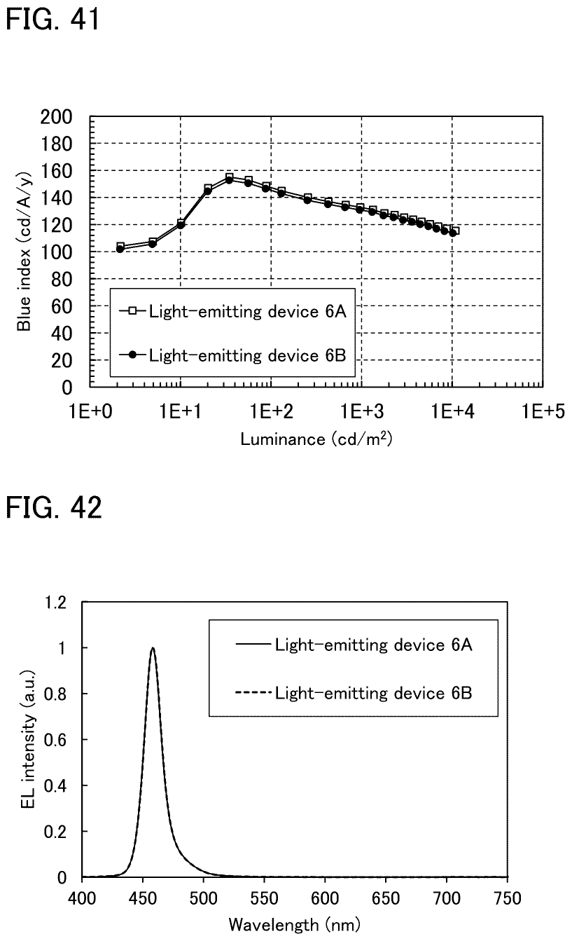

FIG. 37 shows luminance-voltage characteristics of the light-emitting devices, FIG. 38 shows current density-voltage characteristics of the light-emitting devices, FIG. 39 shows current efficiency-luminance characteristics of the light-emitting devices, FIG. 40 shows power efficiency-luminance characteristics of the light-emitting devices, FIG. 41 shows blue index-luminance characteristics of the light-emitting devices, and FIG. 42 shows electroluminescence spectra of the light-emitting devices.

Note that the blue index (BI) is a value obtained by dividing current efficiency (cd/A) by chromaticity y, which is calculated with the CIE1931 color system, and is one of the indicators of characteristics of blue light emission. As the chromaticity y is smaller, the color purity of emitted blue light tends to be higher. With high color purity of blue light, a desired color can be expressed even with a small number of luminance components and the luminance needed for expressing blue is reduced; hence, power consumption can be reduced. Thus, BI that is based on chromaticity y, which is one of the indicators of color purity of blue, is used as a means for showing efficiency of blue light emission in some cases. A light-emitting device with higher BI can be regarded as a blue-light-emitting device having higher efficiency for a display.

The main characteristics of the devices at a luminance of approximately 1000 cd/m² are shown in the table below. The luminance, CIE chromaticity, and emission spectra were measured with a spectroradiometer (SR-UL1R, manufactured by TOPCON TECHNOHOUSE CORPORATION).

TABLE 5

| | | Current | | | | Current | |
|---|---|---|---|---|---|---|---|
| | Voltage | density | Chromaticity | Chromaticity | Luminance | efficiency | BI value |
| | (V) | (mA/cm²) | x | y | (cd/m²) | (cd/A) | (cd/A/y) |
| Device 6A | 4.40 | 17.70 | 0.143 | 0.041 | 968.0 | 5.47 | 133 |
| Device 6B | 4.40 | 17.55 | 0.143 | 0.041 | 948.7 | 5.40 | 131 |

FIG. 37 to FIG. 42 show that the light-emitting devices 6A and 6B are highly efficient light-emitting devices which emit blue light having high color purity with a chromaticity y of approximately 0.04. Furthermore, since low-temperature deposition was possible in the light-emitting device 6B, the light-emitting device 6B was able to be fabricated with high yield.

### <Results of Reliability Test>

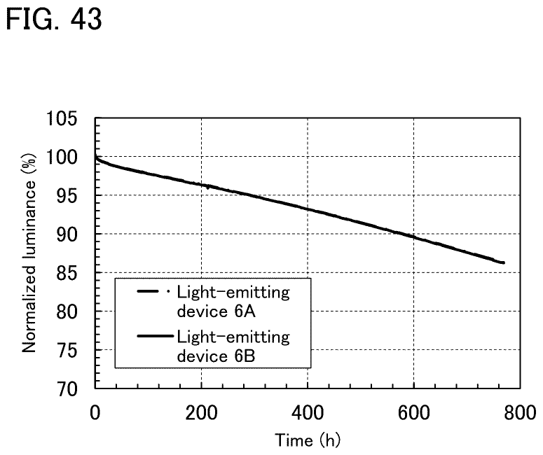

Moreover, a reliability test was conducted on the light-emitting devices 6A and 6B. FIG. 43 shows a time-dependent change in normalized luminance at the time of constant current density driving (50 [mA/cm²]). In FIG. 43, the vertical axis represents the luminance (%) normalized with the luminance at the time of the start of light emission as 100%, and the horizontal axis represent the time (h).

In FIG. 43, LT95 (h), which is the time that has elapsed until the measured luminance decreases to 95% of the initial luminance, of the light-emitting device 6A was 292 hours. In addition, LT95 of the light-emitting device 6B was 291 hours. Accordingly, the light-emitting devices 6A and 6B were found to be long-life devices.

It was found from the above results that light-emitting devices with high emission efficiency and high reliability can be provided with the use of embodiments of the present invention.

### Example 7

In this example, a light-emitting device 7A of one embodiment of the present invention was fabricated.

Specifically, the light-emitting device in which 3-[4-(2-phenanthlenyl)phenyl]-9-(2-phenanthryl)-9H-carbazole (abbreviation: PnCPPn) represented by Structural Formula (102) was used for a cap layer was fabricated.

The structural formulae of organic compounds used in the light-emitting device 7A are shown below.

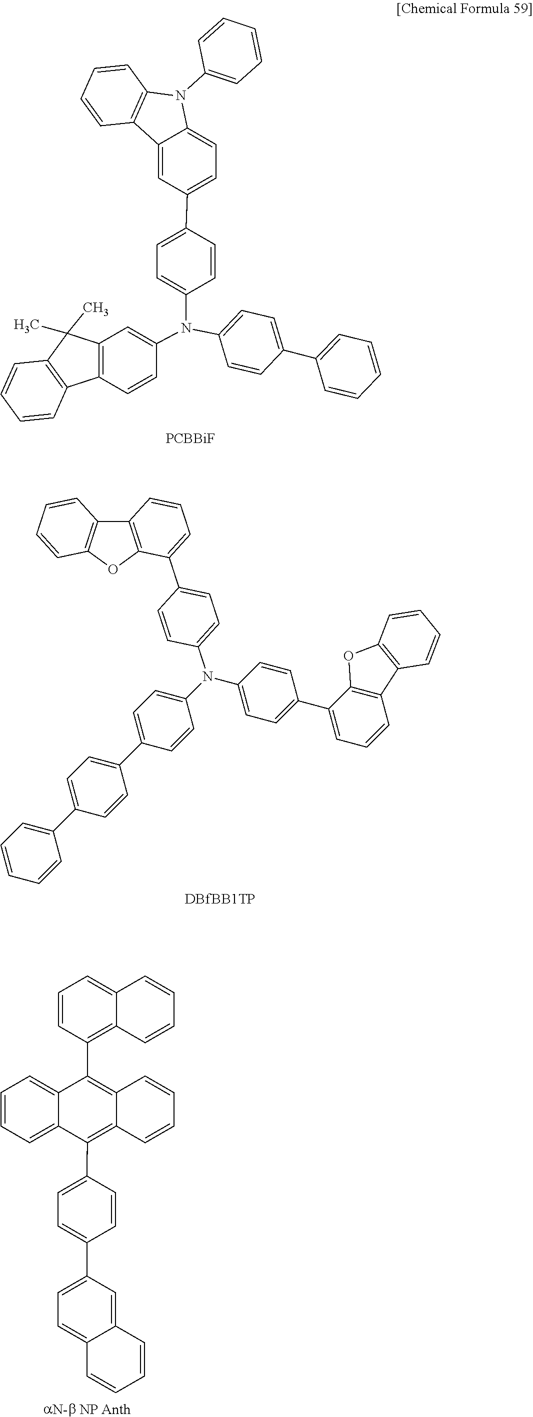

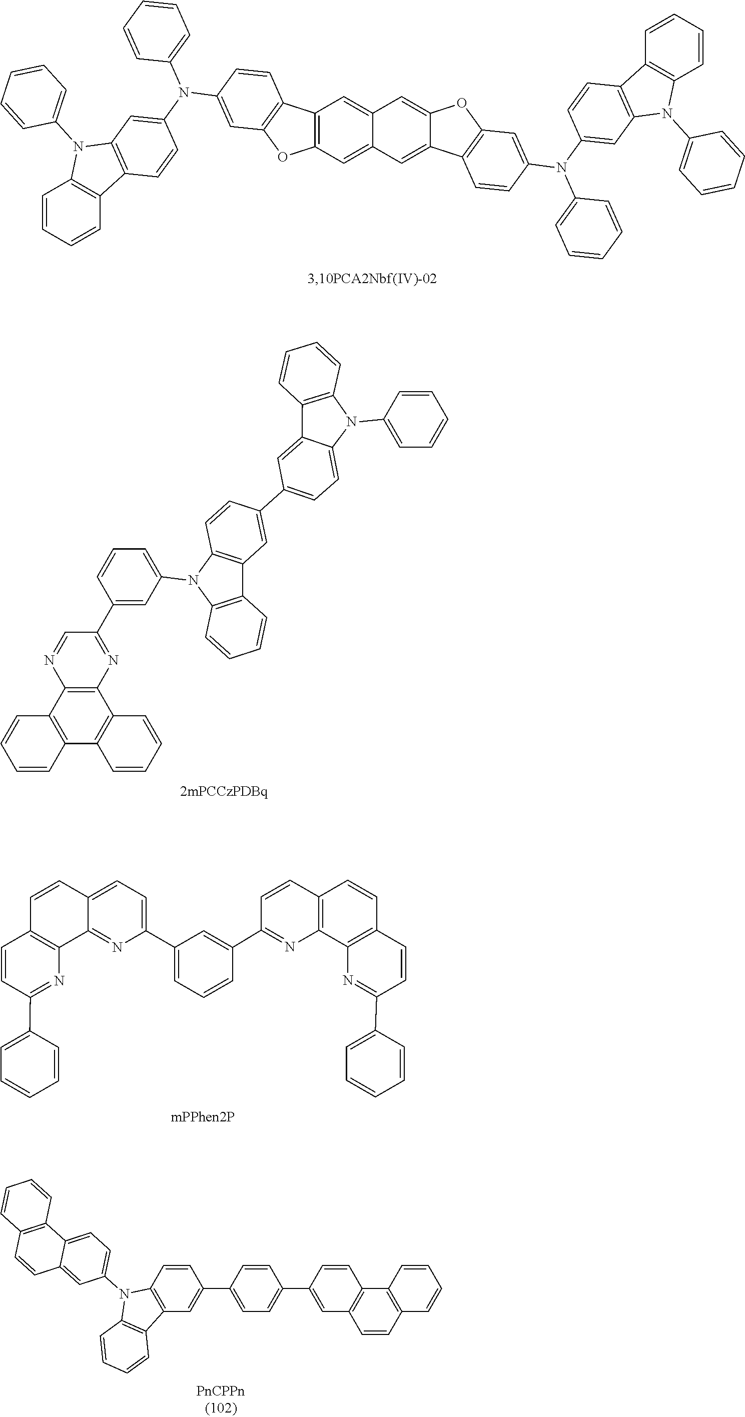 [SMILES: c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1 ; c1ccc(-n2c3ccccc3c3cc(-c4ccc5c(c4)c4ccccc4n5-c4cccc(-c5cnc6c7ccccc7c7ccccc7c6n5)c4)ccc32)cc1 ; c1ccc(-c2ccc3ccc4ccc(-c5cccc(-c6ccc7ccc8ccc(-c9ccccc9)nc8c7n6)c5)nc4c3n2)cc1 ; c1ccc2c(c1)ccc1cc(-c3ccc(-c4ccc5c(c4)c4ccccc4n5-c4ccc5c(ccc6ccccc65)c4)cc3)ccc12]

In the light-emitting device, as illustrated in FIG. 36, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815. Furthermore, the cap layer 809 is provided over the second electrode 802.

### <Method for Fabricating Light-Emitting Device 7A>

Silver (Ag) was deposited over the glass substrate 800 to a thickness of 100 nm by a sputtering method, and then, as a transparent electrode, indium tin oxide containing silicon oxide (ITSO) was deposited to a thickness of 10 nm by a sputtering method, whereby the first electrode 801 was formed. The electrode area was set to 4 mm² (2 mm×2 mm).

Next, in pretreatment for forming the light-emitting device over the substrate, a surface of the substrate was washed with water, and baking was performed at 200° C. for 1 hour. Then, the substrate was transferred into a vacuum evaporation apparatus where the pressure was reduced to approximately 1×10-4 Pa, and vacuum baking was performed at 170° C. for 30 minutes in a heating chamber of the vacuum evaporation apparatus. After that, natural cooling was performed to lower than or equal to 30° C.

Next, the substrate provided with the first electrode 801 was fixed to a substrate holder provided in the vacuum evaporation apparatus such that the surface on which the first electrode 801 was formed faced downward. Over the first electrode 801, N-(biphenyl-4-yl)-N-[4-(9-phenyl-9H-carbazol-3-yl)phenyl]-9,9-dimethyl-9H-fluoren-2-amine (abbreviation: PCBBiF) and an electron acceptor material containing fluorine and having a molecular weight of 672 (OCHD-003) were deposited to a thickness of 10 nm by co-evaporation at the weight ratio of 1:0.03, whereby the hole-injection layer 811 was formed.

Next, PCBBiF was deposited over the hole-injection layer 811 to a thickness of 102.5 nm by evaporation, and then N,N-bis[4-(dibenzofuran-4-yl)phenyl]-4-amino-p-terphenyl (abbreviation: DBfBB1TP) was deposited to a thickness of 10 nm by evaporation, whereby the hole-transport layer 812 was formed. As a material of the hole-transport layer, a material having a lower ordinary refractive index nₒ than the cap layer material of the present application is preferably used. As a method for lowering the refractive index, an alkyl group is preferably included as a substituent of the organic compound, and in particular, a tertiary butyl group or an adamantyl group are preferably included.

Next, over the hole-transport layer 812, 9-(1-naphthyl)-10-[4-(2-naphthyl)phenyl]anthracene (abbreviation: αN-βNPAnth) and N,N-diphenyl-N,N-bis(9-phenyl-9H-carbazol-2-yl)naphtho[2,3-b;6,7-b′]bisbenzofuran-3,10-diamine (abbreviation: 3,10PCA2Nbf(IV)-02) were deposited to a thickness of 25 nm by a co-evaporation method using resistance heating at the weight ratio of 1:0.015, whereby the light-emitting layer 813 was formed.

Next, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited to a thickness of 10 nm by evaporation, and then 2,2′-(1,3-phenylene)bis(9-phenyl-1,10-phenanthroline) (abbreviation: mPPhen2P) was deposited to a thickness of 20 nm by evaporation, whereby the electron-transport layer 814 was formed. The LUMO level of mPPhen2P was −2.71 eV. The LUMO level of 2mPCCzPDBq was −2.98 eV. Note that the LUMO level was measured by a method similar to that of the CV measurement in the other examples.

Next, lithium fluoride (LiF) was deposited to a thickness of 1 nm over the electron-transport layer 814 by evaporation, whereby the electron-injection layer 815 was formed.

Then, over the electron-injection layer 815, silver (Ag) and magnesium (Mg) were deposited to a thickness of 15 nm by co-evaporation such that the volume ratio of Ag to Mg was 1:0.1, whereby the second electrode 802 was formed.

Next, over the second electrode 802, PnCPPn was deposited to a thickness of 80 nm by an evaporation method using resistance heating, whereby the cap layer was formed.

The structure of the light-emitting device 7A is listed in the following table.

TABLE 6

| | Film | |
|---|---|---|
| | thickness | |
| | [nm] | Device 7A |
| Cap layer | 80 | PnCPPn |
| Second electrode | 15 | Ag:Mg (1:0.1) |
| Electron-injection | 1 | LiF |
| layer | | |
| Electron-transport | 20 | mPPhen2P |
| layer | 10 | 2mPCCzPDBq |
| Light-emitting layer | 25 | αN-βNPAnth:3,10PCA2Nbf(IV)-02 |
| | | (1:0.015) |
| Hole-transport layer | 10 | DBfBB1TP |
| | 102.5 | PCBBiF |
| Hole-injection layer | 10 | PCBBiF:OCHD-003 (1:0.03) |
| First electrode | 10 | ITSO |
| | 100 | Ag |

### <Light-Emitting Device Characteristics>

The light-emitting device was sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the device were measured.

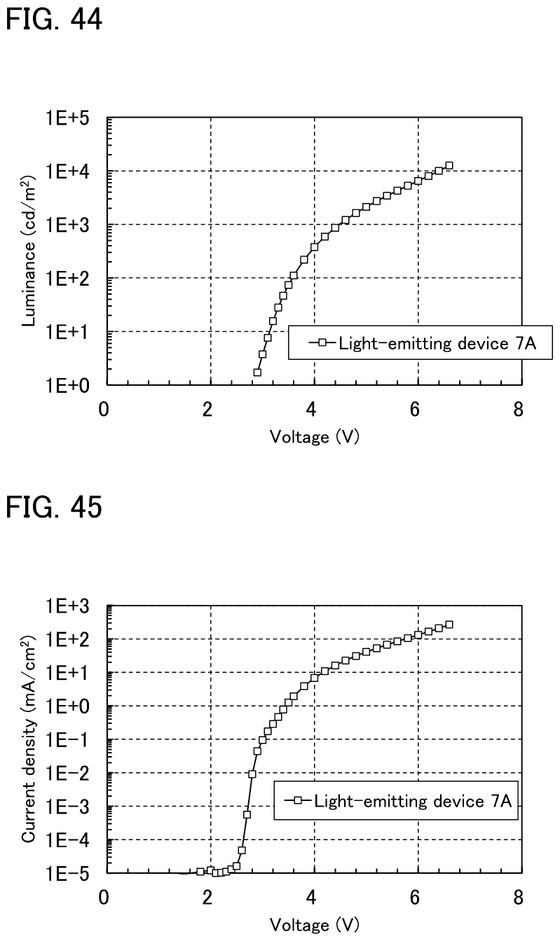

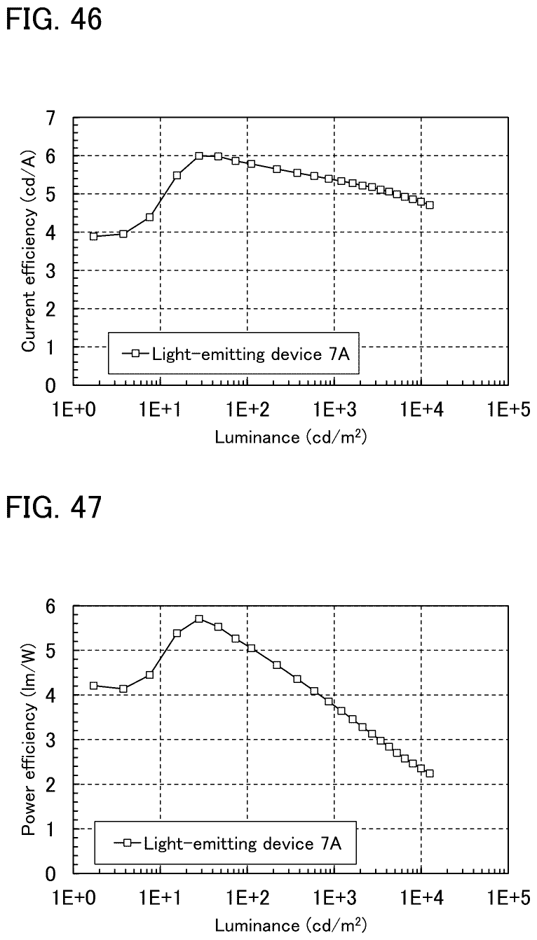

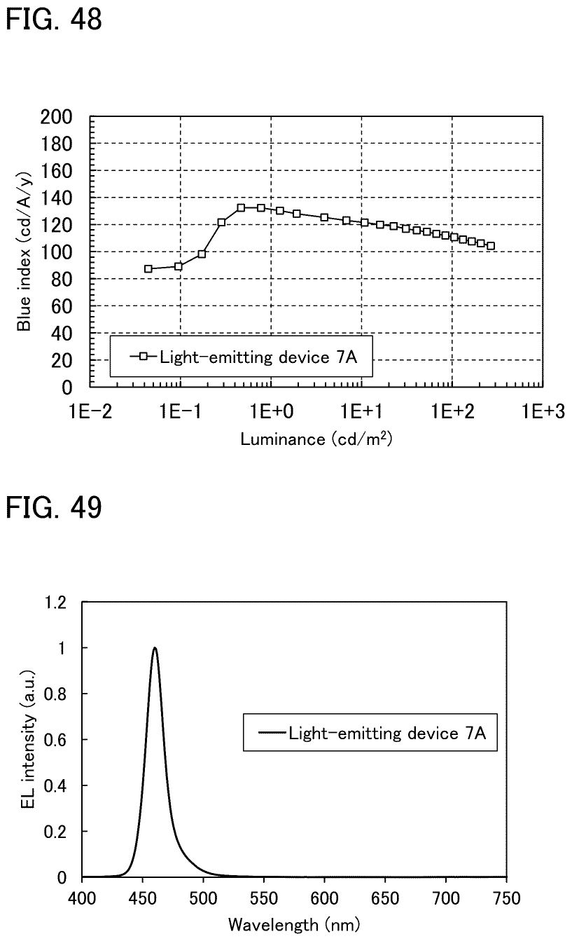

FIG. 44 shows luminance-voltage characteristics of the light-emitting device, FIG. 45 shows current density-voltage characteristics of the light-emitting device, FIG. 46 shows current efficiency-luminance characteristics of the light-emitting device, FIG. 47 shows power efficiency-luminance characteristics of the light-emitting device, FIG. 48 shows blue index-luminance characteristics of the light-emitting device, and FIG. 49 shows an electroluminescence spectrum of the light-emitting device.

The main characteristics of the device at a luminance of approximately 1000 cd/m² are shown in the table below. The luminance, CIE chromaticity, and emission spectrum were measured with a spectroradiometer (SR-UL1R, manufactured by TOPCON TECHNOHOUSE CORPORATION).

TABLE 7

| | | Current | | | | Current | |
|---|---|---|---|---|---|---|---|
| | Voltage | density | Chromaticity | Chromaticity | Luminance | efficiency | BI value |
| | (V) | (mA/cm²) | x | y | (cd/m²) | (cd/A) | (cd/A/y) |
| Device 7A | 4.40 | 16.03 | 0.140 | 0.045 | 864.7 | 5.39 | 120 |

FIG. 44 to FIG. 49 show that the light-emitting device 7A is a highly efficient light-emitting device which emits blue light having high color purity with a chromaticity y of approximately 0.04. Furthermore, since low-temperature deposition was possible in the light-emitting device 7A, the light-emitting device 7A was able to be fabricated with high yield.

### <Results of Reliability Test>

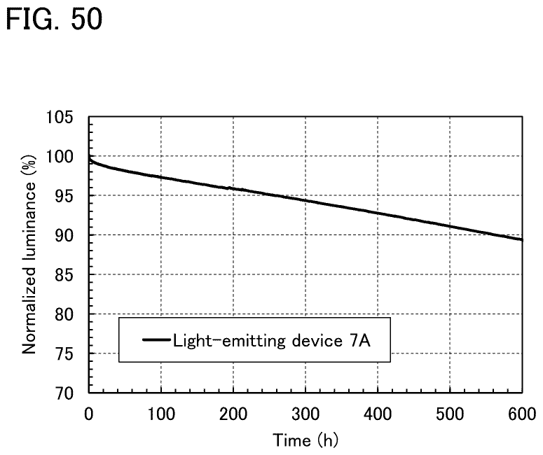

Moreover, a reliability test was conducted on the light-emitting device 7A. FIG. 50 shows a time-dependent change in normalized luminance at the time of constant current density driving (50 [mA/cm²]). In FIG. 50, the vertical axis represents the luminance (%) normalized with the luminance at the time of the start of light emission as 100%, and the horizontal axis represent the time (h).

In FIG. 50, LT95 (h), which is the time that has elapsed until the measured luminance decreases to 95% of the initial luminance, of the light-emitting device 7A was 257 hours; accordingly, the light-emitting device 7A was found to be a long-life device.

It was found from the above results that light-emitting device with high emission efficiency and high reliability can be provided with the use of one embodiment of the present invention.

### Example 8

In this example, light-emitting devices 8 (a light-emitting device 8A, a light-emitting device 8B, a light-emitting device 8C, and a light-emitting device 8D) of embodiments of the present invention were fabricated.

Specifically, the light-emitting devices in which 3-[4-(2,2′-binaphthalen-6-yl)phenyl]-9-phenyl-9H-carbazole (abbreviation: PCP(βN2)) represented by Structural Formula (100), 3-[4-(2-phenanthlenyl)phenyl]-9-(2-phenanthryl)-9H-carbazole (abbreviation: PnCPPn) represented by Structural Formula (102), 3-[4-(2-phenanthlenyl)phenyl]-9-(2-naphthyl)-9H-carbazole (abbreviation: βNCPPn-02) represented by Structural Formula (123), and 3-[4-(2-naphthalenyl)phenyl]-9-[4-(2-naphthalenyl)phenyl]-9H-carbazole (abbreviation: βNPCPβN) represented by Structural Formula (150) were used for cap layers were fabricated.

The structural formulae of organic compounds used in common in the light-emitting devices 8A to 8D are shown below.

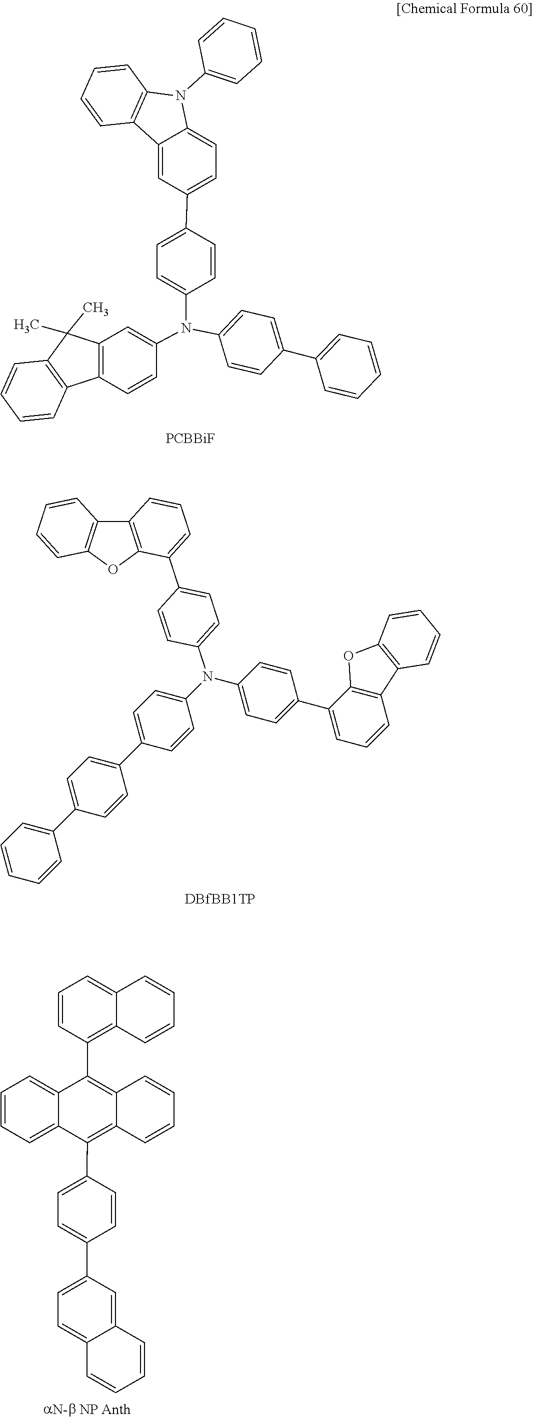

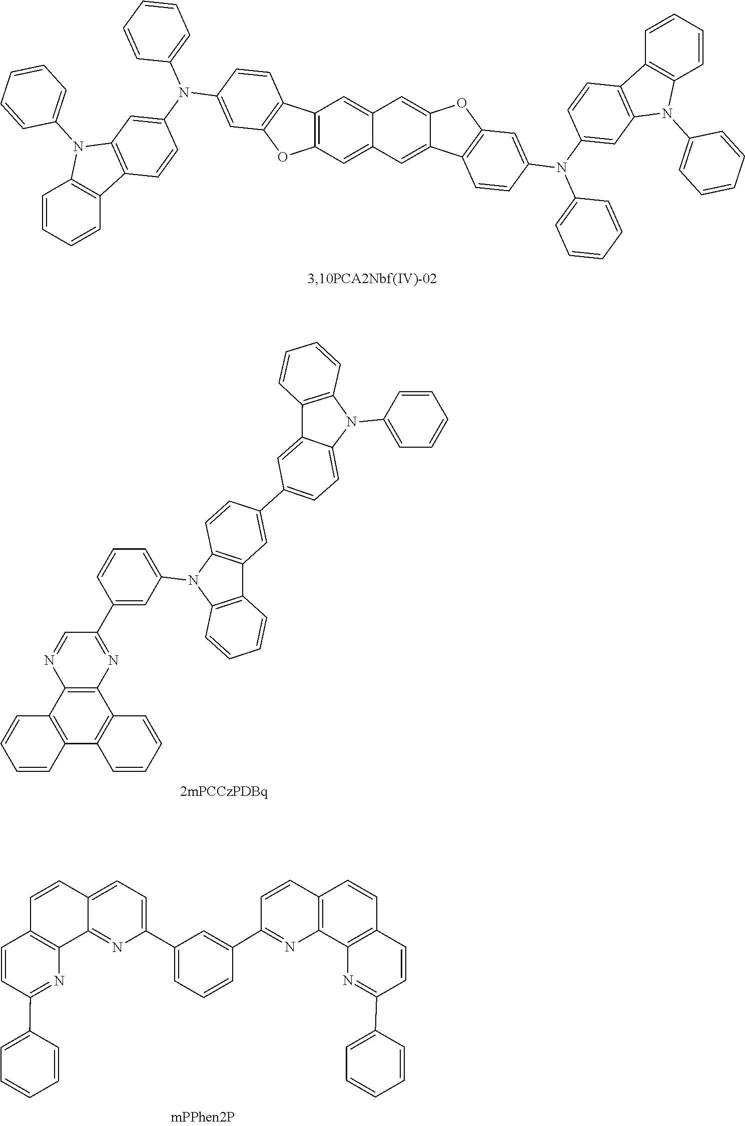 [SMILES: c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1 ; c1ccc(-n2c3ccccc3c3cc(-c4ccc5c(c4)c4ccccc4n5-c4cccc(-c5cnc6c7ccccc7c7ccccc7c6n5)c4)ccc32)cc1 ; c1ccc(-c2ccc3ccc4ccc(-c5cccc(-c6ccc7ccc8ccc(-c9ccccc9)nc8c7n6)c5)nc4c3n2)cc1]

The structural formulae of the organic compounds used as the cap layers in the light-emitting devices 8A to 8D are shown below.

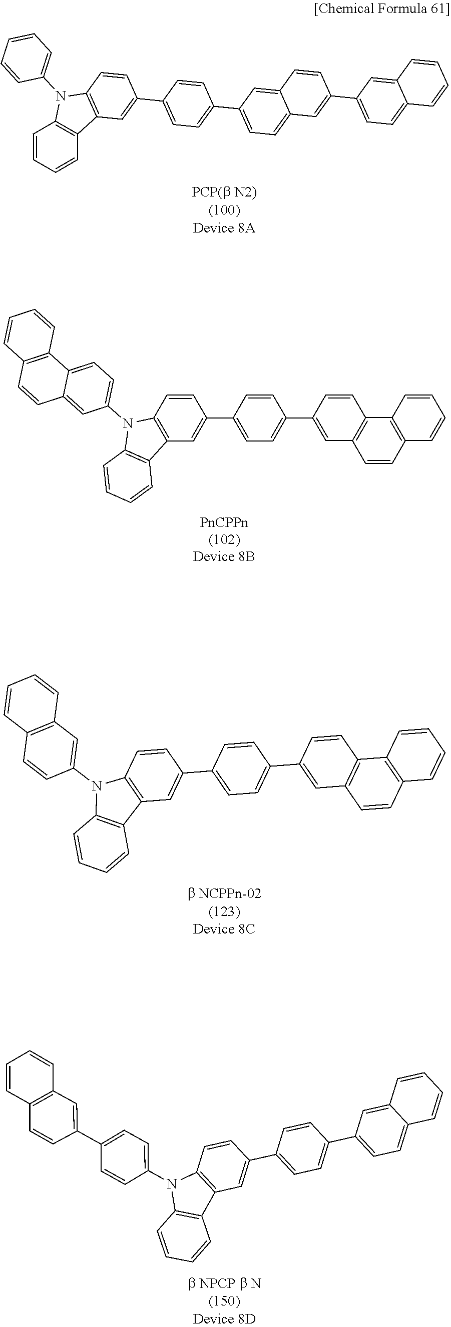 [SMILES: c1ccc(-n2c3ccccc3c3cc(-c4ccc(-c5ccc6cc(-c7ccc8ccccc8c7)ccc6c5)cc4)ccc32)cc1 ; c1ccc2c(c1)ccc1cc(-c3ccc(-c4ccc5c(c4)c4ccccc4n5-c4ccc5c(ccc6ccccc65)c4)cc3)ccc12 ; c1ccc2cc(-n3c4ccccc4c4cc(-c5ccc(-c6ccc7c(ccc8ccccc87)c6)cc5)ccc43)ccc2c1 ; c1ccc2cc(-c3ccc(-c4ccc5c(c4)c4ccccc4n5-c4ccc(-c5ccc6ccccc6c5)cc4)cc3)ccc2c1]

In each of the devices, as illustrated in FIG. 36, the hole-injection layer 811, the hole-transport layer 812, the light-emitting layer 813, the electron-transport layer 814, and the electron-injection layer 815 are stacked in this order over the first electrode 801 formed over the glass substrate 800, and the second electrode 802 is stacked over the electron-injection layer 815. Furthermore, the cap layer 809 is provided over the second electrode 802.

### <Method for Fabricating Light-Emitting Devices 8>

Silver (Ag) was deposited over the glass substrate 800 to a thickness of 100 nm by a sputtering method, and then, as a transparent electrode, indium tin oxide containing silicon oxide (ITSO) was deposited to a thickness of 10 nm by a sputtering method, whereby the first electrode 801 was formed. The electrode area was set to 4 mm² (2 mm×2 mm).

Next, in pretreatment for forming the light-emitting device over the substrate, a surface of the substrate was washed with water, and baking was performed at 200° C. for 1 hour. Then, the substrate was transferred into a vacuum evaporation apparatus where the pressure was reduced to approximately 1×10⁻⁵ Pa, and vacuum baking was performed at 170° C. for 30 minutes in a heating chamber of the vacuum evaporation apparatus. After that, natural cooling was performed to lower than or equal to 30° C.

Next, the substrate provided with the first electrode 801 was fixed to a substrate holder provided in the vacuum evaporation apparatus such that the surface on which the first electrode 801 was formed faced downward. Over the first electrode 801, N-(biphenyl-4-yl)-N-[4-(9-phenyl-9H-carbazol-3-yl)phenyl]-9,9-dimethyl-9H-fluoren-2-amine (abbreviation: PCBBiF) and an electron acceptor material containing fluorine and having a molecular weight of 672 (OCHD-003) were deposited to a thickness of 10 nm by co-evaporation at the weight ratio of 1:0.03, whereby the hole-injection layer 811 was formed.

Next, PCBBiF was deposited over the hole-injection layer 811. Note that the thicknesses of PCBBiF in the light-emitting devices 8A to 8D were independently adjusted so that substantially the same emission colors can be exhibited by the light-emitting devices. Specifically, deposition by evaporation was performed so that the thickness of PCBBiF was 108 nm in the light-emitting device 8A, 102 nm in the light-emitting device 8B, 102 nm in the light-emitting device 8C, and 100 nm in the light-emitting device 8D. Then, N,N-bis[4-(dibenzofuran-4-yl)phenyl]-4-amino-p-terphenyl (abbreviation: DBfBB1TP) was deposited to a thickness of 10 nm by evaporation, whereby the hole-transport layer 812 was formed.

Next, over the hole-transport layer 812, 9-(1-naphthyl)-10-[4-(2-naphthyl)phenyl]anthracene (abbreviation: αN-βNPAnth) and N,N-diphenyl-N,N-bis(9-phenyl-9H-carbazol-2-yl)naphtho[2,3-b;6,7-b′]bisbenzofuran-3,10-diamine (abbreviation: 3,10PCA2Nbf(IV)-02) were deposited to a thickness of 25 nm by a co-evaporation method using resistance heating at the weight ratio of 1:0.015, whereby the light-emitting layer 813 was formed.

Next, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited to a thickness of 15 nm by evaporation, and then 2,2′-(1,3-phenylene)bis(9-phenyl-1,10-phenanthroline) (abbreviation: mPPhen2P) was deposited to a thickness of 10 nm by evaporation, whereby the electron-transport layer 814 was formed.

Next, lithium fluoride (LiF) was deposited to a thickness of 1 nm over the electron-transport layer 814 by evaporation, whereby the electron-injection layer 815 was formed.

Then, over the electron-injection layer 815, silver (Ag) and magnesium (Mg) were deposited to a thickness of 15 nm by co-evaporation such that the volume ratio of Ag to Mg was 1:0.1, whereby the second electrode 802 was formed.

### <<Methods for Fabricating Light-Emitting Devices 8A to 8D>>

Next, over the second electrode 802, the organic compound functioning as the cap layer was deposited by an evaporation method using resistance heating.

In the light-emitting device 8A, PCP(βN2) was deposited to a thickness of 61 nm by evaporation as the cap layer.

In the light-emitting device 8B, PnCPPn was deposited to a thickness of 61 nm by evaporation as the cap layer.

In the light-emitting device 8C, DNCPPn-02 was deposited to a thickness of 63 nm by evaporation as the cap layer.

In the light-emitting device 8D, βNPCPβN was deposited to a thickness of 63 nm by evaporation as the cap layer.

The structures of the light-emitting devices 8A to 8D are listed in the following table.

TABLE 8

| | Film | | | | |
|---|---|---|---|---|---|
| | thickness | | | | |
| | [nm] | Device 8A | Device 8B | Device 8C | Device 8D |
| Cap layer | — | PCP(βN2) | PnCPPn | βNCPPn-02 | βNPCPβN |
| | | 61 nm | 61 nm | 63 nm | 63 nm |

| | | |
|---|---|---|
| Second electrode | 15 | Ag:Mg (1:0.1) |
| Electron-injection layer | 1 | LiF |
| Electron-transport | 10 | mPPhen2P |
| layer | 15 | 2mPCCzPDBq |
| Light-emitting layer | 25 | αN-βNPAnth:3,10PCA2Nbf(IV)-02 |
| | | (1:0.015) |
| Hole-transport layer | 10 | DBfBB1TP |
| | — | PCBBiF |

| | | | |
|---|---|---|---|
| 108 nm | 102 nm | 102 nm | 100 nm |

| | | |
|---|---|---|
| Hole-injection layer | 10 | PCBBiF:OCHD-003 (1:0.03) |
| First electrode | 10 | ITSO |
| | 100 | Ag |

### <Light-Emitting Device Characteristics>

The light-emitting devices were sealed using a glass substrate in a glove box containing a nitrogen atmosphere so as not to be exposed to the air (a sealing material was applied to surround the devices and UV treatment and heat treatment at 80° C. for 1 hour were performed at the time of sealing). Then, the characteristics of the devices were measured.

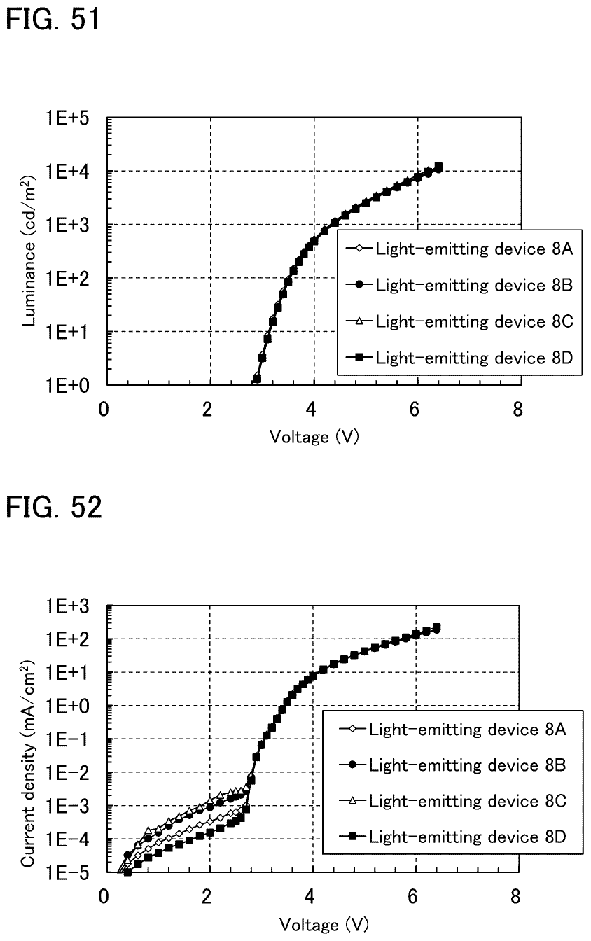

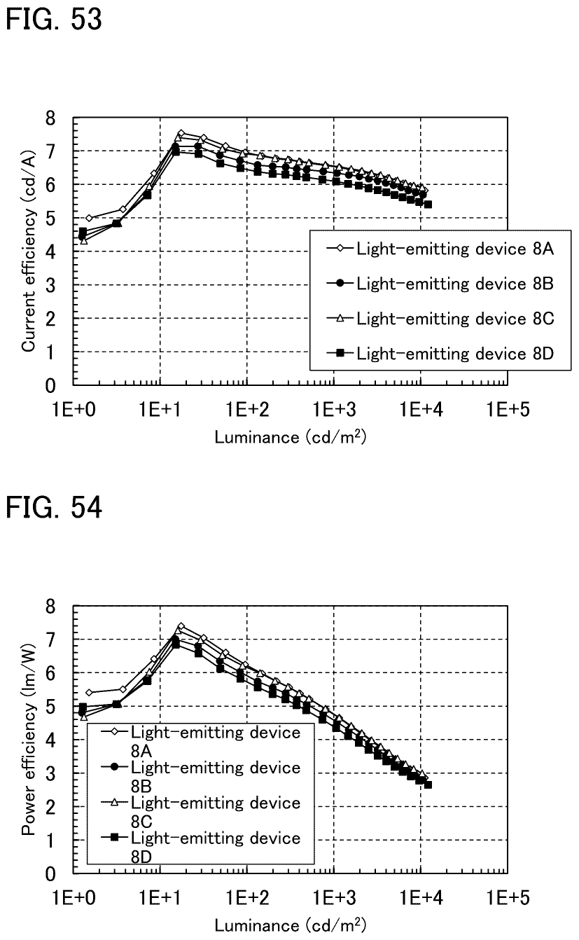

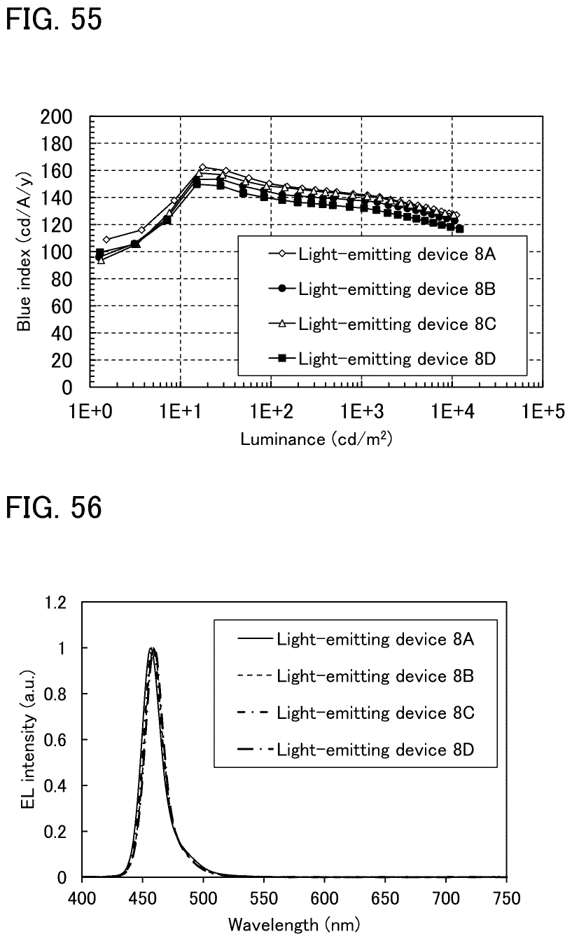

FIG. 51 shows luminance-voltage characteristics of the light-emitting devices, FIG. 52 shows current density-voltage characteristics of the light-emitting devices, FIG. 53 shows current efficiency-luminance characteristics of the light-emitting devices, FIG. 54 shows power efficiency-luminance characteristics of the light-emitting devices, FIG. 55 shows blue index-luminance characteristics of the light-emitting devices, and FIG. 56 shows electroluminescence spectra of the light-emitting devices.

The main characteristics of the light-emitting devices at a luminance of approximately 1000 cd/m² are shown in the table below. The luminance, CIE chromaticity, and emission spectra were measured with a spectroradiometer (SR-UL1R, manufactured by TOPCON TECHNOHOUSE CORPORATION).

TABLE 9

| | | Current | | | | Current | |
|---|---|---|---|---|---|---|---|
| | Voltage | density | Chromaticity | Chromaticity | Luminance | efficiency | BI value |
| | (V) | (mA/cm²) | x | y | (cd/m²) | (cd/A) | (cd/A/y) |
| Device 8A | 4.40 | 17.79 | 0.143 | 0.046 | 1158.0 | 6.51 | 142 |
| Device 8B | 4.40 | 17.21 | 0.141 | 0.046 | 1090.0 | 6.33 | 137 |
| Device 8C | 4.40 | 17.69 | 0.141 | 0.046 | 1154.0 | 6.52 | 141 |
| Device 8D | 4.40 | 17.50 | 0.140 | 0.046 | 1065.0 | 6.09 | 132 |

FIG. 51 to FIG. 56 show that the light-emitting devices 8A to 8D are highly efficient light-emitting devices which emit blue light having high color purity with a chromaticity y of approximately 0.04.

It was found from the above results that using the organic compounds of embodiments of the present invention as the cap layers of light-emitting devices enables the light-emitting devices to have high emission efficiency.

As compared with the light-emitting devices 6A and 6B fabricated in Example 6 and the light-emitting device 7A fabricated in Example 7, the light-emitting devices 8A to 8D showed high current efficiency and high blue indices. This is because the thicknesses of the cap layers were adjusted around 60 nm to maximize optical interference with respect to blue light wavelength. As a result, the efficiency was increased without a significant change in chromaticity.

### Example 9

### Synthesis Example 4

In this synthesis example, a method for synthesizing 3-[4-(2-naphthalenyl)phenyl]-9-[4-(2-naphthalenyl)phenyl]-9H-carbazole (abbreviation: βNPCPβN), which is the organic compound represented by Structural Formula (150) shown in Embodiment 1, is specifically described.

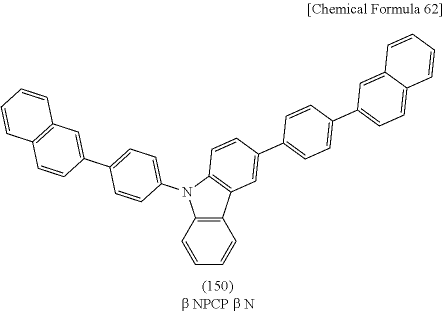 [SMILES: c1ccc2cc(-c3ccc(-c4ccc5c(c4)c4ccccc4n5-c4ccc(-c5ccc6ccccc6c5)cc4)cc3)ccc2c1]

### Step 1: Synthesis of 3-[4-(2-naphthalenyl)phenyl]-9H-carbazole

Step 1 was performed in the following procedure.

Into a 1-L three-neck flask were put 9.84 g (40.0 mmol) of 3-bromocarbazole, 9.92 g (40.0 mmol) of 4-(2-naphthyl)phenylboronic acid, 16.6 g (120 mmol) of potassium carbonate (abbreviation: K₂CO₃), 200 mL of toluene, 40 mL of ethanol, and 60 mL of water. Then, the mixture was degassed while the pressure in the flask was reduced, and the air in the flask was replaced with nitrogen. Next, the mixture in the flask was heated at 40° C. under a nitrogen stream, and 730 mg (2.40 mmol) of tris(2-methylphenyl)phosphine (abbreviation: P(o-tolyl)₃) and 269 mg (1.20 mmol) of palladium acetate were added thereto. Then, the temperature of the mixture was raised to 90° C. and the mixture was stirred for 3 hours while being heated. After the reaction, this mixture was suction-filtered, and the obtained residue was washed with water and ethanol. The resulting solid was dissolved in heated toluene, followed by filtration through Celite. The obtained filtrate was concentrated and recrystallized with toluene, so that 10.7 g of a white solid was obtained in a yield of 72%. A synthesis scheme of Step 1 is shown in (d-1) below.

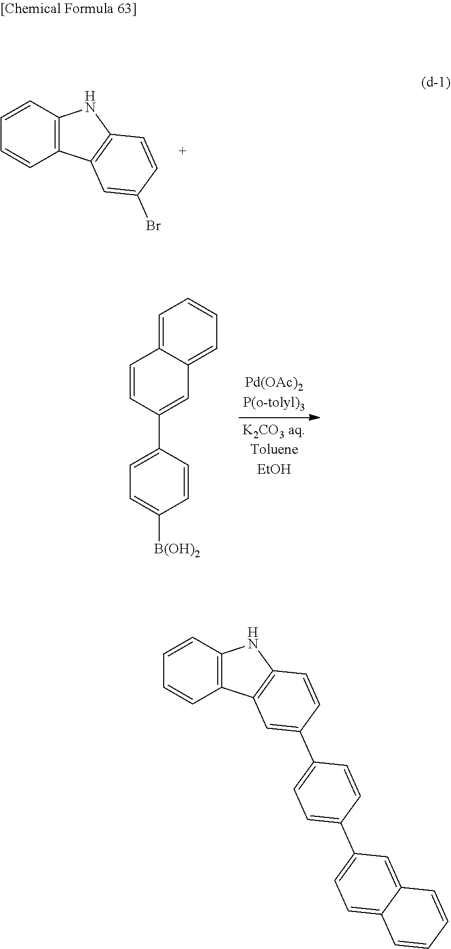 [SMILES: Brc1ccc2[nH]c3ccccc3c2c1 ; OB(O)c1ccc(-c2ccc3ccccc3c2)cc1 ; c1ccc2cc(-c3ccc(-c4ccc5[nH]c6ccccc6c5c4)cc3)ccc2c1]

### Step 2: Synthesis of βNPCPβN

Step 2 was performed in the following procedure.

Into a 200-mL three-neck flask were put 1.85 g (5.00 mmol) 3-[4-(2-naphthalenyl)phenyl]-9H-carbazole obtained in Step 1, 1.42 g (5.00 mmol) of 2-(4-bromophenyl)naphthalene, 0.96 g (10.0 mmol) of sodium tert-butoxide, and 33 mL of xylene. Then, the mixture was degassed while the pressure in the flask was reduced, and the air in the flask was replaced with nitrogen. Next, the mixture in the flask was heated at 40° C. under a nitrogen stream, and 35 mg (0.10 mmol) of di-tert-butyl(2,2-diphenyl-1-methyl-1-cyclopropyl)phosphine (cBRIDP) and 29 mg (0.05 mmol) of bis(dibenzylideneacetone)palladium(0) (Pd(dba)₂) were added thereto. Then, the temperature of the mixture was raised to 120° C. and the mixture was stirred for 6 hours while being heated. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene and ethanol, so that 2.33 g of a white solid was obtained in a yield of 81%.

With a train sublimation method, 2.33 g of the obtained white solid was purified by heating at 310° C. for 45 hours under a pressure of 3.00 Pa with an argon flow rate of 10 mL/min to give 2.00 g of a white solid at a collection rate of 86%. As the result of mass spectrometry, it was confirmed that the target substance βNPCPβN was obtained. A synthesis scheme of Step 2 is shown in (d-2) below.

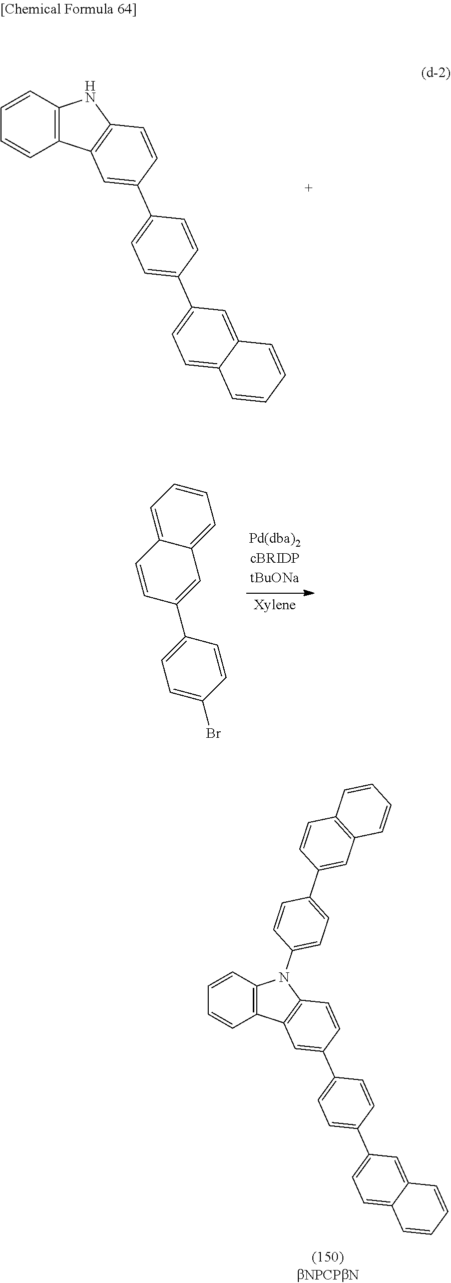 [SMILES: c1ccc2cc(-c3ccc(-c4ccc5[nH]c6ccccc6c5c4)cc3)ccc2c1 ; Brc1ccc(-c2ccc3ccccc3c2)cc1 ; c1ccc2cc(-c3ccc(-c4ccc5c(c4)c4ccccc4n5-c4ccc(-c5ccc6ccccc6c5)cc4)cc3)ccc2c1]

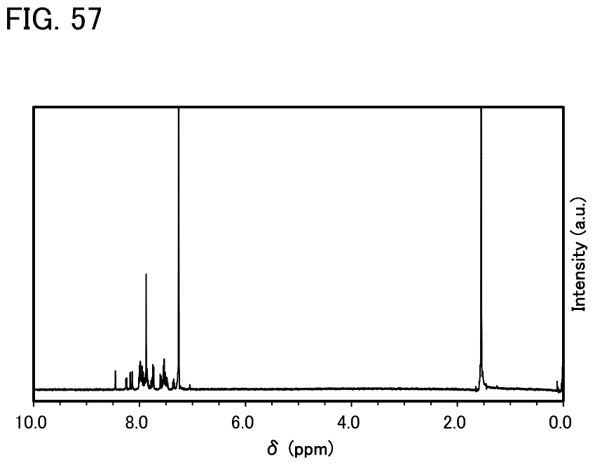

FIG. 57 shows a nuclear magnetic resonance spectroscopy (¹H-NMR) chart of βNPCPβN after purification by sublimation in a deuterated chloroform (abbreviation: CDCl₃) solution. The results also show that βNPCPβN was obtained.

¹H NMR (CDCl₃, 500 MHz): δ=7.36 (t, 1H), 7.47-7.61 (m, 7H), 7.73-8.01 (m, 17H), 8.14 (s, 1H), 8.18 (s, 1H), 8.25 (d, 1H), 8.46 (s, 1H).

### <Measurement of Emission and Absorption Spectra>

The absorption spectrum and the emission spectrum of βNPCPβN were measured. The absorption spectrum was measured with an ultraviolet-visible spectrophotometer (V-770, manufactured by JASCO Corporation). The emission spectrum was measured with a fluorescence spectrophotometer (FP-8600, manufactured by JASCO Corporation).

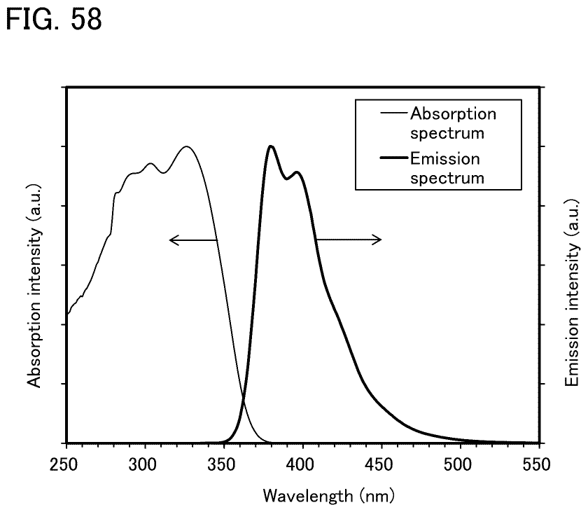

FIG. 58 shows an absorption spectrum and an emission spectrum of βNPCPβN in a toluene solution. The absorption spectrum in the solution state was obtained by subtraction of a measured absorption spectrum of a solvent (toluene) alone in a quartz cell from a measured absorption spectrum of the toluene solution of βNPCPβN in a quartz cell.

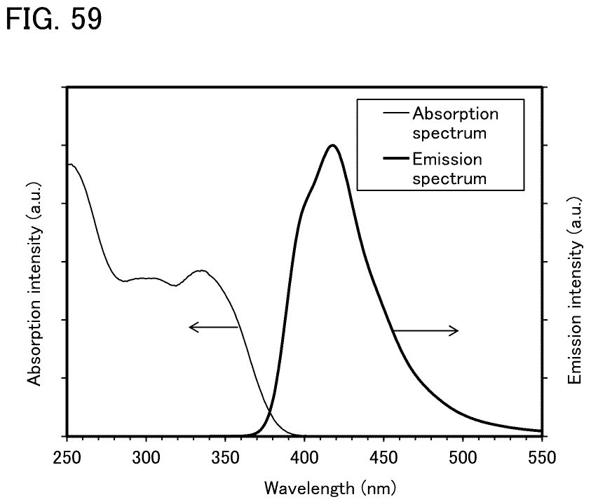

FIG. 59 shows an absorption spectrum and an emission spectrum of a thin film of βNPCPβN. The solid thin film was formed over a quartz substrate by a vacuum evaporation method and another quartz substrate as a counter substrate was used to perform sealing; thus, a measurement sample was fabricated. Note that the emission spectrum was obtained by measuring the sealed sample, and the absorption spectrum was obtained by measuring the sample from which the sealing was removed and the counter substrate was detached. The absorption spectrum of the thin film was obtained by subtraction of an absorption spectrum of the quartz substrate from an absorption spectrum of a βNPCPβN film formed over the quartz substrate. Even after the removal of the sealing at room temperature, any change in film quality was not visually observed from the formed thin film and the stable amorphous film was found to be maintained. This revealed that the compound of the present application was able to form a thin film with excellent stability and the organic device fabricated with the compound of the present application exhibited excellent stability.

As shown in FIG. 58, βNPCPβN in the toluene solution had absorption peaks at around 303 nm and 326 nm and emission peaks at around 379 nm and 396 nm (excitation wavelength: 324 nm). Furthermore, as shown in FIG. 59, βNPCPβN in the thin film had absorption peaks at around 210 nm, 255 nm, 294 nm, 310 nm, and 334 nm, and an emission peak at around 418 nm (excitation wavelength: 330 nm).

These absorption characteristics indicate that no absorption was observed in the visible light range (wavelength range higher than or equal to 430 nm) needed for displays. Thus, βNPCPβN can be suitably used for the cap film in a wavelength range needed for displays without reducing the emission efficiency of a light-emitting element. Furthermore, the obtained emission characteristics show that in the case where βNPCPβN is used as a host in a light-emitting layer, energy can be efficiently transferred to a light-emitting material that emits light with a wavelength higher than or equal to 440 nm and lower than or equal to 700 nm and thus βNPCPβN is suitable for a light-emitting element as well.

### <Measurement of Glass Transition Temperature (T_{g})>

The glass transition temperature (T_{g}) of βNPCPβN was measured. The T_{g} was measured with a differential scanning calorimeter (PYRIS 1 DSC manufactured by PerkinElmer Japan Co., Ltd.) in a state where a powder was put on an aluminum sample pan.

The results showed that the T_{g} of βNPCPβN was 100° C. The T_{g} available for an organic device is higher than or equal to 100° C., preferably higher than or equal to 110° C., further preferably higher than or equal to 120° C., still further preferably higher than or equal to 130° C. Therefore, the measurement results revealed that the compound of the present invention has an excellent thermal property and a thin film formed using the compound is expected to have stable film quality. The use of the compound capable of forming a thin film with stable film quality allows a highly heat-resistant organic device to be provided.

### <Calculation of HOMO and LUMO Levels>

The HOMO level and the LUMO level of βNPCPβN were obtained through cyclic voltammetry (CV) measurement. The calculation method is shown below.

An electrochemical analyzer (ALS model 600A or 600C, manufactured by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (n-Bu₄NClO4; produced by Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, produced by BAS Inc.) was used as a working electrode, a platinum electrode (Pt counter electrode for VC-3 (5 cm), produced by BAS Inc.) was used as an auxiliary electrode, and an Ag/Ag⁺ electrode (RE7 reference electrode for nonaqueous solvent, produced by BAS Inc.) was used as a reference electrode. Note that the measurement was performed at room temperature (20° C. to 25° C.).

The scan speed in the CV measurement was fixed to 0.1 V/sec, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]=−4.94−Ec.

The CV measurement was repeated 100 times, and the oxidation-reduction wave in the hundredth cycle was compared with the oxidation-reduction wave in the first cycle to examine the electrical stability of the compound.

According to the measurement results of the oxidation potential Ea [V] of βNPCPβN, the HOMO level was found to be around −5.75 eV. According to the measurement results of the reduction potential Ec [V] of βNPCPβN, the LUMO level was found to be −2.31 eV. When the oxidation-reduction wave was repeatedly measured, in the Ea measurement, the peak intensity of the oxidation-reduction wave in the hundredth cycle was maintained to be 97% of that of the oxidation-reduction wave in the first cycle, and in the Ec measurement, the peak intensity of the oxidation-reduction wave in the hundredth cycle was maintained to be 97% of that of the oxidation-reduction wave in the first cycle; thus, resistance to oxidation and reduction of βNPCPβN was found to be extremely high.

In consideration of the HOMO and LUMO levels, it is probable that holes and electrons can be favorably given and received, and βNPCPβN can be suitably used for layers that need to transport carriers, such as a hole-transport layer, an electron-transport layer, a light-emitting layer, and a charge-generation layer of a tandem element in an organic device. In particular, since βNPCPβN has a carbazole ring in its molecular structure, it can be suitably used for a hole-transport layer, a light-emitting layer, and a charge-generation layer of a tandem element which are responsible for hole transport.

### <Refractive Index Measurement>

The refractive index of βNPCPβN was measured by a spectroscopic ellipsometer (M-2000U, manufactured by J. A. Woollam Japan). The βNPCPβN film used for the measurement was formed to a thickness of approximately 50 nm over a quartz substrate by a vacuum evaporation method.

At a wavelength of 630 nm, n Ordinary (nₒ) that is the ordinary refractive index was 1.85, n Extra-ordinary (nₑ) that is the extraordinary refractive index was 1.69, and the difference between nₒ and nₑ was 0.16. At a wavelength of 520 nm, nₒ was 1.90, nₑ was 1.72, and the difference between nₒ and nₑ was 0.18. At a wavelength of 450 nm, nₒ was 1.98, nₑ was 1.76, and the difference between nₒ and nₑ was 0.22.

For the cap layer material, the difference between nₒ and nₑ is preferably greater than or equal to 0.1, further preferably greater than or equal to 0.2, still further preferably greater than or equal to 0.3 at any of three wavelengths of 450 nm, 520 nm, and 630 nm or at each of the three wavelengths. Therefore, the measurement results revealed that βNPCPβN can be effectively used as a material of a cap layer provided over a cathode in a light-emitting apparatus.

Thus, it was found from the measurement results of the physical property values that βNPCPβN can be effectively used for a cap layer used over a cathode. Furthermore, the results showed that βNPCPβN can also be effectively used as a light-emitting substance or a host material used in combination with a substance that emits light in the visible range.

### Example 10

### Synthesis Example 5

In this synthesis example, a method for synthesizing 3-[4-(2-phenanthlenyl)phenyl]-9-(2-naphthyl)-9H-carbazole (abbreviation: βNCPPn-02), which is the organic compound represented by Structural Formula (123) shown in Embodiment 1, is specifically described.

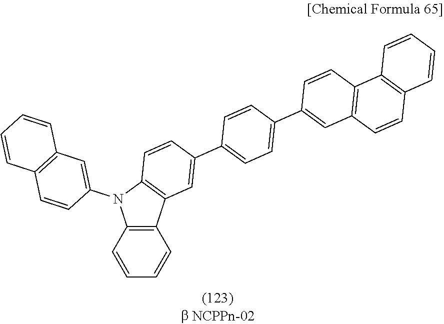 [SMILES: c1ccc2cc(-n3c4ccccc4c4cc(-c5ccc(-c6ccc7c(ccc8ccccc87)c6)cc5)ccc43)ccc2c1]

### Step 1: Synthesis of βNCPPn-02

Step 1 was performed in the following procedure.

Into a 200-mL three-neck flask were put 1.67 g (5.00 mmol) of 2-(4-bromophenyl)-phenanthrene obtained in Step 2 of the above-described synthesis example 3, 2.02 g (6.00 mmol) of [9-(2-naphthalenyl)-9H-carbazol-3-yl]boronic acid, 3.26 g (10.0 mmol) of cesium carbonate (abbreviation: Cs₂CO₃), 1.14 g (7.50 mmol) of cesium fluoride (abbreviation: CsF), and 25 mL of diethylene glycol dimethyl ether. Then, the mixture was degassed while the pressure in the flask was reduced, and the air in the flask was replaced with nitrogen. Next, the mixture in the flask was heated at 40° C. under a nitrogen stream, and 108 mg (0.30 mmol) of di(1-adamantyl)-n-butylphosphine (abbreviation: cataCXium (registered trademark) A) and 34 mg (0.15 mmol) of palladium acetate were added thereto. Then, the temperature of the mixture was raised to 140° C. and the mixture was stirred for 4 hours. After the reaction, this mixture was suction-filtered, and the obtained residue was washed with water and ethanol. The resulting solid was dissolved in heated toluene, followed by suction filtration through a filter medium in which Celite, alumina, and Celite were stacked in this order. The obtained filtrate was concentrated and recrystallized with toluene and ethanol, so that 1.68 g of a white solid was obtained in a yield of 62%.

With a train sublimation method, 1.67 g of the obtained white solid was purified by heating at 300° C. for 48 hours under a pressure of 2.50 Pa with an argon flow rate of 17 mL/min to give 1.19 g of a white solid at a collection rate of 71%. As the result of mass spectrometry, it was confirmed that the target substance βNCPPn-02 was obtained. A synthesis scheme of Step 1 is shown in (e-1) below.

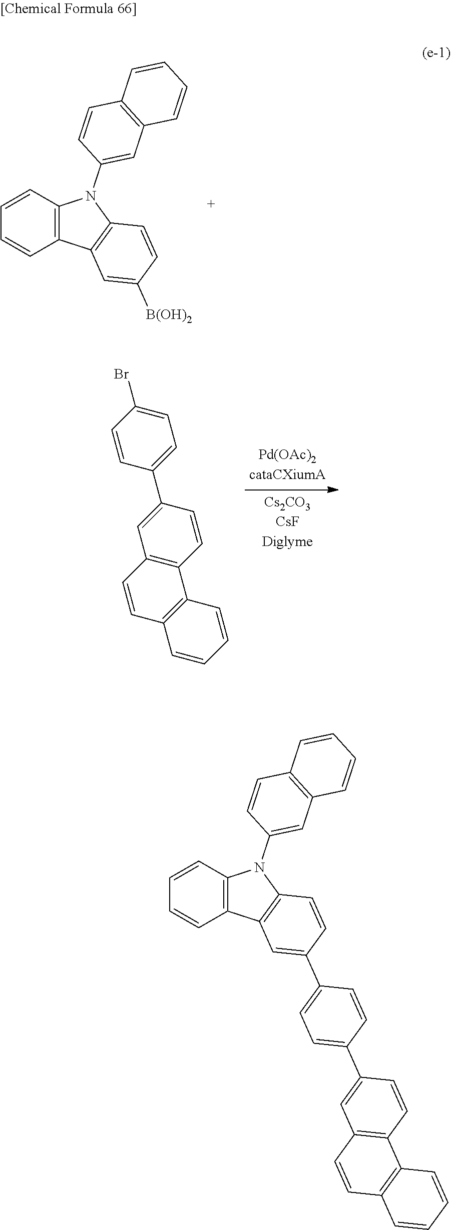 [SMILES: OB(O)c1ccc2c(c1)c1ccccc1n2-c1ccc2ccccc2c1 ; Brc1ccc(-c2ccc3c(ccc4ccccc43)c2)cc1 ; c1ccc2cc(-n3c4ccccc4c4cc(-c5ccc(-c6ccc7c(ccc8ccccc87)c6)cc5)ccc43)ccc2c1]

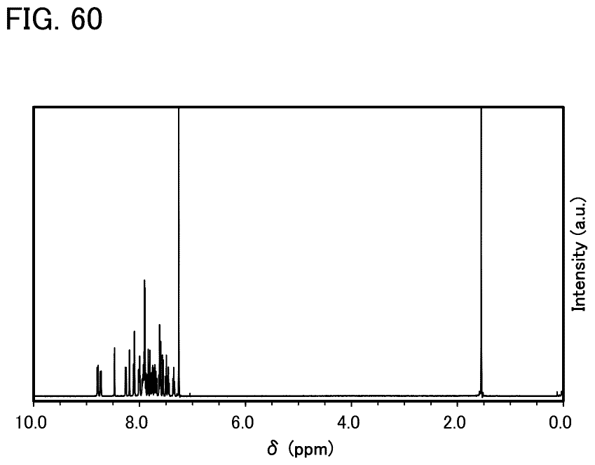

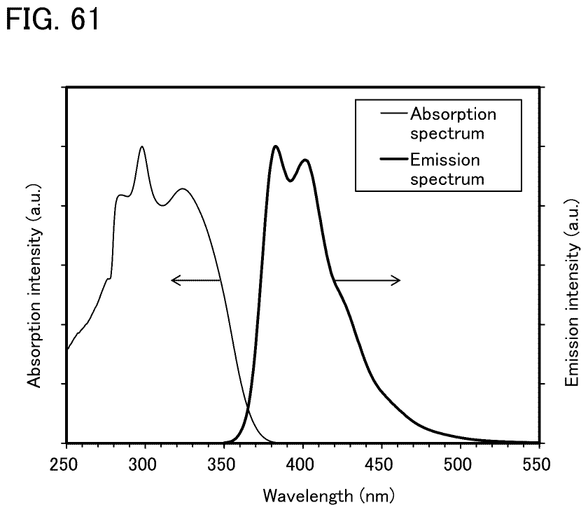

FIG. 60 shows a nuclear magnetic resonance spectroscopy (¹H-NMR) chart of βNCPPn-02 after purification by sublimation in a deuterated chloroform (abbreviation: CDCl₃) solution. The results also show that βNCPPn-02 was obtained.

¹H NMR (CDCl₃, 500 MHz): δ=7.35 (t, 1H), 7.44-7.51 (m, 2H), 7.56 (d, 1H), 7.60-7.64 (m, 3H), 7.67-7.95 (m, 11H), 7.99-8.02 (m, 2H), 8.10-8.12 (m, 2H), 8.19 (s, 1H), 8.26 (d, 1H), 8.47 (s, 1H), 8.73 (d, 1H), 8.79 (d, 1H).

### <Measurement of Emission and Absorption Spectra>

The absorption spectrum and the emission spectrum of βNCPPn-02 were measured. The absorption spectrum was measured with an ultraviolet-visible spectrophotometer (V-770, manufactured by JASCO Corporation). The emission spectrum was measured with a fluorescence spectrophotometer (FP-8600, manufactured by JASCO Corporation).

FIG. 61 shows an absorption spectrum and an emission spectrum of βNCPPn-02 in a toluene solution. The absorption spectrum in the solution state was obtained by subtraction of a measured absorption spectrum of a solvent (toluene) alone in a quartz cell from a measured absorption spectrum of the toluene solution of βNCPPn-02 in a quartz cell.

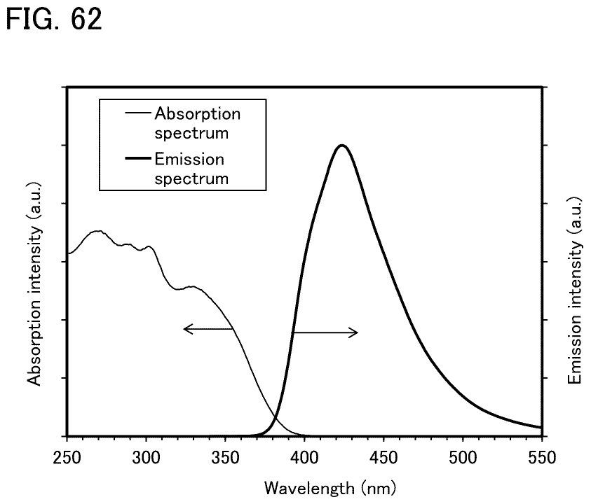

FIG. 62 shows an absorption spectrum and an emission spectrum of a thin film of βNCPPn-02. The solid thin film was formed over a quartz substrate by a vacuum evaporation method and another quartz substrate as a counter substrate was used to perform sealing; thus, a measurement sample was fabricated. Note that the emission spectrum was obtained by measuring the sealed sample, and the absorption spectrum was obtained by measuring the sample from which the sealing was removed and the counter substrate was detached. The absorption spectrum of the thin film was obtained by subtraction of an absorption spectrum of the quartz substrate from an absorption spectrum of a βNCPPn-02 film formed over the quartz substrate. Even after the removal of the sealing at room temperature, any change in film quality was not visually observed from the formed thin film and the stable amorphous film was found to be maintained. This revealed that the compound of the present application was able to form a thin film with excellent stability and the organic device fabricated with the compound of the present application exhibited excellent stability.

As shown in FIG. 61, βNCPPn-02 in the toluene solution had absorption peaks at around 285 nm, 298 nm, and 324 nm and emission peaks at around 383 nm and 401 nm (excitation wavelength: 324 nm). Furthermore, as shown in FIG. 62, βNCPPn-02 in the thin film had absorption peaks at around 217 nm, 271 nm, 287 nm, 300 nm, and 332 nm, and an emission peak at around 424 nm (excitation wavelength: 330 nm).

These absorption characteristics indicate that no absorption was observed in the visible light range (wavelength range higher than or equal to 430 nm) needed for displays. Thus, βNCPPn-02 can be suitably used for the cap film in a wavelength range needed for displays without reducing the emission efficiency of a light-emitting element. Furthermore, the obtained emission characteristics show that in the case where βNCPPn-02 is used as a host in a light-emitting layer, energy can be efficiently transferred to a light-emitting material that emits light with a wavelength higher than or equal to 440 nm and lower than or equal to 700 nm and thus βNCPPn-02 is suitable for a light-emitting element as well.

### <Measurement of Glass Transition Temperature (T_{g})>

The glass transition temperature (T_{g}) of βNCPPn-02 was measured. The T_{g} was measured with a differential scanning calorimeter (PYRIS 1 DSC manufactured by PerkinElmer Japan Co., Ltd.) in a state where a powder was put on an aluminum sample pan.

The results showed that the T_{g} of βNCPPn-02 was 100° C. The T_{g} available for an organic device is higher than or equal to 100° C., preferably higher than or equal to 110° C., further preferably higher than or equal to 120° C., still further preferably higher than or equal to 130° C. Therefore, the measurement results revealed that the compound of the present invention has an excellent thermal property and a thin film formed using the compound is expected to have stable film quality. The use of the compound capable of forming a thin film with stable film quality allows a highly heat-resistant organic device to be provided.

### <Calculation of HOMO and LUMO Levels>

The HOMO level and the LUMO level of βNCPPn-02 were obtained through cyclic voltammetry (CV) measurement. The calculation method is shown below.

An electrochemical analyzer (ALS model 600A or 600C, manufactured by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (n-Bu₄NClO₄; produced by Tokyo Chemical Industry Co., Ltd., catalog No. T0836) as a supporting electrolyte was dissolved at a concentration of 100 mmol/L. Furthermore, the measurement target was also dissolved at a concentration of 2 mmol/L.

A platinum electrode (PTE platinum electrode, produced by BAS Inc.) was used as a working electrode, a platinum electrode (Pt counter electrode for VC-3 (5 cm), produced by BAS Inc.) was used as an auxiliary electrode, and an Ag/Ag⁺ electrode (RE7 reference electrode for nonaqueous solvent, produced by BAS Inc.) was used as a reference electrode. Note that the measurement was performed at room temperature (20° C. to 25° C.).

The scan speed in the CV measurement was fixed to 0.1 V/see, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]=−4.94−Ec.

The CV measurement was repeated 100 times, and the oxidation-reduction wave in the hundredth cycle was compared with the oxidation-reduction wave in the first cycle to examine the electrical stability of the compound.

According to the measurement results of the oxidation potential Ea [V] of βNCPPn-02, the HOMO level was found to be around −5.77 eV. According to the measurement results of the reduction potential Ec [V] of βNCPPn-02, the LUMO level was found to be −2.34 eV. When the oxidation-reduction wave was repeatedly measured, in the Ea measurement, the peak intensity of the oxidation-reduction wave in the hundredth cycle was maintained to be 97% of that of the oxidation-reduction wave in the first cycle, and in the Ec measurement, the peak intensity of the oxidation-reduction wave in the hundredth cycle was maintained to be 99% of that of the oxidation-reduction wave in the first cycle; thus, resistance to oxidation and reduction of βNCPPn-02 was found to be extremely high.

In consideration of the HOMO and LUMO levels, it is probable that holes and electrons can be favorably given and received, and βNCPPn-02 can be suitably used for layers that need to transport carriers, such as a hole-transport layer, an electron-transport layer, a light-emitting layer, and a charge-generation layer of a tandem element in an organic device. In particular, since βNCPPn-02 has a carbazole ring in its molecular structure, it can be suitably used for a hole-transport layer, a light-emitting layer, and a charge-generation layer of a tandem element which are responsible for hole transport.

### <Refractive Index Measurement>

The refractive index of βNCPPn-02 was measured by a spectroscopic ellipsometer (M-2000U, manufactured by J. A. Woollam Japan). The βNCPPn-02 film used for the measurement was formed to a thickness of approximately 50 nm over a quartz substrate by a vacuum evaporation method.

At a wavelength of 630 nm, n Ordinary (nₒ) that is the ordinary refractive index was 1.85, n Extra-ordinary (nₑ) that is the extraordinary refractive index was 1.66, and the difference between nₒ and nₑ was 0.19. At a wavelength of 520 nm, nₒ was 1.90, nₑ was 1.69, and the difference between nₒ and nₑ was 0.21. At a wavelength of 450 nm, nₒ was 1.98, nₑ was 1.73, and the difference between nₒ and nₑ was 0.25.

For the cap layer material, the difference between nₒ and nₑ is preferably greater than or equal to 0.1, further preferably greater than or equal to 0.2, still further preferably greater than or equal to 0.3 at any of three wavelengths of 450 nm, 520 nm, and 630 nm or at each of the three wavelengths. Therefore, the measurement results revealed that βNCPPn-02 can be effectively used as a material of a cap layer provided over a cathode in a light-emitting apparatus.

Thus, it was found from the measurement results of the physical property values that βNCPPn-02 can be effectively used for a cap layer used over a cathode. Furthermore, the results showed that βNCPPn-02 can also be effectively used as a light-emitting substance or a host material used in combination with a substance that emits light in the visible range. This application is based on Japanese Patent Application Serial No. 2023-141744 filed with Japan Patent Office on Aug. 31, 2023, the entire contents of which are hereby incorporated by reference.

What is claimed is:

1. An organic compound represented by General Formula (G1): 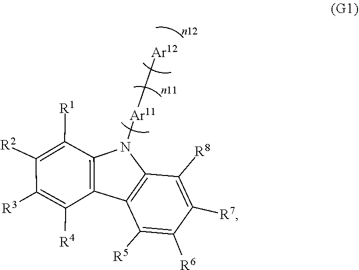
wherein each of R¹ to R⁴ independently represents any of hydrogen, a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1),
wherein each of R⁵ to R⁸ independently represents any of hydrogen, a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3),
wherein at least one of R⁵ to R⁸ represents the substituent represented by General Formula (G1-3),
wherein Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms,
wherein Ar¹² represents a substituted or unsubstituted monovalent aromatic hydrocarbon group having 6 to 30 carbon atoms or a substituted or unsubstituted monovalent aromatic heterocyclic group having 3 to 30 carbon atoms,
wherein n11 represents an integer greater than or equal to zero and less than or equal to three and n12 represents an integer greater than or equal to one and less than or equal to three, 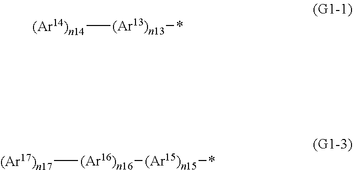
wherein each of Ar¹³, Ar¹, and Ar¹⁶ independently represents a substituted or unsubstituted divalent aromatic hydrocarbon group having 6 to 30 carbon atoms or a substituted or unsubstituted divalent aromatic heterocyclic group having 3 to 30 carbon atoms,
wherein each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms,
wherein at least two of Ar, Ar¹⁶, and Ar¹⁷ represent a monovalent or divalent bicyclic or tricyclic aromatic hydrocarbon group or a monovalent or divalent aromatic heterocyclic group,
wherein n13 represents an integer greater than or equal to zero and less than or equal to three and each of n14, n15, n16, and n17 independently represents an integer greater than or equal to one and less than or equal to three, and
wherein n15+n16+n17>n13+n14 is satisfied.

2. The organic compound according to claim 1, wherein at least two of Ar¹⁵, Ar¹⁶, and Ar¹⁷ represent naphthalene and a 2-position of each of the naphthalenes is bonded to another substituent.

3. The organic compound according to claim 1,
wherein Ar¹⁶ represents naphthalene and Ar¹⁷ represents naphthalene, and
wherein 2,2′-binaphthalene in which 2-positions of the naphthalenes are bonded to each other is included in a partial structure.

4. The organic compound according to claim 1, wherein each of Ar¹¹ to Ar¹⁷ independently represents an aromatic hydrocarbon group.

5. The organic compound according to claim 1, wherein an evaporation temperature is lower than or equal to 330° C.

6. The organic compound according to claim 1, wherein the organic compound is represented by Structural Formula (100) or Structural Formula (101):

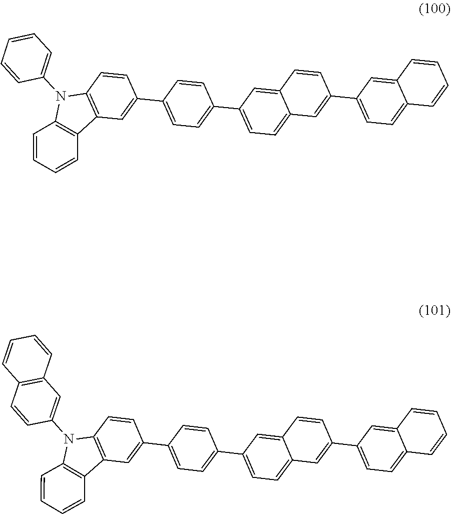 [SMILES: c1ccc(-n2c3ccccc3c3cc(-c4ccc(-c5ccc6cc(-c7ccc8ccccc8c7)ccc6c5)cc4)ccc32)cc1 ; c1ccc2cc(-c3ccc4cc(-c5ccc(-c6ccc7c(c6)c6ccccc6n7-c6ccc7ccccc7c6)cc5)ccc4c3)ccc2c1]

7. The organic compound according to claim 1, wherein the organic compound is an organic compound for a cap layer.

8. A light-emitting device comprising:
a cathode; and
a cap layer over the cathode,
wherein the cap layer comprises an organic compound represented by General Formula (G1): 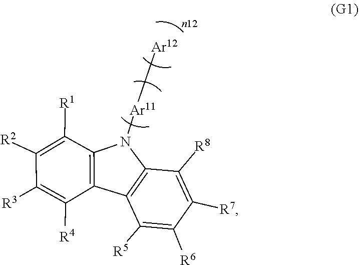
wherein each of R¹ to R⁴ independently represents any of hydrogen, a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1),
wherein each of R⁵ to R⁸ independently represents any of hydrogen, a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-3),
wherein at least one of R⁵ to R⁸ represents the substituent represented by General Formula (G1-3),
wherein Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms,
wherein Ar¹² represents a substituted or unsubstituted monovalent aromatic hydrocarbon group having 6 to 30 carbon atoms or a substituted or unsubstituted monovalent aromatic heterocyclic group having 3 to 30 carbon atoms,
wherein n11 represents an integer greater than or equal to zero and less than or equal to three and n12 represents an integer greater than or equal to one and less than or equal to three, 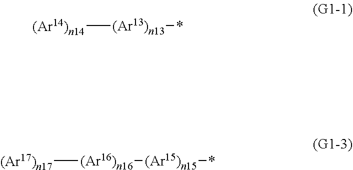
wherein each of Ar¹³, Ar¹, and Ar¹⁶ independently represents a substituted or unsubstituted divalent aromatic hydrocarbon group having 6 to 30 carbon atoms or a substituted or unsubstituted divalent aromatic heterocyclic group having 3 to 30 carbon atoms,
wherein each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms,
wherein at least two of Ar, Ar¹⁶, and Ar¹⁷ represent a monovalent or divalent bicyclic or tricyclic aromatic hydrocarbon group or a monovalent or divalent aromatic heterocyclic group,
wherein each of n13, n15, and n16 independently represents an integer greater than or equal to zero and less than or equal to three and each of n14 and n17 independently represents an integer greater than or equal to one and less than or equal to three, and
wherein n15+n16+n17>n13+n14 is satisfied.

9. A light-emitting device comprising:
a cathode; and
a cap layer over the cathode,
wherein the cap layer comprises an organic compound represented by General Formula (G1): 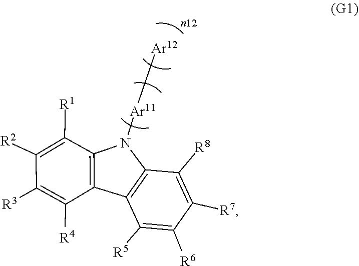
wherein each of R¹ to R⁴ independently represents any of hydrogen, a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-1),
wherein each of R⁵ to R⁸ independently represents any of hydrogen, a substituted or unsubstituted alkyl group having 1 to 6 carbon atoms, a substituted or unsubstituted cycloalkyl group having 3 to 6 carbon atoms, a substituted or unsubstituted aromatic hydrocarbon group having 6 to 30 carbon atoms, and a substituent represented by General Formula (G1-2),
wherein at least one of R⁵ to R⁸ represents the substituent represented by General Formula (G1-2),
wherein Ar¹¹ represents a substituted or unsubstituted divalent aromatic hydrocarbon group having 6 to 30 carbon atoms or a substituted or unsubstituted divalent aromatic heterocyclic group having 3 to 30 carbon atoms,
wherein Ar¹² represents a substituted or unsubstituted monovalent aromatic hydrocarbon group having 14 to 30 carbon atoms or a substituted or unsubstituted monovalent aromatic heterocyclic group having 3 to 30 carbon atoms,
wherein n11 represents an integer greater than or equal to zero and less than or equal to three and n12 represents an integer greater than or equal to one and less than or equal to three, 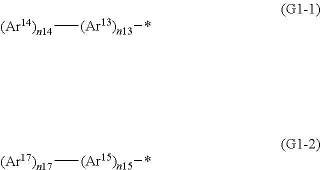
wherein each of Ar¹³ and Ar¹⁵ independently represents a substituted or unsubstituted divalent aromatic hydrocarbon group having 6 to 30 carbon atoms or a substituted or unsubstituted divalent aromatic heterocyclic group having 3 to 30 carbon atoms,
wherein each of Ar¹⁴ and Ar¹⁷ independently represents a substituted or unsubstituted monovalent aromatic hydrocarbon group having 6 to 30 carbon atoms or a substituted or unsubstituted monovalent aromatic heterocyclic group having 3 to 30 carbon atoms,
wherein at least one of Ar¹⁵ and Ar¹⁷ represents phenanthrene,
wherein when Ar¹⁷ represents phenanthrene, a 2-position or a 3-position of the phenanthrene is bonded to another group,
wherein n13 represents an integer greater than or equal to zero and less than or equal to three and each of n14, n15, and n17 independently represents an integer greater than or equal to one and less than or equal to three, and
wherein n15+n17>n13+n14 is satisfied.

10. The light-emitting device according to claim 8, wherein the organic compound has an evaporation temperature of lower than or equal to 330° C.

11. The light-emitting device according to claim 8, wherein the organic compound is represented by Structural Formula (100), Structural Formula (101), Structural Formula (102), Structural Formula (123), or Structural Formula (150):

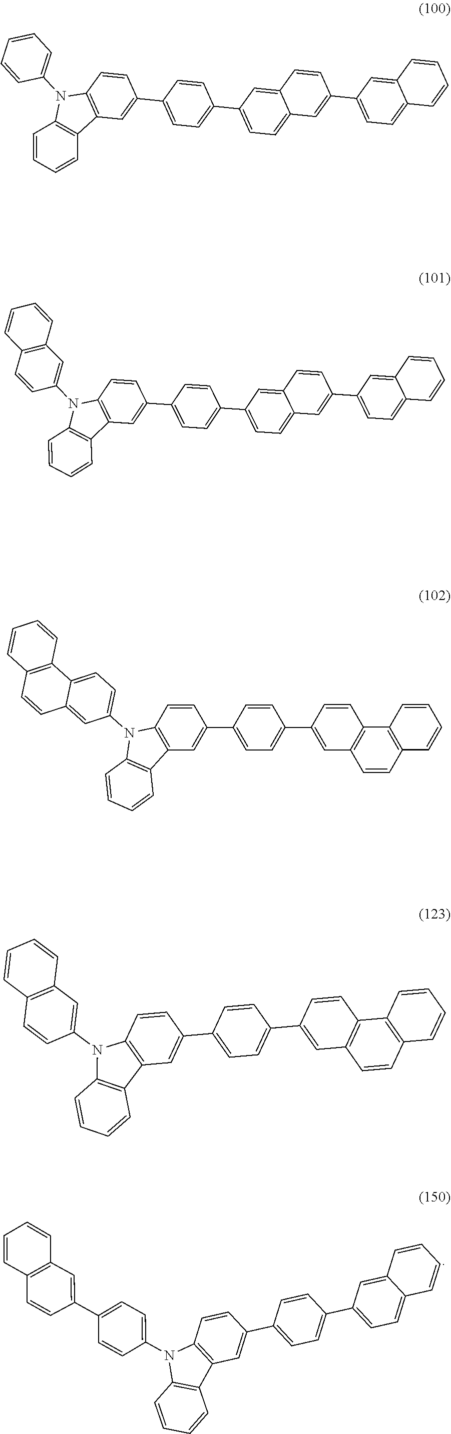 [SMILES: c1ccc(-n2c3ccccc3c3cc(-c4ccc(-c5ccc6cc(-c7ccc8ccccc8c7)ccc6c5)cc4)ccc32)cc1 ; c1ccc2cc(-c3ccc4cc(-c5ccc(-c6ccc7c(c6)c6ccccc6n7-c6ccc7ccccc7c6)cc5)ccc4c3)ccc2c1 ; c1ccc2c(c1)ccc1cc(-c3ccc(-c4ccc5c(c4)c4ccccc4n5-c4ccc5c(ccc6ccccc65)c4)cc3)ccc12 ; c1ccc2cc(-n3c4ccccc4c4cc(-c5ccc(-c6ccc7c(ccc8ccccc87)c6)cc5)ccc43)ccc2c1 ; c1ccc2cc(-c3ccc(-c4ccc5c(c4)c4ccccc4n5-c4ccc(-c5ccc6ccccc6c5)cc4)cc3)ccc2c1]

12. The light-emitting device according to claim 8, wherein the organic compound has an ordinary refractive index nₒ at 450 nm of higher than or equal to 1.9 and an ordinary extinction coefficient kₒ at 450 nm of lower than or equal to 0.2.

13. The light-emitting device according to claim 8, wherein the organic compound has an ordinary refractive index nₒ at 520 nm of higher than or equal to 1.8 and an ordinary extinction coefficient kₒ at 520 nm of lower than or equal to 0.2.

14. The light-emitting device according to claim 8, wherein the organic compound has an ordinary refractive index nₒ at 630 nm of higher than or equal to 1.75 and an ordinary extinction coefficient kₒ at 630 nm of lower than or equal to 0.2.

15. The light-emitting device according to claim 8, wherein a difference between an ordinary refractive index nₒ and an extraordinary refractive index nₑ at a wavelength higher than or equal to 360 nm and lower than or equal to 830 nm of the organic compound is greater than or equal to 0.1 and less than or equal to 0.4.

16. An electronic device comprising the light-emitting device according to claim 8.

17. The light-emitting device according to claim 9, wherein the organic compound has an evaporation temperature of lower than or equal to 330° C.

18. An electronic device comprising the light-emitting device according to claim 9.
